(19) United States
(12) Patent Application Publication
(10) Pub. No.: US 2026/0014202 A1
(43) Pub. Date: Jan. 15, 2026

(54) THERAPEUTIC METHODS USING ANTIBODY DRUG CONJUGATES (ADCS)

(71) Applicant: Vor Biopharma Inc., Cambridge, MA (US)

(72) Inventors: Anthony Boitano, Newton, MA (US); Michael Cooke, Boston, MA (US); Jennifer Lynn Proctor, Medford, MA (US); Charlotte Fenton McDonagh, Winchester, MA (US)

(21) Appl. No.: 19/067,310

(22) Filed: Feb. 28, 2025

Related U.S. Application Data
(63) Continuation of application No. 17/111,816, filed on Dec. 4, 2020, now abandoned.
Application No. 17/111,816 is a continuation of application No. PCT/IB2019/054748, filed on Jun. 7, 2019.
(60) Provisional application No. 62/682,154, filed on Jun. 7, 2018.
(60) Provisional application No. 62/841,702, filed on May 1, 2019.

(51) Int. Cl. A61P 37/00; A61K 35/28; A61K 39/00; A61K 47/65; A61K 47/68; C07K 16/28

(52) U.S. Cl. CPC A61K 35/28; A61K 47/65; A61K 47/6831; A61K 47/6849; A61P 37/00; C07K 16/2803; A61K 2039/505; A61K 2039/545

(57) ABSTRACT

The invention relates to dosing regimens for antibody drug conjugates, as well as methods of administering said dosing regimens to patients suffering from or at risk of various diseases and conditions such as autoimmune diseases, cancers, and Graft versus Host Disease (GvHD), among others, by administration of an antibody drug conjugate (ADC), capable of binding an antigen expressed by a hematopoietic cell, such as a hematopoietic stem cell, an immune cell or a cancer cell.

### RELATED APPLICATIONS

This application is a continuation of U.S. application Ser. No. 17/111,816, filed Dec. 4, 2020, which is a continuation of PCT Application No. PCT/IB2019/054748, filed on Jun. 7, 2019, which claims priority to U.S. Provisional Application No. 62/682,154, filed on Jun. 7, 2018, and to U.S. Provisional Application No. 62/841,702, filed on May 1, 2019. The contents of the aforementioned applications are incorporated by reference herein in their entirety.

### SEQUENCE LISTING

The present application contains a Sequence Listing that has been submitted electronically in XML format and is hereby incorporated by reference in its entirety. Said XML copy, created on Sep. 10, 2025, is named 118216_1500US_C2_0234_9_SL.xml and is 370,178 bytes in size.

### BACKGROUND OF THE INVENTION

Despite advances in the medicinal arts, there remains a demand for promoting the engraftment of exogenous hematopoietic stem cells and for preventing or treating pathologies of the hematopoietic system, such as diseases of a particular blood cell, metabolic disorders, cancers, and autoimmune conditions, among others. While the administration of antibody drug conjugates (ADCs) may possess significant therapeutic potential, a limitation that has hindered their use in the clinic has been complications associated with toxicity while the drug remains effective. Most ADCs to date have been used to treat cancer by killing tumor cells. The use of ADCs for conditioning therapy combined with stem cell transplantation presents unique challenges, including toxicity and efficacy. In addition, the ADC must be effective for a window of time in which the target cells are ablated prior to the transplanted cells taking hold in the patient. There is currently a need for dosing regimens for ADCs for use in methods for promoting the engraftment of exogenous hematopoietic stem cell grafts and for preventing or treating patients suffering from or at risk of various diseases and conditions such as autoimmune diseases, cancers, and Graft-versus-Host-Disease (GvHD).

### SUMMARY OF THE INVENTION

Described herein are compositions and methods involving antibody drug conjugate (ADC) dosing regimens for promoting the engraftment of exogenous hematopoietic stem cell grafts as well as for treating patients suffering from or at risk of various diseases and conditions such as autoimmune diseases, cancers, and Graft-versus-Host-Disease (GvHD). The methods disclosed herein address the complex challenges of administering an ADC, e.g., an anti-CD117 ADC, for conditioning such that the ADC is effective for ablating endogenous cells while reducing toxicity to the subject.

In one embodiment, the present invention provides a method of depleting a population of target cells in a human patient, wherein the method comprises a dosing regimen comprising the steps of administering to the patient a first dose of an antibody drug conjugate (ADC) on day 1; and administering to the patient a second dose of the ADC after the first dose; wherein the ADC comprises an antibody, or antigen-binding fragment thereof, conjugated to an amatoxin via a linker, wherein the ADC is specific for a cell surface marker, and wherein the dosing regimen induces a decrease in the population of the target cells in a human patient. In one embodiment, the first dose is selected from the group consisting of about 2-12 mg of the ADC, about 2-11 mg of the ADC, about 2-10 mg of the ADC, about 2-9 mg of the ADC, about 2-8 mg of the ADC, about 2-7 mg of the ADC, about 2-6 mg of the ADC, about 2-5 mg of the ADC, about 2-4 mg of the ADC, and about 2-3 mg of the ADC. In another embodiment, the first dose comprises about 1.9-11.5 mg of the ADC. In yet another embodiment, the second dose is within 10% by weight of the first dose. In another embodiment, the second dose is administered between 1-24 hours after the first dose is administered. In another embodiment, the second dose is administered between 1-12 days after the first dose is administered. In another embodiment, the target cell is a stem cell. In yet another embodiment, target cell is an immune cell. In another embodiment, the target cell is a disease causing cell. In yet another embodiment, the cell marker is selected from the group consisting of CD117, CD45, CD2, CD5, CD252, CD134, and CD137.

In one embodiment, an ADC used in the methods disclosed herein comprises an anti-CD117 antibody, or antigen binding portion thereof, comprises a heavy chain variable region as set forth in the amino acid sequence of SEQ ID NO: 9, and a light chain variable region as set forth in the amino acid sequence of SEQ ID NO: 10. In one embodiment, an ADC used in the methods disclosed herein comprises an anti-CD117 antibody, or antigen binding portion thereof, comprises a heavy chain variable region as set forth in the amino acid sequence of SEQ ID NO: 9, and a light chain variable region as set forth in the amino acid sequence of SEQ ID NO: 11. In one embodiment, an ADC used in the methods disclosed herein comprises an anti-CD117 antibody, or antigen binding portion thereof, comprises a heavy chain variable region as set forth in the amino acid sequence of SEQ ID NO: 9, and a light chain variable region as set forth in the amino acid sequence of SEQ ID NO: 12. In one embodiment, an ADC used in the methods disclosed herein comprises an anti-CD117 antibody, or antigen binding portion thereof, comprises a heavy chain variable region as set forth in the amino acid sequence of SEQ ID NO: 9, and a light chain variable region as set forth in the amino acid sequence of SEQ ID NO: 13. In one embodiment, an ADC used in the methods disclosed herein comprises an anti-CD117 antibody, or antigen binding portion thereof, comprises a heavy chain variable region as set forth in the amino acid sequence of SEQ ID NO: 9, and a light chain variable region as set forth in the amino acid sequence of SEQ ID NO: 14. In one embodiment, an ADC used in the methods disclosed herein comprises an anti-CD117 antibody, or antigen binding portion thereof, comprises a heavy chain variable region as set forth in the amino acid sequence of SEQ ID NO: 9, and a light chain variable region as set forth in the amino acid sequence of SEQ ID NO: 15. In one embodiment, an ADC used in the methods disclosed herein comprises an anti-CD117 antibody, or antigen binding portion thereof, comprises a heavy chain variable region as set forth in the amino acid sequence of SEQ ID NO: 9, and a light chain variable region as set forth in the amino acid sequence of SEQ ID NO: 16. In one embodiment, an ADC used in the methods disclosed herein comprises an anti-CD117 antibody, or antigen binding portion thereof, comprises a heavy chain variable region as set forth in the amino acid sequence of SEQ ID NO: 9, and a light chain variable region as set forth in the amino acid sequence of SEQ ID NO: 17. In one embodiment, an ADC used in the methods disclosed herein comprises an anti-CD117 antibody, or antigen binding portion thereof, comprises a heavy chain variable region as set forth in the amino acid sequence of SEQ ID NO: 9, and a light chain variable region as set forth in the amino acid sequence of SEQ ID NO: 18. In one embodiment, an ADC used in the methods disclosed herein comprises an anti-CD117 antibody, or antigen binding portion thereof, comprises a heavy chain variable region as set forth in the amino acid sequence of SEQ ID NO: 9, and a light chain variable region as set forth in the amino acid sequence of SEQ ID NO: 19. In one embodiment, an ADC used in the methods disclosed herein comprises an anti-CD117 antibody, or antigen binding portion thereof, comprises a heavy chain variable region as set forth in the amino acid sequence of SEQ ID NO: 9, and a light chain variable region as set forth in the amino acid sequence of SEQ ID NO: 20. In one embodiment, an ADC used in the methods disclosed herein comprises an anti-CD117 antibody, or antigen binding portion thereof, comprises a heavy chain variable region as set forth in the amino acid sequence of SEQ ID NO: 9, and a light chain variable region as set forth in the amino acid sequence of SEQ ID NO: 21. In one embodiment, an ADC used in the methods disclosed herein comprises an anti-CD117 antibody, or antigen binding portion thereof, comprises a heavy chain variable region as set forth in the amino acid sequence of SEQ ID NO: 9, and a light chain variable region as set forth in the amino acid sequence of SEQ ID NO: 22. In one embodiment, an ADC used in the methods disclosed herein comprises an anti-CD117 antibody, or antigen binding portion thereof, comprises a heavy chain variable region as set forth in the amino acid sequence of SEQ ID NO: 9, and a light chain variable region as set forth in the amino acid sequence of SEQ ID NO: 23. In one embodiment, an ADC used in the methods disclosed herein comprises an anti-CD117 antibody, or antigen binding portion thereof, comprises a heavy chain variable region as set forth in the amino acid sequence of SEQ ID NO: 9, and a light chain variable region as set forth in the amino acid sequence of SEQ ID NO: 24. In one embodiment, an ADC used in the methods disclosed herein comprises an anti-CD117 antibody, or antigen binding portion thereof, comprises a heavy chain variable region as set forth in the amino acid sequence of SEQ ID NO: 9, and a light chain variable region as set forth in the amino acid sequence of SEQ ID NO: 25. In one embodiment, an ADC used in the methods disclosed herein comprises an anti-CD117 antibody, or antigen binding portion thereof, comprises a heavy chain variable region as set forth in the amino acid sequence of SEQ ID NO: 26, and a light chain variable region as set forth in the amino acid sequence of SEQ ID NO: 27. In one embodiment, an ADC used in the methods disclosed herein comprises an anti-CD117 antibody, or antigen binding portion thereof, comprises a heavy chain variable region as set forth in the amino acid sequence of SEQ ID NO: 28, and a light chain variable region as set forth in the amino acid sequence of SEQ ID NO: 29. In one embodiment, an ADC used in the methods disclosed herein comprises an anti-CD117 antibody, or antigen binding portion thereof, comprises a heavy chain variable region as set forth in the amino acid sequence of SEQ ID NO: 30, and a light chain variable region as set forth in the amino acid sequence of SEQ ID NO: 31. In one embodiment, an ADC used in the methods disclosed herein comprises an anti-CD117 antibody, or antigen binding portion thereof, comprises a heavy chain variable region as set forth in the amino acid sequence of SEQ ID NO: 32, and a light chain variable region as set forth in the amino acid sequence of SEQ ID NO: 33. In one embodiment, an ADC used in the methods disclosed herein comprises an anti-CD117 antibody, or antigen binding portion thereof, comprises a heavy chain variable region as set forth in the amino acid sequence of SEQ ID NO: 34, and a light chain variable region as set forth in the amino acid sequence of SEQ ID NO: 35. In one embodiment, an ADC used in the methods disclosed herein comprises an anti-CD117 antibody, or antigen binding portion thereof, comprises a heavy chain variable region as set forth in the amino acid sequence of SEQ ID NO: 36, and a light chain variable region as set forth in the amino acid sequence of SEQ ID NO: 37. In one embodiment, an ADC used in the methods disclosed herein comprises an anti-CD117 antibody, or antigen binding portion thereof, comprises a heavy chain variable region as set forth in the amino acid sequence of SEQ ID NO: 38, and a light chain variable region as set forth in the amino acid sequence of SEQ ID NO: 39. In one embodiment, an ADC used in the methods disclosed herein comprises an anti-CD117 antibody, or antigen binding portion thereof, comprises a heavy chain variable region as set forth in the amino acid sequence of SEQ ID NO: 40, and a light chain variable region as set forth in the amino acid sequence of SEQ ID NO: 41. In one embodiment, an ADC used in the methods disclosed herein comprises an anti-CD117 antibody, or antigen binding portion thereof, comprises a heavy chain variable region as set forth in the amino acid sequence of SEQ ID NO: 42, and a light chain variable region as set forth in the amino acid sequence of SEQ ID NO: 43. In one embodiment, an ADC used in the methods disclosed herein comprises an anti-CD117 antibody, or antigen binding portion thereof, comprises a heavy chain variable region as set forth in the amino acid sequence of SEQ ID NO: 34, and a light chain variable region as set forth in the amino acid sequence of SEQ ID NO: 44. In one embodiment, an ADC used in the methods disclosed herein comprises an anti-CD117 antibody, or antigen binding portion thereof, comprises a heavy chain variable region as set forth in the amino acid sequence of SEQ ID NO: 45, and a light chain variable region as set forth in the amino acid sequence of SEQ ID NO: 46. In one embodiment, an ADC used in the methods disclosed herein comprises an anti-CD117 antibody, or antigen binding portion thereof, comprises a heavy chain variable region as set forth in the amino acid sequence of SEQ ID NO: 47, and a light chain variable region as set forth in the amino acid sequence of SEQ ID NO: 48. In one embodiment, an ADC used in the methods disclosed herein comprises an anti-CD117 antibody, or antigen binding portion thereof, comprises a heavy chain variable region as set forth in the amino acid sequence of SEQ ID NO: 49, and a light chain variable region as set forth in the amino acid sequence of SEQ ID NO: 50. In one embodiment, an ADC used in the methods disclosed herein comprises an anti-CD117 antibody, or antigen binding portion thereof, comprises a heavy chain variable region as set forth in the amino acid sequence of SEQ ID NO: 51, and a light chain variable region as set forth in the amino acid sequence of SEQ ID NO: 52. In one embodiment, an ADC used in the methods disclosed herein comprises an anti-CD117 antibody, or antigen binding portion thereof, comprises a heavy chain variable region as set forth in the amino acid sequence of SEQ ID NO: 53, and a light chain variable region as set forth in the amino acid sequence of SEQ ID NO: 54. In one embodiment, an ADC used in the methods disclosed herein comprises an anti-CD117 antibody, or antigen binding portion thereof, comprises a heavy chain variable region as set forth in the amino acid sequence of SEQ ID NO: 55, and a light chain variable region as set forth in the amino acid sequence of SEQ ID NO: 56. In one embodiment, an ADC used in the methods disclosed herein comprises an anti-CD117 antibody, or antigen binding portion thereof, comprises a heavy chain variable region as set forth in the amino acid sequence of SEQ ID NO: 57, and a light chain variable region as set forth in the amino acid sequence of SEQ ID NO: 58. In one embodiment, an ADC used in the methods disclosed herein comprises an anti-CD117 antibody, or antigen binding portion thereof, comprises a heavy chain variable region as set forth in the amino acid sequence of SEQ ID NO: 59, and a light chain variable region as set forth in the amino acid sequence of SEQ ID NO: 60. In one embodiment, an ADC used in the methods disclosed herein comprises an anti-CD117 antibody, or antigen binding portion thereof, comprises a heavy chain variable region as set forth in the amino acid sequence of SEQ ID NO: 61, and a light chain variable region as set forth in the amino acid sequence of SEQ ID NO: 62. In one embodiment, an ADC used in the methods disclosed herein comprises an anti-CD117 antibody, or antigen binding portion thereof, comprises a heavy chain variable region as set forth in the amino acid sequence of SEQ ID NO: 63, and a light chain variable region as set forth in the amino acid sequence of SEQ ID NO: 52. In one embodiment, an ADC used in the methods disclosed herein comprises an anti-CD117 antibody, or antigen binding portion thereof, comprises a heavy chain variable region as set forth in the amino acid sequence of SEQ ID NO: 64, and a light chain variable region as set forth in the amino acid sequence of SEQ ID NO: 65. In one embodiment, an ADC used in the methods disclosed herein comprises an anti-CD117 antibody, or antigen binding portion thereof, comprises a heavy chain variable region as set forth in the amino acid sequence of SEQ ID NO: 66, and a light chain variable region as set forth in the amino acid sequence of SEQ ID NO: 67. In one embodiment, an ADC used in the methods disclosed herein comprises an anti-CD117 antibody, or antigen binding portion thereof, comprises a heavy chain variable region as set forth in the amino acid sequence of SEQ ID NO: 68, and a light chain variable region as set forth in the amino acid sequence of SEQ ID NO: 69. In one embodiment, an ADC used in the methods disclosed herein comprises an anti-CD117 antibody, or antigen binding portion thereof, comprises a heavy chain variable region as set forth in the amino acid sequence of SEQ ID NO: 70, and a light chain variable region as set forth in the amino acid sequence of SEQ ID NO: 71. In one embodiment, an ADC used in the methods disclosed herein comprises an anti-CD117 antibody, or antigen binding portion thereof, comprises a heavy chain variable region as set forth in the amino acid sequence of SEQ ID NO: 72, and a light chain variable region as set forth in the amino acid sequence of SEQ ID NO: 73. In one embodiment, an ADC used in the methods disclosed herein comprises an anti-CD117 antibody, or antigen binding portion thereof, comprises a heavy chain variable region as set forth in the amino acid sequence of SEQ ID NO: 74, and a light chain variable region as set forth in the amino acid sequence of SEQ ID NO: 75. In one embodiment, an ADC used in the methods disclosed herein comprises an anti-CD117 antibody, or antigen binding portion thereof, comprises a heavy chain variable region as set forth in the amino acid sequence of SEQ ID NO: 76, and a light chain variable region as set forth in the amino acid sequence of SEQ ID NO: 77. In one embodiment, an ADC used in the methods disclosed herein comprises an anti-CD117 antibody, or antigen binding portion thereof, comprises a heavy chain variable region as set forth in the amino acid sequence of SEQ ID NO: 78, and a light chain variable region as set forth in the amino acid sequence of SEQ ID NO: 79. In one embodiment, an ADC used in the methods disclosed herein comprises an anti-CD117 antibody, or antigen binding portion thereof, comprises a heavy chain variable region as set forth in the amino acid sequence of SEQ ID NO: 80, and a light chain variable region as set forth in the amino acid sequence of SEQ ID NO: 81. In one embodiment, an ADC used in the methods disclosed herein comprises an anti-CD117 antibody, or antigen binding portion thereof, comprises a heavy chain variable region as set forth in the amino acid sequence of SEQ ID NO: 82, and a light chain variable region as set forth in the amino acid sequence of SEQ ID NO: 83. In one embodiment, an ADC used in the methods disclosed herein comprises an anti-CD117 antibody, or antigen binding portion thereof, comprises a heavy chain variable region as set forth in the amino acid sequence of SEQ ID NO: 84, and a light chain variable region as set forth in the amino acid sequence of SEQ ID NO: 85. In one embodiment, an ADC used in the methods disclosed herein comprises an anti-CD117 antibody, or antigen binding portion thereof, comprises a heavy chain variable region as set forth in the amino acid sequence of SEQ ID NO: 86, and a light chain variable region as set forth in the amino acid sequence of SEQ ID NO: 87. In one embodiment, an ADC used in the methods disclosed herein comprises an anti-CD117 antibody, or antigen binding portion thereof, comprises a heavy chain variable region as set forth in the amino acid sequence of SEQ ID NO: 88, and a light chain variable region as set forth in the amino acid sequence of SEQ ID NO: 89. In one embodiment, an ADC used in the methods disclosed herein comprises an anti-CD117 antibody, or antigen binding portion thereof, comprises a heavy chain variable region as set forth in the amino acid sequence of SEQ ID NO: 9, and a light chain variable region as set forth in the amino acid sequence of SEQ ID NO: 90. In one embodiment, an ADC used in the methods disclosed herein comprises an anti-CD117 antibody, or antigen binding portion thereof, comprises a heavy chain variable region as set forth in the amino acid sequence of SEQ ID NO: 9, and a light chain variable region as set forth in the amino acid sequence of SEQ ID NO: 91. In one embodiment, an ADC used in the methods disclosed herein comprises an anti-CD117 antibody, or antigen binding portion thereof, comprises a heavy chain variable region as set forth in the amino acid sequence of SEQ ID NO: 9, and a light chain variable region as set forth in the amino acid sequence of SEQ ID NO: 92. In one embodiment, an ADC used in the methods disclosed herein comprises an anti-CD117 antibody, or antigen binding portion thereof, comprises a heavy chain variable region as set forth in the amino acid sequence of SEQ ID NO: 9, and a light chain variable region as set forth in the amino acid sequence of SEQ ID NO: 93. In one embodiment, an ADC used in the methods disclosed herein comprises an anti-CD117 antibody, or antigen binding portion thereof, comprises a heavy chain variable region as set forth in the amino acid sequence of SEQ ID NO: 9, and a light chain variable region as set forth in the amino acid sequence of SEQ ID NO: 98. In one embodiment, an ADC used in the methods disclosed herein comprises an anti-CD117 antibody, or antigen binding portion thereof, comprises a heavy chain variable region as set forth in the amino acid sequence of SEQ ID NO: 9, and a light chain variable region as set forth in the amino acid sequence of SEQ ID NO: 99. In one embodiment, an ADC used in the methods disclosed herein comprises an anti-CD117 antibody, or antigen binding portion thereof, comprises a heavy chain variable region as set forth in the amino acid sequence of SEQ ID NO: 9, and a light chain variable region as set forth in the amino acid sequence of SEQ ID NO: 101. In one embodiment, an ADC used in the methods disclosed herein comprises an anti-CD117 antibody, or antigen binding portion thereof, comprises a heavy chain variable region as set forth in the amino acid sequence of SEQ ID NO: 9, and a light chain variable region as set forth in the amino acid sequence of SEQ ID NO: 102. In one embodiment, an ADC used in the methods disclosed herein comprises an anti-CD117 antibody, or antigen binding portion thereof, comprises a heavy chain variable region as set forth in the amino acid sequence of SEQ ID NO: 9, and a light chain variable region as set forth in the amino acid sequence of SEQ ID NO: 104. In one embodiment, an ADC used in the methods disclosed herein comprises an anti-CD117 antibody, or antigen binding portion thereof, comprises a heavy chain variable region as set forth in the amino acid sequence of SEQ ID NO: 9, and a light chain variable region as set forth in the amino acid sequence of SEQ ID NO: 105. In one embodiment, an ADC used in the methods disclosed herein comprises an anti-CD117 antibody, or antigen binding portion thereof, comprises a heavy chain variable region as set forth in the amino acid sequence of SEQ ID NO: 9, and a light chain variable region as set forth in the amino acid sequence of SEQ ID NO: 105. In one embodiment, an ADC used in the methods disclosed herein comprises an anti-CD117 antibody, or antigen binding portion thereof, comprises a heavy chain variable region as set forth in the amino acid sequence of SEQ ID NO: 106, and a light chain variable region as set forth in the amino acid sequence of SEQ ID NO: 107. In one embodiment, an ADC used in the methods disclosed herein comprises an anti-CD117 antibody, or antigen binding portion thereof, comprises a heavy chain variable region as set forth in the amino acid sequence of SEQ ID NO: 114, and a light chain variable region as set forth in the amino acid sequence of SEQ ID NO: 115. In one embodiment, an ADC used in the methods disclosed herein comprises an anti-CD117 antibody, or antigen binding portion thereof, comprises a heavy chain variable region as set forth in the amino acid sequence of SEQ ID NO: 106, and a light chain variable region as set forth in the amino acid sequence of SEQ ID NO: 119. In one embodiment, an ADC used in the methods disclosed herein comprises an anti-CD117 antibody, or antigen binding portion thereof, comprises a heavy chain variable region as set forth in the amino acid sequence of SEQ ID NO: 121, and a light chain variable region as set forth in the amino acid sequence of SEQ ID NO: 119. In one embodiment, an ADC used in the methods disclosed herein comprises an anti-CD117 antibody, or antigen binding portion thereof, comprises a heavy chain variable region as set forth in the amino acid sequence of SEQ ID NO: 123, and a light chain variable region as set forth in the amino acid sequence of SEQ ID NO: 117. In one embodiment, an ADC used in the methods disclosed herein comprises an anti-CD117 antibody, or antigen binding portion thereof, comprises a heavy chain variable region as set forth in the amino acid sequence of SEQ ID NO: 124, and a light chain variable region as set forth in the amino acid sequence of SEQ ID NO: 125. In one embodiment, an ADC used in the methods disclosed herein comprises an anti-CD117 antibody, or antigen binding portion thereof, comprises a heavy chain variable region as set forth in the amino acid sequence of SEQ ID NO: 9, and a light chain variable region as set forth in the amino acid sequence of SEQ ID NO: 125. In one embodiment, an ADC used in the methods disclosed herein comprises an anti-CD117 antibody, or antigen binding portion thereof, comprises a heavy chain variable region as set forth in the amino acid sequence of SEQ ID NO: 9, and a light chain variable region as set forth in the amino acid sequence of SEQ ID NO: 126. In one embodiment, an ADC used in the methods disclosed herein comprises an anti-CD117 antibody, or antigen binding portion thereof, comprises a heavy chain variable region as set forth in the amino acid sequence of SEQ ID NO: 124, and a light chain variable region as set forth in the amino acid sequence of SEQ ID NO: 127. In one embodiment, an ADC used in the methods disclosed herein comprises an anti-CD117 antibody, or antigen binding portion thereof, comprises a heavy chain variable region as set forth in the amino acid sequence of SEQ ID NO: 124, and a light chain variable region as set forth in the amino acid sequence of SEQ ID NO: 128. In one embodiment, an ADC used in the methods disclosed herein comprises an anti-CD117 antibody, or antigen binding portion thereof, comprises a heavy chain variable region as set forth in the amino acid sequence of SEQ ID NO: 150, and a light chain variable region as set forth in the amino acid sequence of SEQ ID NO: 151. In one embodiment, an ADC used in the methods disclosed herein comprises an anti-CD117 antibody, or antigen binding portion thereof, comprises a heavy chain variable region as set forth in the amino acid sequence of SEQ ID NO: 158, and a light chain variable region as set forth in the amino acid sequence of SEQ ID NO: 159 In one embodiment, an ADC used in the methods disclosed herein comprises an anti-CD117 antibody, or antigen binding portion thereof, comprises a heavy chain variable region as set forth in the amino acid sequence of SEQ ID NO: 165, and a light chain variable region as set forth in the amino acid sequence of SEQ ID NO: 159. In one embodiment, an ADC used in the methods disclosed herein comprises an anti-CD117 antibody, or antigen binding portion thereof, comprises a heavy chain variable region as set forth in the amino acid sequence of SEQ ID NO: 168, and a light chain variable region as set forth in the amino acid sequence of SEQ ID NO: 159. In one embodiment, an ADC used in the methods disclosed herein comprises an anti-CD117 antibody, or antigen binding portion thereof, comprises a heavy chain variable region as set forth in the amino acid sequence of SEQ ID NO: 171, and a light chain variable region as set forth in the amino acid sequence of SEQ ID NO: 159. In one embodiment, an ADC used in the methods disclosed herein comprises an anti-CD117 antibody, or antigen binding portion thereof, comprises a heavy chain variable region as set forth in the amino acid sequence of SEQ ID NO: 173, and a light chain variable region as set forth in the amino acid sequence of SEQ ID NO: 174. In one embodiment, an ADC used in the methods disclosed herein comprises an anti-CD117 antibody, or antigen binding portion thereof, comprises a heavy chain variable region as set forth in the amino acid sequence of SEQ ID NO: 180, and a light chain variable region as set forth in the amino acid sequence of SEQ ID NO: 174. In one embodiment, an ADC used in the methods disclosed herein comprises an anti-CD117 antibody, or antigen binding portion thereof, comprises a heavy chain variable region as set forth in the amino acid sequence of SEQ ID NO: 183, and a light chain variable region as set forth in the amino acid sequence of SEQ ID NO: 151. In one embodiment, an ADC used in the methods disclosed herein comprises an anti-CD117 antibody, or antigen binding portion thereof, comprises a heavy chain variable region as set forth in the amino acid sequence of SEQ ID NO: 186, and a light chain variable region as set forth in the amino acid sequence of SEQ ID NO: 151. In one embodiment, an ADC used in the methods disclosed herein comprises an anti-CD117 antibody, or antigen binding portion thereof, comprises a heavy chain variable region as set forth in the amino acid sequence of SEQ ID NO: 189, and a light chain variable region as set forth in the amino acid sequence of SEQ ID NO: 151.

In one embodiment, an ADC used in the methods disclosed herein comprises an anti-CD117 antibody, or antigen binding portion thereof, as described in US 2019-0153114 A1, which is incorporated by reference in its entirety. In another embodiment, an ADC used in the methods disclosed herein comprises an anti-CD117 antibody, or antigen binding portion thereof, as described in US 2019-0144558 A1, which is incorporated by reference in its entirety.

In one embodiment, the present invention provides a method of depleting a population of target cells in a human patient, wherein the method comprises a dosing regimen comprising the steps of administering to the patient a first dose of an antibody drug conjugate (ADC) on day 1; and administering to the patient a second dose of the ADC after the first dose; wherein the ADC comprises an antibody, or antigen-binding fragment thereof, conjugated to an amatoxin via a linker, wherein the ADC is specific for a cell surface marker, and wherein the dosing regimen induces a decrease in the population of the target cells in a human patient. In one embodiment, the first dose is selected from the group consisting of about 0.03-0.29 mg/kg of the ADC, about 0.03-0.25 mg/kg of the ADC, about 0.03-0.20 mg/kg of the ADC, about 0.03-0.15 mg/kg of the ADC, about 0.03-0.10 mg/kg of the ADC, about 0.05-0.10 mg/kg of the ADC, and about 0.05-0.07 mg/kg of the ADC. In another embodiment, the first dose comprises about 0.03-0.19 mg/kg of the ADC. In yet another embodiment, the second dose is within 10% by weight of the first dose. Alternatively, the first and second dose can be the same dose, either as a same fixed dose or a same dose by calculation of the same mg/kg.

In another embodiment, the second dose is administered between 1-24 hours after the first dose is administered. In yet another embodiment, the second dose is administered between 1-12 days after the first dose is administered. In another embodiment, the target cell is a stem cell. In other embodiments, the target cell is an immune cell. In another embodiment, the target cell is a disease causing cell. In another embodiment, the cell marker is selected from the group consisting of CD117, CD45, CD2, CD5, and CD137.

In one embodiment, the present invention provides a method of depleting a population of target cells in a human patient for transplant conditioning, wherein the method comprises a dosing regimen comprising the steps of administering to the patient a first dose of an antibody drug conjugate (ADC) on day 1; and administering to the patient a second dose of the ADC after the first dose; wherein the ADC comprises an antibody, or antigen-binding fragment thereof, conjugated to a toxin via a linker, wherein the ADC is specific for a cell surface marker, and wherein the dosing regimen induces a decrease in the population of the target cells in a human patient for transplant conditioning. In another embodiment, the toxin is an antimitotic agent or an RNA polymerase inhibitor. In another embodiment, the RNA polymerase inhibitor is an amatoxin. In another embodiment, the RNA polymerase inhibitor is an amanitin. In another embodiment, the amanitin is selected from the group consisting of α-amanitin, β-amanitin, γ-amanitin, ε-amanitin, amanin, amaninamide, amanullin, amanullinic acid, and proamanullin. In yet another embodiment, the amatoxin is represented by formula (I)

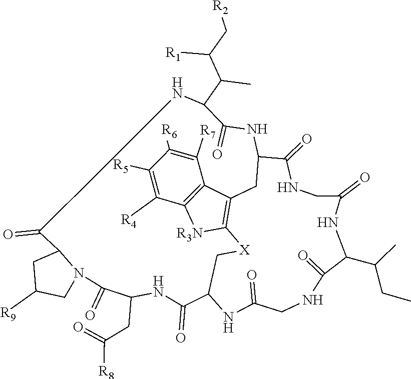

wherein R₁ is H, OH, OR_{A}, or OR_{C};
R₂ is H, OH, OR_{B}, or OR_{C};
R_{A} and R_{B}, together with the oxygen atoms to which they are bound, combine to form an optionally substituted 5-membered heterocyclolalkyl group;
R₃ is H, R_{C}, or R_{D};
R₄, R₅, R₆, and R₇ are each independently H, OH, OR_{C}, OR_{D}, R_{C}, or R_{D};
R₈ is OH, NH₂, OR_{C}, OR_{D}, NHR_{C}, or NR_{C}R_{D};
R₉ is H, OH, OR_{C}, or OR_{D};
X is —S—, —S(O)—, or —SO₂—;
R_{C} is -L-Z;
R_{D} is substituted C₁-C₆ alkyl, substituted C₁-C₆ heteroalkyl, substituted C₂-C₆ alkenyl, substituted C₂-C₆ heteroalkenyl, substituted C₂-C₆ alkynyl, substituted C₂-C₆ heteroalkynyl, substituted cycloalkyl, substituted heterocycloalkyl, substituted aryl, or substituted heteroaryl;
L is a peptide containing linker; and
Z is a chemical moiety formed from a coupling reaction between a reactive substituent present on L and a reactive substituent present within the anti-CD137 antibody or antigen-binding fragment thereof. In another embodiment, the antimitotic agent is maytansine or auristatin. In another embodiment, the auristatin is monomethyl auristatin F (MMAF) or monomethyl auristatin E (MMAE). In another embodiment, the linker of the ADC is N-beta-maleimidopropyl-Val-Ala-para-aminobenzyl (BMP-Val-Ala-PAB). In another embodiment, wherein the second dose is administered about 1 hour after the first dose is administered. In another embodiment, the second dose is administered about 2 hours after the first dose is administered. In another embodiment, the second dose is administered about 3 hours after the first dose is administered. In another embodiment, the second dose is administered about 4 hours after the first dose is administered. In another embodiment, the second dose is administered about 5 hours after the first dose is administered. In another embodiment, the second dose is administered about 6 hours after the first dose is administered. In another embodiment, the second dose is administered about 7 hours after the first dose is administered. In another embodiment, the second dose is administered about 8 hours after the first dose is administered. In another embodiment, the second dose is administered about 1 day after the first dose is administered. In another embodiment, the second dose is administered about 2 days after the first dose is administered. In another embodiment, the second dose is administered about 3 days after the first dose is administered.

In one embodiment, the present invention provides a method of depleting a population of target cells in a human patient, wherein the method comprises a dosing regimen comprising the steps of: (a) administering to the patient a first dose of an antibody drug conjugate (ADC) on day 1; and (b) administering to the patient a second dose of the ADC after the first dose; wherein the ADC comprises an antibody, or antigen-binding fragment thereof, conjugated to a cytotoxin (e.g., an amatoxin) via a linker, wherein the ADC is specific for a cell surface marker, and wherein the dosing regimen induces a decrease in the population of the target cells in a human patient. In one embodiment, the first dose is selected from the group consisting of about 2-12 mg of the ADC, about 2-11 mg of the ADC, about 2-10 mg of the ADC, about 2-9 mg of the ADC, about 2-8 mg of the ADC, about 2-7 mg of the ADC, about 2-6 mg of the ADC, about 2-5 mg of the ADC, about 2-4 mg of the ADC, and about 2-3 mg of the ADC. In another embodiment, the first dose comprises about 1.9-11.5 mg of the ADC. In some embodiments, the second dose is within 10% by weight of the first dose or wherein the second dose is the same as the first dose. In yet other embodiments, the second dose is administered between 1-24 hours after the first dose is administered. In other embodiments, the second dose is administered between 1-12 days after the first dose is administered. In another embodiment, the target cell is a stem cell. In another embodiment, the target cell is an immune cell. In yet another embodiment, the target cell is a disease causing cell. In certain embodiments, the cell marker is selected from the group consisting of CD117, CD45, CD2, CD5, CD252, CD134, and CD137. In another embodiment, the antibody, or antigen-binding fragment thereof, comprises a heavy chain variable region having an amino acid sequence as set forth in SEQ ID NO: 106 and a light chain variable region having an amino acid sequence as set forth in SEQ ID NO: 107. In yet another embodiment, the antibody, or antigen-binding fragment thereof, comprises a heavy chain variable region having an amino acid sequence as set forth in SEQ ID NO: 150 and a light chain variable region having an amino acid sequence as set forth in SEQ ID NO: 151.

In one embodiment, the present invention provides a method of depleting a population of target cells in a human patient, wherein the method comprises a dosing regimen comprising the steps of: (a) administering to the patient a first dose of an antibody drug conjugate (ADC) on day 1, wherein the first dose is selected from the group consisting of about 0.03-0.29 mg/kg of the ADC, about 0.03-0.25 mg/kg of the ADC, about 0.03-0.20 mg/kg of the ADC, about 0.03-0.15 mg/kg of the ADC, about 0.03-0.10 mg/kg of the ADC, about 0.05-0.10 mg/kg of the ADC, and about 0.05-0.07 mg/kg of the ADC; and (b) administering to the patient a second dose of the ADC after the first dose; wherein the ADC comprises an antibody, or antigen-binding fragment thereof, conjugated to a cytotoxin (e.g., an amatoxin) via a linker, wherein the ADC is specific for a cell surface marker, and wherein the dosing regimen induces a decrease in the population of the target cells in a human patient. In one embodiment, the first dose is about 0.03-0.29 mg/kg. In another embodiment, the first dose comprises about 0.03-0.19 mg/kg of the ADC. In another embodiment, the second dose is within 10% by weight of the first dose or wherein the second dose is the same by weight of the first dose. In yet another embodiment, the second dose is administered between 1-24 hours after the first dose is administered. In some embodiments, the second dose is administered between 1-12 days after the first dose is administered. In another embodiment, the target cell is a stem cell. In another embodiment, the target cell is an immune cell. In yet another embodiment, the target cell is a disease causing cell. In certain embodiments, the cell marker is selected from the group consisting of CD117, CD45, CD2, CD5, CD252, and CD137. In yet other embodiments, the antibody, or antigen-binding fragment thereof, is an anti-CD117 antibody comprising a heavy chain variable region having an amino acid sequence as set forth in SEQ ID NO: 106 and a light chain variable region having an amino acid sequence as set forth in SEQ ID NO: 107. In another embodiment, the antibody, or antigen-binding fragment thereof, is an anti-CD117 antibody comprising a heavy chain variable region having an amino acid sequence as set forth in SEQ ID NO: 150 and a light chain variable region having an amino acid sequence as set forth in SEQ ID NO: 151.

In one embodiment, the invention provides a method of depleting a population of target cells in a human patient, wherein the method comprises a dosing regimen comprising: administering to the patient a second dose of an antibody drug conjugate (ADC) within 1 to 14 days after the patient was administered a first dose of the ADC; wherein the ADC comprises an antibody, or antigen-binding fragment thereof, conjugated to cytotoxin via a linker; wherein the ADC is specific for a cell surface marker; wherein the dosing regimen induces a decrease in the population of the target cells in a human patient; and wherein at least one of the patient's blood AST, ALT, or LDH levels does not reach a toxic level between administration of the first dose and 14 days after administration of the first dose to the patient.

In one embodiment, the invention provides a method of depleting a population of target cells in a human patient, wherein the method comprises a dosing regimen comprising: administering to the patient a second dose of an antibody drug conjugate (ADC) within 1 to 14 days after the patient was administered a first dose of the ADC; wherein the ADC comprises an antibody, or antigen-binding fragment thereof, conjugated to cytotoxin via a linker; wherein the ADC is specific for a cell surface marker; wherein the dosing regimen induces a decrease in the population of the target cells in a human patient; and wherein at least one of the patient's blood AST, ALT, or LDH levels does not rise more than 3-fold above normal levels between administration of the first dose and 14 days after administration of the first dose to the patient.

In one embodiment, the present invention provides a method of depleting a population of target cells in a human patient for transplant conditioning, wherein the method comprises a dosing regimen comprising the steps of: (a) administering to the patient a first dose of an antibody drug conjugate (ADC) on day 1; and (b) administering to the patient a second dose of the ADC after the first dose; wherein the ADC comprises an antibody, or antigen-binding fragment thereof, conjugated to a cytotoxin via a linker, wherein the ADC is specific for a cell surface marker selected from CD117, CD45, CD137, CD2, CD252, CD134, or CD5, and wherein the dosing regimen induces a decrease in the population of the target cells in a human patient for transplant conditioning. In one embodiment, the toxin is an antimitotic agent or an RNA polymerase inhibitor. In some embodiments, the RNA polymerase inhibitor is an amatoxin. In yet another embodiment, the RNA polymerase inhibitor is an amanitin. In some embodiments, the amanitin is selected from the group consisting of α-amanitin, β-amanitin, γ-amanitin, ε-amanitin, amanin, amaninamide, amanullin, amanullinic acid, and proamanullin. In yet another embodiment, the amatoxin is represented by formula (I)

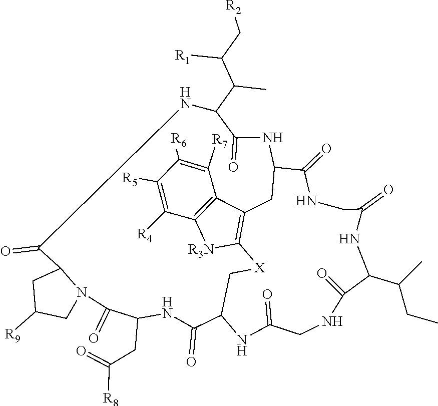

wherein R₁ is H, OH, OR_{A}, or OR_{C};
R₂ is H, OH, OR_{B}, or OR_{C};
R_{A} and R_{B}, together with the oxygen atoms to which they are bound, combine to form an optionally substituted 5-membered heterocyclolalkyl group;
R₃ is H, R_{C}, or R_{D};
R₄, R₅, R₆, and R₇ are each independently H, OH, OR_{C}, OR_{D}, R_{C}, or R_{D};
R₈ is OH, NH₂, OR_{C}, OR_{D}, NHR_{C}, or NR_{C}R_{D};
R₉ is H, OH, OR_{C}, or OR_{D};
X is —S—, —S(O)—, or —SO₂—;
R_{C} is -L-Z;
R_{D} is substituted C₁-C₆ alkyl, substituted C₁-C₆ heteroalkyl, substituted C₂-C₆ alkenyl, substituted C₂-C₆ heteroalkenyl, substituted C₂-C₆ alkynyl, substituted C₂-C₆ heteroalkynyl, substituted cycloalkyl, substituted heterocycloalkyl, substituted aryl, or substituted heteroaryl;
L is a peptide containing linker; and
Z is a chemical moiety formed from a coupling reaction between a reactive substituent present on L and a reactive substituent present within the antibody or antigen-binding fragment thereof.

In one embodiment, the cytotoxin is a PBD.

In some embodiments, the antimitotic agent is maytansine or auristatin. In yet another embodiment, the auristatin is monomethyl auristatin F (MMAF) or monomethyl auristatin E (MMAE). In some embodiments, the HSC cell surface marker is CD117. In some embodiments, the antibody binds CD117 and a) comprises a heavy chain variable region having an amino acid sequence as set forth in SEQ ID NO: 106 and a light chain variable region having an amino acid sequence as set forth in SEQ ID NO: 107; or b) comprises a heavy chain variable region having an amino acid sequence as set forth in SEQ ID NO: 150 and a light chain variable region having an amino acid sequence as set forth in SEQ ID NO: 151. In other embodiments, the antibody comprises an Fc region comprising a D265C mutation (according to EU index). In some embodiments, the antibody comprises an H435A mutation (according to EU index). In other embodiments, the linker of the ADC is N-beta-maleimidopropyl-Val-Ala-para-aminobenzyl (BMP-Val-Ala-PAB). In some embodiments, the second dose is administered about 1 hour after the first dose is administered. In some embodiments, the second dose is administered about 2 hours after the first dose is administered. In some embodiments, the second dose is administered about 3 hours after the first dose is administered. In other embodiments, the second dose is administered about 4 hours after the first dose is administered. In some embodiments, the second dose is administered about 5 hours after the first dose is administered. In some embodiments, the second dose is administered about 6 hours after the first dose is administered. In some embodiments, the second dose is administered about 7 hours after the first dose is administered. In other embodiments, the second dose is administered about 8 hours after the first dose is administered. In some embodiments, the second dose is administered about 1 day after the first dose is administered. In some embodiments, the second dose is administered about 2 days after the first dose is administered. In other embodiments, the second dose is administered about 3 days after the first dose is administered. In yet other embodiments, the patient further receives a stem cell transplant when the ADC is substantially cleared from the blood of the patient. In some embodiments, the dosing regimen for administering the ADC consists essentially of steps a) and b).

In yet other embodiments, the method further comprises administering a cell transplantation to the human patient.

In some embodiments, the cell transplant is a hematopoietic stem cell (HSC) transplantation. In yet other embodiments, the method is non-myeloablative.

In some embodiments, a liver marker determined from a patient sample does not reach a toxic level. In some embodiments, a liver marker determined from a patient sample does not rise above normal levels (e.g., a reference range), does not rise more than 1.5-fold above normal levels, does not rise more than 3-fold above normal levels, does not rise more than 5-fold above normal levels, or does not rise more than 10-fold above normal levels. In some embodiments, the liver marker is AST, LDH, or ALT. In another embodiment, a liver marker determined from the patient is elevated for no more than 7 days following administration of the first dose. In other embodiments, a liver marker determined from the patient is elevated for no more than 6 days following administration of the first dose. In some embodiments, a liver marker determined from the patient is elevated for no more than 5 days following administration of the first dose. In some embodiments, the liver marker is AST, ALT, or LDH.

### BRIEF DESCRIPTION OF THE FIGURES

FIG. 1 graphically depicts the results of an assay detecting the reticulocyte count (10⁹/μL) as a function of days post initial dose administration of varying doses of the ADC 1 antibody drug conjugate (ADC) versus controls (i.e., PBS and an isotype ADC control). The dashed horizonal lines in FIG. 1 represent the normal reticulocyte count range.

FIG. 2 graphically depicts the results of an assay detecting the platelet count (10³/μL) as a function of days post initial dose administration of varying doses of the ADC 1 antibody drug conjugate (ADC) versus controls (i.e., PBS and an isotype ADC control). The dashed horizonal lines in FIG. 2 represent the normal platelet count range.

FIGS. 3A-3C graphically depict the results of an assay detecting the levels of (FIG. 3A) plasma alanine aminotransaminase (ALT; in U/L) (FIG. 3B), plasma lactate dehydrogenase (LDH; in U/L) and (FIG. 3C) plasma aspartate aminotransaminase (AST; in U/L) as a function of days post initial dose administration of varying doses of the ADC 1 antibody drug conjugate (ADC) versus controls (i.e., PBS and an isotype ADC control). The dashed horizonal lines in FIGS. 3A, 3B, and 3C represent normal ALT, LDH, and AST level ranges, respectively, FIG. 3D graphically depicts hematopoietic stem and progenitor cell number in bone marrow following treatment with varying doses of ADC 1, a fast half-life antibody drug conjugate, in comparison to a wild-type half-life version of ADC 1 and various controls. In the experiment described in FIG. 3D, the bone aspirate was taken on day 15 for single doses and day 18 for the multi-dose (i.e., 0.1 mg/kg, Q3D×2 (0.1/0.1)).

FIG. 3E graphically depicts hematopoietic stem and progenitor cell depletion in bone marrow following treatment with ADC 1 in comparison to various controls. In the experiment described in FIG. 3E, the bone aspirate was taken on day 15 for single doses and day 18 for the multi-dose regimens.

FIG. 3F graphically depicts the plasma concentration of ADC 1 following the administration of varying doses of the ADC 1 as a function of time (hours).

FIG. 4A graphically depicts the results of an assay detecting the reticulocyte count (10⁹/μL) as a function of days post initial dose administration of varying doses of the ADC 2 antibody drug conjugate (ADC) and a dose of the ADC 3. The dashed horizonal lines in FIG. 4A represent the normal reticulocyte count range.

FIG. 4B graphically depict the results of an assay detecting the levels of plasma aspartate aminotransaminase (AST; in U/L) as a function of days post initial dose administration of varying doses of the ADC 2 and ADC 3. The dashed horizonal lines in FIG. 4B represent the normal AST level range.

FIG. 4C graphically depicts the plasma concentration of ADC 2 and ADC 3 following the administration of varying doses of the ADC 2 and ADC 3 as a function of time (hours).

FIG. 5 depicts an exemplary compound of Formula (Z′-L-D).

### DETAILED DESCRIPTION

The invention provides dosing regimens for antibody drug conjugates, as well as methods of administering said dosing regimens to patients suffering from or at risk of various diseases and conditions such as autoimmune diseases, cancers, and Graft versus Host Disease (GvHD), among others, by administration of an antibody drug conjugate (ADC), capable of binding an antigen expressed by a hematopoietic cell, such as a hematopoietic stem cell, an immune cell or a cancer cell.

### Definitions

As used herein, the term “about” refers to a value that is within 10% above or below the value being described.

As used herein, the term “amatoxin” refers to a member of the amatoxin family of peptides produced by Amanita phalloides mushrooms, or derivative thereof, such as a variant or derivative thereof capable of inhibiting RNA polymerase II activity. Amatoxins useful in conjunction with the compositions and methods described herein include compounds such, as but not limited to, compounds of Formulas (II), (III), (IIIa), and (IIIb), e.g., α-amanitin, β-amanitin, γ-amanitin, ε-amanitin, amanin, amaninamide, amanullin, amanullinic acid, or proamanullin. As described herein, amatoxins may be conjugated to an antibody, or antigen-binding fragment thereof, for instance, by way of a linker moiety (L) (thus forming an ADC). Exemplary methods of amatoxin conjugation and linkers useful for such processes are described below. Exemplary linker-containing amatoxins useful for conjugation to an antibody, or antigen-binding fragment, in accordance with the compositions and methods are also described herein.

As used herein, the term “conjugate” or “antibody drug conjugate” or “ADC” refers to an antibody which is linked to a cytotoxin. An ADC is formed by the chemical bonding of a reactive functional group of one molecule, such as an antibody or antigen-binding fragment thereof, with an appropriately reactive functional group of another molecule, such as a cytotoxin described herein. Conjugates may include a linker between the two molecules bound to one another, e.g., between an antibody and a cytotoxin. Examples of linkers that can be used for the formation of a conjugate include peptide-containing linkers, such as those that contain naturally occurring or non-naturally occurring amino acids, such as D-amino acids. Linkers can be prepared using a variety of strategies described herein and known in the art. Depending on the reactive components therein, a linker may be cleaved, for example, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012).

As used herein, the term “antibody” refers to an immunoglobulin molecule that specifically binds to, or is immunologically reactive with, a particular antigen. An antibody includes, but is not limited to, monoclonal antibodies, polyclonal antibodies, multispecific antibodies (e.g., bispecific antibodies), and antibody fragments so long as they exhibit the desired antigen-binding activity.

Generally, antibodies comprise heavy and light chains containing antigen binding regions. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH, and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

An “intact” or “full length” antibody, as used herein, refers to an antibody having two heavy (H) chain polypeptides and two light (L) chain polypeptides interconnected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH, and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

As used herein, the term “complementarity determining region” (CDR) refers to a hypervariable region found both in the light chain and the heavy chain variable domains of an antibody. The more highly conserved portions of variable domains are referred to as framework regions (FRs). The amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The antibodies described herein may contain modifications in these hybrid hypervariable positions. The variable domains of native heavy and light chains each contain four framework regions that primarily adopt a β-sheet configuration, connected by three CDRs, which form loops that connect, and in some cases form part of, the β-sheet structure. The CDRs in each chain are held together in close proximity by the framework regions in the order FR1-CDR1-FR2-CDR2-FR3-CDR3-FR4 and, with the CDRs from the other antibody chains, contribute to the formation of the target binding site of antibodies (see Kabat et al., Sequences of Proteins of Immunological Interest, National Institute of Health, Bethesda, MD., 1987). In certain embodiments, numbering of immunoglobulin amino acid residues is performed according to the immunoglobulin amino acid residue numbering system of Kabat et al., unless otherwise indicated (although any antibody numbering scheme, including, but not limited to IMGT and Chothia, can be utilized).

The term “antigen-binding fragment,” as used herein, refers to one or more fragments of an antibody that retain the ability to specifically bind to a target antigen. The antigen-binding function of an antibody can be performed by fragments of a full-length antibody. The antibody fragments can be, for example, a Fab, F(ab′)₂, scFv, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the V_{L}, V_{H}, C_{L}, and C_{H}1 domains; (ii) a F(ab′)₂ fragment, a bivalent fragment containing two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb including V_{H} and V_{L} domains; (vi) a dAb fragment that consists of a V_{H} domain (see, e.g., Ward et al., Nature 341:544-546, 1989); (vii) a dAb which consists of a V_{H} or a V_{L} domain; (viii) an isolated complementarity determining region (CDR); and (ix) a combination of two or more (e.g., two, three, four, five, or six) isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single chain Fv (scFv); see, for example, Bird et al., Science 242:423-426, 1988 and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in certain cases, by chemical peptide synthesis procedures known in the art.

As used herein, the term “anti-CD117 antibody” or “an antibody that binds to CD117” refers to an antibody that is capable of binding CD117, e.g., human CD117 (hCD117) with sufficient affinity such that the antibody is useful as a diagnostic and/or therapeutic agent in targeting CD117. The amino acid sequence of human CD117 is described in SEQ ID NO: 232.

As used herein, the term “anti-CD137 antibody” or “an antibody that binds to CD137” refers to an antibody that is capable of binding CD137, e.g., human CD137 (hCD137) with sufficient affinity such that the antibody is useful as a diagnostic and/or therapeutic agent in targeting CD137. The amino acid sequence of human CD137 is described in SEQ ID NO: 233.

As used herein, the term “anti-CD45 antibody” or “an antibody that binds to CD45” refers to an antibody that is capable of binding CD45, e.g., human CD45 (hCD45), with sufficient affinity such that the antibody is useful as a diagnostic and/or therapeutic agent in targeting CD45. The amino acid sequence of various human CD45 isoforms are described in SEQ ID NO: 236 (i.e., CD45RO isoform), SEQ ID NO: 237 (i.e., CD45RA isoform), SEQ ID NO: 238 (i.e., CD45RB isoform), and SEQ ID NO: 239 (i.e., CD45RC isoform).

As used herein, the term “anti-CD5 antibody” or “an antibody that binds to CD5” refers to an antibody that is capable of binding CD5, human CD5 (hCD5), with sufficient affinity such that the antibody is useful as a diagnostic and/or therapeutic agent in targeting CD5. The amino acid sequence of human CD5 is described in SEQ ID NO: 234.

As used herein, the term “anti-CD2 antibody” or “an antibody that binds to CD2” refers to an antibody that is capable of binding CD2, e.g., human CD2 (hCD2), with sufficient affinity such that the antibody is useful as a diagnostic and/or therapeutic agent in targeting CD2. The amino acid sequence of human CD2 is described in SEQ ID NO: 235.

As used herein, the terms “condition” and “conditioning” refer to processes by which a patient is prepared for receipt of a transplant, e.g., a transplant containing hematopoietic stem cells (HSCs). Such procedures promote the engraftment of a hematopoietic stem cell transplant (for instance, as inferred from a sustained increase in the quantity of viable hematopoietic stem cells within a blood sample isolated from a patient following a conditioning procedure and subsequent hematopoietic stem cell transplantation). According to the methods described herein, a patient may be conditioned for hematopoietic stem cell transplant therapy by administration to the patient of an ADC capable of binding an antigen expressed by hematopoietic stem cells. As described herein, the antibody may be covalently conjugated to a cytotoxin so as to form an ADC. Administration of an ADC capable of binding one or more of the foregoing antigens to a patient in need of hematopoietic stem cell transplant therapy can promote the engraftment of a hematopoietic stem cell graft, for example, by selectively depleting endogenous hematopoietic stem cells, thereby creating a vacancy filled by an exogenous hematopoietic stem cell transplant.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method.

As used herein, “CRU (competitive repopulating unit)” refers to a unit of measure of long-term engrafting stem cells, which can be detected after in-vivo transplantation.

As used herein, the term “donor” refers to a human or animal from which one or more cells are isolated prior to administration of the cells, or progeny thereof, into a recipient. The one or more cells may be, for example, a population of hematopoietic stem cells.

As used herein, the term “endogenous” describes a substance, such as a molecule, cell, tissue, or organ (e.g., a hematopoietic stem cell or a cell of hematopoietic lineage, such as a megakaryocyte, thrombocyte, platelet, erythrocyte, mast cell, myeoblast, basophil, neutrophil, eosinophil, microglial cell, granulocyte, monocyte, osteoclast, antigen-presenting cell, macrophage, dendritic cell, natural killer cell, T-lymphocyte, or B-lymphocyte) that is found naturally in a particular organism, such as a human patient.

As used herein, the term “engraftment potential” is used to refer to the ability of hematopoietic stem and progenitor cells to repopulate a tissue, whether such cells are naturally circulating or are provided by transplantation. The term encompasses all events surrounding or leading up to engraftment, such as tissue homing of cells and colonization of cells within the tissue of interest. The engraftment efficiency or rate of engraftment can be evaluated or quantified using any clinically acceptable parameter as known to those of skill in the art and can include, for example, assessment of competitive repopulating units (CRU); incorporation or expression of a marker in tissue(s) into which stem cells have homed, colonized, or become engrafted; or by evaluation of the progress of a subject through disease progression, survival of hematopoietic stem and progenitor cells, or survival of a recipient. Engraftment can also be determined by measuring white blood cell counts in peripheral blood during a post-transplant period. Engraftment can also be assessed by measuring recovery of marrow cells by donor cells in a bone marrow aspirate sample.

As used herein, the term “exogenous” describes a substance, such as a molecule, cell, tissue, or organ (e.g., a hematopoietic stem cell or a cell of hematopoietic lineage, such as a megakaryocyte, thrombocyte, platelet, erythrocyte, mast cell, myeoblast, basophil, neutrophil, eosinophil, microglial cell, granulocyte, monocyte, osteoclast, antigen-presenting cell, macrophage, dendritic cell, natural killer cell, T-lymphocyte, or B-lymphocyte) that is not found naturally in a particular organism, such as a human patient. Exogenous substances include those that are provided from an external source to an organism or to cultured matter extracted therefrom.

As used herein, the term “hematopoietic stem cells” (“HSCs”) refers to immature blood cells having the capacity to self-renew and to differentiate into mature blood cells containing diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Such cells may include CD34⁺ cells. CD34⁺ cells are immature cells that express the CD34 cell surface marker.

As used herein, the term “hematopoietic stem cell functional potential” refers to the functional properties of hematopoietic stem cells which include 1) multi-potency (which refers to the ability to differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells), 2) self-renewal (which refers to the ability of hematopoietic stem cells to give rise to daughter cells that have equivalent potential as the mother cell, and further that this ability can repeatedly occur throughout the lifetime of an individual without exhaustion), and 3) the ability of hematopoietic stem cells or progeny thereof to be reintroduced into a transplant recipient whereupon they home to the hematopoietic stem cell niche and re-establish productive and sustained hematopoiesis.

As used herein, the term “human antibody” is intended to include antibodies having variable and constant regions derived from human germline immunoglobulin sequences. A human antibody may include amino acid residues not encoded by human germline immunoglobulin sequences (e.g., mutations introduced by random or site-specific mutagenesis in vitro or during gene rearrangement or by somatic mutation in vivo). However, the term “human antibody”, as used herein, is not intended to include antibodies in which CDR sequences derived from the germline of another mammalian species, such as a mouse, have been grafted onto human framework sequences. A human antibody can be produced in a human cell (for example, by recombinant expression) or by a non-human animal or a prokaryotic or eukaryotic cell that is capable of expressing functionally rearranged human immunoglobulin (such as heavy chain and/or light chain) genes. When a human antibody is a single chain antibody, it can include a linker peptide that is not found in native human antibodies. For example, an Fv can contain a linker peptide, such as two to about eight glycine or other amino acid residues, which connects the variable region of the heavy chain and the variable region of the light chain. Such linker peptides are considered to be of human origin. Human antibodies can be made by a variety of methods known in the art including phage display methods using antibody libraries derived from human immunoglobulin sequences. Human antibodies can also be produced using transgenic mice that are incapable of expressing functional endogenous immunoglobulins, but which can express human immunoglobulin genes (see, for example, PCT Publication Nos. WO 1998/24893; WO 1992/01047; WO 1996/34096; WO 1996/33735; U.S. Pat. Nos. 5,413,923; 5,625,126; 5,633,425; 5,569,825; 5,661,016; 5,545,806; 5,814,318; 5,885,793; 5,916,771; and 5,939,598).

As used herein, patients that are “in need of” a hematopoietic stem cell transplant include patients that exhibit a defect or deficiency in one or more blood cell types, as well as patients having a stem cell disorder, autoimmune disease, cancer, or other pathology described herein. Hematopoietic stem cells generally exhibit 1) multi-potency, and can thus differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells), 2) self-renewal, and can thus give rise to daughter cells that have equivalent potential as the mother cell, and 3) the ability to be reintroduced into a transplant recipient whereupon they home to the hematopoietic stem cell niche and re-establish productive and sustained hematopoiesis. Hematopoietic stem cells can thus be administered to a patient defective or deficient in one or more cell types of the hematopoietic lineage in order to re-constitute the defective or deficient population of cells in vivo. For example, the patient may be suffering from cancer, and the deficiency may be caused by administration of a chemotherapeutic agent or other medicament that depletes, either selectively or non-specifically, the cancerous cell population. Additionally or alternatively, the patient may be suffering from a hemoglobinopathy (e.g., a non-malignant hemoglobinopathy), such as sickle cell anemia, thalassemia, Fanconi anemia, aplastic anemia, and Wiskott-Aldrich syndrome. The subject may be one that is suffering from adenosine deaminase severe combined immunodeficiency (ADA SCID), HIV/AIDS, metachromatic leukodystrophy, Diamond-Blackfan anemia, and Schwachman-Diamond syndrome. The subject may have or be affected by an inherited blood disorder (e.g., sickle cell anemia) or an autoimmune disorder. Additionally or alternatively, the subject may have or be affected by a malignancy, such as neuroblastoma or a hematologic cancer. For instance, the subject may have a leukemia, lymphoma, or myeloma. In some embodiments, the subject has acute myeloid leukemia, acute lymphoid leukemia, chronic myeloid leukemia, chronic lymphoid leukemia, multiple myeloma, diffuse large B-cell lymphoma, or non-Hodgkin's lymphoma. In some embodiments, the subject has myelodysplastic syndrome. In some embodiments, the subject has an autoimmune disease, such as scleroderma, multiple sclerosis, ulcerative colitis, Crohn's disease, Type 1 diabetes, or another autoimmune pathology described herein. In some embodiments, the subject is in need of chimeric antigen receptor T-cell (CART) therapy. In some embodiments, the subject has or is otherwise affected by a metabolic storage disorder. The subject may suffer or otherwise be affected by a metabolic disorder selected from the group consisting of glycogen storage diseases, mucopolysaccharidoses, Gaucher's Disease, Hurlers Disease, sphingolipidoses, metachromatic leukodystrophy, or any other diseases or disorders which may benefit from the treatments and therapies disclosed herein and including, without limitation, severe combined immunodeficiency, Wiscott-Aldrich syndrome, hyper immunoglobulin M (IgM) syndrome, Chediak-Higashi disease, hereditary lymphohistiocytosis, osteopetrosis, osteogenesis imperfecta, storage diseases, thalassemia major, sickle cell disease, systemic sclerosis, systemic lupus erythematosus, multiple sclerosis, juvenile rheumatoid arthritis and those diseases, or disorders described in “Bone Marrow Transplantation for Non-Malignant Disease,” ASH Education Book, 1:319-338 (2000), the disclosure of which is incorporated herein by reference in its entirety as it pertains to pathologies that may be treated by administration of hematopoietic stem cell transplant therapy. Additionally or alternatively, a patient “in need of” a hematopoietic stem cell transplant may one that is or is not suffering from one of the foregoing pathologies, but nonetheless exhibits a reduced level (e.g., as compared to that of an otherwise healthy subject) of one or more endogenous cell types within the hematopoietic lineage, such as megakaryocytes, thrombocytes, platelets, erythrocytes, mast cells, myeoblasts, basophils, neutrophils, eosinophils, microglia, granulocytes, monocytes, osteoclasts, antigen-presenting cells, macrophages, dendritic cells, natural killer cells, T-lymphocytes, and B-lymphocytes. One of skill in the art can readily determine whether one's level of one or more of the foregoing cell types, or other blood cell type, is reduced with respect to an otherwise healthy subject, for instance, by way of flow cytometry and fluorescence activated cell sorting (FACS) methods, among other procedures, known in the art.

As used herein, the term “recipient” refers to a patient that receives a transplant, such as a transplant containing a population of hematopoietic stem cells. The transplanted cells administered to a recipient may be, e.g., autologous, syngeneic, or allogeneic cells.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) taken from a subject.

As used herein, the term “scFv” refers to a single chain Fv antibody in which the variable domains of the heavy chain and the light chain from an antibody have been joined to form one chain. scFv fragments contain a single polypeptide chain that includes the variable region of an antibody light chain (VL) (e.g., CDR-L1, CDR-L2, and/or CDR-L3) and the variable region of an antibody heavy chain (V_{H}) (e.g., CDR-H1, CDR-H2, and/or CDR-H3) separated by a linker. The linker that joins the V_{L} and V_{H} regions of a scFv fragment can be a peptide linker composed of proteinogenic amino acids.

As used herein, the terms “subject” and “patient” refer to an organism, such as a human, that receives treatment for a particular disease or condition as described herein. For instance, a patient, such as a human patient, may receive treatment prior to hematopoietic stem cell transplant therapy in order to promote the engraftment of exogenous hematopoietic stem cells.

As used herein, the phrase “substantially cleared from the blood” refers to a point in time following administration of a therapeutic agent to a patient when the concentration of the therapeutic agent in a blood sample isolated from the patient is such that the therapeutic agent is not detectable by conventional means (for instance, such that the therapeutic agent is not detectable above the noise threshold of the device or assay used to detect the therapeutic agent). A variety of techniques known in the art can be used to detect antibodies, antibody fragments, and protein ligands, such as ELISA-based detection assays known in the art or described herein. Additional assays that can be used to detect antibodies, or antibody fragments, include immunoprecipitation techniques and immunoblot assays, among others known in the art.

As used herein, the phrase “stem cell disorder” broadly refers to any disease, disorder, or condition that may be treated or cured by conditioning a subject's target tissues, and/or by ablating an endogenous stem cell population in a target tissue (e.g., ablating an endogenous hematopoietic stem or progenitor cell population from a subject's bone marrow tissue) and/or by engrafting or transplanting stem cells in a subject's target tissues. For example, Type I diabetes has been shown to be cured by hematopoietic stem cell transplant and may benefit from conditioning in accordance with the compositions and methods described herein. Additional disorders that can be treated using the compositions and methods described herein include, without limitation, sickle cell anemia, thalassemias, Fanconi anemia, aplastic anemia, Wiskott-Aldrich syndrome, ADA SCID, HIV/AIDS, metachromatic leukodystrophy, Diamond-Blackfan anemia, and Schwachman-Diamond syndrome. Additional diseases that may be treated using the patient conditioning and/or hematopoietic stem cell transplant methods described herein include inherited blood disorders (e.g., sickle cell anemia) and autoimmune disorders, such as scleroderma, multiple sclerosis, ulcerative colitis, and Crohn's disease. Additional diseases that may be treated using the conditioning and/or transplantation methods described herein include a malignancy, such as a neuroblastoma or a hematologic cancers, such as leukemia, lymphoma, and myeloma. For instance, the cancer may be acute myeloid leukemia, acute lymphoid leukemia, chronic myeloid leukemia, chronic lymphoid leukemia, multiple myeloma, diffuse large B-cell lymphoma, or non-Hodgkin's lymphoma. Additional diseases treatable using the conditioning and/or transplantation methods described herein include myelodysplastic syndrome. In some embodiments, the subject has or is otherwise affected by a metabolic storage disorder. For example, the subject may suffer or otherwise be affected by a metabolic disorder selected from the group consisting of glycogen storage diseases, mucopolysaccharidoses, Gaucher's Disease, Hurlers Disease, sphingolipidoses, metachromatic leukodystrophy, or any other diseases or disorders which may benefit from the treatments and therapies disclosed herein and including, without limitation, severe combined immunodeficiency, Wiscott-Aldrich syndrome, hyper immunoglobulin M (IgM) syndrome, Chediak-Higashi disease, hereditary lymphohistiocytosis, osteopetrosis, osteogenesis imperfecta, storage diseases, thalassemia major, sickle cell disease, systemic sclerosis, systemic lupus erythematosus, multiple sclerosis, juvenile rheumatoid arthritis and those diseases, or disorders described in “Bone Marrow Transplantation for Non-Malignant Disease,” ASH Education Book, 1:319-338 (2000), the disclosure of which is incorporated herein by reference in its entirety as it pertains to pathologies that may be treated by administration of hematopoietic stem cell transplant therapy.

As used herein, the terms “treat” or “treatment” refers to reducing the severity and/or frequency of disease symptoms, eliminating disease symptoms and/or the underlying cause of said symptoms, reducing the frequency or likelihood of disease symptoms and/or their underlying cause, and improving or remediating damage caused, directly or indirectly, by disease. Beneficial or desired clinical results include, but are not limited to, promoting the engraftment of exogenous hematopoietic cells in a patient following antibody conditioning therapy as described herein and subsequent hematopoietic stem cell transplant therapy. Additional beneficial results include an increase in the cell count or relative concentration of hematopoietic stem cells in a patient in need of a hematopoietic stem cell transplant following conditioning therapy and subsequent administration of an exogenous hematopoietic stem cell graft to the patient. Beneficial results of therapy described herein may also include an increase in the cell count or relative concentration of one or more cells of hematopoietic lineage, such as a megakaryocyte, thrombocyte, platelet, erythrocyte, mast cell, myeoblast, basophil, neutrophil, eosinophil, microglial cell, granulocyte, monocyte, osteoclast, antigen-presenting cell, macrophage, dendritic cell, natural killer cell, T-lymphocyte, or B-lymphocyte, following conditioning therapy and subsequent hematopoietic stem cell transplant therapy. Additional beneficial results may include the reduction in quantity of a disease-causing cell population, such as a population of cancer cells or autoimmune cells. Insofar as the methods of the present invention are directed to preventing disorders, it is understood that the term “prevent” does not require that the disease state be completely thwarted. Rather, as used herein, the term preventing refers to the ability of the skilled artisan to identify a population that is susceptible to disorders, such that administration of the compounds of the present invention may occur prior to onset of a disease. The term does not imply that the disease state is completely avoided.

As used herein, “maximum plasma concentration” or “Cₘₐₓ” means the highest observed concentration of a substance (for example, an antibody or an ADC as described herein) in the plasma of a subject (e.g., a non-human primate) after administration of the substance to the subject.

As used herein, “Area Under the Curve” or “AUC” is the area under the curve in a plot of the concentration of a substance (for example, an antibody or an ADC as described herein) in plasma against time. AUC can be a measure of the integral of the instantaneous concentrations during a time interval and has the units mass×time/volume. AUC is typically calculated by the non-compartmental method (e.g. the trapezoidal method, such as linear or linear-log), or by the compartmental method. AUC is usually given for the time interval zero to infinity, and other time intervals are indicated (for example AUC_{(t1,t2)} where t1 and t2 are the starting and finishing times for the interval). Thus, as used herein “AUC_{(0-inf)}” refers to AUC from t=0, over an infinite time period.

As used herein, “Tₘₐₓ” refers to the observed time for reaching the maximum concentration of a substance in plasma of a subject (e.g., a non-human primate) after administration of that substance to the subject.

The term “acyl” as used herein refers to —C(═O)R, wherein R is hydrogen (“aldehyde”), C₁-C₁₂ alkyl, C₂-C₁₂ alkenyl, C₂-C₁₂ alkynyl, C₃-C₇ carbocyclyl, C₆-C₂₀ aryl, 5-10 membered heteroaryl, or 5-10 membered heterocyclyl, as defined herein. Non-limiting examples include formyl, acetyl, propanoyl, benzoyl, and acryloyl.

The term “C₁-C₁₂ alkyl” as used herein refers to a straight chain or branched, saturated hydrocarbon having from 1 to 12 carbon atoms. Representative C₁-C₁₂ alkyl groups include, but are not limited to, -methyl, -ethyl, -n-propyl, -n-butyl, -n-pentyl, and -n-hexyl; while branched C₁-C₁₂ alkyls include, but are not limited to, -isopropyl, -sec-butyl, -isobutyl, -tert-butyl, -isopentyl, and 2-methylbutyl. A C₁-C₁₂ alkyl group can be unsubstituted or substituted.

The term “alkenyl” as used herein refers to C₂-C₁₂ hydrocarbon containing normal, secondary, or tertiary carbon atoms with at least one site of unsaturation, i.e., a carbon-carbon, sp²double bond. Examples include, but are not limited to: ethylene or vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, and the like. An alkenyl group can be unsubstituted or substituted.

“Alkynyl” as used herein refers to a C₂-C₁₂ hydrocarbon containing normal, secondary, or tertiary carbon atoms with at least one site of unsaturation, i.e., a carbon-carbon, sp triple bond. Examples include, but are not limited to acetylenic and propargyl. An alkynyl group can be unsubstituted or substituted.

“Aryl” as used herein refers to a C₆-C₂₀ carbocyclic aromatic group. Examples of aryl groups include, but are not limited to, phenyl, naphthyl and anthracenyl. An aryl group can be unsubstituted or substituted.

“Arylalkyl” as used herein refers to an acyclic alkyl radical in which one of the hydrogen atoms bonded to a carbon atom, typically a terminal or sp³ carbon atom, is replaced with an aryl radical. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. The arylalkyl group comprises 6 to 20 carbon atoms, e.g. the alkyl moiety, including alkanyl, alkenyl or alkynyl groups, of the arylalkyl group is 1 to 6 carbon atoms and the aryl moiety is 5 to 14 carbon atoms. An alkaryl group can be unsubstituted or substituted.

“Cycloalkyl” as used herein refers to a saturated carbocyclic radical, which may be mono- or bicyclic. Cycloalkyl groups include a ring having 3 to 7 carbon atoms as a monocycle or 7 to 12 carbon atoms as a bicycle. Examples of monocyclic cycloalkyl groups include cyclopropyl, cyclobutyl, cyclopentyl, cyclohexyl, cycloheptyl, and cyclooctyl. A cycloalkyl group can be unsubstituted or substituted.

“Cycloalkenyl” as used herein refers to an unsaturated carbocyclic radical, which may be mono- or bicyclic. Cycloalkenyl groups include a ring having 3 to 6 carbon atoms as a monocycle or 7 to 12 carbon atoms as a bicycle. Examples of monocyclic cycloalkenyl groups include 1-cyclopent-1-enyl, 1-cyclopent-2-enyl, 1-cyclopent-3-enyl, 1-cyclohex-1-enyl, 1-cyclohex-2-enyl, and 1-cyclohex-3-enyl. A cycloalkenyl group can be unsubstituted or substituted.

“Heteroaralkyl” as used herein refers to an acyclic alkyl radical in which one of the hydrogen atoms bonded to a carbon atom, typically a terminal or sp³ carbon atom, is replaced with a heteroaryl radical. Typical heteroarylalkyl groups include, but are not limited to, 2-benzimidazolylmethyl, 2-furylethyl, and the like. The heteroarylalkyl group comprises 6 to 20 carbon atoms, e.g. the alkyl moiety, including alkanyl, alkenyl or alkynyl groups, of the heteroarylalkyl group is 1 to 6 carbon atoms and the heteroaryl moiety is 5 to 14 carbon atoms and 1 to 3 heteroatoms selected from N, O, P, and S. The heteroaryl moiety of the heteroarylalkyl group may be a monocycle having 3 to 7 ring members (2 to 6 carbon atoms or a bicycle having 7 to 10 ring members (4 to 9 carbon atoms and 1 to 3 heteroatoms selected from N, O, P, and S), for example: a bicyclo[4,5], [5,5], [5,6], or [6,6] system.

“Heteroaryl” and “heterocycloalkyl” as used herein refer to an aromatic or non-aromatic ring system, respectively, in which one or more ring atoms is a heteroatom, e.g. nitrogen, oxygen, and sulfur. The heteroaryl or heterocycloalkyl radical comprises 2 to 20 carbon atoms and 1 to 3 heteroatoms selected from N, O, P, and S. A heteroaryl or heterocycloalkyl may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S), for example: a bicyclo[4,5], [5,5], [5,6], or [6,6] system. Heteroaryl and heterocycloalkyl can be unsubstituted or substituted.

Heteroaryl and heterocycloalkyl groups are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566.

Examples of heteroaryl groups include by way of example and not limitation pyridyl, thiazolyl, tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, benzotriazolyl, benzisoxazolyl, and isatinoyl.

Examples of heterocycloalkyls include by way of example and not limitation dihydroypyridyl, tetrahydropyridyl (piperidyl), tetrahydrothiophenyl, piperidinyl, 4-piperidonyl, pyrrolidinyl, 2-pyrrolidonyl, tetrahydrofuranyl, tetrahydropyranyl, bis-tetrahydropyranyl, tetrahydroquinolinyl, tetrahydroisoquinolinyl, decahydroquinolinyl, octahydroisoquinolinyl, piperazinyl, quinuclidinyl, and morpholinyl.

By way of example and not limitation, carbon bonded heteroaryls and heterocycloalkyls are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heteroaryls and heterocycloalkyls are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or beta-carboline. Still more typically, nitrogen bonded heterocycles include 1-aziridyl, 1-azetedyl, 1-pyrrolyl, 1-imidazolyl, 1-pyrazolyl, and 1-piperidinyl.

“Substituted” as used herein and as applied to any of the above alkyl, alkenyl, alkynyl, aryl, arylalkyl, cycloalkyl, heteroaryl, heterocyclyl, and the like, means that one or more hydrogen atoms are each independently replaced with a substituent. Typical substituents include, but are not limited to, —X, —R, —OH, —OR, —SH, —SR, NH₂, —NHR, —N(R)₂, —N⁺(R)₃, —CX₃, —CN, —OCN, —SCN, —NCO, —NCS, —NO, —NO₂, —N₃, —NC(═O)H, —NC(═O)R, —C(═O)H, —C(═O)R, —C(═O)NH₂, —C(═O)N(R)₂, —SO₃—, —SO₃H, —S(═O)₂R, —OS(═O)₂OR, —S(═O)₂NH₂, —S(═O)₂N(R)₂, —S(═O)R, —OP(═O)(OH)₂, —OP(═O)(OR)₂, —P(═O)(OR)₂, —PO₃, —PO₃H₂, —C(═O)X, —C(═S)R, —CO₂H, —CO₂R, —CO₂—, —C(═S)OR, —C(═O)SR, —C(═S)SR, —C(═O)NH₂, —C(═O)N(R)₂, —C(═S)NH₂, —C(═S)N(R)₂, —C(═NH)NH₂, and —C(═NR)N(R)₂; wherein each X is independently selected for each occasion from F, C, Br, and I; and each R is independently selected for each occasion from C₁-C₁₂ alkyl, C₆-C₂₀ aryl, C₃-C₁₄ heterocycloalkyl or heteroaryl, protecting group and prodrug moiety. Wherever a group is described as “optionally substituted,” that group can be substituted with one or more of the above substituents, independently for each occasion.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene,” “alkenylene,” “arylene,” “heterocycloalkylene,” and the like.

Wherever a substituent is depicted as a di-radical (i.e., has two points of attachment to the rest of the molecule), it is to be understood that the substituent can be attached in any directional configuration unless otherwise indicated.

### Conditioning Methods Using Antibody Drug Conjugates (ADCs)

Antibody drug conjugates (ADCs) as described herein can be administered to a human patient (e.g., a human patient suffering from cancer, an autoimmune disease, in need of hematopoietic stem cell transplant therapy, or at risk of GvHD) for conditioning prior to stem cell transplantation. The methods described herein can be used to deliver an ADC to a human patient in a conditioning dosing regimen that reduces toxicity and serious adverse events, yet remains effective as a conditioning agent. As described in the examples below, by dividing the ADC dose into two or more doses (referred to herein as a “multi-dose” or interchangeably as a “fractionated dose”), toxicity can be reduced while the ADC remains effective for treatment, e.g., conditioning. In one embodiment, the dose amounts for each delivery in the dosing regimen of the invention are about the same.

The sections that follow provide a description of ADCs that can be administered to a patient in need of a transplantation, e.g., hematopoietic stem cell transplant, in order to promote engraftment of hematopoietic stem cells, as well as methods of administering such therapeutics to a patient prior to hematopoietic stem cell transplantation.

In the case of a conditioning therapy prior to hematopoietic stem cell transplantation, the ADC can be administered to the patient at a time that optimally promotes engraftment of the exogenous hematopoietic stem cells, for instance, from 1 hour to 1 week (e.g., 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 2 days, 3 days, 4 days, 5 days, 6 days, or 7 days) or more prior to administration of the exogenous hematopoietic stem cell transplant.

In accordance with the methods disclosed herein, the ADC is administered to the human patient in a conditioning therapy in two doses that are separated in time. In one embodiment, the first dose is administered on day 1 and the second dose is administered about 1 hour, about 2 hours, about 3 hours, about 4 hours, about 5 hours, about 6 hours, about 7 hours, about 8 hours, about 9 hours, about 10 hours, about 11 hours, or about 12 hours after the first dose is administered. In another embodiment, the first dose is administered on day 1 and the second dose is administered on day 2, on day 3, on day 4, on day 5, on day 6, on day 7, on day 8, on day 9, on day 10, on day 11, or on day 12 of the dosing regimen.

The dose of the ADC can be either a fixed dose (mg) or a weight based dose (mg/kg), or a combination thereof. For example, the dose of the ADC can be 0.03-0.29 mg/kg, about 0.03-0.25 mg/kg, about 0.03-0.20 mg/kg, about 0.03-0.15 mg/kg, about 0.03-0.10 mg/kg, about 0.05-0.10 mg/kg, or about 0.05-0.07 mg/kg. In other embodiments, the dose of the ADC administered to the subject is about 0.03-0.6 mg/kg; about 0.03-0.55 mg/kg; about 0.03-0.5 mg/kg; about 0.03-0.45 mg/kg; about 0.03-0.4 mg/kg; about 0.03-0.35 mg/kg; about 0.03-0.3 mg/kg; about 0.03-0.25 mg/kg; about 0.03-0.2 mg/kg; about 0.03-0.15 mg/kg; about 0.03-0.1 mg/kg; about 0.1-0.6 mg/kg; about 0.1-0.55 mg/kg; about 0.1-0.5 mg/kg; about 0.1-0.45 mg/kg; about 0.1-0.4 mg/kg; about 0.1-0.35 mg/kg; about 0.1-0.3 mg/kg; about 0.1-0.25 mg/kg; about 0.1-0.2 mg/kg; about 0.1-0.15 mg/kg; about 0.15-0.6 mg/kg; about 0.15-0.55 mg/kg; about 0.15-0.5 mg/kg; about 0.15-0.45 mg/kg; about 0.15-0.4 mg/kg; about 0.15-0.35 mg/kg; about 0.15-0.3 mg/kg; about 0.15-0.25 mg/kg; or about 0.15-0.2 mg/kg.

In one embodiment, the dose of the ADC is about 1 mg/kg. In one embodiment, the dose of the ADC is about 0.01-1 mg/kg; about 0.01-0.95 mg/kg; about 0.01-0.9 mg/kg; about 0.01-0.85 mg/kg; about 0.01-0.8 mg/kg; about 0.01-0.75 mg/kg; about 0.01-0.7 mg/kg; about 0.01-0.65 mg/kg; about 0.01-0.6 mg/kg; about 0.01-0.55 mg/kg; about 0.01-0.5 mg/kg; about 0.01-0.45 mg/kg; about 0.01-0.4 mg/kg; about 0.01-0.35 mg/kg; about 0.01-0.2 mg/kg; about 0.01-0.15 mg/kg; about 0.01-0.1 mg/kg; or about 0.01-0.05 mg/kg.

In one embodiment, the dose of the ADC is about 2 mg/kg. In one embodiment, the dose of the ADC is about 0.01-2 mg/kg; about 0.01-1.8 mg/kg; about 0.01-1.6 mg/kg; about 0.01-1.4 mg/kg; or about 0.01-1.2 mg/kg. In one embodiment, the dose of the ADC is about 0.05-2 mg/kg; about 0.05-1.8 mg/kg; about 0.05-1.6 mg/kg; about 0.05-1.4 mg/kg; or about 0.05-1.2 mg/kg.

In one embodiment, the dose of the ADC is about 3 mg/kg. In one embodiment, the dose of the ADC is about 0.01-3 mg/kg; about 0.01-2.8 mg/kg; about 0.01-2.6 mg/kg; about 0.01-2.4 mg/kg; or about 0.01-2.2 mg/kg. In one embodiment, the dose of the ADC is about 0.05-3 mg/kg; about 0.05-2.8 mg/kg; about 0.05-2.6 mg/kg; about 0.05-2.4 mg/kg; or about 0.05-2.2 mg/kg.

In one embodiment, the dose of the ADC is about 4 mg/kg. In one embodiment, the dose of the ADC is about 0.01-4 mg/kg; about 0.01-3.8 mg/kg; about 0.01-3.6 mg/kg; about 0.01-3.4 mg/kg; or about 0.01-3.2 mg/kg. In one embodiment, the dose of the ADC is about 0.05-4 mg/kg; about 0.05-3.8 mg/kg; about 0.05-3.6 mg/kg; about 0.05-3.4 mg/kg; or about 0.05-3.2 mg/kg.

In one embodiment, the dose of the ADC is about 5 mg/kg. In one embodiment, the dose of the ADC is about 0.01-5 mg/kg; about 0.01-4.8 mg/kg; about 0.01-4.6 mg/kg; about 0.01-4.4 mg/kg; or about 0.01-4.2 mg/kg. In one embodiment, the dose of the ADC is about 0.05-5 mg/kg; about 0.05-4.8 mg/kg; about 0.05-4.6 mg/kg; about 0.05-4.4 mg/kg; or about 0.05-4.2 mg/kg.

In one embodiment, the dose of the ADC is about 0.2-0.8 mg/kg. In one embodiment, the dose of the ADC is about 0.2-0.7 mg/kg; about 0.2-0.6 mg/kg; about 0.3-0.7 mg/kg; or about 0.3-0.6 mg/kg.

In one embodiment, the dose of the ADC is about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.15 mg/kg, about 0.2 mg/kg, about 0.25 mg/kg, about 0.3 mg/kg, about 0.35 mg/kg, about 0.4 mg/kg, about 0.45 mg/kg, about 0.5 mg/kg, about 0.55 mg/kg, about 0.6 mg/kg, about 0.65 mg/kg, about 0.7 mg/kg, about 0.75 mg/kg, about 0.8 mg/kg, about 0.85 mg/kg, about 0.9 mg/kg, about 0.95 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, or about 5 mg/kg.

In one embodiment, the ADC is administered to the patient at a dose of about 0.1 mg/kg. In one embodiment, the ADC is administered to the patient at a dose of about 0.3 mg/kg. In one embodiment, the ADC is administered to the patient at a dose of about 0.6 mg/kg. In one embodiment, the ADC is administered to the patient at a dose of about 1.0 mg/kg.

In yet another embodiment, the ADC is administered to the human patient at a fixed dose of about 2-12 mg, about 2-11 mg, about 2-10 mg, about 2-9 mg, about 2-8 mg, about 2-7 mg, about 2-6 mg, about 2-5 mg, about 2-4 mg, or 2-3 mg. In one embodiment, a dose of the ADC is about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, or about 12 mg, In one embodiment, a dose of the ADC is about 0.01 mg, about 0.02 mg, about 0.03 mg, about 0.04 mg, about 0.05 mg, about 0.06 mg, about 0.07 mg, about 0.08 mg, about 0.09 mg, about 1 mg, about 1.5 mg, about 2 mg, about 2.5 mg, about 3 mg, about 3.5 mg, about 4 mg, about 4.5 mg, about 5 mg. Ranges of the aforementioned doses are included in the dose amounts that can be used in the methods disclosed here as well.

In one embodiment, the dosing regimen includes administration of 0.1 mg/kg of the ADC on days 1 and 3. In another embodiment, the dosing regimen includes administration of 0.2 mg/kg of the ADC on days 1 and 3. In another embodiment, the dosing regimen includes administration of 0.3 mg/kg of the ADC on days 1 and 3. In yet another embodiment, the dosing regimen includes administration of 0.3 mg/kg of the ADC on day 1 and 0.2 mg/kg of the ADC on day 3. Alternatively, the dosing regimen includes administration of about 0.1 mg/kg Q3D×2; or about 0.3 mg/kg Q3D×2; or about 0.6 mg/kg Q3D×2.

Doses used in the dosing regimen of the invention may also be calculated according to the monkey doses described in the examples herein using standard methods known in the art (e.g., U.S. Department of Health and Human Services Food and Drug Administration Center for Drug Evaluation and Research (CDER). “Guidance for Industry Estimating the Maximum Safe Starting Dose in Initial Clinical Trials for Therapeutics in Adult Healthy Volunteers.” July 2005; http://www.fda.gov/cder/guidance/index.htm). For example, in some embodiments, a human equivalent dose (HED) may be derived from animal dosage data using a conversion factor. As one non-limiting example, Nair and Jacob, J. Basic Clin. Pharma. (2016) 7:27-31 disclose methods of extrapolation of dose between species. For instance, in one non-limiting example, HED may be derived from a rhesus monkey dose by multiplying the rhesus monkey dose by about 0.324. In another non-limiting example, HED may be derived from a mouse dose by multiplying the mouse dose by about 0.081.

Pharmacodynamic parameters, such as AUC, Cₘₐₓ and Tₘₐₓ may also be used to characterize the present invention. A skilled artisan will understand the various methods for measuring and calculating the pharmacokinetic (for example, but not limited to, AUC, Cₘₐₓ and Tₘₐₓ) parameters described herein. Furthermore, the skilled artisan will understand the various methods for making statistical comparisons (for example, but not limited to, comparisons of change from baseline to post-treatment and/or comparisons among treatment groups) and/or analysis of the pharmacokinetic parameters described herein.

In one embodiment, there is provided an ADC dosing regimen for use in the methods described herein, wherein the dosing regimen provides an AUC_{(0-inf)} value of between 1-500 μg/mL hour, as determined by methods known to one of skill in the art. In one embodiment, the dosing regimen provides an AUC_{(0-inf)} value of between about 1-50 μg/mL hour, of between about 50-100 μg/mL hour, of between about 100-150 μg/mL hour, of between about 150-200 μg/mL hour, of between about 200-250 μg/mL hour, of between about 250-300 μg/mL hour, of between about 300-350 μg/mL hour, of between about 350-400 μg/mL hour, of between about 400-450 μg/mL hour, of between about 450-500 μg/mL hour, as determined by methods known to one of skill in the art. In other embodiments, the dosing regimen provides an AUC_{(0-inf)} value including, but are not limited to about 10 μg/mL hour, about 20 μg/mL hour, about 30 μg/mL hour, about 40 μg/mL hour, about 50 μg/mL hour, about 60 μg/mL hour, about 70 μg/mL hour, about 80 μg/mL hour, about 90 μg/mL hour, about 100 μg/mL hour, about 110 μg/mL hour, about 120 μg/mL hour, about 130 μg/mL hour, about 140 μg/mL hour, about 150 μg/mL hour, about 160 μg/mL hour, about 170 μg/mL hour, about 180 μg/mL hour, about 190 μg/mL hour, about 200 μg/mL hour, about 210 μg/mL hour, about 220 μg/mL hour, about 230 μg/mL hour, about 240 μg/mL hour, about 250 μg/mL hour, about 260 μg/mL hour, about 270 μg/mL hour, about 280 μg/mL hour, about 290 μg/mL hour, about 300 μg/mL hour, about 310 μg/mL hour, about 320 μg/mL hour, about 330 μg/mL hour, about 340 μg/mL hour, about 350 μg/mL hour, about 360 μg/mL hour, about 370 μg/mL hour, about 380 μg/mL hour, about 390 μg/mL hour, about 400 μg/mL hour, about 410 μg/mL hour, about 420 μg/mL hour, about 430 μg/mL hour, about 440 μg/mL hour, about 450 μg/mL hour, about 460 μg/mL hour, about 470 μg/mL hour, about 480 μg/mL hour, about 490 μg/mL hour, about 500 μg/mL hour, as determined by methods known to one of skill in the art. In another embodiment, all of these values and ranges may be ±10%.

In another embodiment, there is provided an ADC dosing regimen for use in the methods described herein, wherein the dosing regimen provides a Cₘₐₓ value of between 0.1-20 μg/mL, as determined by methods known to one of skill in the art. In one embodiment, the dosing regimen provides a Cₘₐₓ value of between about 0.1-0.5 μg/mL, of between about 0.5-1.0 μg/mL, of between about 1.0-1.5 μg/mL, of between about 1.5-2.0 μg/mL, of between about 2.0-2.5 μg/mL, of between about 2.5-3.0 μg/mL, of between about 3.0-3.5 μg/mL, of between about 3.5-4.0 μg/mL, of between about 4.0-4.5 μg/mL, of between about 4.5-5.0 μg/mL, of between about 5.0-5.5 μg/mL, of between about 5.5-6.0 μg/mL, of between about 6.0-6.5 μg/mL, of between about 6.5-7.0 μg/mL, of between about 7.0-7.5 μg/mL, of between about 7.5-8.0 μg/mL, of between about 8.0-8.5 μg/mL, of between about 8.5-9.0 μg/mL, of between about 9.0-9.5 μg/mL, of between about 9.5-10.0 μg/mL, of between about 10.0-10.5 μg/mL, of between about 10.5-11.0 μg/mL, of between about 11.0-11.5 μg/mL, of between about 11.5-12.0 μg/mL, of between about 12.0-12.5 μg/mL, of between about 12.5-13.0 μg/mL, of between about 13.0-13.5 μg/mL, of between about 13.5-14.0 μg/mL, of between about 14.0-14.5 μg/mL, of between about 14.5-15.0 μg/mL, of between about 15.0-15.5 μg/mL, of between about 15.5-16.0 μg/mL, of between about 16.0-16.5 μg/mL, of between about 16.5-17.0 μg/mL, of between about 17.0-17.5 μg/mL, of between about 18.5-19.0 μg/mL, of between about 19.0-19.5 μg/mL, of between about 19.5-20.0 μg/mL, as determined by methods known to one of skill in the art. In other embodiments, the dosing regimen provides a Cₘₐₓ value including, but are not limited to about 0.1 μg/mL, about 0.5 μg/mL, about 1.0 μg/mL, about 1.5 μg/mL, about 2.0 μg/mL, about 2.5 μg/mL, about 3.0 μg/mL, about 3.5 μg/mL, about 4.0 μg/mL, about 4.5 μg/mL, about 5.0 μg/mL, about 5.5 μg/mL, about 6.0 μg/mL, about 6.5 μg/mL, about 7.0 μg/mL, about 7.5 μg/mL, about 8.0 μg/mL, about 8.5 μg/mL, about 9.0 μg/mL, about 9.5 μg/mL, about 10.0 μg/mL, about 10.5 μg/mL, about 11.0 μg/mL, about 11.5 μg/mL, about 12.0 μg/mL, about 12.5 μg/mL, about 13.0 μg/mL, about 13.5 μg/mL, about 14.0 μg/mL, about 14.5 μg/mL, about 15.0 μg/mL, about 15.5 μg/mL, about 16.0 μg/mL, about 16.5 μg/mL, about 17.0 μg/mL, about 17.5 μg/mL, about 18.0 μg/mL, about 18.5 μg/mL, about 19.0 μg/mL, about 19.5 μg/mL, about 20.0 μg/mL, as determined by methods known to one of skill in the art. In another embodiment, all of these values and ranges may be ±10%.

In another embodiment there is provided an antibody or an ADC dosing regimen for use in the methods described herein, wherein the dosing regimen provides both a AUC_{(0-inf)} value (or range) and a Cₘₐₓ value (or range), characterized by the values and ranges of values are described above.

In one embodiment, the invention thus provides compositions and methods of promoting the engraftment of transplanted hematopoietic stem cells by administration of an ADC capable of binding an antigen expressed by T cells. This administration can cause the selective depletion of a population of endogenous T cells, such as CD4+ and CD8+ T cells. This selective depletion of T cells can, in turn, prevent graft rejection following transplantation of an exogenous (for instance, an autologous, allogeneic, or syngeneic) hematopoietic stem cell graft. For instance, the selective depletion of CD4+ and/or CD8+ T cells using an ADC as described herein can attenuate a T cell-mediated immune response that may occur against a transplanted hematopoietic stem cell graft. The invention is based in part on the discovery that ADCs can be administered according to a certain low toxicity protocol to a patient in need of hematopoietic stem cell transplant therapy in order to promote the survival and engraftment potential of transplanted hematopoietic stem cells and reduce toxicity of the ADC to the patient.

Engraftment of hematopoietic stem cell transplants due to the administration of an ADC described herein can manifest in a variety of empirical measurements. For instance, engraftment of transplanted hematopoietic stem cells can be evaluated by assessing the quantity of competitive repopulating units (CRU) present within the bone marrow of a patient following administration of an antibody or antigen-binding fragment thereof capable of binding an antigen described herein and subsequent administration of a hematopoietic stem cell transplant. Additionally, one can observe engraftment of a hematopoietic stem cell transplant by incorporating a reporter gene, such as an enzyme that catalyzes a chemical reaction yielding a fluorescent, chromophoric, or luminescent product, into a vector with which the donor hematopoietic stem cells have been transfected and subsequently monitoring the corresponding signal in a tissue into which the hematopoietic stem cells have homed, such as the bone marrow. One can also observe hematopoietic stem cell engraftment by evaluation of the quantity and survival of hematopoietic stem and progenitor cells, for instance, as determined by fluorescence activated cell sorting (FACS) analysis methods known in the art. Engraftment can also be determined by measuring white blood cell counts in peripheral blood during a post-transplant period, and/or by measuring recovery of marrow cells by donor cells in a bone marrow aspirate sample.

Pharmaceutical formulations comprising ADCs as described herein can be prepared by mixing such ADC with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

For reasons described herein, it is challenging to achieve an appropriate dosing scheme for a conditioning therapy (e.g., conditioning prior to hematopoietic stem cell transplantation) that allows for both efficient on-target cell depletion (e.g., reticulocytes, platelets, and/or hematopoietic stem cells and progenitor cells) while minimizing significant off-target toxicity (e.g., significant increases in the levels of certain liver proteins), particularly when using a fast (i.e., short) half-life ADC. This problem is further complicated upon recognizing that the effectiveness of the dosing scheme for a conditioning therapy would require lower maximum plasma concentrations of the ADCs while maintaining similar on-target exposure over a sufficient period of time, a problem that is potentially further complicated by the target receptor internalization into the cell and the rate of release of the cytotoxin from the ADC.

In one embodiment, the methods disclosed herein minimize liver toxicity in the patient receiving the ADC for conditioning. For example, in certain embodiments, the methods disclosed herein result in a liver marker level remaining below a known toxic level in the patient for more than 24 hours, 48 hours, 72 hours, or 96 hours. In other embodiments, the methods disclosed herein result in a liver marker level remaining within a reference range in the patient for more than 24 hours, 48 hours, 72 hours, or 96 hours. In certain embodiments, the methods disclosed herein result in a liver marker level rising not more than 1.5-fold above a reference range, not more than 3-fold above a reference range, not more than 5-fold above a reference range, or not more than 10-fold above a reference range for more than 24 hours, 48 hours, 72 hours, or 96 hours. Examples of liver markers that can be used to test for toxicity include alanine aminotransaminase (ALT), lactate dehydrogenase (LDH), and aspartate aminotransaminase (AST). In certain embodiments, administration of an ADC as described herein, i.e., where two doses are administered instead of a single dose, results in a transient increase in a liver marker, e.g., AST, LDH, and/or ALT. In some instances, an elevated level of a liver marker indicating toxicity may be reached, but within a certain time period, e.g., about 12 hours, about 18 hours, about 24 hours, about 36 hours, about 48 hours, about 72 hours, above 3 days, about 3.5 days, about 4 days, about 4.5 days, about 5 days, about 5.5 days, about 6 days, about 6.5 days, about 7 days, about 7.5 days, or less than a week, the liver marker level returns to a normal level not associated with liver toxicity. For example, in a human (average adult male), a normal, non-toxic level of ALT is 7 to 55 units per liter (U/L); and a normal, non-toxic level of AST is 8 to 48 U/L. In certain embodiments, at least one of the patient's blood AST, ALT, or LDH levels does not reach a toxic level between administration of a first dose of the ADC and 14 days after administration of the first dose to the patient. For example, the patient may be administered a first dose and subsequently a second dose, a third dose, a fourth dose, or more doses within, e.g., 5, 10, or 14 days of being administered the first dose, yet at least one of the patient's blood AST, ALT, or LDH levels does not reach a toxic level between administration of a first dose of the ADC and 14 days after administration of the first dose to the patient.

In certain embodiments, at least one of the patient's blood AST, ALT, or LDH levels does not rise above normal levels, does not rise more than 1.5-fold above normal levels, does not rise more than 3-fold above normal levels, does not rise more than 5-fold above normal levels, or does not rise more than 10-fold above normal levels.

The present invention includes dosing regimens that reduce adverse events and toxicity using ADCs that are capable of binding an antigen expressed by a hematopoietic cell, such as a hematopoietic stem cell, an immune cell or a cancer cell. Examples of such antigens include, but are not limited to, CD117, CD2, CD5, CD45, CD252, CD134, and CD137.

### Anti-CD117 Antibodies

In one embodiment, the methods disclosed herein include administration of an anti-CD117 ADC comprising an anti-CD117 antibody conjugated to a cytotoxin via a linker. Antibodies, antigen-binding fragments thereof, described herein bind to CD117, such as GNNK+ CD117, and can be used as therapeutic agents to (i) directly treat cancers and autoimmune diseases characterized by CD117+ cells and (ii) promote the engraftment of transplanted hematopoietic stem cells in a patient in need of transplant therapy. These therapeutic activities can be caused, for instance, by the binding of anti-CD117 antibodies, or antigen-binding fragments thereof, to CD117 (e.g., GNNK+ CD117) expressed on the surface of a cell, such as a cancer cell, autoimmune cell, or hematopoietic stem cell and subsequently inducing cell death. The depletion of endogenous hematopoietic stem cells can provide a niche toward which transplanted hematopoietic stem cells can home, and subsequently establish productive hematopoiesis. In this way, transplanted hematopoietic stem cells may successfully engraft in a patient, such as human patient suffering from a stem cell disorder described herein.

Antibodies and antigen-binding fragments capable of binding human CD117 (also referred to as c-Kit, mRNA NCBI Reference Sequence: NM_000222.2, Protein NCBI Reference Sequence: NP_000213.1 (see, i.e., SEQ ID NO: 232), including those capable of binding GNNK+ CD117, can be used in conjunction with the compositions and methods described herein in order to condition a patient for hematopoietic stem cell transplant therapy. Polymorphisms affecting the coding region or extracellular domain of CD117 in a significant percentage of the population are not currently well-known in non-oncology indications. There are at least four isoforms of CD117 that have been identified, with the potential of additional isoforms expressed in tumor cells. Two of the CD117 isoforms are located on the intracellular domain of the protein, and two are present in the external juxtamembrane region. The two extracellular isoforms, GNNK+ and GNNK−, differ in the presence (GNNK+) or absence (GNNK−) of a 4 amino acid sequence. These isoforms are reported to have the same affinity for the ligand (SCF), but ligand binding to the GNNK− isoform was reported to increase internalization and degradation. The GNNK+ isoform can be used as an immunogen in order to generate antibodies capable of binding CD117, as antibodies generated against this isoform will be inclusive of the GNNK+ and GNNK− proteins.

In one embodiment, an anti-CD117 antibody that may be used in the methods and compositions (including ADCs) described herein is Antibody 77 (Ab77). The heavy chain variable region (VH) amino acid sequence of Antibody 77 (Ab77) is provided below as SEQ ID NO: 9.

(SEQ ID NO: 9)
QVQLVQSGAAVKKPGESLKISCKGSGYRFTTYWIGWVRQMPGK
GLEWMGIIYPGDSDTRYSPSFQGQVTISAGKSISTAYLQWSSL
KASDTAMYYCARHGRGYNGYEGAFDIWGQGTMVTVSS

The VH CDR amino acid sequences of Ab77 are underlined above and are as follows:

(VH CDR1; SEQ ID NO: 94)
TYWIG;
(VH CDR2; SEQ ID NO: 2)
IIYPGDSDTRYSPSFQG;
and
(VH CDR3; SEQ ID NO: 3)
HGRGYNGYEGAFDI.

The light chain variable region (VL) amino acid sequence of Ab77 is provided below as SEQ ID NO 93.

(SEQ ID NO: 93)
DIQLTQSPSSLSASVGDRVTITCRASQGVISALAWYQQKP
GKAPKLLIYDASILESGVPSRFSGSGSGTDFTLTISSLQP
EDFATYYCQQFNSYPLTFGGGTKVEIK

The VL CDR amino acid sequences of Ab77 are underlined below and are as follows: RASQGVISALA (VL CDR1; SEQ ID NO: 95); DASILES (VL CDR2; SEQ ID NO: 96); and QQFNSYPLT (VL CDR3; SEQ ID NO: 97).

In another embodiment, an anti-CD117 antibody that may be used in the methods and compositions (including ADCs) described herein is Antibody 79 (Ab79). The heavy chain variable region (VH) amino acid sequence of Ab79 is provided below as SEQ ID NO: 9.

(SEQ ID NO: 9)
QVQLVQSGAAVKKPGESLKISCKGSGYRFTTYWIGWVRQMP
GKGLEWMGIIYPGDSDTRYSPSFQGQVTISAGKSISTAYLQ
WSSLKASDTAMYYCARHGRGYNGYEGAFDIWGQGTMVTVSS

The VH CDR amino acid sequences of Ab79 are underlined below and are as follows:

(VH CDR1; SEQ ID NO: 94)
TYWIG;
(VH CDR2; SEQ ID NO: 2)
IIYPGDSDTRYSPSFQG;
and
(VH CDR3; SEQ ID NO: 3)
HGRGYNGYEGAFDI.

The light chain variable region (VL) amino acid sequence of Ab79 is provided below as SEQ ID NO: 99.

(SEQ ID NO: 99)
DIQLTQSPSSLSASVGDRVTITCRASQGVGSALAWYQQKPGKAP
KLLIYDASILESGVPSRFSGSGSGTDFTLTISSLQPEDFATYYC
QQFNSYPLTFGGGTKVEIK

The VL CDR amino acid sequences of Ab79 are underlined below and are as follows: RASQGVGSALA (VL CDR1; SEQ ID NO: 100); DASILES (VL CDR2; SEQ ID NO: 96); and QQFNSYPLT (VL CDR3; SEQ ID NO: 97).

In another embodiment, an anti-CD117 antibody that may be used in the methods and compositions (including ADCs) described herein is Antibody 81 (Ab81). The heavy chain variable region (VH) amino acid sequence of Ab81 is provided below as SEQ ID NO: 9.

(SEQ ID NO: 9)
QVQLVQSGAAVKKPGESLKISCKGSGYRFTTYWIGWVRQMPGKG
LEWMGIIYPGDSDTRYSPSFQGQVTISAGKSISTAYLQWSSLKA
SDTAMYYCARHGRGYNGYEGAFDIWGQGTMVTVSS

The VH CDR amino acid sequences of Ab81 are underlined below and are as follows:

(VH CDR1; SEQ ID NO: 94)
TYWIG;
(VH CDR2; SEQ ID NO: 2)
IIYPGDSDTRYSPSFQG;
and
(VH CDR3; SEQ ID NO: 3)
HGRGYNGYEGAFDI.

The light chain variable region (VL) amino acid sequence of Ab81 is provided below as SEQ ID NO: 102.

(SEQ ID NO: 102)
DIQLTQSPSSLSASVGDRVTITCRASQGVISALAWYQQKPGKAP
KLLIYDASTLESGVPSRFSGSGSGTDFTLTISSLQPEDFATYYC
QQFNSYPLTFGGGTKVEIK

The VL CDR amino acid sequences of Ab81 are underlined below and are as follows:

(VL CDR1; SEQ ID NO: 95)
RASQGVISALA;
(VL CDR2; SEQ ID NO: 103)
DASTLES;
and
(VL CDR3; SEQ ID NO: 97)
QQFNSYPLT.

In another embodiment, an anti-CD117 antibody that may be used in the methods and compositions (including ADCs) described herein is Antibody 85 (Ab85). The heavy chain variable region (VH) amino acid sequence of Ab85 is provided below as SEQ ID NO: 106.

(SEQ ID NO: 106)
EVQLVQSGAEVKKPGESLKISCKGSGYSFTNYWIGWVRQMPGK
GLEWMAIINPRDSDTRYRPSFQGQVTISADKSISTAYLQWSSL
KASDTAMYYCARHGRGYEGYEGAFDIWGQGTLVTVSS

The VH CDR amino acid sequences of Ab85 are underlined below and are as follows:

(VH CDR1; SEQ ID NO: 108)
NYWIG;
(VH CDR2; SEQ ID NO: 109)
IINPRDSDTRYRPSFQG;
and
(VH CDR3; SEQ ID NO: 110)
HGRGYEGYEGAFDI.

The light chain variable region (VL) amino acid sequence of Ab85 is provided below as SEQ ID NO: 107.

(SEQ ID NO: 107)
DIQMTQSPSSLSASVGDRVTITCRSSQGIRSDLGWYQQKPGKAPKL
LIYDASNLETGVPSRFSGSGSGTDFTLTISSLOPEDFATYYCQQAN
GFPLTFGGGTKVEIK

The VL CDR amino acid sequences of Ab85 are underlined below and are as follows:

(VL CDR1; SEQ ID NO: 111)
RSSQGIRSDLG;
(VL CDR2; SEQ ID NO: 112)
DASNLET;
and
(VL CDR3; SEQ ID NO: 113)
QQANGFPLT.

In another embodiment, an anti-CD117 antibody that may be used in the methods and compositions (including ADCs) described herein is Antibody 86 (Ab86). The heavy chain variable region (VH) amino acid sequence of Ab86 is provided below as SEQ ID NO: 114.

(SEQ ID NO: 114)
EVQLVQSGAEVKKPGESLKISCKGSGYSFTNYWIGWVRQMPGKGLE
WMGIIYPGDSDIRYSPSLQGQVTISVDTSTSTAYLQWNSLKPSDTA
MYYCARHGRGYNGYEGAFDIWGQGTLVTVSS

The VH CDR amino acid sequences Ab86 are underlined below and are as follows:

(VH CDR1; SEQ ID NO: 108)
NYWIG;
(VH CDR2; SEQ ID NO: 116)
IIYPGDSDIRYSPSLQG;
and
(VH CDR3; SEQ ID NO: 3)
HGRGYNGYEGAFDI.

The light chain variable region (VL) amino acid sequence of Ab86 is provided below as SEQ ID NO 115.

(SEQ ID NO: 115)
DIQMTQSPSSLSASVGDRVTITCRASQGIGDSLAWYQQKPGKAPKLLIYD
ASNLETGVPSRFSGSGSGTDFTLTISSLQPEDFATYYCQQLNGYPITFGQ
GTKVEIK

The VL CDR amino acid sequences of Ab86 are underlined below and are as follows:

(VL CDR1; SEQ ID NO: 117)
RASQGIGDSLA;
(VL CDR2; SEQ ID NO: 112)
DASNLET;
and
(VL CDR3; SEQ ID NO: 118)
QQLNGYPIT.

In another embodiment, an anti-CD117 antibody that may be used in the methods and compositions (including ADCs) described herein is Antibody 87 (Ab87). The heavy chain variable region (VH) amino acid sequence of Ab87 is provided below as SEQ ID NO: 106.

(SEQ ID NO: 106)
EVQLVQSGAEVKKPGESLKISCKGSGYSFTNYWIGWVRQMPGKGLEWMAI
INPRDSDTRYRPSFQGQVTISADKSISTAYLQWSSLKASDTAMYYCARHG
RGYEGYEGAFDIWGQGTLVTVSS

The VH CDR amino acid sequences of Ab87 are underlined below and are as follows:

(VH CDR1; SEQ ID NO: 108)
NYWIG;
(VH CDR2; SEQ ID NO: 109)
IINPRDSDTRYRPSFQG;
and
(VH CDR3; SEQ ID NO: 110)
HGRGYEGYEGAFDI.

The light chain variable region (VL) amino acid sequence of Ab87 is provided below as SEQ ID NO: 119.

(SEQ ID NO: 119)
DIQMTQSPSSLSASVGDRVTITCRASQGIRNDLGWYQQKPGKAPKLLIYD
ASSLESGVPSRFSGSGSGTDFTLTISSLQPEDFATYYCQQLNGYPITFGQ
GTKVEIK

The VL CDR amino acid sequences of Ab87 are underlined below and are as follows:

(VL CDR1; SEQ ID NO: 120)
RASQGIRNDLG;
(VL CDR2; SEQ ID NO: 5)
DASSLES;
and
(VL CDR3; SEQ ID NO: 118)
QQLNGYPIT.

In another embodiment, an anti-CD117 antibody that may be used in the methods and compositions (including ADCs) described herein is Antibody 88 (Ab88). The heavy chain variable region (VH) amino acid sequence of Ab88 is provided below as SEQ ID NO: 121.

(SEQ ID NO: 121)
EVQLVQSGAEVKKPGESLKISCKGSGYSFTNYWIGWVRQMPGKGLEWMGI
IYPGDSLTRYSPSFQGQVTISADKSISTAYLQWSSLKASDTAMYYCARHG
RGYNGYEGAFDIWGQGTLVTVSS

The VH CDR amino acid sequences of Ab88 are underlined below and are as follows:

(VH CDR1; SEQ ID NO: 108)
NYWIG;
(VH CDR2; SEQ ID NO: 122)
IIYPGDSLTRYSPSFQG;
and
(VH CDR3; SEQ ID NO: 3)
HGRGYNGYEGAFDI.

The light chain variable region (VL) amino acid sequence of Ab88 is provided below as SEQ ID NO: 119.

(SEQ ID NO: 119)
DIQMTQSPSSLSASVGDRVTITCRASQGIRNDLGWYQQKPGKAPKLLIYD
ASSLESGVPSRFSGSGSGTDFTLTISSLOPEDFATYYCQQLNGYPITFGQ
GTKVEIK

The VL CDR amino acid sequences of Ab88 are underlined below and are as follows:

(VL CDR1; SEQ ID NO: 120)
RASQGIRNDLG;
(VL CDR2; SEQ ID NO: 5)
DASSLES;
and
(VL CDR3; SEQ ID NO: 118)
QQLNGYPIT.

In another embodiment, an anti-CD117 antibody that may be used in the methods and compositions (including ADCs) described herein is Antibody 89 (Ab89). The heavy chain variable region (VH) amino acid sequence of Ab89 is provided below as SEQ ID NO: 123.

(SEQ ID NO: 123)
EVQLVQSGAEVKKPGESLKISCKGSGYSFTNYWIGWVRQMPGKGLEWMGI
IYPGDSDTRYSPSFQGQVTISADKSISTAYLQWSSLKASDTAMYYCARHG
RGYNGYEGAFDIWGQGTLVTVSS

The VH CDR amino acid sequences of Ab89 are underlined below and are as follows:

(VH CDR1; SEQ ID NO: 108)
NYWIG;
(VH CDR2; SEQ ID NO: 2)
IIYPGDSDTRYSPSFQG;
and
(VH CDR3; SEQ ID NO: 3)
HGRGYNGYEGAFDI.

The light chain variable region (VL) amino acid sequence of Ab89 is provided below as SEQ ID NO: 115.

(SEQ ID NO: 115)
DIQMTQSPSSLSASVGDRVTITCRASQGIGDSLAWYQQKPGKAPKLLIYD
ASNLETGVPSRFSGSGSGTDFTLTISSLQPEDFATYYCQQLNGYPITFGQ
GTKVEIK

The VL CDR amino acid sequences of Ab89 are underlined below and are as follows:

(VL CDR1; SEQ ID NO: 117)
RASQGIGDSLA;
(VL CDR2; SEQ ID NO: 112)
DASNLET;
and
(VL CDR3; SEQ ID NO: 118)
QQLNGYPIT.

The following antibodies were also identified as preferred anti-CD117 antibodies: HC-245/LC-245 (i.e., Ab245), HC-246/LC-246 (i.e., Ab246), HC-247/LC-247 (i.e., Ab247), HC-248/LC-248 (i.e., Ab248), and HC-249/LC-249 (i.e., Ab249).

TABLE 1

| Antibody | Back- | |
|---|---|---|
| name | bone | Amino acid sequence |
| HC-245 | hlgG1 | EVQLVQSGAEVKKPGESLKISCKGSGYRFTTSWIGWV |
| | | RQMPGKGLEWMGIIYPGDSDTRYSPSFQGQVTISADK |
| | | SISTAYLQWSSLKASDTAMYYCARHGLGYNGYEGAFDI |
| | | WGQGTLVTVSS (SEQ ID NO: 124) |
| LC-245 | hKappa | DIQMTQSPSSLSASVGDRVTITCRASQGIGSALAWYQQ |
| | | KPGKAPKLLIYDASTLESGVPSRFSGSGSGTDFTLTISS |
| | | LQPEDFATYYCQQFNGYPLTFGQGTRLEIK (SEQ ID |
| | | NO: 125) |
| HC-246 | hlgG1 | QVQLVQSGAAVKKPGESLKISCKGSGYRFTTYWIGWV |
| | | RQMPGKGLEWMGIIYPGDSDTRYSPSFQGQVTISAGK |
| | | SISTAYLQWSSLKASDTAMYYCARHGRGYNGYEGAFDI |
| | | WGQGTMVTVSS (SEQ ID NO: 9) |
| LC-246 | hKappa | DIQMTQSPSSLSASVGDRVTITCRASQGIGSALAWYQQ |
| | | KPGKAPKLLIYDASTLESGVPSRFSGSGSGTDFTLTISS |
| | | LQPEDFATYYCQQFNGYPLTFGQGTRLEIK (SEQ ID |
| | | NO: 125) |
| HC-247 | hlgG1 | QVQLVQSGAAVKKPGESLKISCKGSGYRFTTYWIGWV |
| | | RQMPGKGLEWMGIIYPGDSDTRYSPSFQGQVTISAGK |
| | | SISTAYLQWSSLKASDTAMYYCARHGRGYNGYEGAFDI |
| | | WGQGTMVTVSS (SEQ ID NO: 9) |
| LC-247 | hKappa | DIQMTQSPSSLSASVGDRVTITCRASRGISDYLAWYQQ |
| | | KPGKAPKLLIYDASNLETGVPSRFSGSGSGTDFTLTISS |
| | | LQPEDFATYYCQQANSFPITFGQGTRLEIK (SEQ ID |
| | | NO: 126) |
| HC-248 | hlgG1 | EVQLVQSGAEVKKPGESLKISCKGSGYRFTTSWIGWV |
| | | RQMPGKGLEWMGIIYPGDSDTRYSPSFQGQVTISADK |
| | | SISTAYLQWSSLKASDTAMYYCARHGLGYNGYEGAFDI |
| | | WGQGTLVTVSS (SEQ ID NO: 124) |
| LC-248 | hKappa | DIQMTQSPSSLSASVGDRVTITCRASQGIGSALAWYQQ |
| | | KPGKAPKLLIYDASTLESGVPSRFSGSGSGTDFTLTISS |
| | | LQPEDFATYYCQQLNGYPLTFGQGTRLEIK (SEQ ID |
| | | NO: 127) |
| HC-249 | hlgG1 | EVQLVQSGAEVKKPGESLKISCKGSGYRFTTSWIGWV |
| | | RQMPGKGLEWMGIIYPGDSDTRYSPSFQGQVTISADK |
| | | SISTAYLQWSSLKASDTAMYYCARHGLGYNGYEGAFDI |
| | | WGQGTLVTVSS (SEQ ID NO: 124) |
| LC-249 | hKappa | DIQMTQSPSSLSASVGDRVTITCRASQGIGSALAWYQQ |
| | | KPGKAPKLLIYDASNLETGVPSRFSGSGSGTDFTLTISS |
| | | LQPEDFATYYCQQLNGYPLTFGQGTRLEIK (SEQ ID |
| | | NO: 128) |

Additional anti-CD117 antibodies that can be used in conjunction with the patient conditioning methods described herein include those described in U.S. Pat. No. 7,915,391, which describes, e.g., humanized SR-1 antibodies; U.S. Pat. No. 5,808,002, which describes, e.g., the anti-CD117 A3C6E2 antibody, as well as those described in, for example, WO 2015/050959, which describes anti-CD117 antibodies that bind epitopes containing Pro317, Asn320, Glu329, Val331, Asp332, Lus358, Glue360, Glue376, His378, and/or Thr380 of human CD117; and US 2012/0288506 (also published as U.S. Pat. No. 8,552,157), which describes, e.g., the anti-CD117 antibody CK6, having the CDR sequences of:
a CDR-H1 having the amino acid sequence SYWIG (SEQ ID NO: 1);
a CDR-H2 having the amino acid sequence IIYPGDSDTRYSPSFQG (SEQ ID NO: 2);
a CDR-H3 having the amino acid sequence HGRGYNGYEGAFDI (SEQ ID NO: 3);
a CDR-L1 having the amino acid sequence RASQGISSALA (SEQ ID NO: 4);
a CDR-L2 having the amino acid sequence DASSLES (SEQ ID NO: 5); and
a CDR-L3 having the amino acid sequence CQQFNSYPLT (SEQ ID NO: 6).

### Anti-CD137 Antibodies

In some embodiments, the methods described herein include ADCs comprising an antibody, or fragment thereof, that specifically binds to the extracellular domain of human CD137. The extracellular domain of human CD137 and has the following amino acid sequence:

(SEQ ID NO: 233)
MGNSCYNIVATLLLVLNFERTRSLQDPCSNCPAGTFCDNNRNQICSPCPP
NSESSAGGQRTCDICRQCKGVERTRKECSSTSNAECDCTPGFHCLGAGCS
MCEQDCKQGQELTKKGCKDCCFGTFNDQKRGICRPWTNCSLDGKSVLVNG
TKERDVVCGPSPADLSPGASSVTPPAPAREPGHSPQIISFFLALTSTALL
FLLFFLTLRFSVVKRGRKKLLYIFKQPFMRPVQTTQEEDGCSCRFPEEEE
GGCEL

In one embodiment, the methods disclosed herein include administration of an anti-CD137 ADC comprising an anti-CD137 antibody conjugated to a cytotoxin via a linker. For example, ADCs capable of binding CD137 can be used as therapeutic agents to deplete allo-reactive T cells in a human patient who received an allogenic transplant. Additionally it has been discovered that antibodies, and antigen-binding fragments thereof, capable of binding CD137 can be used as therapeutic agents to prevent or reduce the risk of GvHD in a patient suffering from or at risk for GvHD. CD137 is also referred to as 4-1 BB.

T cells have been shown to express CD137, as this antigen is a transmembrane TNF receptor superfamily of costimulatory molecules and is expressed on a variety of hematopoietic cells and promotes T cell activation and regulates proliferation and survival of T cells (see, e.g., Cannons et al., J. Immunol. 167:1313-1324, 2001, the disclosure of which is incorporated herein by reference as it pertains to the expression of CD137 by T cells). Antibodies, antigen-binding fragments thereof, and ligands can be identified using techniques known in the art and described herein, such as by immunization, computational modeling techniques, and in vitro selection methods, such as the phage display and cell-based display platforms described below.

In one embodiment, an anti-CD137 antibody that may be used in the methods as described herein is the murine anti-CD137 antibody BBK2 (Thermo Fisher; MS621 PABX) or an anti-CD137 antibody comprising antigen binding regions corresponding to the BBK2 antibody. The BBK2 antibody (which may also be referred to as a BBK-2 antibody or an anti-4-1 BB antibody), is a mouse monoclonal antibody (IgG1, kappa) that binds to the ectodomain of human 4-1 BB recombinant protein (4-1 BB is also known as CD137). In certain embodiments, the methods and compositions of the disclosure include an anti-CD137 antibody comprising the binding regions (e.g., the CDRs) of the BBK2 antibody. In another embodiment, the methods and compositions of the disclosure comprise an antibody that competitively inhibits the binding of the BBK2 antibody to its epitope on CD137. In certain embodiments, the anti-CD137 antibody is humanized BBK2 or chimeric BBK2.

In one embodiment, the methods described herein include a chimeric anti-CD137 (ch-BBK2) antibody comprising the variable heavy and light chain regions of BBK2. In certain embodiments, the chimeric BBK2 antibody is an IgG1 antibody comprising human constant regions. The heavy chain amino acid sequence of ch-BBK2 is described in SEQ ID NO: 214, and the light chain amino acid sequence of ch-BBK2 is described in SEQ ID NO: 215. The CDR regions (CDR1, CDR2, and CDR3) of each of the heavy and light chain sequences are described in bold below. The variable regions are italicized.

(SEQ ID NO: 214)
QVQLQQPGAELVRPGASVKLSCKASGYTFTSYWINWVKQRPGQGLEWIGN
IYPSDSYTNYNQKFKDKATLTVDKSSNTVYMQLNSPTSEDSAVYYCTRNG
VEGYPHYYAMEYWGQGTSVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLS
PGK
(SEQ ID NO: 215)
DIQMTQTTSALSASLGDRVTIGCRASQDLSNHLYWYQQKPDGTVKLLIYY
TSRLHSGVPSRFSGSGSGTDYSLTIRNLEQEDVATYFCQQGYTLPYTFGG
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

The foregoing CDR regions (and BBK2 antibody) are described in Lee et al. (2002) European J of Immunogenetics 29(5):449-452. Thus, in one embodiment, the VH CDR amino acid sequences of anti-CD137 antibody BBK2 (including ch-BBK2) are as follows: SGYTFTSYW (VH CDR1; SEQ ID NO: 252); NIYPSDSYT (VH CDR2; SEQ ID NO: 253) and TRNGVEGYPHYYAME (VH CDR3; SEQ ID NO: 254). The VL CDR amino acid sequences of anti-CD137 antibody BBK2 (including ch-BBK2) are as follows: SQDLSNH (VL CDR1; SEQ ID NO: 255); YYTS (VL CDR2; SEQ ID NO: 256) and CQQGYTLPY (VL CDR3; SEQ ID NO: 257).

Alternatively, the CDR regions of BBK2 can be defined according to Kabat numbering. CDRs as defined by Kabat numbering are described below for each of the heavy and light chain sequences (described in bold below). The variable regions of BBK2 are italicized.

(SEQ ID NO: 214; ch-BBK2 heavy chain)
QVQLQQPGAELVRPGASVKLSCKASGYTFTSYWINWVKQRPGQGLEWIGN
IYPSDSYTNYNQKFKDKATLTVDKSSNTVYMQLNSPTSEDSAVYYCTRNG
VEGYPHYYAMEYWGQGTSVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLS
PGK
(SEQ ID NO: 215; ch-BBK2 light chain)
DIQMTQTTSALSASLGDRVTIGCRASQDLSNHLYWYQQKPDGTVKLLIYY
TSRLHSGVPSRFSGSGSGTDYSLTIRNLEQEDVATYFCQQGYTLPYTFGG
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

Thus, in one embodiment, the VH CDR amino acid sequences of anti-CD137 antibody BBK2 (including ch-BBK2) are as follows: SYWIN (VH CDR1; SEQ ID NO: 218); NIYPSDSYTNYNQKFKD (VH CDR2; SEQ ID NO: 219) and NGVEGYPHYYAMEY (VH CDR3; SEQ ID NO: 220), and the VL CDR amino acid sequences of anti-CD137 antibody BBK2 (including ch-BBK2) are as follows: RASQDLSNHLY (VL CDR1; SEQ ID NO: 221); YTSRLHS (VL CDR2; SEQ ID NO: 222) and QQGYTLPYT (VL CDR3; SEQ ID NO: 223).

The heavy chain variable region of BBK2 is
(SEQ ID NO: 216)
QVQLQQPGAELVRPGASVKLSCKASGYTFTSYWINWVKQRPGQGLEWIGN
IYPSDSYTNYNQKFKDKATLTVDKSSNTVYMQLNSPTSEDSAVYYCTRNG
VEGYPHYYAMEYWGQGTSVTVSS.
The light chain variable region of BBK2 is
(SEQ ID NO: 217)
DIQMTQTTSALSASLGDRVTIGCRASQDLSNHLYWYQQKPDGTVKLLIYY
TSRLHSGVPSRFSGSGSGTDYSLTIRNLEQEDVATYFCQQGYTLPYTFGG
GTKLEIK.

Anti-CD137 antibodies (including anti-CD137 ADCs) can comprise the heavy and light chain variable region amino acid sequences set forth above.

In one embodiment, the anti-CD137 antibody, e.g., a chimeric (ch-BBK2) antibody or a humanized BBK2 antibody, comprises a heavy chain variable region comprising the BBK2 heavy chain CDRs and comprises a light chain variable region comprising the BBK2 light chain CDRs.

In one embodiment, the anti-CD137 antibody, e.g., a chimeric (ch-BBK2) antibody or a chimeric BBK2 antibody, comprises a heavy chain variable region comprising the BBK2 heavy chain CDRs and comprises a light chain variable region comprising the BBK2 light chain CDRs.

Thus, BBK2, humanized BBK2, or chimeric BBK2 antibodies can be used in the anti-CD137 ADCs and methods described herein. Each of these antibodies can be conjugated to any of the cytotoxin described below using methods known in the art and those described herein.

### Anti-CD5 Antibodies

In some embodiments, the methods described herein include ADCs comprising an antibody, or fragment thereof, that specifically binds to human CD5. Human CD5 is also referred to as LEU1 or T1. Human CD5 is a type-I transmembrane glycoprotein found on the surface of thymocytes, T lymphocytes and a subset of B lymphocytes. Two isoforms of human CD5 have been identified. Isoform 1 contains 438 amino acids and is described in Jones. et al. (1988) Nature 323 (6086), 346-349 and below (NCBI Reference Sequence: NP_001333385.1):

(SEQ ID NO: 234)
MVCSQSWGRSSKQWEDPSQASKVCQRLNCGVPLSLGPFLVTYTPQSSIIC
YGQLGSFSNCSHSRNDMCHSLGLTCLEPQKTTPPTTRPPPTTTPEPTAPP
RLOLVAQSGGQHCAGVVEFYSGSLGGTISYEAQDKTQDLENFLCNNLQCG
SFLKHLPETEAGRAQDPGEPREHQPLPIQWKIQNSSCTSLEHCFRKIKPQ
KSGRVLALLCSGFQPKVQSRLVGGSSICEGTVEVRQGAQWAALCDSSSAR
SSLRWEEVCREQQCGSVNSYRVLDAGDPTSRGLFCPHQKLSQCHELWERN
SYCKKVFVTCQDPNPAGLAAGTVASIILALVLLVVLLVVCGPLAYKKLVK
KFRQKKQRQWIGPTGMNQNMSFHRNHTATVRSHAENPTASHVDNEYSQPP
RNSHLSAYPALEGALHRSSMQPDNSSDSDYDLHGAQRL

In one embodiment, the methods disclosed herein include administration of an anti-CD5 ADC comprising an anti-CD5 antibody conjugated to a cytotoxin via a linker. Thus, anti-CD5 ADCs can be used as conditioning agents to promote the engraftment of transplanted hematopoietic stem cells in a patient in need of transplant therapy by preventing or reducing the likelihood of immune cell-mediated graft rejection. For instance, anti-CD5 ADCs can bind cell-surface CD5 expressed by immune cells such as T cells, B cells, or NK cells that cross-react with, and mount an immune response against, non-self hematopoietic stem cell antigens, such as non-self MHC antigens expressed by a hematopoietic stem cell graft. The binding of such antibodies, and antigen-binding fragments, to hematopoietic stem cell-specific CD5+ immune cells can induce death of the bound immune cell, for instance, by antibody-dependent cell-mediated cytotoxicity or by the action of a cytotoxic agent that is conjugated to the antibody, or the antigen-binding fragment thereof. The depletion of a population of CD5+ immune cells that cross-react with non-self hematopoietic stem cells can thus facilitate the engraftment of hematopoietic stem cell transplants in a patient in need thereof by attenuating the ability of the recipient's immune system to mount an immune response against the incoming graft. In this way, a patient suffering from a stem cell disorder, cancer, autoimmune disease, or other blood disorder described herein can be treated, as a hematopoietic stem cell transplant can be provided to a subject in order to repopulate a lineage of cells that is defective and/or deficient in the subject. The subject may be deficient in a population of cells due to, for instance, chemotherapy that has been administered to the subject with the aim of eradicating cancerous cells but that has, in the process, depleted healthy hematopoietic cells as well.

In one embodiment, an anti-CD5 antibody that may be used in the methods and compositions (including ADCs) described herein is Antibody 5D7v (Ab5D7v) The heavy chain variable region (VH) amino acid sequence of Ab5D7v is provided below.

(SEQ ID NO: 224)
QVTLKESGPVLVKPTETLTLTCTFSGFSLSTSGMGVGWIRQAPGKGLEWV
AHIWWDDDVYYNPSLKSRLTITKDASKDQVSLKLSSVTAADTAVYYCVRR
RATGTGFDYWGQGTLVTVSS

The VH CDR amino acid sequences of Ab5D7v are underlined below and are as follows:

(VH CDR1; SEQ ID NO: 226)
FSLSTSGMG;
(VH CDR2; SEQ ID NO: 227)
WWDDD;
and
(VH CDR3; SEQ ID NO: 228)
RRATGTGFDY.

The light chain variable region (VL) amino acid sequence of Ab5D7v is provided below:

(SEQ ID NO: 225)
NIVMTQSPSSLSASVGDRVTITCQASQDVGTAVAWYQQKPDQSPKLLIYW
TSTRHTGVPDRFTGSGSGTDFTLTISSLQPEDIATYFCHQYNSYNTFGSG
TKLEIK

The VL CDR amino acid sequences of Ab5D7v are underlined below and are as follows:

(VL CDR1; SEQ ID NO: 229)
QDVGTA;
(VL CDR2; SEQ ID NO: 230)
WTSTRHT;
and
(VL CDR3; SEQ ID NO: 231)
YNSYNT.

In another embodiment, an anti-CD5 antibody that may be used in the methods and compositions (including ADCs) described herein is the 5D7 antibody (see, e.g., US 20080254027A1, the disclosure of which is incorporated herein by reference). In another embodiment, an anti-CD5 antibody that may be used in the methods and compositions (including ADCs) described herein is a variant of the 5D7 antibody (see, e.g., US 20080254027A1, the disclosure of which is incorporated herein by reference).

### Anti-CD2 Antibodies

In some embodiments, the methods described herein include ADCs comprising an antibody, or fragment thereof, that specifically binds to human CD2. Human CD2 is also referred to as T-cell Surface Antigen T11/Leu-5, T11, CD2 antigen (p50), and Sheep Red Blood Cell Receptor (SRBC). CD2 is expressed on T cells. Two isoforms of human CD2 have been identified. Isoform 1 contains 351 amino acids is described in Seed, B. et al. (1987) 84: 3365-69 (see also Sewell et al. (1986) 83: 8718-22) and below (NCBI Reference Sequence: NP_001758.2):

(SEQ ID NO: 235)
msfpckfvas fllifnvssk gavskeitna letwgalgqd
inldipsfqm sddiddikwe ktsdkkkiaq frkeketfke
kdtyklfkng tlkikhlktd dqdiykvsiy dtkgknvlek
ifdlkiqerv skpkiswtci nttltcevmn gtdpelnlyq
dgkhlklsqr vithkwttsl sakfkctagn kvskessvep
vscpekgldi yliigicggg sllmvfvall vfyitkrkkq
rsrrndeele trahrvatee rgrkphqipa stpqnpatsq
hpppppghrs qapshrpppp ghrvqhqpqk rppapsgtqv
hqqkgpplpr prvqpkpphg aaenslspss n

A second isoform of CD2 is 377 amino acids and is identified herein as NCBI Reference Sequence: NP_001315538.1.

In one embodiment, the methods disclosed herein include administration of an anti-CD2 ADC comprising an anti-CD2 antibody conjugated to a cytotoxin via a linker. ADCs capable of binding CD2 can be used as therapeutic agents to promote the engraftment of transplanted hematopoietic stem cells in a patient in need of transplant therapy by preventing or reducing the likelihood of immune cell-mediated graft rejection. For instance, anti-CD2 ADCs, can bind cell-surface CD2 expressed by immune cells such as T cells or NK cells that cross-react with, and mount an immune response against, one or more non-self hematopoietic stem cell antigens, such as one or more non-self MHC antigens expressed by the hematopoietic stem cells. The binding of such antibodies, and antigen-binding fragments, to hematopoietic stem cell-specific CD2+ immune cells can induce death of the bound immune cell, for instance, by antibody-dependent cell-mediated cytotoxicity or by the action of a cytotoxic agent that is conjugated to the antibody, or the antigen-binding fragment thereof. The depletion of a population of CD2+ immune cells that cross-react with non-self hematopoietic stem cells can thus facilitate the engraftment of hematopoietic stem cell transplants in a patient in need thereof by attenuating the ability of the recipient's immune system to mount an immune response against the incoming graft. In this way, a patient suffering from a stem cell disorder, cancer, autoimmune disease, or other blood disorder described herein can be treated, as a hematopoietic stem cell transplant can be provided to a subject in order to repopulate a lineage of cells that is defective and/or deficient in the subject. The subject may be deficient in a population of cells due to, for instance, chemotherapy that has been administered to the subject with the aim of eradicating cancerous cells but that has, in the process, depleted healthy hematopoietic cells as well.

In one embodiment, an ADC used in the methods described herein may comprise an anti-CD2 antibody as described herein. For example, in one embodiment, the anti-CD2 antibody comprises a heavy chain variable region (VH) amino acid sequence and a light chain variable region (VL) amino acid sequence as provided below:

AbA anti-CD2 (VH) amino acid sequence
(SEQ ID NO: 240)
QVQLVQSGAEVKKPGASVKVSCKASGYTFTEYYMYWVRQAPGQGLELMGR
IDPEDGSIDYVEKFKKKVTLTADTSSSTAYMELSSLTSDDTAVYYCARGK
FNYRFAYWGQGTLVTVSS
AbA Anti-CD2 (VL) amino acid sequence
(SEQ ID NO: 241)
DVVMTQSPPSLLVTLGQPASISCRSSQSLLHSSGNTYLNWLLQRPGQSPQ
PLIYLVSKLESGVPDRFSGSGSGTDFTLKISGVEAEDVGVYYCMQFTHYP
YTFGQGTKLEIK

In another embodiment, the anti-CD2 antibody comprises a heavy chain variable region (VH) amino acid sequence and a light chain variable region (VL) amino acid sequence as provided below:

AbB anti-CD2 (VH) amino acid sequence
(SEQ ID NO: 242)
QVQLVQSGAEVQRPGASVKVSCKASGYIFTEYYMYWVRQAPGQGLELVGR
IDPEDGSIDYVEKFKKKVTLTADTSSSTAYMELSSLTSDDTAVYYCARGK
FNYRFAYWGQGTLVTVSS
AbB Anti-CD2 (VL) amino acid sequence
(SEQ ID NO: 243)
DVVMTQSPPSLLVTLGQPASISCRSSQSLLHSSGNTYLNWLLQRPGQSPQ
PLIYLVSKLESGVPDRFSGSGSGTDFTLKISGVEAEDVGVYYCMQFTHYP
YTFGQGTKLEIK

In another embodiment, the anti-CD2 antibody comprises a heavy chain variable region (VH) amino acid sequence and a light chain variable region (VL) amino acid sequence as provided below:

AbC anti-CD2 (VH) amino acid sequence
(SEQ ID NO: 244)
EVKLVESGGGLVKPGGSLKLSCAASGFTFSSYDMSWVRQTPEKRLEWVAS
ISGGGFLYYLDSVKGRFTISRDNARNILYLHMTSLRSEDTAMYYCARSSY
GEIMDYWGQGTSVTVSS
AbC Anti-CD2 (VL) amino acid sequence
(SEQ ID NO: 245)
DILLTQSPAILSVSPGERVSFSCRASQRIGTSIHWYQQRTTGSPRLLIKY
ASESISGIPSRFSGSGSGTDFTLSINSVESEDVADYYCQQSHGWPFTFGG
GTKLEIE

In another embodiment, the anti-CD2 antibody comprises a heavy chain variable region (VH) amino acid sequence and a light chain variable region (VL) amino acid sequence as provided below:

AbD anti-CD2 (VH) amino acid sequence
(SEQ ID NO: 246)
EVKLVESGGGLVKPGGSLKLSCAASGFTFSSYDMSWVRQTPEKRLEWVAS
ISGGGFLYYLDSVKGRFTISRDNARNILYLHMTSLRSEDTAMYYCARSSY
GELMDYWGQGTSVTVSS
AbD Anti-CD2 (VL) amino acid sequence
(SEQ ID NO: 246)
DILLTQSPAILSVSPGERVSFSCRASQRIGTSIHWYQQRTTGSPRLLIKY
ASESISGIPSRFSGSGSGTDFTLSINSVESEDVADYYCQQSHGWPFTFGG
GTKLEIE

### Anti-CD45 Antibodies

Antibodies and antigen-binding fragments capable of binding human CD45 (mRNA NCBI Reference Sequence: NM_080921.3, Protein NCBI Reference Sequence: NP_563578.2), including those capable of binding the isoform CD45RO, can be used in conjunction with the compositions and methods disclosed herein, such as to promote engraftment of hematopoietic stem cell grafts in a patient in need of hematopoietic stem cell transplant therapy. Multiple isoforms of CD45 arise from the alternative splicing of 34 exons in the primary transcript. Splicing of exons 4, 5, 6, and potentially 7 give rise to multiple CD45 variations. Selective exon expression is observed in the CD45 isoforms described below.

### Exon Expression in Various Human CD45 Isoforms

| CD45 Isoform | Exon Expression Pattern |
|---|---|
| CD45RA (SEQ ID NO: 237) | Expresses exon 4 only |
| CD45RB (SEQ ID NO: 238) | Expresses exon 5 only |
| CD45RC (SEQ ID NO: 239) | Expresses exon 6 only |
| CD45RO (SEQ ID NO: 236) | Does not express exons 4-6 |

Alternative splicing can result in individual exons or combinations of exons expressed in various isoforms of the CD45 protein (for example, CD45RA, CD45RAB, CD45RABC). In contrast, CD45RO lacks expression of exons 4-6 and is generated from a combination of exons 1-3 and 7-34. There is evidence that exon 7 can also be excluded from the protein, resulting in splicing together of exons 1-3 and 8-34. This protein, designated E3-8, has been detected at the mRNA level but has not been currently identified by flow cytometry.

CD45RO is currently the only known CD45 isoform expressed on hematopoietic stem cells. CD45RA and CD45RABC have not been detected or are excluded from the phenotype of hematopoietic stem cells. There is evidence from studies conducted in mice that CD45RB is expressed on fetal hematopoietic stem cells, but it is not present on adult bone marrow hematopoietic stem cells. Notably, CD45RC has a high rate of polymorphism in exon 6 found within Asian populations (a polymorphism at exon 6 in CD45RC is found in approximately 25% of the Japanese population). This polymorphism leads to high expression of CD45RO and decreased levels of CD45RA, CD45RB, and CD45RC. Additionally, CD45RA variants (such as CD45RAB and CD45RAC) exhibit a polymorphism in exon 4 that has been associated with autoimmune disease.

The presence of CD45RO on hematopoietic stem cells and its comparatively limited expression on other immune cells (such as T and B lymphocyte subsets and various myeloid cells) renders CD45RO a particularly well-suited target for conditioning therapy for patients in need of a hematopoietic stem cell transplant. As CD45RO only lacks expression of exons 4, 5, and 6, its use as an immunogen enables the screening of pan CD45 Abs and CD45RO-specific antibodies. Thus, in one embodiment, an anti-CD45 ADC used in the methods disclosed herein specifically binds to human CD45RO.

Anti-CD45 antibodies that can be used in conjunction with the patient conditioning methods described herein include anti-CD45 antibodies, and antigen-binding portions thereof. Antigen-binding portions of antibodies are well known in the art, and can readily be constructed based on the antigen-binding region of the antibody. In exemplary embodiments, the anti-CD45 antibody used in conjunction with the conditioning methods described herein can be a monoclonal antibody or antigen-binding fragment thereof, a polyclonal antibody or antigen-binding fragment thereof, a humanized antibody or antigen-binding fragment thereof, a fully human antibody or antigen-binding fragment thereof, a chimeric antibody or antigen-binding fragment thereof, a bispecific antibody or antigen-binding fragment thereof, a dual-variable immunoglobulin domain, a single-chain Fv molecule (scFv), a diabody, a triabody, a nanobody, an antibody-like protein scaffold, a Fv fragment, a Fab fragment, a F(ab′)2 molecule, or a tandem di-scFv. Exemplary anti-CD45 antibodies which may be used in whole or in part in the ADCs or methods described herein are provided below.

In one embodiment, the anti-CD45 antibody is or is derived from clone H130, which is commercially available from BIOLEGEND® (San Diego, CA), or a humanized variant thereof. Humanization of antibodies can be performed by replacing framework residues and constant region residues of a non-human antibody with those of a germline human antibody according to procedures known in the art (as described, for instance, in Example 7, below). Additional anti-CD45 antibodies that can be used in conjunction with the methods described herein include the anti-CD45 antibodies ab10558, EP322Y, MEM-28, ab10559, 0.N.125, F10-89-4, Hle-1, 2B11, YTH24.5, PD7/26/16, F10-89-4, 1B7, ab154885, B-A11, phosphor S1007, ab170444, EP350, Y321, GA90, D3/9, X1 6/99, and LT45, which are commercially available from ABCAM® (Cambridge, MA), as well as humanized variants thereof. Further anti-CD45 antibodies that may be used in conjunction with the patient conditioning procedures described herein include anti-CD45 antibody HPA000440, which is commercially available from SIGMA-ALDRICH® (St. Louis, MO), and humanized variants thereof. Additional anti-CD45 antibodies that can be used in conjunction with the patient conditioning methods described herein include murine monoclonal antibody BC8, which is described, for instance, in Matthews et al., Blood 78:1864-1874, 1991, the disclosure of which is incorporated herein by reference as it pertains to anti-CD45 antibodies, as well as humanized variants thereof. Further anti-CD45 antibodies that can be used in conjunction with the methods described herein include monoclonal antibody YAML568, which is described, for instance, in Glatting et al., J. Nucl. Med. 8:1335-1341, 2006, the disclosure of which is incorporated herein by reference as it pertains to anti-CD45 antibodies, as well as humanized variants thereof. Additional anti-CD45 antibodies that can be used in conjunction with the patient conditioning procedures described herein include monoclonal antibodies YTH54.12 and YTH25.4, which are described, for instance, in Brenner et al., Ann. N.Y. Acad. Sci. 996:80-88, 2003, the disclosure of which is incorporated herein by reference as it pertains to anti-CD45 antibodies, as well as humanized variants thereof. Additional anti-CD45 antibodies for use with the patient conditioning methods described herein include UCHL1, 2H4, SN130, MD4.3, MBI, and MT2, which are described, for instance, in Brown et al., Immunology 64:331-336, 1998, the disclosure of which is incorporated herein by reference as it pertains to anti-CD45 antibodies, as well as humanized variants thereof. Additional anti-CD45 antibodies that can be used in conjunction with the methods described herein include those produced and released from American Type Culture Collection (ATCC) Accession Nos. RA3-6132, RA3-2C2, and TIB122, as well as monoclonal antibodies C363.16A, and 13/2, which are described, for instance, in Johnson et al., J. Exp. Med. 169:1179-1184, 1989, the disclosure of which is incorporated herein by reference as it pertains to anti-CD45 antibodies, as well as humanized variants thereof. Further anti-CD45 antibodies that can be used in conjunction with the patient conditioning methods described herein include the monoclonal antibodies AHN-12.1, AHN-12, AHN-12.2, AHN-12.3, AHN-12.4, HLe-1, and KC56(T200), which are described, for instance, in Harvath et al., J. Immunol. 146:949-957, 1991, the disclosure of which is incorporated herein by reference as it pertains to anti-CD45 antibodies, as well as humanized variants thereof.

Additional anti-CD45 antibodies that can be used in conjunction with the patient conditioning methods described herein include those described, for example, in U.S. Pat. No. 7,265,212 (which describes, e.g., anti-CD45 antibodies 39E11, 16C9, and 1 G10, among other clones); U.S. Pat. No. 7,160,987 (which describe, e.g., anti-CD45 antibodies produced and released by ATCC Accession No. HB-11873, such as monoclonal antibody 6G3); and U.S. Pat. No. 6,099,838 (which describes, e.g., anti-CD45 antibody MT3, as well as antibodies produced and released by ATCC Accession Nos. HB220 (also designated MB23G2) and HB223), as well as US 2004/0096901 and US 2008/0003224 (which describes, e.g., anti-CD45 antibodies produced and released by ATCC Accession No. PTA-7339, such as monoclonal antibody 17.1), the disclosures of each of which are incorporated herein by reference as they pertain to anti-CD45 antibodies.

Further anti-CD45 antibodies that can be used in conjunction with the patient conditioning methods described herein include antibodies produced and released from ATCC Accession Nos. MB4B4, MB23G2, 14.8, GAP 8.3, 74-9-3, 1/24.D6, 9.4, 4B2, M1/9.3.4.HL.2, as well as humanized and/or affinity-matured variants thereof. Affinity maturation can be performed, for instance, using in vitro display techniques described herein or known in the art, such as phage display, as described in Example 6, below.

Additional anti-CD45 antibodies that can be used in conjunction with the patient conditioning methods described herein include anti-CD45 antibody T29/33, which is described, for instance, in Morikawa et al., Int. J. Hematol. 54:495-504, 1991, the disclosure of which is incorporated herein by reference as it pertains to anti-CD45 antibodies.

In certain embodiments, the anti-CD45 antibody is selected from apamistamab (also known 90Y-BC8, Iomab-B, BC8; as described in, e.g., US20170326259, WO2017155937, and Orozco et al. Blood. 127.3 (2016): 352-359.) or BC8-B10 (as described, e.g., in Li et al. PloS one 13.10 (2018): e0205135.), each of which is incorporated by reference. Other anti-CD45 antibodies have been described, for example, in WO2003/048327, WO2016/016442, US2017/0226209, US2016/0152733, U.S. Pat. No. 9,701,756; US2011/0076270, or U.S. Pat. No. 7,825,222, each of which is incorporated by reference in its entirety.

For example, in one embodiment, the anti-CD45 antibody, or antigen-binding fragment thereof, comprising binding regions, e.g., CDRs, variable regions, corresponding to those of apamistamab. The heavy chain variable region (VH) amino acid sequence of apamistamab is set forth in SEQ ID NO: 250. The light chain variable region (VL) amino acid sequence of apamistamab is described in SEQ ID NO: 251. In other embodiments, an anti-CD45 antibody, or antigen-binding portion thereof, comprises a variable heavy chain comprising the amino acid residues set forth in SEQ ID NO: 250, and a light chain variable region as set forth in SEQ ID NO: 251. In one embodiment, the anti-CD45 antibody comprises a heavy chain comprising a CDR1, CDR2 and CDR3 of apamistamab, and a light chain variable region comprising a CDR1, CDR2 and CDR3 of apamistamab.

In another embodiment, the antibody, or antigen-binding fragment thereof, comprises a heavy chain variable region that comprises an amino acid sequence having at least 95% identity to an anti-CD45 antibody herein, e.g., at least 95%, 96%, 97%, 98%, 99%, or 100% identity to an anti-CD45 antibody herein. In certain embodiments, an antibody comprises a modified heavy chain (HC) variable region comprising an HC variable domain of an anti-CD45 antibody herein, or a variant thereof, which variant (i) differs from the anti-CD45 antibody in 1, 2, 3, 4 or 5 amino acids substitutions, additions or deletions; (ii) differs from the anti-CD45 antibody in at most 5, 4, 3, 2, or 1 amino acids substitutions, additions or deletions; (iii) differs from the anti-CD45 antibody in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to the anti-CD45 antibody, wherein in any of (i)-(iv), an amino acid substitution may be a conservative amino acid substitution or a non-conservative amino acid substitution; and wherein the modified heavy chain variable region can have an enhanced biological activity relative to the heavy chain variable region of the anti-CD45 antibody, while retaining the CD45 binding specificity of the antibody.

The disclosures of each of the foregoing publications are incorporated herein by reference in their entirety. Antibodies and antigen-binding fragments that may be used in conjunction with the compositions and methods described herein include the above-described antibodies and antigen-binding fragments thereof, as well as humanized variants of those non-human antibodies and antigen-binding fragments described above and antibodies or antigen-binding fragments that bind the same epitope as those described above, as assessed, for instance, by way of a competitive CD45 binding assay.

In one embodiment, the methods disclosed herein include administration of an anti-CD45 ADC comprising an anti-CD45 antibody conjugated to a cytotoxin via a linker.

### Anti-CD252 Antibodies

In some embodiments, the methods described herein include ADCs comprising an antibody, or fragment thereof, that specifically binds to human CD252 (also referred to as OX40 ligand (OX40L)), Protein NCBI Reference Sequence: NP_003317.1; Uniprot Accession No: P23510;). Using the methods disclosed herein, an anti-CD252 ADC can be used as a therapeutic agent to prevent and treat GVHD. Anti-CD252 antibodies can be used alone or conjugated to a cytotoxin as an antibody drug conjugate (ADC).

In one embodiment, an anti-CD252 antibody that may be used in the methods and compositions (including ADCs) described herein is the murine monoclonal anti-CD252 antibody 11C3.1 or an anti-CD252 antibody comprising antigen binding regions corresponding to the 11C3.1 antibody. 11C3.1 (sold by Biolegend Cat. No. 326302 (date Feb. 27, 2019)).

In one embodiment, an anti-CD252 antibody comprises a heavy chain comprising a CDR1, CDR2 and CDR3 of anti-CD252 antibody 11C3.1, and a light chain variable region comprising a CDR1, CDR2 and CDR3 of anti-CD252 antibody 11C3.1. In another embodiment, an anti-CD252 antibody used in the compositions and methods disclosed herein is a humanized 11C3.1 antibody.

In one embodiment, an anti-CD252 antibody that may be used in the methods and compositions (including ADCs) described herein is the murine monoclonal anti-CD252 antibody 159403 or an anti-CD252 antibody comprising antigen binding regions corresponding to the 159403 antibody. 159403 (sold by R&D Systems, Catalog #MAB10541 (date Feb. 27, 2019)).

In one embodiment, an anti-CD252 antibody comprises a heavy chain comprising a CDR1, CDR2 and CDR3 of anti-CD252 antibody 159403, and a light chain variable region comprising a CDR1, CDR2 and CDR3 of anti-CD252 antibody 159403. In another embodiment, an anti-CD252 antibody used in the compositions and methods disclosed herein is a humanized 159403 antibody.

In one embodiment, an anti-CD252 antibody that may be used in the methods and compositions (including ADCs) described herein is the murine monoclonal anti-CD252 antibody 159408 or an anti-CD252 antibody comprising antigen binding regions corresponding to the 159408 antibody. 159408 (sold by R&D Systems, Catalog #MAB1054 (date Feb. 27, 2019)).

In one embodiment, an anti-CD252 antibody comprises a heavy chain comprising a CDR1, CDR2 and CDR3 of anti-CD252 antibody 159408, and a light chain variable region comprising a CDR1, CDR2 and CDR3 of anti-CD252 antibody 159408. In another embodiment, an anti-CD252 antibody used in the compositions and methods disclosed herein is a humanized 159408 antibody.

In one embodiment, an anti-CD252 antibody that may be used in the methods and compositions (including ADCs) described herein is the murine monoclonal anti-CD252 antibody MM0505-8S23 or an anti-CD252 antibody comprising antigen binding regions corresponding to the MM0505-8S23 antibody. MM0505-8S23 (sold by Novus, Catalog #NBP2-11969 (date Feb. 27, 2019)). This antibody was produced from a hybridoma (mouse myeloma fused with spleen cells from a mouse immunized with human TNFSF4, also called OX40 ligand.

In one embodiment, an anti-CD252 antibody comprises a heavy chain comprising a CDR1, CDR2 and CDR3 of anti-CD252 antibody MM0505-8S23, and a light chain variable region comprising a CDR1, CDR2 and CDR3 of anti-CD252 antibody MM0505-8S23. In another embodiment, an anti-CD252 antibody used in the compositions and methods disclosed herein is a humanized MM0505-8S23 antibody.

In one embodiment, an anti-CD252 antibody that may be used in the methods and compositions (including ADCs) described herein is the rabbit monoclonal anti-CD252 antibody oxelumab or an anti-CD252 antibody comprising antigen binding regions corresponding to the oxelumab antibody. Oxelumab (sold by Novus, Catalog #NBP2-52687-0.1 (date Feb. 27, 2019)).

The antibodies or antigen binding fragments thereof described herein and used in the ADCs described herein may include modifications and/or mutations that alter the properties of the antibodies and/or fragments, such as those that increase half-life, increase or decrease ADCC, etc., as is known in the art.

### Fc Variants

In one embodiment, the antibodies or antigen binding fragments thereof described herein, comprise a variant Fc region, wherein said variant Fc region comprises at least one amino acid modification relative to a wild-type Fc region, such that said molecule has an altered affinity for an FcgammaR. Certain amino acid positions within the Fc region are known through crystallography studies to make a direct contact with FcγR. Specifically amino acids 234-239 (hinge region), amino acids 265-269 (B/C loop), amino acids 297-299 (C′/E loop), and amino acids 327-332 (F/G) loop. (see Sondermann et al., 2000 Nature, 406: 267-273). For example, amino acid substitutions at amino acid positions 234 and 235 of the Fc region have been identified as decreasing affinity of an IgG antibody for binding to an Fc receptor, particularly an Fc gamma receptor (FcγR). In one embodiment, an anti-CD117 antibody described herein comprises an Fc region comprising an amino acid substitution at L234 and/or L235, e.g., L234A and L235A (EU index). Thus, the antibodies or antigen binding fragments thereof described herein may comprise variant Fc regions comprising modification of at least one residue that makes a direct contact with an FcγR based on structural and crystallographic analysis. In one embodiment, the Fc region of the antibodies or antigen binding fragments thereof described herein (or Fc containing fragment thereof) comprises an amino acid substitution at amino acid 265 according to the EU index as in Kabat et al., Sequences of Proteins of Immunological Interest, 5th Ed. Public Health Service, NH1, MD (1991), expressly incorporated herein by references. The “EU index as in Kabat” or “EU index” refers to the numbering of the human IgG1 EU antibody and is used herein in reference to Fc amino acid positions unless otherwise indicated.

In one embodiment, the Fc region comprises a D265A mutation. In one embodiment, the Fc region comprises a D265C mutation.

In some embodiments, the Fc region of the antibodies or antigen binding fragments thereof described herein comprise an amino acid substitution at amino acid 234 according to the EU index as in Kabat. In one embodiment, the Fc region comprises a L234A mutation. In some embodiments, the Fc region of the antibodies or antigen binding fragments thereof described herein comprise an amino acid substitution at amino acid 235 according to the EU index as in Kabat. In one embodiment, the Fc region comprises a L235A mutation. In yet another embodiment, the Fc region comprises a L234A and L235A mutation. In a further embodiment, the Fc region comprises a D265C, L234A, and L235A mutation.

In some embodiments, the Fc region of the antibodies or antigen binding fragments thereof described herein comprise an amino acid substitution at amino acid 239 according to the EU index as in Kabat. In one embodiment, the Fc region comprises a S239C mutation.

In certain aspects a variant IgG Fc domain comprises one or more amino acid substitutions resulting in decreased or ablated binding affinity for an FcgammaR and/or Clq as compared to the wild type Fc domain not comprising the one or more amino acid substitutions. Fc binding interactions are essential for a variety of effector functions and downstream signaling events including, but not limited to, antibody dependent cell-mediated cytotoxicity (ADCC) and complement dependent cytotoxicity (CDC). Accordingly, in certain aspects, an antibody comprising a modified Fc region (e.g., comprising a L234A, L235A, and a D265C mutation) has substantially reduced or abolished effector functions.

Affinity to an Fc region can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay or other mechanism of kinetics-based assay (e.g., BIACORE™ analysis or Octet™ analysis (forteBIO)), and other methods such as indirect binding assays, competitive binding assays fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions. One example of a competitive binding assay is a radioimmunoassay comprising the incubation of labeled antigen with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound in the presence of increasing amounts of an unlabeled second antibody.

In one embodiment, the antibodies or antigen binding fragments thereof described herein comprise an Fc region comprising L235A, L235A, and D265C (EU index). The antibodies of used herein may be further engineered to further modulate antibody half-life by introducing additional Fc mutations, such as those described for example in (Dall'Acqua et al. (2006) J Biol Chem 281: 23514-24), (Zalevsky et al. (2010) Nat Biotechnol 28: 157-9), (Hinton et al. (2004) J Biol Chem 279: 6213-6), (Hinton et al. (2006) J Immunol 176: 346-56), (Shields et al. (2001) J Biol Chem 276: 6591-604), (Petkova et al. (2006) Int Immunol 18: 1759-69), (Datta-Mannan et al. (2007) Drug Metab Dispos 35: 86-94), (Vaccaro et al. (2005) Nat Biotechnol 23: 1283-8), (Yeung et al. (2010) Cancer Res 70: 3269-77) and (Kim et al. (1999) Eur J Immunol 29: 2819-25), and include positions 250, 252, 253, 254, 256, 257, 307, 376, 380, 428, 434 and 435. Exemplary mutations that may be made singularly or in combination are T250Q, M252Y, 1253A, S254T, T256E, P2571, T307A, D376V, E380A, M428L, H433K, N434S, N434A, N434H, N434F, H435A and H435R mutations.

Thus, in one embodiment, the Fc region comprises a mutation resulting in a decrease in half life. An antibody having a short half life may be advantageous in certain instances where the antibody is expected to function as a short-lived therapeutic, e.g., the conditioning step described herein where the antibody is administered followed by HSCs. Ideally, the antibody would be substantially cleared prior to delivery of the HSCs. In one embodiment, the Fc regions comprises a mutation at position 435 (EU index according to Kabat). In one embodiment, the mutation is an H435A mutation.

In one embodiment, antibodies or antigen binding fragments thereof described herein comprise an Fc region comprising L235A, L235A, D265C, and H435A (EU index).

In one embodiment, the antibodies or antigen binding fragments thereof described herein have a half life of equal to or less than about 24 hours, equal to or less than about 22 hours, equal to or less than about 20 hours, equal to or less than about 18 hours, equal to or less than about 16 hours, equal to or less than about 14 hours, equal to or less than about 13 hours, equal to or less than about 12 hours, equal to or less than about 11 hours, equal to or less than about 10 hours, equal to or less than about 9 hours, equal to or less than about 8 hours, equal to or less than about 7 hours, equal to or less than about 6 hours, equal to or less than about 5 hours, equal to or less than about 4 hours, equal to or less than about 3 hours, equal to or less than about 2 hours, or equal to or less than about 1 hour. In one embodiment, the half life of the antibody is between about 10 hours to 24 hours; between about 11 hours to 22 hours; between about 12 hours to 20 hours; between about 14 hours to 18 hours; or between about 15 hours to 16 hours.

### Antibody Drug Conjugates (ADCs)

Antibody Drug Conjugates (ADCs) used in the methods disclosed herein include ADCs including cytotoxins and linkers as set forth below. In particular, the compounds include an antibody (or an antigen-binding fragment thereof) conjugated (i.e., covalently attached by a linker) to a cytotoxic moiety (or cytotoxin), wherein the cytotoxic moiety, when not conjugated to an antibody, has a cytotoxic or cytostatic effect. In various embodiments, the cytotoxic moiety exhibits reduced or no cytotoxicity when bound in a conjugate, but resumes cytotoxicity after cleavage from the linker. In various embodiments, the cytotoxic moiety maintains cytotoxicity without cleavage from the linker. In some embodiments, the cytotoxic molecule is conjugated to a cell internalizing antibody, or antigen-binding fragment thereof as disclosed herein, such that following the cellular uptake of the antibody, or fragment thereof, the cytotoxin may access its intracellular target and, e.g., mediate hematopoietic cell death.

Drug-antibody conjugates (ADCs) of the present invention therefore may be of the general Formula I, wherein an antibody or antigen-binding fragment thereof (Ab) is conjugated (covalently linked) to linker (L), through a chemical moiety (Z), to a cytotoxic moiety (“drug,” D).

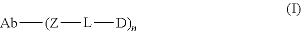

Accordingly, the antibody or antigen-binding fragment thereof may be conjugated to a number of drug moieties as indicated by integer n, which represents the average number of cytotoxins per antibody, which may range, e.g., from about 1 to about 20. In some embodiments, n is from 1 to 4. In some embodiments, n is 1. The average number of drug moieties per antibody in preparations of ADC from conjugation reactions may be characterized by conventional means such as mass spectroscopy, ELISA assay, and HPLC. The quantitative distribution of ADC in terms of n may also be determined. In some instances, separation, purification, and characterization of homogeneous ADC where n is a certain value from ADC with other drug loadings may be achieved by means such as reverse phase HPLC or electrophoresis.

For some antibody-drug conjugates, n may be limited by the number of attachment sites on the antibody. For example, where the attachment is a cysteine thiol, an antibody may have only one or several cysteine thiol groups, or may have only one or several sufficiently reactive thiol groups through which a linker may be attached. Generally, antibodies do not contain many free and reactive cysteine thiol groups which may be linked to a drug moiety; primarily, cysteine thiol residues in antibodies exist as disulfide bridges. In certain embodiments, an antibody may be reduced with a reducing agent such as dithiothreitol (DTT) or tricarbonylethylphosphine (TCEP), under partial or total reducing conditions, to generate reactive cysteine thiol groups. In certain embodiments, higher drug loading, e.g. n>5, may cause aggregation, insolubility, toxicity, or loss of cellular permeability of certain antibody-drug conjugates.

In certain embodiments, fewer than the theoretical maximum of drug moieties are conjugated to an antibody during a conjugation reaction. An antibody may contain, for example, lysine residues that do not react with the drug-linker intermediate or linker reagent, as discussed below. Only the most reactive lysine groups may react with an amine-reactive linker reagent. In certain embodiments, an antibody is subjected to denaturing conditions to reveal reactive nucleophilic groups such as lysine or cysteine.

The loading (drug/antibody ratio) of an ADC may be controlled in different ways, e.g., by: (i) limiting the molar excess of drug-linker intermediate or linker reagent relative to antibody, (ii) limiting the conjugation reaction time or temperature, (iii) partial or limiting reductive conditions for cysteine thiol modification, (iv) engineering by recombinant techniques the amino acid sequence of the antibody such that the number and position of cysteine residues is modified for control of the number and/or position of linker-drug attachments.

### Cytotoxins

Cytotoxins (drugs) suitable for use with the compositions and methods described herein include DNA-intercalating agents, (e.g., anthracyclines), agents capable of disrupting the mitotic spindle apparatus (e.g., vinca alkaloids, maytansine, maytansinoids, and derivatives thereof), RNA polymerase inhibitors (e.g., an amatoxin, such as α-amanitin, and derivatives thereof), and agents capable of disrupting protein biosynthesis (e.g., agents that exhibit rRNA N-glycosidase activity, such as saporin and ricin A-chain), among others known in the art.

In some embodiments, the cytotoxin is a microtubule-binding agent (for instance, maytansine or a maytansinoid), an amatoxin, pseudomonas exotoxin A, deBouganin, diphtheria toxin, saporin, an auristatin, an anthracycline, a calicheamicin, irinotecan, SN-38, a duocarmycin, a pyrrolobenzodiazepine, a pyrrolobenzodiazepine dimer, an indolinobenzodiazepine, an indolinobenzodiazepine dimer, or a variant thereof, or another cytotoxic compound described herein or known in the art.

In some embodiments, the cytotoxin of the antibody-drug conjugate is an RNA polymerase inhibitor. In some embodiments, the RNA polymerase inhibitor is an amatoxin or derivative thereof. In some embodiments, the cytotoxin of the antibody-drug conjugate as disclosed herein is an amatoxin or derivative thereof, such as an α-amanitin, β-amanitin, γ-amanitin, ε-amanitin, amanin, amaninamide, amanullin, amanullinic acid, proamanullin or a derivative thereof.

### Amatoxins

In some embodiments, the RNA polymerase inhibitor is an amatoxin or derivative thereof. In some embodiments, the cytotoxin of the antibody-drug conjugate as disclosed herein is an amatoxin or derivative thereof, such as an α-amanitin, s-amanitin, γ-amanitin, ε-amanitin, amanin, amaninamide, amanullin, amanullinic acid, proamanullin or a derivative thereof. Structures of the various naturally occurring amatoxins are represented by formula II and accompanying Table 2, and are disclosed in, e.g., Zanotti et al., Int. J. Peptide Protein Res. 30, 1987, 450-459.

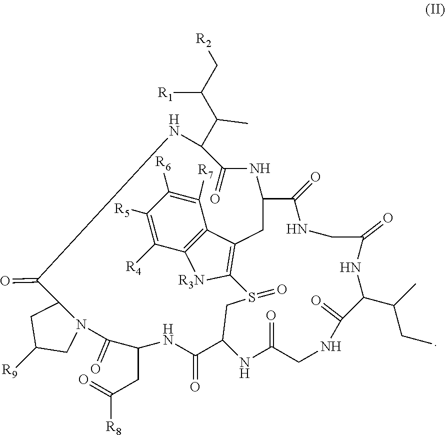

TABLE 2
Amatoxin structure table.

| Name | R₁ | R₂ | R₃, R₄ | R₅ | R₆, R₇ | R₈ | R₉ |
|---|---|---|---|---|---|---|---|
| α-amanitin | OH | OH | H | OH | H | NH₃ | OH |
| β-amanitin | OH | OH | H | OH | H | OH | OH |
| γ-amanitin | OH | H | H | OH | H | NH₃ | OH |
| ε-amanitin | OH | H | H | OH | H | OH | OH |
| Amanin | OH | OH | H | H | H | OH | OH |
| Amaninamide | OH | OH | H | H | H | NH₃ | OH |
| Amanullin | H | H | H | OH | H | NH₃ | OH |
| Amanullinic | H | H | H | OH | H | OH | OH |
| acid | | | | | | | |
| Proamanullin | H | H | H | OH | H | NH₃ | H |

Many positions on amatoxins or derivatives thereof can serve as the position to covalently bond the linking moiety L, and, hence the antibodies or antigen-binding fragments thereof. In some embodiments, the cytotoxin in the ADC of Formula I is an amatoxin or derivative thereof represented by formula (III):

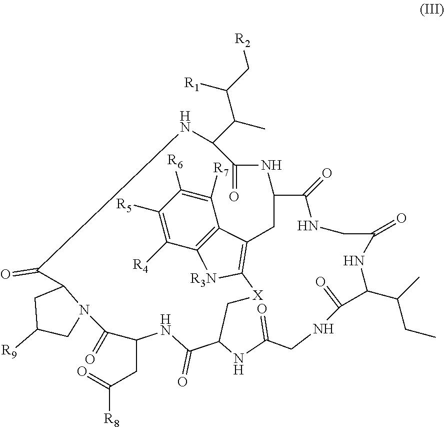

wherein:
R₁ is H, OH, OR_{A}, or OR_{C};
R₂ is H, OH, OR_{B}, or OR_{C};
   R_{A} and R_{B}, when present, together with the oxygen atoms to which they are bound, combine to form a 5-membered heterocycloalkyl group;
R₃ is H, R_{C}, or R_{D};
each of R₄, R₅, R₆, and Rz is independently H, OH, OR_{C}, OR_{D}, R_{C}, or R_{D};
R₈ is OH, NH₂, OR_{C}, OR_{D}, NHR_{C}, or NR_{C}R_{D};
R₉ is H, OH, OR_{C}, or OR_{D};
Q is —S—, —S(O)—, or —SO₂—;
   R_{C} is -L-Z′ or -L-Z-Ab, wherein L is a linker, Z′ is a reactive moiety, and Z is a chemical moiety resulting from a coupling reaction of Z′ with a functional group on Ab; and
   R_{D} is C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, cycloalkyl, heterocycloalkyl, aryl, heteroaryl, or a combination, wherein each C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, cycloalkyl, heterocycloalkyl, aryl, or heteroaryl is optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

In some embodiments, the cytotoxin is an amatoxin represented by formula (IIIA):

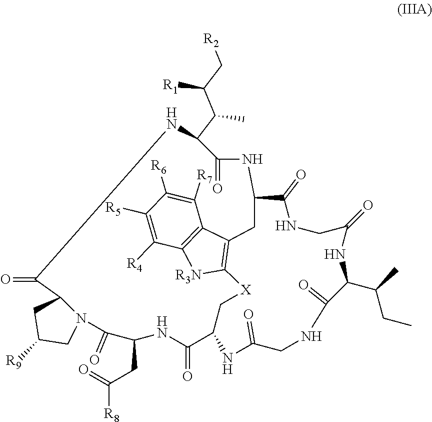

In some embodiments, the amatoxin contains one R_{C} substituent.

In some embodiments, R_{A} and R_{B}, together with the oxygen atoms to which they are bound, combine to form a 5-membered heterocycloalkyl group of formula:

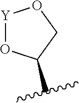

wherein Y is —(C═O)—, —(C═S)—, —(C═NR_{E})—, or —(CR_{E}R_{E′})—; and
wherein R_{E} and R_{E′} are each independently H, C₁-C₆ alkylene-R_{C}, C₁-C₆ heteroalkylene-R_{C}, C₂-C₆ alkenylene-R_{C}, C₂-C₆ heteroalkenylene-R_{C}, C₂-C₆ alkynylene-R_{C}, C₂-C₆ heteroalkynylene-R_{C}, cycloalkylene-R_{C}, heterocycloalkylene-R_{C}, arylene-R_{C}, or heteroarylene-R_{C}, or a combination thereof; wherein each C₁-C₆ alkylene-R_{C}, C₁-C₆ heteroalkylene-R_{C}, C₂-C₆ alkenylene-R_{C}, C₂-C₆ heteroalkenylene-R_{C}, C₂-C₆ alkynylene-R_{C}, C₂-C₆ heteroalkynylene-R_{C}, cycloalkylene-R_{C}, heterocycloalkylene-R_{C}, arylene-R_{C}, or heteroarylene-R_{C} is optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

In some embodiments, the cytotoxin is an amatoxin or derivative thereof is represented by formula IIIA, wherein
R₁ is H, OH, OR_{A}, or OR_{C};
R₂ is H, OH, OR_{B}, or OR_{C};
   R_{A} and R_{B}, when present, together with the oxygen atoms to which they are bound, combine to form:

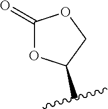

wherein R₃ is H or R_{C}.

In some embodiments, the cytotoxin is an amatoxin or derivative thereof is represented by formula IIIA, wherein
R₁ is H, OH, OR_{A}, or OR_{C};
R₂ is H, OH, OR_{B}, or OR_{C};
   R_{A} and R_{B}, when present, together with the oxygen atoms to which they are bound, combine to form:

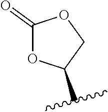

wherein
R₃ is H or R_{C};
R₄ and R₅ are each independently H, OH, OR_{C}, R_{C}, or OR_{D};
R₆ and R₇ are each H;
R₈ is OH, NH₂, OR_{C}, or NHR_{C};
R₉ is H or OH; and
wherein R_{c} and R_{D} are as defined above.

In some embodiments, the cytotoxin is an amatoxin or derivative thereof is represented by formula IIIA, wherein:
R₁ is H, OH, or OR_{A};
R₂ is H, OH, or OR_{B};
   R_{A} and R_{B}, when present, together with the oxygen atoms to which they are bound, combine to form:

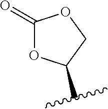

wherein
R₃, R₄, R₆, and R₇ are each H;
R₅ is OR_{C};
R₈ is OH or NH₂;
R₉ is H or OH;
Q is —S—, —S(O)—, or —SO₂—; and
wherein R_{C} and R_{D} are as defined above. Such amatoxin conjugates are described, for example, in U.S. Patent Application Publication No. 2016/0002298, the disclosure of which is incorporated herein by reference in its entirety.

In some embodiments, the cytotoxin is an amatoxin or derivative thereof is represented by formula IIIA, wherein:
R₁ and R₂ are each independently H or OH;
   R₃ is R_{C};
R₄, R₆, and R₇ are each H;
R₅ is H, OH, or OC₁-C₆ alkyl;
R₈ is OH or NH₂;
R₉ is H or OH;
Q is —S—, —S(O)—, or —SO₂—; and
wherein R_{C} and R_{D} are as defined above. Such amatoxin conjugates are described, for example, in U.S. Patent Application Publication No. 2014/0294865, the disclosure of which is incorporated herein by reference in its entirety.

In some embodiments, the cytotoxin is an amatoxin or derivative thereof represented by formula IIIA, wherein:
R₁ and R₂ are each independently H or OH;
   R₃, R₆, and R₇ are each H;
R₄ and R₅ are each independently H, OH, OR_{C}, or R_{C};
R₈ is OH or NH₂;
R₉ is H or OH;
Q is —S—, —S(O)—, or —SO₂—; and
wherein R_{c} and R_{D} are as defined above. Such amatoxin-linker conjugates are described, for example, in U.S. Patent Application Publication No. 2015/0218220, the disclosure of which is incorporated herein by reference in its entirety.

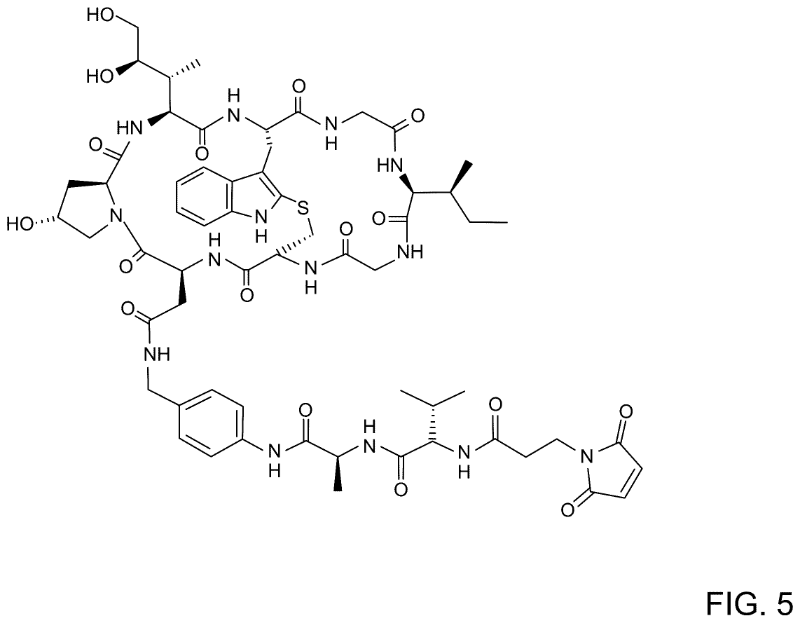

In one embodiment, an amatoxin-linker set forth in FIG. 5 (Z′-L-D), is conjugated to an ADC for use in the methods disclosed herein.

In some embodiments, the cytotoxin is an amatoxin or derivative thereof represented by formula IIIA, wherein:
R₁ and R₂ are each independently H or OH;
   R₃, R₆, and R₇ are each H;
R₄ and R₅ are each independently H or OH;
R₈ is OH, NH₂, OR_{C}, or NHR_{C};
R₉ is H or OH;
Q is —S—, —S(O)—, or —SO₂—; and
wherein R_{C} and R_{D} are as defined above. Such amatoxin conjugates are described, for example, in U.S. Pat. Nos. 9,233,173 and 9,399,681, the disclosures of each of which are incorporated herein by reference in their entirety.

In some embodiments, the cytotoxin is an amatoxin or derivative thereof represented by formula IIIB:

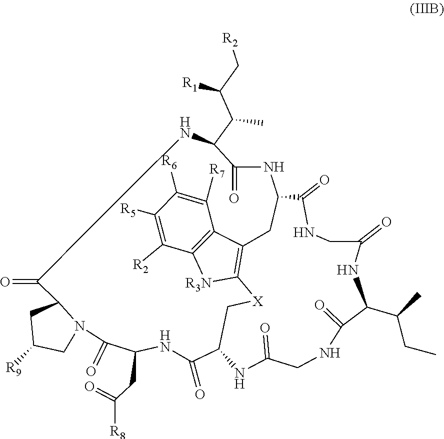

wherein:
R₁ is H, OH, OR_{A}, or OR_{C};
R₂ is H, OH, OR_{B}, or OR_{C};
   R_{A} and R_{B}, when present, together with the oxygen atoms to which they are bound, combine to form a 5-membered heterocycloalkyl group;
R₃ is H, R_{C}, or R_{D};
each of R₄, R₅, R₆, and R₇ is independently H, OH, OR_{C}, OR_{D}, R_{C}, or R_{D};
R₈ is OH, NH₂, OR_{C}, OR_{D}, NHR_{C}, or NR_{C}R_{D};
R₉ is H, OH, OR_{C}, or OR_{D};
Q is —S—, —S(O)—, or —SO₂—;
   R_{C} is -L-Z′ or -L-Z-Ab, wherein L is a linker, Z′ is a reactive moiety, and Z is a chemical moiety resulting from a coupling reaction of Z′ with a functional group on Ab; and
R_{D} is C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, cycloalkyl, heterocycloalkyl, aryl, heteroaryl, or a combination, wherein each C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, cycloalkyl, heterocycloalkyl, aryl, or heteroaryl is optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

In some embodiments, R_{A} and R_{B}, together with the oxygen atoms to which they are bound, combine to form a 5-membered heterocycloalkyl of formula:

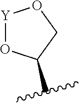

wherein Y is —(C═O)—, —(C═S)—, —(C═NR_{E})—, or —(CR_{E}R_{E′})—; and
wherein R_{E} and R_{E′} are each independently H, C₁-C₆ alkylene-R_{C}, C₁-C₆ heteroalkylene-R_{C}, C₂-C₆ alkenylene-R_{C}, C₂-C₆ heteroalkenylene-R_{C}, C₂-C₆ alkynylene-R_{C}, C₂-C₆ heteroalkynylene-R_{C}, cycloalkylene-R_{C}, heterocycloalkylene-R_{C}, arylene-R_{C}, or heteroarylene-R_{C}, or a combination thereof, wherein each C₁-C₆ alkylene-R_{C}, C₁-C₆ heteroalkylene-R_{C}, C₂-C₆ alkenylene-R_{C}, C₂-C₆ heteroalkenylene-R_{C}, C₂-C₆ alkynylene-R_{C}, C₂-C₆ heteroalkynylene-R_{C}, cycloalkylene-R_{C}, heterocycloalkylene-R_{C}, arylene-R_{C}, or heteroarylene-R_{C} is optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

In some embodiments, the antibodies or antigen-binding fragments thereof as described herein are conjugated to an amatoxin-linker conjugate, or derivative thereof, represented by formula IIIB, wherein
R₁ is H, OH, OR_{A}, or OR_{C};
R₂ is H, OH, OR_{B}, or OR_{C};
   R_{A} and R_{B}, when present, together with the oxygen atoms to which they are bound, combine to form a 5-membered heterocycloalkyl of formula:

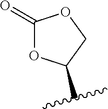

wherein R₃ is H or R_{C}.

In some embodiments, the cytotoxin is an amatoxin or derivative thereof represented by formula IIIB, wherein
R₁ is H, OH, OR_{A}, or OR_{C};
R₂ is H, OH, OR_{B}, or OR_{C};
   R_{A} and R_{B}, when present, together with the oxygen atoms to which they are bound, combine to form a 5-membered heterocycloalkyl group of formula:

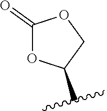

wherein
   R₃ is H or R_{C};
R₄ and R₅ are each independently H, OH, OR_{C}, R_{C}, or OR_{D};
R₆ and R₇ are each H;
R₈ is OH, NH₂, OR_{C}, or NHR_{C};
R₉ is H or OH; and
wherein R_{C} and R_{D} are as defined above.

In some embodiments, the cytotoxin is an amatoxin or derivative thereof, represented by formula IIIB, wherein:
R₁ is H, OH, or OR_{A};
R₂ is H, OH, or OR_{B};
   R_{A} and R_{B}, when present, together with the oxygen atoms to which they are bound, combine to form a 5-membered heterocycloalkyl group of formula:

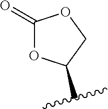

wherein
   R₃, R₄, R₆, and R₇ are each H;
R₅ is OR_{C};
R₈ is OH or NH₂;
R₉ is H or OH;
Q is —S—, —S(O)—, or —SO₂—; and
wherein R_{C} and R_{D} are as defined above. Such amatoxin-linker conjugates are described, for example, in U.S. Patent Application Publication No. 2016/0002298, the disclosure of which is incorporated herein by reference in its entirety.

In some embodiments, the cytotoxin is an amatoxin or derivative thereof, represented by formula IIIB, wherein:
R₁ and R₂ are each independently H or OH;
   R₃ is R_{C};
R₄, R₆, and R₇ are each H;
R₅ is H, OH, or OC₁-C₆ alkyl;
R₈ is OH or NH₂;
R₉ is H or OH;
Q is —S—, —S(O)—, or —SO₂—; and
wherein R_{C} and R_{D} are as defined above. Such amatoxin conjugates are described, for example, in U.S. Patent Application Publication No. 2014/0294865, the disclosure of which is incorporated herein by reference in its entirety.

In some embodiments, the cytotoxin is an amatoxin or derivative thereof, represented by formula IIIB, wherein:
R₁ and R₂ are each independently H or OH;
   R₃, R₆, and R₇ are each H;
R₄ and R₅ are each independently H, OH, OR_{C}, or R_{C};
R₈ is OH or NH₂;
R₉ is H or OH;
Q is —S—, —S(O)—, or —SO₂—; and
wherein R_{C} and R_{D} are as defined above. Such amatoxin-linker conjugates are described, for example, in U.S. Patent Application Publication No. 2015/0218220, the disclosure of which is incorporated herein by reference in its entirety.

In some embodiments, the cytotoxin is an amatoxin or derivative thereof, represented by formula IIIB, wherein:
R₁ and R₂ are each independently H or OH;
   R₃, R₆, and R₇ are each H;
R₄ and R₅ are each independently H or OH;
R₈ is OH, NH₂, OR_{C}, or NHR_{C};
R₉ is H or OH;
Q is —S—, —S(O)—, or —SO₂—; and
wherein R_{C} and R_{D} are as defined above. Such amatoxin-linker conjugates are described, for example, in U.S. Pat. Nos. 9,233,173 and 9,399,681, the disclosures of each of which are incorporated herein by reference in their entirety.

### Auristatins

Antibodies and antigen-binding fragments thereof described herein can be conjugated to a cytotoxin that is an auristatin (U.S. Pat. Nos. 5,635,483; 5,780,588). Auristatins are anti-mitotic agents that interfere with microtubule dynamics, GTP hydrolysis, and nuclear and cellular division (Woyke et al (2001) Antimicrob. Agents and Chemother. 45(12):3580-3584) and have anticancer (U.S. Pat. No. 5,663,149) and antifungal activity (Pettit et al (1998) Antimicrob. Agents Chemother. 42:2961-2965). (U.S. Pat. Nos. 5,635,483; 5,780,588). The auristatin drug moiety may be attached to the antibody through the N (amino) terminus or the C (carboxyl) terminus of the peptidic drug moiety (WO 02/088172).

Exemplary auristatin embodiments include the N-terminus linked monomethylauristatin drug moieties DE and DF, disclosed in Senter et al, Proceedings of the American Association for Cancer Research, Volume 45, Abstract Number 623, presented Mar. 28, 2004, the disclosure of which is expressly incorporated by reference in its entirety.

An exemplary auristatin embodiment is MMAE, wherein the wavy line indicates the point of covalent attachment to the linker of an antibody-linker conjugate (-L-Z-Ab or -L-Z′, as described herein).

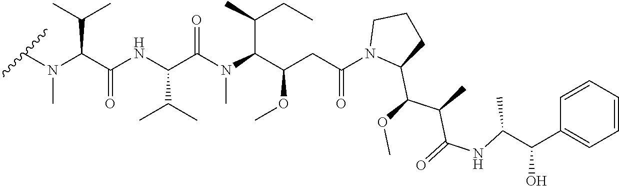 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

Another exemplary auristatin embodiment is MMAF, wherein the wavy line indicates the point of covalent attachment to the linker of an antibody-linker conjugate (-L-Z-Ab or -L-Z′, as described herein), as disclosed in US 2005/0238649:

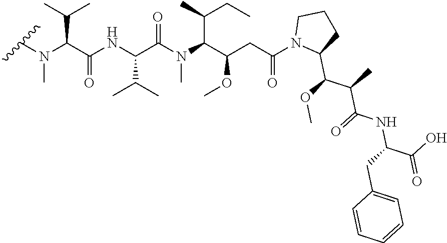 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)C(=O)O)OC)C(C)C)C(C)C]

Auristatins may be prepared according to the methods of: U.S. Pat. Nos. 5,635,483; 5,780,588; Pettit et al (1989) J. Am. Chem. Soc. 111:5463-5465; Pettit et al (1998) Anti-Cancer Drug Design 13:243-277; Pettit, G. R., et al. Synthesis, 1996, 719-725; Pettit et al (1996) J. Chem. Soc. Perkin Trans. 15:859-863; and Doronina (2003) Nat. Biotechnol. 21(7):778-784.

### Maytansinoids

Antibodies and antigen-binding fragments thereof described herein can be conjugated to a cytotoxin that is a microtubule binding agent. In some embodiments, the microtubule binding agent is a maytansine, a maytansinoid or a maytansinoid analog. Maytansinoids are mitototic inhibitors which bind microtubules and act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533. Maytansinoid drug moieties are attractive drug moieties in antibody drug conjugates because they are: (i) relatively accessible to prepare by fermentation or chemical modification, derivatization of fermentation products, (ii) amenable to derivatization with functional groups suitable for conjugation through the non-disulfide linkers to antibodies, (iii) stable in plasma, and (iv) effective against a variety of tumor cell lines.

Examples of suitable maytansinoids include esters of maytansinol, synthetic maytansinol, and maytansinol analogs and derivatives. Included herein are any cytotoxins that inhibit microtubule formation and that are highly toxic to mammalian cells, as are maytansinoids, maytansinol, and maytansinol analogs, and derivatives.

Examples of suitable maytansinol esters include those having a modified aromatic ring and those having modifications at other positions. Such suitable maytansinoids are disclosed in U.S. Pat. Nos. 4,137,230; 4,151,042; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,362,663; 4,364,866; 4,424,219; 4,450,254; 4,322,348; 4,362,663; 4,371,533; 5,208,020; 5,416,064; 5,475,092; 5,585,499; 5,846,545; 6,333,410; 7,276,497; and 7,473,796, the disclosures of each of which are incorporated herein by reference as they pertain to maytansinoids and derivatives thereof.

In some embodiments, the antibody-drug conjugates (ADCs) of the present disclosure utilize the thiol-containing maytansinoid (DM1), formally termed N^{2′}-deacetyl-N^{2′}-(3-mercapto-1-oxopropyl)-maytansine, as the cytotoxic agent. DM1 is represented by the following structural formula V:

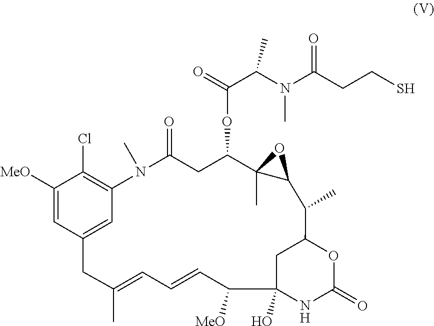 [SMILES: COc1cc2cc(c1Cl)N(C)C(=O)C[C@H](OC(=O)[C@H](C)N(C)C(=O)CCS)[C@]1(C)O[C@H]1[C@H](C)C1C[C@@](O)(NC(=O)O1)[C@H](OC)/C=C/C=C(\C)C2]

In another embodiment, the conjugates of the present invention utilize the thiol-containing maytansinoid N^{2′}-deacetyl-N^{2′}(4-methyl-4-mercapto-1-oxopentyl)-maytansine (e.g., DM4) as the cytotoxic agent. DM4 is represented by the following structural formula VI:

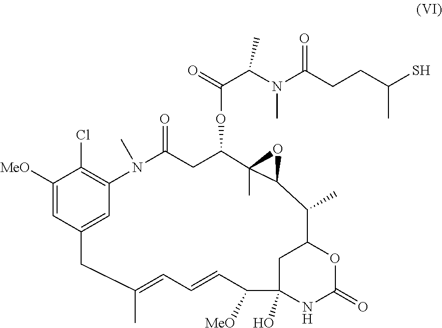 [SMILES: COc1cc2cc(c1Cl)N(C)C(=O)C[C@H](OC(=O)[C@H](C)N(C)C(=O)CCC(C)S)[C@]1(C)O[C@H]1[C@H](C)C1C[C@@](O)(NC(=O)O1)[C@H](OC)/C=C/C=C(\C)C2]

Another maytansinoid comprising a side chain that contains a sterically hindered thiol bond is N^{2′}-deacetyl-N²′(4-mercapto-1-oxopentyl)-maytansine (termed DM3), represented by the following structural formula VII:

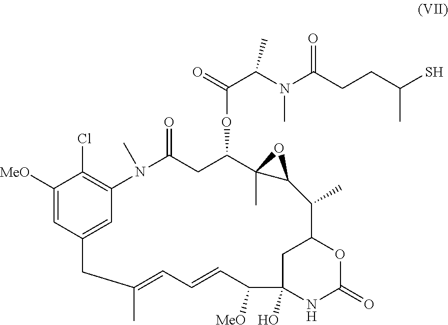 [SMILES: COc1cc2cc(c1Cl)N(C)C(=O)C[C@H](OC(=O)[C@H](C)N(C)C(=O)CCC(C)S)[C@]1(C)O[C@H]1[C@H](C)C1C[C@@](O)(NC(=O)O1)[C@H](OC)/C=C/C=C(\C)C2]

Each of the maytansinoids taught in U.S. Pat. Nos. 5,208,020 and 7,276,497, can also be used in the conjugates of the present disclosure. In this regard, the entire disclosure of U.S. Pat. Nos. 5,208,020 and 7,276,697 is incorporated herein by reference.

Many positions on maytansinoids can serve as the position to covalently bond the linking moiety and, hence the antibodies or antigen-binding fragments thereof (-L-Z-Ab or -L-Z′, as described herein). For example, the C-3 position having a hydroxyl group, the C-14 position modified with hydroxymethyl, the C-15 position modified with hydroxy and the C-20 position having a hydroxy group are all expected to be useful. In some embodiments, the C-3 position serves as the position to covalently bond the linker moiety, and in some particular embodiments, the C-3 position of maytansinol serves as the position to covalently bond the linking moiety. There are many linking groups known in the art for making antibody-maytansinoid conjugates, including, for example, those disclosed in U.S. Pat. Nos. 5,208,020, 6,441,163, and EP Patent No. 0425235 B1; Chari et al., Cancer Research 52:127-131 (1992); and U.S. 2005/0169933 A1, the disclosures of which are hereby expressly incorporated by reference. Additional linking groups are described and exemplified herein.

The present invention also includes various isomers and mixtures of maytansinoids and conjugates. Certain compounds and conjugates of the present invention may exist in various stereoisomeric, enantiomeric, and diastereomeric forms. Several descriptions for producing such antibody-maytansinoid conjugates are provided in U.S. Pat. Nos. 5,208,020; 5,416,064; 6,333,410; 6,441,163; 6,716,821; and 7,368,565, each of which is incorporated herein in its entirety.

### Anthracyclines

In other embodiments, the antibodies and antigen-binding fragments thereof described herein can be conjugated to a cytotoxin that is an anthracycline molecule. Anthracyclines are antibiotic compounds that exhibit cytotoxic activity. Studies have indicated that anthracyclines may operate to kill cells by a number of different mechanisms including: 1) intercalation of the drug molecules into the DNA of the cell thereby inhibiting DNA-dependent nucleic acid synthesis; 2) production by the drug of free radicals which then react with cellular macromolecules to cause damage to the cells or 3) interactions of the drug molecules with the cell membrane [see, e.g., C. Peterson et al., “Transport And Storage Of Anthracycline In Experimental Systems And Human Leukemia” in Anthracycline Antibiotics In Cancer Therapy; N. R. Bachur, “Free Radical Damage” id. at pp. 97-102]. Because of their cytotoxic potential anthracyclines have been used in the treatment of numerous cancers such as leukemia, breast carcinoma, lung carcinoma, ovarian adenocarcinoma and sarcomas [see e.g., P. H-Wiernik, in Anthracycline: Current Status And New Developments p 11]. Commonly used anthracyclines include doxorubicin, epirubicin, idarubicin and daunomycin.

The anthracycline analog, doxorubicin (ADRIAMYCINO) is thought to interact with DNA by intercalation and inhibition of the progression of the enzyme topoisomerase II, which unwinds DNA for transcription. Doxorubicin stabilizes the topoisomerase II complex after it has broken the DNA chain for replication, preventing the DNA double helix from being resealed and thereby stopping the process of replication. Doxorubicin and daunorubicin (DAUNOMYCIN) are prototype cytotoxic natural product anthracycline chemotherapeutics (Sessa et al., (2007) Cardiovasc. Toxicol. 7:75-79).

### Pyrrolobenzodiazepines (PBDs)

In other embodiments, an antibody, or antigen-binding fragment thereof, described herein can be conjugated to a cytotoxin that is a pyrrolobenzodiazepine (PBD) or a cytotoxin that comprises a PBD. PBDs are natural products produced by certain actinomycetes and have been shown to be sequence selective DNA alkylating compounds. PBD cytotoxins include, but are not limited to, anthramycin, dimeric PBDs, and those disclosed in, for example, Hartley, JA (2011) The development of pyrrolobenzodiazepines as antitumor agents. Expert Opin Inv Drug, 20(6), 733-744 and Antonow D, Thurston DE (2011) Synthesis of DNA-interactive pyrrolo[2,1-c][1,4]benzodiazepines (PBDs). Chem Rev 111: 2815-2864.

PBDs are of the general structure:

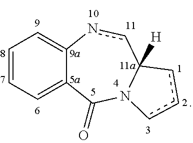

In some embodiments, the cytotoxin used in the invention is a dimer represented by the structural formula:

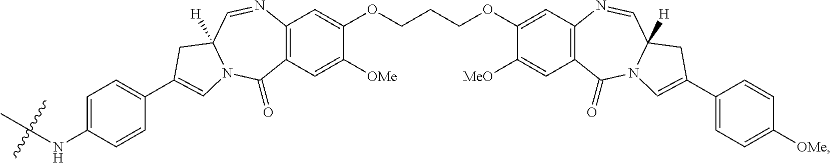 [SMILES: *Nc1ccc(C2=CN3C(=O)c4cc(OC)c(OCCCOc5cc6c(cc5OC)C(=O)N5C=C(c7ccc(OC)cc7)C[C@H]5C=N6)cc4N=C[C@@H]3C2)cc1]

wherein the wavy line indicates the point of covalent attachment to the linker of the ADO as described herein.

In some embodiments, the cytotoxin used in the invention is a dimer represented by the structural formula:

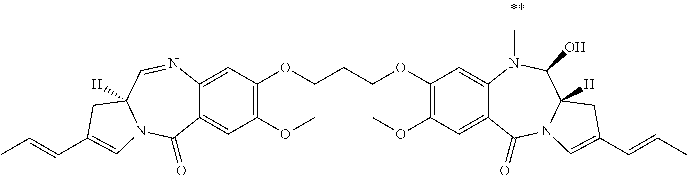 [SMILES: C/C=C/C1=CN2C(=O)c3cc(OC)c(OCCCOc4cc5c(cc4OC)C(=O)N4C=C(/C=C/C)C[C@H]4[C@H](O)N5C)cc3N=C[C@@H]2C1]

wherein the “**” indicates the point of covalent attachment to the linker of the ADC as described herein.

In some embodiments, the cytotoxin used in the invention is a dimer represented by the structural formula:

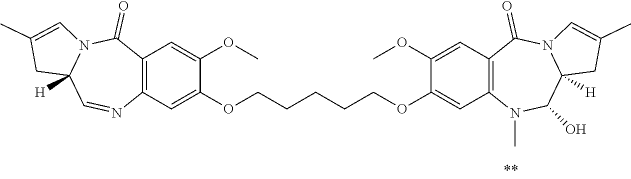

wherein the “**” indicates the point of covalent attachment to the linker of the ADC as described herein.

In some embodiments, the linker and cytotoxin portion of a contemplated ADC, i.e., the “linker-cytotoxin” portion of a contemplated ADC, may be selected from talirine or tesirine.

### Calicheamicin

In other embodiments, the antibodies and antigen-binding fragments thereof described herein can be conjugated to a cytotoxin that is a calicheamicin molecule. The calicheamicin family of antibiotics are capable of producing double-stranded DNA breaks at sub-picomolar concentrations. For the preparation of conjugates of the calicheamicin family, see U.S. Pat. Nos. 5,712,374; 5,714,586; 5,739,116; 5,767,285; 5,770,701; 5,770,710; 5,773,001; and 5,877,296 (all to American Cyanamid Company). Structural analogues of calicheamicin which may be used include, but are not limited to, those disclosed in, for example, Hinman et al., Cancer Research 53:3336-3342 (1993), Lode et al., Cancer Research 58:2925-2928 (1998), and the aforementioned U.S. patents to American Cyanamid.

### Additional Cytotoxins

In other embodiments, the antibodies and antigen-binding fragments thereof described herein can be conjugated to a cytotoxin other than or in addition to those cytotoxins disclosed herein above. Additional cytotoxins suitable for use with the compositions and methods described herein include, without limitation, 5-ethynyluracil, abiraterone, acylfulvene, adecypenol, adozelesin, aldesleukin, altretamine, ambamustine, amidox, amifostine, aminolevulinic acid, amrubicin, amsacrine, anagrelide, anastrozole, andrographolide, angiogenesis inhibitors, antarelix, anti-dorsalizing morphogenetic protein-1, antiandrogen, prostatic carcinoma, antiestrogen, antineoplaston, antisense oligonucleotides, aphidicolin glycinate, apoptosis gene modulators, apoptosis regulators, apurinic acid, asulacrine, atamestane, atrimustine, axinastatin 1, axinastatin 2, axinastatin 3, azasetron, azatoxin, azatyrosine, baccatin III derivatives, balanol, batimastat, BCR/ABL antagonists, benzochlorins, benzoylstaurosporine, beta lactam derivatives, beta-alethine, betaclamycin B, betulinic acid, bFGF inhibitors, bicalutamide, bisantrene, bisaziridinylspermine, bisnafide, bistratene A, bizelesin, breflate, bleomycin A2, bleomycin B2, bropirimine, budotitane, buthionine sulfoximine, calcipotriol, calphostin C, camptothecin derivatives (e.g., 10-hydroxy-camptothecin), capecitabine, carboxamide-amino-triazole, carboxyamidotriazole, carzelesin, casein kinase inhibitors, castanospermine, cecropin B, cetrorelix, chlorins, chloroquinoxaline sulfonamide, cicaprost, cis-porphyrin, cladribine, clomifene and analogues thereof, clotrimazole, collismycin A, collismycin B, combretastatin A4, combretastatin analogues, conagenin, crambescidin 816, crisnatol, cryptophycin 8, cryptophycin A derivatives, curacin A, cyclopentanthraquinones, cycloplatam, cypemycin, cytarabine ocfosfate, cytolytic factor, cytostatin, dacliximab, decitabine, dehydrodidemnin B, 2′deoxycoformycin (DCF), deslorelin, dexifosfamide, dexrazoxane, dexverapamil, diaziquone, didemnin B, didox, diethylnorspermine, dihydro-5-azacytidine, dihydrotaxol, dioxamycin, diphenyl spiromustine, discodermolide, docosanol, dolasetron, doxifluridine, droloxifene, dronabinol, duocarmycin SA, ebselen, ecomustine, edelfosine, edrecolomab, eflornithine, elemene, emitefur, epothilones, epithilones, epristeride, estramustine and analogues thereof, etoposide, etoposide 4′-phosphate (also referred to as etopofos), exemestane, fadrozole, fazarabine, fenretinide, filgrastim, finasteride, flavopiridol, flezelastine, fluasterone, fludarabine, fluorodaunorunicin hydrochloride, forfenimex, formestane, fostriecin, fotemustine, gadolinium texaphyrin, gallium nitrate, galocitabine, ganirelix, gelatinase inhibitors, gemcitabine, glutathione inhibitors, hepsulfam, homoharringtonine (HHT), hypericin, ibandronic acid, idoxifene, idramantone, ilmofosine, ilomastat, imidazoacridones, imiquimod, immunostimulant peptides, iobenguane, iododoxorubicin, ipomeanol, irinotecan, iroplact, irsogladine, isobengazole, jasplakinolide, kahalalide F, lamellarin-N triacetate, lanreotide, leinamycin, lenograstim, lentinan sulfate, leptolstatin, letrozole, lipophilic platinum compounds, lissoclinamide 7, lobaplatin, lometrexol, lonidamine, losoxantrone, loxoribine, lurtotecan, lutetium texaphyrin, lysofylline, masoprocol, maspin, matrix metalloproteinase inhibitors, menogaril, rnerbarone, meterelin, methioninase, metoclopramide, MIF inhibitor, ifepristone, miltefosine, mirimostim, mithracin, mitoguazone, mitolactol, mitomycin and analogues thereof, mitonafide, mitoxantrone, mofarotene, molgramostim, mycaperoxide B, myriaporone, N-acetyldinaline, N-substituted benzamides, nafarelin, nagrestip, napavin, naphterpin, nartograstim, nedaplatin, nemorubicin, neridronic acid, nilutamide, nisamycin, nitrullyn, octreotide, okicenone, onapristone, ondansetron, oracin, ormaplatin, oxaliplatin, oxaunomycin, paclitaxel and analogues thereof, palauamine, palmitoylrhizoxin, pamidronic acid, panaxytriol, panomifene, parabactin, pazelliptine, pegaspargase, peldesine, pentosan polysulfate sodium, pentostatin, pentrozole, perflubron, perfosfamide, phenazinomycin, picibanil, pirarubicin, piritrexim, podophyllotoxin, porfiromycin, purine nucleoside phosphorylase inhibitors, raltitrexed, rhizoxin, rogletimide, rohitukine, rubiginone B1, ruboxyl, safingol, saintopin, sarcophytol A, sargramostim, sobuzoxane, sonermin, sparfosic acid, spicamycin D, spiromustine, stipiamide, sulfinosine, tallimustine, tegafur, temozolomide, teniposide, thaliblastine, thiocoraline, tirapazamine, topotecan, topsentin, triciribine, trimetrexate, veramine, vinorelbine, vinxaltine, vorozole, zeniplatin, and zilascorb, among others.

### Linkers for Chemical Conjugation

The term “Linker” as used herein means a divalent chemical moiety comprising a covalent bond or a chain of atoms that covalently attaches an antibody or fragment thereof (Ab) to a drug moiety (D) to form antibody-drug conjugates (ADC) of formula I. Suitable linkers have two reactive termini, one for conjugation to an antibody and the other for conjugation to a cytotoxin. The antibody conjugation reactive terminus of the linker (reactive moiety, Z′) is typically a site that is capable of conjugation to the antibody through a cysteine thiol or lysine amine group on the antibody, and so is typically a thiol-reactive group such as a double bond (as in maleimide) or a leaving group such as a chloro, bromo, iodo, or an R-sulfanyl group, or an amine-reactive group such as a carboxyl group; while the antibody conjugation reactive terminus of the linker is typically a site that is capable of conjugation to the cytotoxin through formation of an amide bond with a basic amine or carboxyl group on the cytotoxin, and so is typically a carboxyl or basic amine group. When the term “linker” is used in describing the linker in conjugated form, one or both of the reactive termini will be absent (such as reactive moiety Z′, having been converted to chemical moiety Z) or incomplete (such as being only the carbonyl of the carboxylic acid) because of the formation of the bonds between the linker and/or the cytotoxin, and between the linker and/or the antibody or antigen-binding fragment thereof. Such conjugation reactions are described further herein below.

A variety of linkers can be used to conjugate the antibodies, antigen-binding fragments, and ligands described to a cytotoxic molecule. In some embodiments, the linker is cleavable under intracellular conditions, such that cleavage of the linker releases the drug unit from the antibody in the intracellular environment. In yet other embodiments, the linker unit is not cleavable and the drug is released, for example, by antibody degradation. The linkers useful for the present ADCs are preferably stable extracellularly, prevent aggregation of ADC molecules and keep the ADC freely soluble in aqueous media and in a monomeric state. Before transport or delivery into a cell, the ADC is preferably stable and remains intact, i.e. the antibody remains linked to the drug moiety. The linkers are stable outside the target cell and may be cleaved at some efficacious rate inside the cell. An effective linker will: (i) maintain the specific binding properties of the antibody; (ii) allow intracellular delivery of the conjugate or drug moiety; (iii) remain stable and intact, i.e. not cleaved, until the conjugate has been delivered or transported to its targeted site; and (iv) maintain a cytotoxic, cell-killing effect or a cytostatic effect of the cytotoxic moiety. Stability of the ADC may be measured by standard analytical techniques such as mass spectroscopy, HPLC, and the separation/analysis technique LC/MS. Covalent attachment of the antibody and the drug moiety requires the linker to have two reactive functional groups, i.e. bivalency in a reactive sense. Bivalent linker reagents which are useful to attach two or more functional or biologically active moieties, such as peptides, nucleic acids, drugs, toxins, antibodies, haptens, and reporter groups are known, and methods have been described their resulting conjugates (Hermanson, G. T. (1996) Bioconjugate Techniques; Academic Press: New York, p. 234-242).

Suitable cleavable linkers include those that may be cleaved, for instance, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012, the disclosure of which is incorporated herein by reference as it pertains to linkers suitable for covalent conjugation). Suitable cleavable linkers may include, for example, chemical moieties such as a hydrazine, a disulfide, a thioether or a dipeptide.

Linkers hydrolyzable under acidic conditions include, for example, hydrazones, semicarbazones, thiosemicarbazones, cis-aconitic amides, orthoesters, acetals, ketals, or the like. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661, the disclosure of each of which is incorporated herein by reference in its entirety as it pertains to linkers suitable for covalent conjugation. Such linkers are relatively stable under neutral pH conditions, such as those in the blood, but are unstable at below pH 5.5 or 5.0, the approximate pH of the lysosome.

Linkers cleavable under reducing conditions include, for example, a disulfide. A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935, the disclosure of each of which is incorporated herein by reference in its entirety as it pertains to linkers suitable for covalent conjugation.

Linkers susceptible to enzymatic hydrolysis can be, e.g., a peptide-containing linker that is cleaved by an intracellular peptidase or protease enzyme, including, but not limited to, a lysosomal or endosomal protease. One advantage of using intracellular proteolytic release of the therapeutic agent is that the agent is typically attenuated when conjugated and the serum stabilities of the conjugates are typically high. In some embodiments, the peptidyl linker is at least two amino acids long or at least three amino acids long. Exemplary amino acid linkers include a dipeptide, a tripeptide, a tetrapeptide or a pentapeptide. Examples of suitable peptides include those containing amino acids such as Valine, Alanine, Citrulline (Cit), Phenylalanine, Lysine, Leucine, and Glycine. Amino acid residues which comprise an amino acid linker component include those occurring naturally, as well as minor amino acids and non-naturally occurring amino acid analogs, such as citrulline. Exemplary dipeptides include valine-citrulline (vc or val-cit) and alanine-phenylalanine (af or ala-phe). Exemplary tripeptides include glycine-valine-citrulline (gly-val-cit) and glycine-glycine-glycine (gly-gly-gly). In some embodiments, the linker includes a dipeptide such as Val-Cit, Ala-Val, or Phe-Lys, Val-Lys, Ala-Lys, Phe-Cit, Leu-Cit, Ile-Cit, Phe-Arg, or Trp-Cit. Linkers containing dipeptides such as Val-Cit or Phe-Lys are disclosed in, for example, U.S. Pat. No. 6,214,345, the disclosure of which is incorporated herein by reference in its entirety as it pertains to linkers suitable for covalent conjugation. In some embodiments, the linker includes a dipeptide selected from Val-Ala and Val-Cit.

Linkers suitable for conjugating the antibodies, antigen-binding fragments, and ligands described herein to a cytotoxic molecule include those capable of releasing a cytotoxin by a 1,6-elimination process. Chemical moieties capable of this elimination process include the p-aminobenzyl (PAB) group, 6-maleimidohexanoic acid, pH-sensitive carbonates, and other reagents as described in Jain et al., Pharm. Res. 32:3526-3540, 2015, the disclosure of which is incorporated herein by reference in its entirety as it pertains to linkers suitable for covalent conjugation.

In some embodiments, the linker includes a “self-immolative” group such as the aforementioned PAB or PABC (para-aminobenzyloxycarbonyl), which are disclosed in, for example, Carl et al., J. Med. Chem. (1981) 24:479-480; Chakravarty et al (1983) J. Med. Chem. 26:638-644; U.S. Pat. No. 6,214,345; US20030130189; US20030096743; U.S. Pat. No. 6,759,509; US20040052793; U.S. Pat. Nos. 6,218,519; 6,835,807; 6,268,488; US20040018194; WO98/13059; US20040052793; U.S. Pat. Nos. 6,677,435; 5,621,002; US20040121940; WO2004/032828). Other such chemical moieties capable of this process (“self-immolative linkers”) include methylene carbamates and heteroaryl groups such as aminothiazoles, aminoimidazoles, aminopyrimidines, and the like. Linkers containing such heterocyclic self-immolative groups are disclosed in, for example, U.S. Patent Publication Nos. 20160303254 and 20150079114, and U.S. Pat. No. 7,754,681; Hay et al. (1999) Bioorg. Med. Chem. Lett. 9:2237; US 2005/0256030; de Groot et al (2001) J. Org. Chem. 66:8815-8830; and U.S. Pat. No. 7,223,837. In some embodiments, a dipeptide is used in combination with a self-immolative linker.

Linkers suitable for use herein further may include one or more groups selected from C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, heteroarylene, and combinations thereof, each of which may be optionally substituted. Non-limiting examples of such groups include (CH₂)ₚ, (CH₂CH₂O)ₚ, and —(C═O)(CH₂)ₚ— units, wherein p is an integer from 1-6, independently selected for each occasion.

Suitable linkers may contain groups having solubility enhancing properties. Linkers including the (CH₂CH₂O)ₚ unit (polyethylene glycol, PEG), for example, can enhance solubility, as can alkyl chains substituted with amino, sulfonic acid, phosphonic acid or phosphoric acid residues. Linkers including such moieties are disclosed in, for example, U.S. Pat. Nos. 8,236,319 and 9,504,756, the disclosure of each of which is incorporated herein by reference in its entirety as it pertains to linkers suitable for covalent conjugation. Further solubility enhancing groups include, for example, acyl and carbamoyl sulfamide groups, having the structure:

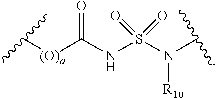

wherein a is 0 or 1; and
R¹⁰ is selected from the group consisting of hydrogen, C₁-C₂₄ alkyl groups, C₃-C₂₄ cycloalkyl groups, C₁-C₂₄ (hetero)aryl groups, C₁-C₂₄ alkyl(hetero)aryl groups and C₁-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, each of which may be optionally substituted and/or optionally interrupted by one or more heteroatoms selected from O, S and NR¹¹R¹², wherein R¹¹ and R¹² are independently selected from the group consisting of hydrogen and C₁-C₄ alkyl groups; or R¹⁰ is a cytotoxin, wherein the cytotoxin is optionally connected to N via a spacer moiety. Linkers containing such groups are described, for example, in U.S. Pat. No. 9,636,421 and U.S. Patent Application Publication No. 2017/0298145, the disclosures of which are incorporated herein by reference in their entirety as they pertain to linkers suitable for covalent conjugation to cytotoxins and antibodies or antigen-binding fragments thereof.

In some embodiments, the linker may include one or more of a hydrazine, a disulfide, a thioether, a dipeptide, a p-aminobenzyl (PAB) group, a heterocyclic self-immolative group, an optionally substituted C₁-C₆ alkyl, an optionally substituted C₁-C₆ heteroalkyl, an optionally substituted C₂-C₆ alkenyl, an optionally substituted C₂-C₆ heteroalkenyl, an optionally substituted C₂-C₆ alkynyl, an optionally substituted C₂-C₆ heteroalkynyl, an optionally substituted C₃-C₆ cycloalkyl, an optionally substituted heterocycloalkyl, an optionally substituted aryl, an optionally substituted heteroaryl, a solubility enhancing group, acyl, —(C═O)—, or —(CH₂CH₂O)ₚ— group, wherein p is an integer from 1-6. One of skill in the art will recognize that one or more of the groups listed may be present in the form of a bivalent (diradical) species, e.g., C₁-C₆ alkylene and the like.

In some embodiments, the linker includes a p-aminobenzyl group (PAB). In one embodiment, the p-aminobenzyl group is disposed between the cytotoxic drug and a protease cleavage site in the linker. In one embodiment, the p-aminobenzyl group is part of a p-aminobenzyloxycarbonyl unit. In one embodiment, the p-aminobenzyl group is part of a p-aminobenzylamido unit.

In some embodiments, the linker comprises PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB.

In some embodiments, the linker comprises a combination of one or more of a peptide, oligosaccharide, —(CH₂)ₚ—, —(CH₂CH₂O)ₚ—, PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB.

In some embodiments, the linker comprises a —(C═O)(CH₂)ₚ— unit, wherein p is an integer from 1-6.

In one specific embodiment, the linker comprises the structure

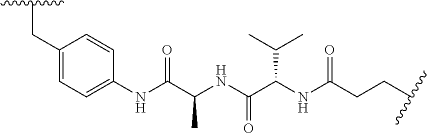 [SMILES: *CCC(=O)N[C@H](C(=O)N[C@@H](C)C(=O)Nc1ccc(C*)cc1)C(C)C]

wherein the wavy lines indicate attachment points to the cytotoxin and the reactive moiety Z′. In another specific embodiment, the linker comprises the structure

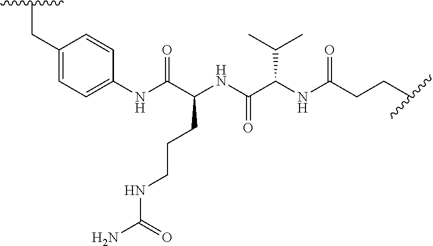 [SMILES: *CCC(=O)N[C@H](C(=O)N[C@@H](CCCNC(N)=O)C(=O)Nc1ccc(C*)cc1)C(C)C]

wherein the wavy lines indicate attachment points to the cytotoxin and the reactive moiety Z′. Such PAB-dipeptide-propionyl linkers are disclosed in, e.g., Patent Application Publication No. WO2017/149077, which is incorporated by reference herein in its entirety. Further, the cytotoxins disclosed in WO2017/149077 are incorporated by reference herein.

It will be recognized by one of skill in the art that any one or more of the chemical groups, moieties and features disclosed herein may be combined in multiple ways to form linkers useful for conjugation of the antibodies and cytotoxins as disclosed herein. Further linkers useful in conjunction with the compositions and methods described herein, are described, for example, in U.S. Patent Application Publication No. 2015/0218220, the disclosure of which is incorporated herein by reference in its entirety.

In certain embodiments, an intermediate, which is the precursor of the linker, is reacted with the drug moiety under appropriate conditions, In certain embodiments, reactive groups are used on the drug and/or the intermediate or linker. The product of the reaction between the drug and the intermediate, or the derivatized drug, is subsequently reacted with the antibody or antigen-binding fragment under appropriate conditions. Alternatively, the linker or intermediate may first be reacted with the antibody or a derivatized antibody, and then reacted with the drug or derivatized drug. Such conjugation reactions will now be described more fully.

A number of different reactions are available for covalent attachment of linkers or drug-linker conjugates to the antibody or antigen-binding fragment thereof. Suitable attachment points on the antibody molecule include the amine groups of lysine, the free carboxylic acid groups of glutamic acid and aspartic acid, the sulfhydryl groups of cysteine, and the various moieties of the aromatic amino acids. For instance, non-specific covalent attachment may be undertaken using a carbodiimide reaction to link a carboxy (or amino) group on a compound to an amino (or carboxy) group on an antibody moiety. Additionally, bifunctional agents such as dialdehydes or imidoesters may also be used to link the amino group on a compound to an amino group on an antibody moiety. Also available for attachment of drugs to binding agents is the Schiff base reaction. This method involves the periodate oxidation of a drug that contains glycol or hydroxy groups, thus forming an aldehyde which is then reacted with the binding agent. Attachment occurs via formation of a Schiff base with amino groups of the binding agent. Isothiocyanates may also be used as coupling agents for covalently attaching drugs to binding agents. Other techniques are known to the skilled artisan and within the scope of the present disclosure.

Linkers useful in for conjugation to the antibodies or antigen-binding fragments as described herein include, without limitation, linkers containing chemical moieties Z formed by coupling reactions as depicted in Table 3, below. Curved lines designate points of attachment to the antibody or antigen-binding fragment, and the cytotoxic molecule, respectively.

TABLE 3
Exemplary chemical moieties Z formed by coupling reactions
in the formulation of antibody-drug conjugates

| Exemplary | |
|---|---|
| Coupling | |
| Reactions | Chemical Moiety Z Formed by Coupling Reactions |
| [3 + 2] Cycloaddition | 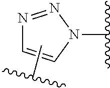 |
| [3 + 2] Cycloaddition | 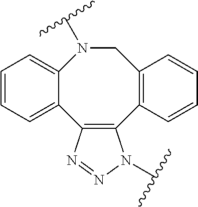 [SMILES: *N1Cc2ccccc2-c2c(nnn2*)-c2ccccc21] |
| [3 + 2] Cycloaddition, Esterification | 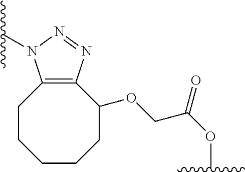 [SMILES: *OC(=O)COC1CCCCCc2c1nnn2*] |
| [3 + 2] Cycloaddition, Esterification | 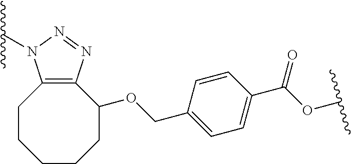 [SMILES: *OC(=O)c1ccc(COC2CCCCCc3c2nnn3*)cc1] |
| [3 + 2] Cycloaddition, Esterification | 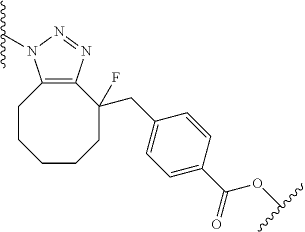 [SMILES: *OC(=O)c1ccc(CC2(F)CCCCCc3c2nnn3*)cc1] |
| [3 + 2] Cycloaddition, Esterification | 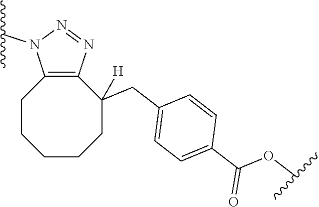 [SMILES: *OC(=O)c1ccc(CC2CCCCCc3c2nnn3*)cc1] |
| [3 + 2] Cycloaddition, Esterification | 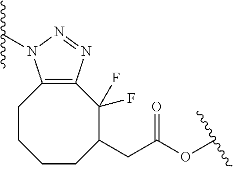 [SMILES: *OC(=O)CC1CCCCc2c(nnn2*)C1(F)F] |
| [3 + 2] Cycloaddition, Esterification | 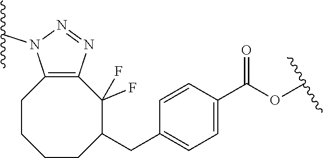 [SMILES: *OC(=O)c1ccc(CC2CCCCc3c(nnn3*)C2(F)F)cc1] |
| [3 + 2] Cycloaddition, Esterification | 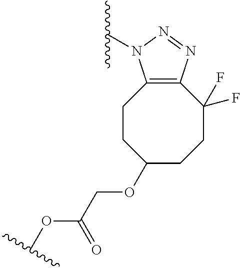 [SMILES: *OC(=O)COC1CCc2c(nnn2*)C(F)(F)CC1] |
| [3 + 2] Cycloaddition, Esterification | 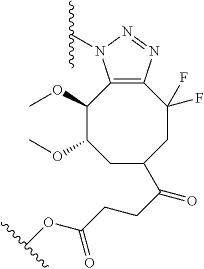 [SMILES: *OC(=O)CCC(=O)C1C[C@H](OC)[C@@H](OC)c2c(nnn2*)C(F)(F)C1] |
| [3 + 2] Cycloaddition, Esterification | 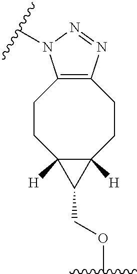 [SMILES: *OC[C@@H]1[C@@H]2CCc3c(nnn3*)CC[C@@H]21] |
| [3 + 2] Cycloaddition, Esterification | 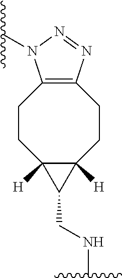 [SMILES: *NC[C@@H]1[C@@H]2CCc3c(nnn3*)CC[C@@H]21] |
| [3 + 2] Cycloaddition, Esterification | 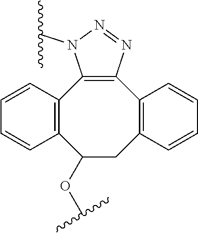 [SMILES: *OC1Cc2ccccc2-c2nnn(*)c2-c2ccccc21] |
| [3 + 2] Cycloaddition, Esterification | 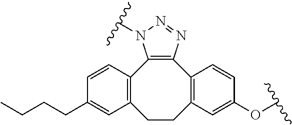 [SMILES: *Oc1ccc2c(c1)CCc1cc(CCCC)ccc1-c1c-2nnn1*] |
| [3 + 2] Cycloaddition | 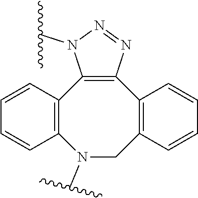 [SMILES: *N1Cc2ccccc2-c2nnn(*)c2-c2ccccc21] |
| Michael addition | 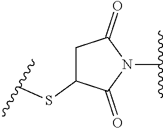 [SMILES: *SC1CC(=O)N(*)C1=O] |
| Michael addition | 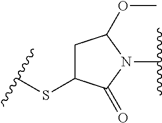 [SMILES: *SC1CC(OC)N(*)C1=O] |
| Imine condensation, Amidation | 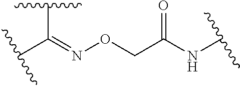 [SMILES: *NC(=O)CON=C(*)*] |
| Imine condensation | 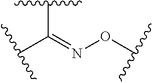 [SMILES: *ON=C(*)*] |
| Disulfide formation | 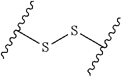 [SMILES: *SS*] |
| Thio alkylation | 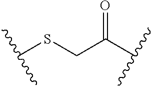 [SMILES: *SCC(*)=O] |
| Condensation, Michael addition | 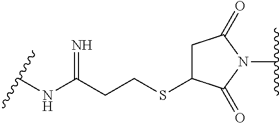 [SMILES: *NC(=N)CCSC1CC(=O)N(*)C1=O] |

One of skill in the art will recognize that a reactive substituent Z′ attached to the linker and a reactive substituent on the antibody or antigen-binding fragment thereof, are engaged in the covalent coupling reaction to produce the chemical moiety Z, and will recognize the reactive moiety Z′. Therefore, antibody-drug conjugates useful in conjunction with the methods described herein may be formed by the reaction of an antibody, or antigen-binding fragment thereof, with a linker or cytotoxin-linker conjugate, as described herein, the linker or cytotoxin-linker conjugate including a reactive substituent Z′, suitable for reaction with a reactive substituent on the antibody, or antigen-binding fragment thereof, to form the chemical moiety Z.

As depicted in Table 3, examples of suitably reactive substituents on the linker and antibody or antigen-binding fragment thereof include a nucleophile/electrophile pair (e.g., a thiol/haloalkyl pair, an amine/carbonyl pair, or a thiol/α,β-unsaturated carbonyl pair, and the like), a diene/dienophile pair (e.g., an azide/alkyne pair, or a diene/α,β-unsaturated carbonyl pair, among others), and the like. Coupling reactions between the reactive substitutents to form the chemical moiety Z include, without limitation, thiol alkylation, hydroxyl alkylation, amine alkylation, amine or hydroxylamine condensation, hydrazine formation, amidation, esterification, disulfide formation, cycloaddition (e.g., [4+2]Diels-Alder cycloaddition, [3+2] Huisgen cycloaddition, among others), nucleophilic aromatic substitution, electrophilic aromatic substitution, and other reactive modalities known in the art or described herein. Preferably, the linker contains an electrophilic functional group for reaction with a nucleophilic functional group on the antibody, or antigen-binding fragment thereof.

Reactive substituents that may be present within an antibody, or antigen-binding fragment thereof, as disclosed herein include, without limitation, nucleophilic groups such as (i)N-terminal amine groups, (ii) side chain amine groups, e.g. lysine, (iii) side chain thiol groups, e.g. cysteine, and (iv) sugar hydroxyl or amino groups where the antibody is glycosylated. Reactive substituents that may be present within an antibody, or antigen-binding fragment thereof, as disclosed herein include, without limitation, hydroxyl moieties of serine, threonine, and tyrosine residues; amino moieties of lysine residues; carboxyl moieties of aspartic acid and glutamic acid residues; and thiol moieties of cysteine residues, as well as propargyl, azido, haloaryl (e.g., fluoroaryl), haloheteroaryl (e.g., fluoroheteroaryl), haloalkyl, and haloheteroalkyl moieties of non-naturally occurring amino acids. In some embodiments, the reactive substituents present within an antibody, or antigen-binding fragment thereof as disclosed herein include, are amine or thiol moieties. Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol. Reactive thiol groups may be introduced into the antibody (or fragment thereof) by introducing one, two, three, four, or more cysteine residues (e.g., preparing mutant antibodies comprising one or more non-native cysteine amino acid residues). U.S. Pat. No. 7,521,541 teaches engineering antibodies by introduction of reactive cysteine amino acids.

In some embodiments, the reactive moiety Z′ attached to the linker is a nucleophilic group which is reactive with an electrophilic group present on an antibody. Useful electrophilic groups on an antibody include, but are not limited to, aldehyde and ketone carbonyl groups. The heteroatom of a nucleophilic group can react with an electrophilic group on an antibody and form a covalent bond to the antibody. Useful nucleophilic groups include, but are not limited to, hydrazide, oxime, amino, hydroxyl, hydrazine, thiosemicarbazone, hydrazine carboxylate, and arylhydrazide.

In some embodiments, Z is the product of a reaction between reactive nucleophilic substituents present within the antibodies, or antigen-binding fragments thereof, such as amine and thiol moieties, and a reactive electrophilic substituent Z′. For instance, Z′ may be a Michael acceptor (e.g., maleimide), activated ester, electron-deficient carbonyl compound, and aldehyde, among others.

For instance, linkers suitable for the synthesis of drug-antibody and drug-ligand conjugates include, without limitation, reactive substituents Z′ such as maleimide or haloalkyl groups. These may be attached to the linker by reagents such as succinimidyl 4-(N-maleimidomethyl)-cyclohexane-L-carboxylate (SMCC), N-succinimidyl iodoacetate (SIA), sulfo-SMCC, m-maleimidobenzoyl-N-hydroxysuccinimidyl ester (MBS), sulfo-MBS, and succinimidyl iodoacetate, among others described, in for instance, Liu et al., 18:690-697, 1979, the disclosure of which is incorporated herein by reference as it pertains to linkers for chemical conjugation.

In some embodiments, the reactive substituent Z′ attached to linker L is a maleimide, azide, or alkyne. An example of a maleimide-containing linker is the non-cleavable maleimidocaproyl-based linker, which is particularly useful for the conjugation of microtubule-disrupting agents such as auristatins. Such linkers are described by Doronina et al., Bioconjugate Chem. 17:14-24, 2006, the disclosure of which is incorporated herein by reference as it pertains to linkers for chemical conjugation.

In some embodiments, the reactive substituent Z′ is —(C═O)— or —NH(C═O)—, such that the linker may be joined to the antibody, or antigen-binding fragment thereof, by an amide or urea moiety, respectively, resulting from reaction of the —(C═O)— or —NH(C═O)— group with an amino group of the antibody or antigen-binding fragment thereof.

In some embodiments, the reactive substituent is an N-maleimidyl group, halogenated N-alkylamido group, sulfonyloxy N-alkylamido group, carbonate group, sulfonyl halide group, thiol group or derivative thereof, alkynyl group comprising an internal carbon-carbon triple bond, (het-ero)cycloalkynyl group, bicyclo[6.1.0]non-4-yn-9-yl group, alkenyl group comprising an internal carbon-carbon double bond, cycloalkenyl group, tetrazinyl group, azido group, phosphine group, nitrile oxide group, nitrone group, nitrile imine group, diazo group, ketone group, (O-alkyl)hydroxylamino group, hydrazine group, halogenated N-maleimidyl group, 1,1-bis (sulfonylmethyl)methylcarbonyl group or elimination derivatives thereof, carbonyl halide group, or an allenamide group, each of which may be optionally substituted. In some embodiments, the reactive substituent comprises a cycloalkene group, a cycloalkyne group, or an optionally substituted (hetero)cycloalkynyl group.

Non-limiting examples of amatoxin-linker conjugates containing a reactive substituent Z′ suitable for reaction with a reactive residue on the antibody or antigen-binding fragment thereof include, without limitation, 7′C-(4-(6-(maleimido)hexanoyl)piperazin-1-yl)-amatoxin; 7′C-(4-(6-(maleimido)hexanamido)piperidin-1-yl)-amatoxin; 7′C-(4-(6-(6-(maleimido)hexanamido)hexanoyl)piperazin-1-yl)-amatoxin; 7′C-(4-(4-((maleimido)methyl)cyclohexanecarbonyl)piperazin-1-yl)-amatoxin; 7′C-(4-(6-(4-((maleimido)methyl)cyclohexanecarboxamido)hexanoyl)piperazin-1-yl)-amatoxin; 7′C-(4-(2-(6-(maleimido)hexanamido)ethyl)piperidin-1-yl)-amatoxin; 7′C-(4-(2-(6-(6-(maleimido)hexanamido)hexanamido)ethyl)piperidin-1-yl)-amatoxin; 7′C-(4-(2-(4-((maleimido)methyl)cyclohexanecarboxamido)ethyl)piperidin-1-yl)-amatoxin; 7′C-(4-(2-(6-(4-((maleimido)methyl)cyclohexanecarboxamido)hexanamido)ethyl)piperidin-1-yl)-amatoxin; 7′C-(4-(2-(3-carboxypropanamido)ethyl)piperidin-1-yl)-amatoxin; 7′C-(4-(2-(2-bromoacetamido)ethyl)piperidin-1-yl)-amatoxin; 7′C-(4-(2-(3-(pyridin-2-yldisulfanyl)propanamido)ethyl)piperidin-1-yl)-amatoxin; 7′C-(4-(2-(4-(maleimido)butanamido)ethyl)piperidin-1-yl)-amatoxin; 7′C-(4-(2-(maleimido)acetyl)piperazin-1-yl)-amatoxin; 7′C-(4-(3-(maleimido)propanoyl)piperazin-1-yl)-amatoxin; 7′C-(4-(4-(maleimido)butanoyl)piperazin-1-yl)-amatoxin; 7′C-(4-(2-(6-(4-((maleimido)methyl)cyclohexanecarboxamido)hexanamido)ethyl)piperidin-11-yl)-amatoxin; 7′C-(3-((6-(maleimido)hexanamido)methyl)pyrrolidin-1-yl)-amatoxin; 7′C-(3-((6-(6-(maleimido)hexanamido)hexanamido)methyl)pyrrolidin-1-yl)-amatoxin; 7′C-(3-((4-((maleimido)methyl)cyclohexanecarboxamido)methyl)pyrrolidin-1-yl)-amatoxin; 7′C-(3-((6-((4-(maleimido)methyl)cyclohexanecarboxamido)hexanamido)methyl)pyrrolidin-1-yl)-amatoxin; 7′C-(4-(2-(6-(2-(aminooxy)acetamido)hexanamido)ethyl)piperidin-1-yl)-amatoxin; 7′C-(4-(2-(4-(2-(aminooxy)acetamido)butanamido)ethyl)piperidin-1-yl)-amatoxin; 7′C-(4-(4-(2-(aminooxy)acetamido)butanoyl)piperazin-1-yl)-amatoxin; 7′C-(4-(6-(2-(aminooxy)acetamido)hexanoyl)piperazin-1-yl)-amatoxin; 7′C-((4-(6-(maleimido)hexanamido)piperidin-1-yl)methyl)-amatoxin; 7′C-((4-(2-(6-(maleimido)hexanamido)ethyl)piperidin-1-yl)methyl)-amatoxin; 7′C-((4-(6-(maleimido)hexanoyl)piperazin-1-yl)methyl)-amatoxin; (R)-7′C-((3-((6-(maleimido)hexanamido)methyl)pyrrolidin-1-yl)methyl)-amatoxin; (S)-7′C-((3-((6-(maleimido)hexanamido)methyl)pyrrolidin-1-yl)methyl)-amatoxin; 7′C-((4-(2-(6-(6-(maleimido)hexanamido)hexanamido)ethyl)piperidin-1-yl)methyl)-amatoxin; 7′C-((4-(2-(4-((maleimido)methyl)cyclohexanecarboxamido)ethyl)piperidin-1-yl)methyl)-amatoxin; 7′C-((4-(2-(6-(4-((maleimido)methyl)cyclohexanecarboxamido)hexanamido)ethyl)piperidin-11-yl)methyl)-amatoxin; 7′C-((4-(2-(6-(maleimido)hexanamido)ethyl)piperazin-1-yl)methyl)-amatoxin; 7′C-((4-(2-(6-(6-(maleimido)hexanamido)hexanamido)ethyl)piperazin-1-yl)methyl)-amatoxin; 7′C-((4-(2-(4-((maleimido)methyl)cyclohexanecarboxamido)ethyl)piperazin-1-yl)methyl)-amatoxin; 7′C-((4-(2-(6-(4-((maleimido)methyl)cyclohexanecarboxamido)hexanamido)ethyl)piperazin-1-yl)methyl)-amatoxin; 7′C-((3-((6-(6-(maleimido)hexanamido)hexanamido)-S-methyl)pyrrolidin-1-yl)methyl)-amatoxin; 7′C-((3-((6-(6-(maleimido)hexanamido)hexanamido)-R-methyl)pyrrolidin-1-yl)methyl)-amatoxin; 7′C-((3-((4-((maleimido)methyl)cyclohexanecarboxamido)-S-methyl)pyrrolidin-1-yl)methyl)-amatoxin; 7′C-((3-((4-((maleimido)methyl)cyclohexanecarboxamido)-R-methyl)pyrrolidin-1-yl)methyl)-amatoxin; 7′C-((3-((6-(4-((maleimido)methyl)cyclohexanecarboxamido)hexanamido)methyl)pyrrolidin-1-yl)methyl)-amatoxin; 7′C-((4-(2-(3-carboxypropanamido)ethyl)piperazin-1-yl)methyl)-amatoxin; 7′C-((4-(6-(6-(maleimido)hexanamido)hexanoyl)piperazin-1-yl)methyl)-amatoxin; 7′C-((4-(6-(4-((maleimido)methyl)cyclohexanecarboxamido)hexanoyl)piperazin-1-yl)methyl)-amatoxin; 7′C-((4-(2-(maleimido)acetyl)piperazin-1-yl)methyl)-amatoxin; 7′C-((4-(3-(maleimido)propanoyl)piperazin-1-yl)methyl)-amatoxin; 7′C-((4-(4-(maleimido)butanoyl)piperazin-1-yl)methyl)-amatoxin; 7′C-((4-(2-(2-(maleimido)acetamido)ethyl)piperidin-1-yl)methyl)-amatoxin; 7′C-((4-(2-(4-(maleimido)butanamido)ethyl)piperidin-1-yl)methyl)-amatoxin; 7′C-((4-(2-(6-(4-((maleimido)methyl)cyclohexanecarboxamido)hexanamido)ethyl)piperidin-1-yl)methyl)-amatoxin; 7′C-((3-((6-(maleimido)hexanamido)methyl)azetidin-1-yl)methyl)-amatoxin; 7′C-((3-(2-(6-(maleimido)hexanamido)ethyl)azetidin-1-yl)methyl)-amatoxin; 7′C-((3-((4-((maleimido)methyl)cyclohexanecarboxamido)methyl)azetidin-1-yl)methyl)-amatoxin; 7′C-((3-(2-(4-((maleimido)methyl)cyclohexanecarboxamido)ethyl)azetidin-1yl)methyl)-amatoxin; 7′C-((3-(2-(6-(4-((maleimido)methyl)cyclohexanecarboxamido)hexanamido)ethyl)azetidin-1-yl)methyl)-amatoxin; 7′C-(((2-(6-(maleimido)-N-methylhexanamido)ethyl)(methyl)amino)methyl)-amatoxin; 7′C-(((4-(6-(maleimido)-N-methylhexanamido)butyl(methyl)amino)methyl)-amatoxin; 7′C-((2-(2-(6-(maleimido)hexanamido)ethyl)aziridin-1-yl)methyl)-amatoxin; 7′C-((2-(2-(6-(4-((maleimido)methyl)cyclohexanecarboxamido)hexanamido)ethyl)aziridin-1-yl)methyl)-amatoxin; 7′C-((4-(6-(6-(2-(aminooxy)acetamido)hexanamido)hexanoyl)piperazin-1-yl)methyl)-amatoxin; 7′C-((4-(1-(aminooxy)-2-oxo-6,9,12,15-tetraoxa-3-azaheptadecan-17-oyl)piperazin-1-yl)methyl)-amatoxin; 7′C-((4-(2-(2-(aminooxy)acetamido)acetyl)piperazin-1-yl)methyl)-amatoxin; 7′C-((4-(3-(2-(aminooxy)acetamido)propanoyl)piperazin-1-yl)methyl)-amatoxin; 7′C-((4-(4-(2-(aminooxy)acetamido)butanoyl)piperazin-1-yl)methyl)-amatoxin; 7′C-((4-(2-(6-(2-(aminooxy)acetamido)hexanamido)ethyl)piperidin-1-yl)methyl)-amatoxin; 7′C-((4-(2-(2-(2-(aminooxy)acetamido)acetamido)ethyl)piperidin-1-yl)methyl)-amatoxin; 7′C-((4-(2-(4-(2-(aminooxy)acetamido)butanamido)ethyl)piperidin-1-yl)methyl)-amatoxin; 7′C-((4-(20-(aminooxy)-4,19-dioxo-6,9,12,15-tetraoxa-3,18-diazaicosyl)piperidin-1-yl)methyl)-amatoxin; 7′C-(((2-(6-(2-(aminooxy)acetamido)-N-methylhexanamido)ethyl)(methyl)amino)methyl)-amatoxin; 7′C-(((4-(6-(2-(aminooxy)acetamido)-N-methylhexanamido)butyl)(methyl)amino)methyl)-amatoxin; 7′C-((3-((6-(4-((maleimido)methyl)cyclohexanecarboxamido)hexanamido)methyl)pyrrolidin-1-yl)-S-methyl)-amatoxin; 7′C-((3-((6-(4-((maleimido)methyl)cyclohexanecarboxamido)hexanamido)-R-methyl)pyrrolidin-1-yl)methyl)-amatoxin; 7′C-((4-(2-(2-bromoacetamido)ethyl)piperazin-1-yl)methyl)-amatoxin; 7′C-((4-(2-(2-bromoacetamido)ethyl)piperidin-1-yl)methyl)-amatoxin; 7′C-((4-(2-(3-(pyridine-2-yldisulfanyl)propanamido)ethyl)piperidin-1-yl)methyl)-amatoxin; 6′O-(6-(6-(maleimido)hexanamido)hexyl)-amatoxin; 6′O-(5-(4-((maleimido)methyl)cyclohexanecarboxamido)pentyl)-amatoxin; 6′O-(2-((6-(maleimido)hexyl)oxy)-2-oxoethyl)-amatoxin; 6′O-((6-(maleimido)hexyl)carbamoyl)-amatoxin; 6′O-((6-(4-((maleimido)methyl)cyclohexanecarboxamido)hexyl)carbamoyl)-amatoxin; 6′O-(6-(2-bromoacetamido)hexyl)-amatoxin; 7′C-(4-(6-(azido)hexanamido)piperidin-1-yl)-amatoxin; 7′C-(4-(hex-5-ynoylamino)piperidin-1-yl)-amatoxin; 7′C-(4-(2-(6-(maleimido)hexanamido)ethyl)piperazin-1-yl)-amatoxin; 7′C-(4-(2-(6-(6-(maleimido)hexanamido)hexanamido)ethyl)piperazin-1-yl)-amatoxin; 6′O-(6-(6-(11,12-didehydro-5,6-dihydro-dibenz[b,f]azocin-5-yl)-6-oxohexanamido)hexyl)-amatoxin; 6′O-(6-(hex-5-ynoylamino)hexyl)-amatoxin; 6′O-(6-(2-(aminooxy)acetylamido)hexyl)-amatoxin; 6′O-((6-aminooxy)hexyl)-amatoxin; and 6′O-(6-(2-iodoacetamido)hexyl)-amatoxin.

In some embodiments, an amatoxin as disclosed herein is conjugated to a linker-reactive moiety -L-Z′ having the following formula:

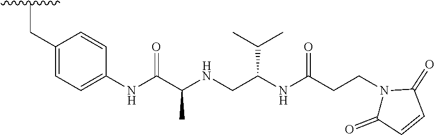 [SMILES: *Cc1ccc(NC(=O)[C@H](C)NC[C@@H](NC(=O)CCN2C(=O)C=CC2=O)C(C)C)cc1]

In some embodiments, an amatoxin as disclosed herein is conjugated to a linker-reactive moiety -L-Z′ having the following formula:

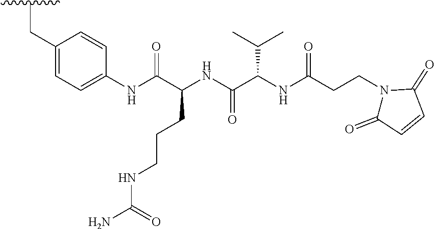 [SMILES: *Cc1ccc(NC(=O)[C@H](CCCNC(N)=O)NC(=O)[C@@H](NC(=O)CCN2C(=O)C=CC2=O)C(C)C)cc1]

The foregoing linker moieties and amatoxin-linker conjugates, among others useful in conjunction with the compositions and methods described herein, are described, for example, in U.S. Patent Application Publication No. 2015/0218220 and Patent Application Publication No. WO2017/149077, the disclosure of each of which is incorporated herein by reference in its entirety.

### Preparation of Antibody-Drug Conjugates

In the ADCs of formula I as disclosed herein, an antibody or antigen binding fragment thereof is conjugated to one or more cytotoxic drug moieties (D), e.g. about 1 to about 20 drug moieties per antibody, through a linker L and a chemical moiety Z as disclosed herein. The ADCs of the present disclosure may be prepared by several routes, employing organic chemistry reactions, conditions, and reagents known to those skilled in the art, including: (1) reaction of a reactive substituent of an antibody or antigen binding fragment thereof with a bivalent linker reagent to form Ab-Z-L as described herein above, followed by reaction with a drug moiety D; or (2) reaction of a reactive substituent of a drug moiety with a bivalent linker reagent to form D-L-Z′, followed by reaction with a reactive substituent of an antibody or antigen binding fragment thereof as described herein above. Additional methods for preparing ADC are described herein.

In another aspect, the antibody or antigen binding fragment thereof has one or more lysine residues that can be chemically modified to introduce one or more sulfhydryl groups. The ADC is then formed by conjugation through the sulfhydryl group's sulfur atom as described herein above.

The reagents that can be used to modify lysine include, but are not limited to, N-succinimidyl S-acetylthioacetate (SATA) and 2-Iminothiolane hydrochloride (Traut's Reagent).

In another aspect, the antibody or antigen binding fragment thereof can have one or more carbohydrate groups that can be chemically modified to have one or more sulfhydryl groups. The ADC is then formed by conjugation through the sulfhydryl group's sulfur atom as described herein above.

In yet another aspect, the antibody can have one or more carbohydrate groups that can be oxidized to provide an aldehyde (—CHO) group (see, for e.g., Laguzza, et al., J. Med. Chem. 1989, 32(3), 548-55). The ADC is then formed by conjugation through the corresponding aldehyde as described herein above. Other protocols for the modification of proteins for the attachment or association of cytotoxins are described in Coligan et al., Current Protocols in Protein Science, vol. 2, John Wiley & Sons (2002), incorporated herein by reference.

Methods for the conjugation of linker-drug moieties to cell-targeted proteins such as antibodies, immunoglobulins or fragments thereof are found, for example, in U.S. Pat. Nos. 5,208,020; 6,441,163; WO2005037992; WO2005081711; and WO2006/034488, all of which are hereby expressly incorporated by reference in their entirety.

Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

### EXAMPLES

The below examples describe studies where elevated liver enzymes in non-human primates at high doses of CD117-amanitin ADC were observed, suggesting toxicity. The toxicity, however, was abrogated when the same dosage was administered in two or more doses on separate days. The magnitude of the reduction in liver enzyme levels far exceeded the reduction in the dose—for example, liver enzymes were elevated, e.g., by 100 fold above normal, when a single dose of ADC dosage (e.g., 0.6 mg/kg) was administered, but splitting the dose into two equal parts (e.g., 2×0.3 mg/kg) did not merely soften the magnitude of the elevation in liver enzymes. Rather, the liver enzyme levels returned to normal, i.e., toxicity is averted despite administering in total the same amount of drug. Importantly, equivalent efficacy is observed under both dosing regimens (single dose vs split doses).

The ADCs used in the following examples include various anti-CD117 antibodies conjugated to a cytotoxin (D) via a linker (L). Specifically, the ADCs were synthesized from conjugation of the various anti-CD117 antibodies to the compound depicted in FIG. 5. More specifically, ADC 1 is an anti-CD117 antibody, i.e., CK6, characterized by a fast (i.e., short) half-life with a modified Fc region comprising D265C and H435A Fc mutations. ADC 2 is an anti-CD117 antibody comprising a heavy chain variable region as set forth in SEQ ID NO: 106, and a light chain variable region as set forth in SEQ ID NO: 107, and is characterized by a fast (i.e., short) half-life with a modified Fc region comprising D265C and H435A Fc mutations. ADC 3 is an anti-CD117 antibody comprising a heavy chain variable region as set forth in SEQ ID NO: 106, and a light chain variable region as set forth in SEQ ID NO: 107, and is characterized by a fast (i.e., short) half-life with a modified Fc region comprising D265C, H435A, and the LALA Fc mutations.

### Example 1. Analysis of Reticulocyte Cell Count in an In Vivo Dose Escalation Study

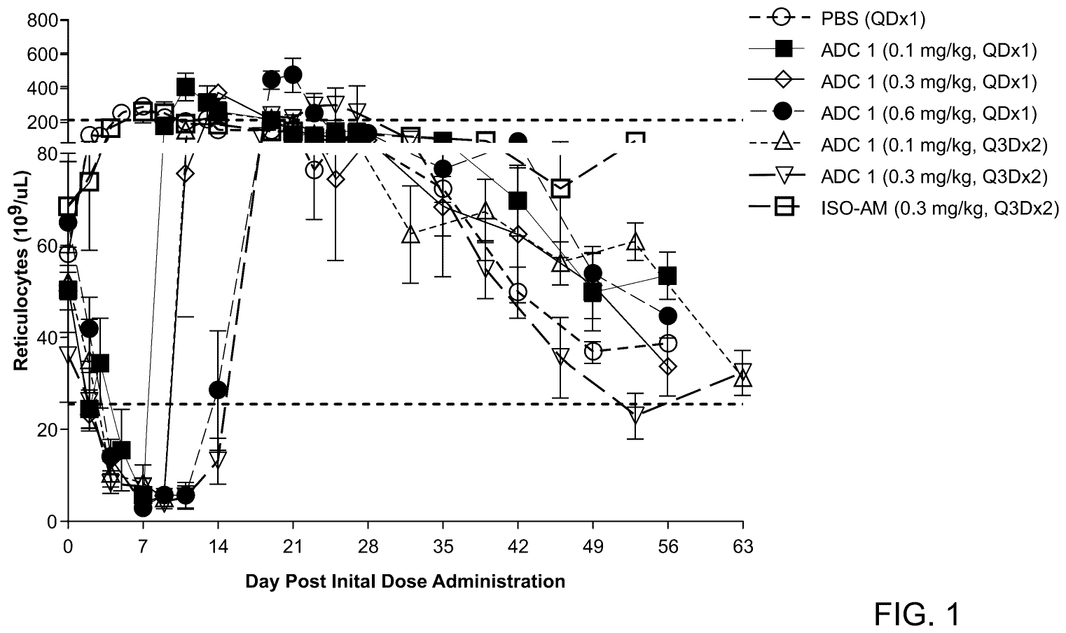

The antibody drug conjugate ADC 1 was used in this Example. Cohorts of cynomolgus monkeys (3 monkeys per cohort) were administered, via one hour infusion, varying doses of the ADC 1 (i.e., 0.1 mg/kg QD×1 (single dose); 0.3 mg/kg QD×1 (single dose); 0.6 mg/kg QD×1 (single dose); 0.1 mg/kg Q3D×2 (multi-dose); 0.3 mg/kg Q3D×2 (multi-dose); or a control (PBS (QD×1 (single dose)) or isotype ADC (0.3 mg/kg QD×1 (single dose))) on day 1. The multi-dose cohorts were administered either 0.1 mg/kg of the ADC or 0.3 mg/kg of the ADC on days 1 and 3. Reticulocyte cell count (10⁹/μL) from whole blood was measured using a hematology analyzer and was graphically represented as a function of days post initial dose administration as shown in FIG. 1.

The results in FIG. 1 indicate a transient decrease in reticulocyte count upon administration of the ADC 1 (i.e., 0.1 mg/kg dose-0.6 mg/kg doses, 0.1 mg/kg Q3D×2 (multi-dose) and 0.3 mg/kg Q3D×2 (multi-dose)) compared to the baseline reticulocyte count (i.e., PBS) for all cohorts dosed with the ADC 1. The results also indicate that administration of the multi-dose regimen of ADC 1 (i.e., 0.1 mg/kg Q3D×2) is equivalent in reticulocyte depletion to the administration of a single 0.3 mg/kg dose of ADC 1.

Similar results were obtained in experiments performed using a fast half-life Ab85 antibody ADC (a heavy chain variable region as set forth in SEQ ID NO: 106, and a light chain variable region as set forth in SEQ ID NO: 107; data not shown) and a fast half-life Ab67 antibody ADC (a heavy chain variable region as set forth in SEQ ID NO: 150, and a light chain variable region as set forth in SEQ ID NO: 151; data not shown).

### Example 2. Analysis of Platelet Cell Count in an In Vivo Dose Escalation Study

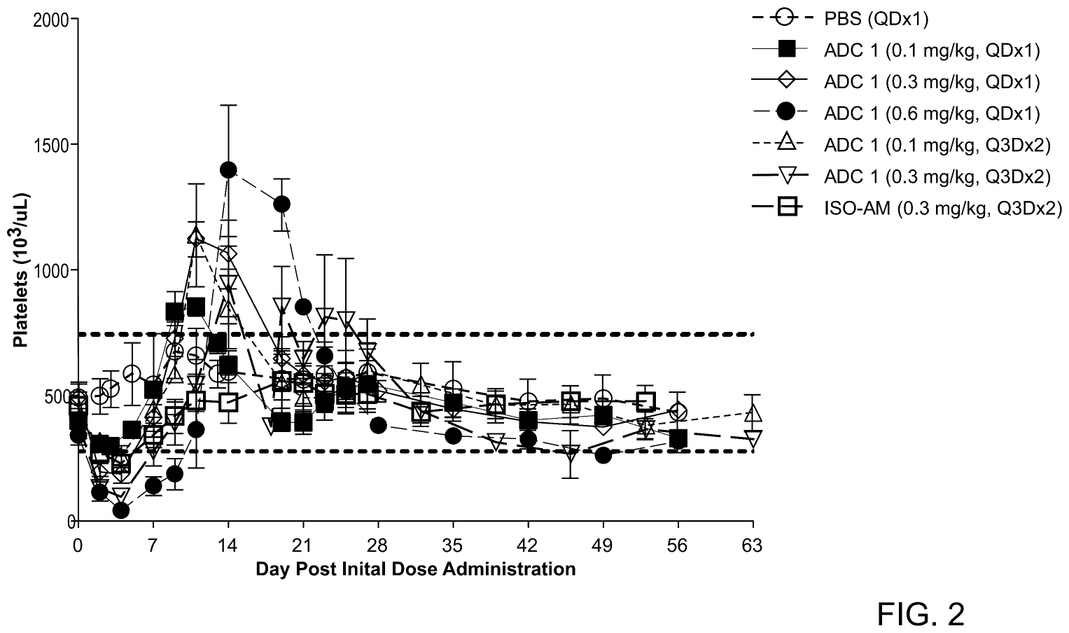

The antibody drug conjugate ADC 1 was used in this Example. Cohorts of cynomolgus monkeys (3 monkeys per cohort) were administered, via one hour infusion, varying doses of the ADC 1 (i.e., 0.1 mg/kg QD×1 (single dose); 0.3 mg/kg QD×1 (single dose); 0.6 mg/kg QD×1 (single dose); 0.1 mg/kg Q3D×2 (multi-dose); 0.3 mg/kg Q3D×2 (multi-dose); or a control (PBS (QD×1 (single dose)) or isotype ADC (0.3 mg/kg QD×1 (single dose))) on day 1. The multi-dose cohorts were administered either 0.1 mg/kg of the ADC or 0.3 mg/kg of the ADC on days 1 and 3. Platelet cell count (10³/μL) from whole blood was measured using a hematology analyzer and was graphically represented as a function of days post initial dose administration as shown in FIG. 2.

The results in FIG. 2 indicate a transient decrease in platelet count upon administration of the ADC 1 (i.e., 0.1 mg/kg dose-0.6 mg/kg doses, 0.1 mg/kg Q3D×2 (multi-dose) and 0.3 mg/kg Q3D×2 (multi-dose)) compared to the baseline platelet count (i.e., PBS). The results also indicate that the decrease in platelet count for the 0.1 mg/kg Q3D×2 (multi-dose) is equivalent to (i.e., no worse than) the decrease in platelet count observed for the 0.1 mg/kg dose (single dose). In addition, the results indicate that administration of the multi-dose regimen of ADC 1 (i.e., 0.3 mg/kg Q3D×2) resulted in a similar transient platelet depletion to the administration of a single 0.6 mg/kg dose of ADC 1.

Similar results were obtained in experiments performed using a fast half-life Ab85 antibody ADC (a heavy chain variable region as set forth in SEQ ID NO: 106, and a light chain variable region as set forth in SEQ ID NO: 107; data not shown) and a fast half-life Ab67 antibody ADC (a heavy chain variable region as set forth in SEQ ID NO: 150, and a light chain variable region as set forth in SEQ ID NO: 151; data not shown).

### Example 3. Analysis of Plasma ALT, LDH, and AST in an In Vivo Dose Escalation Study

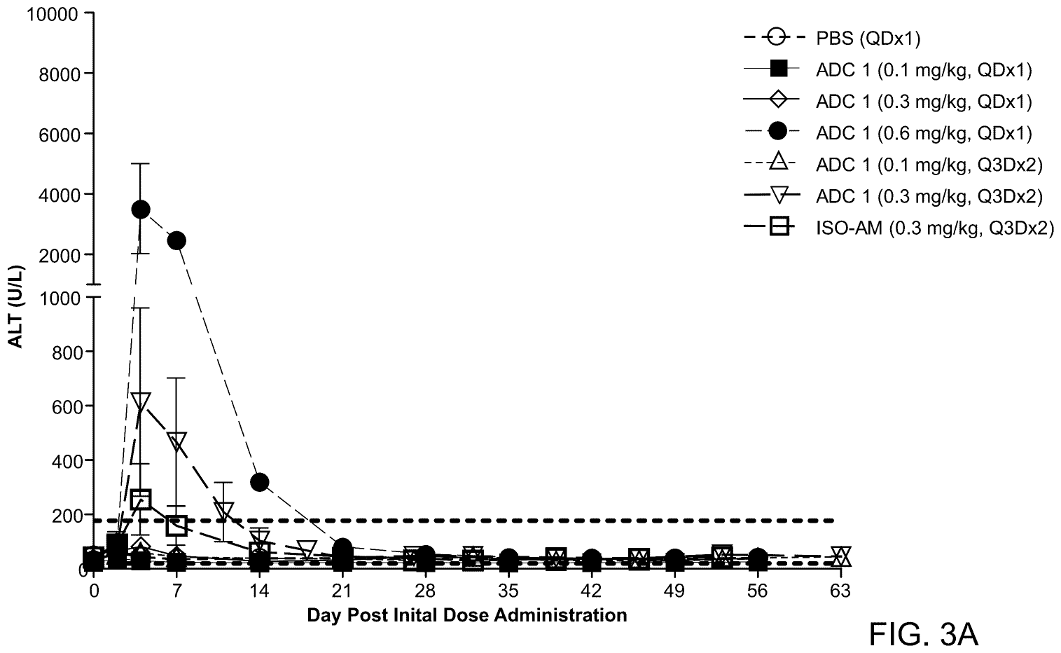

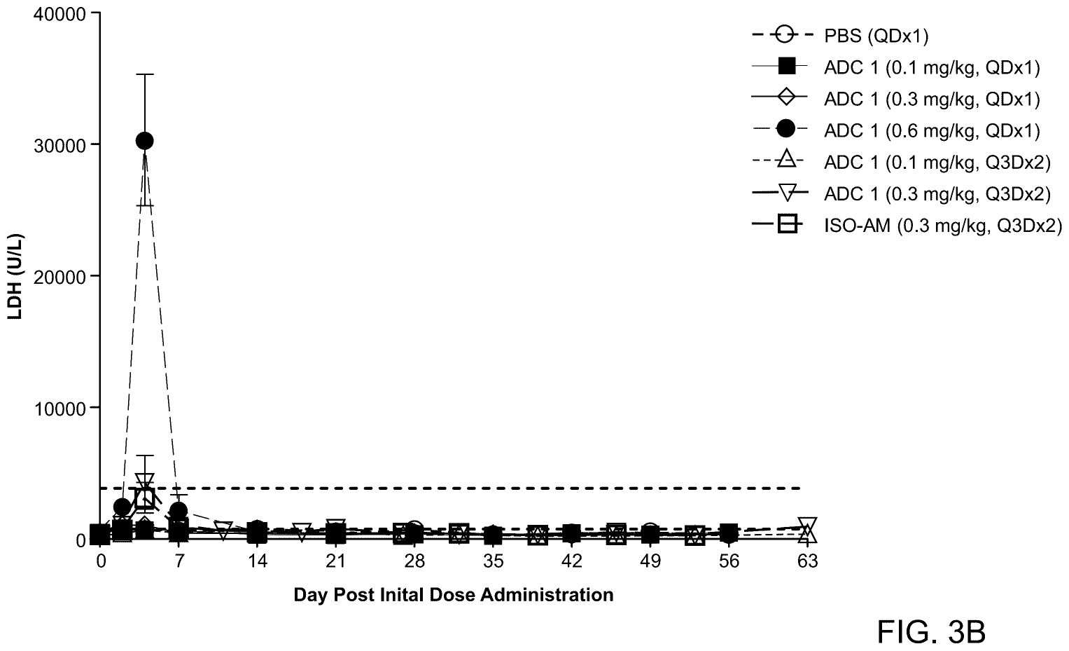

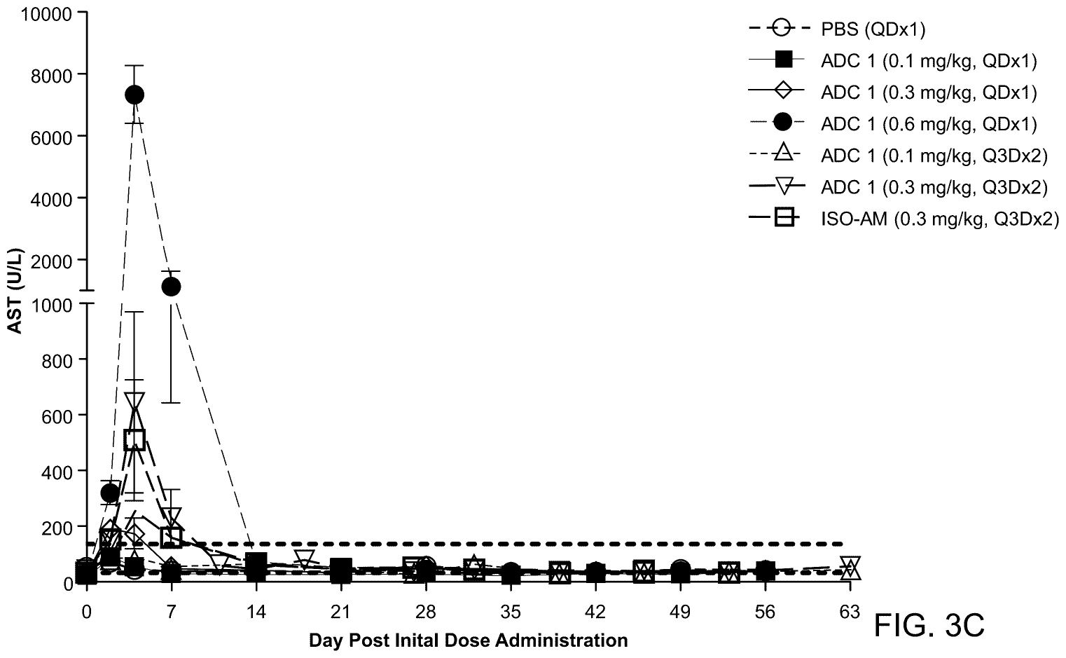

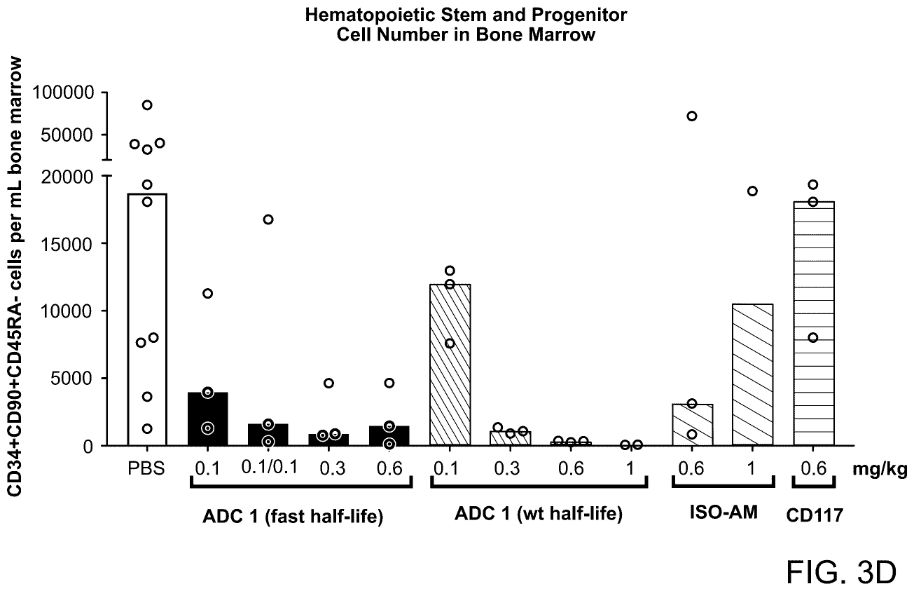

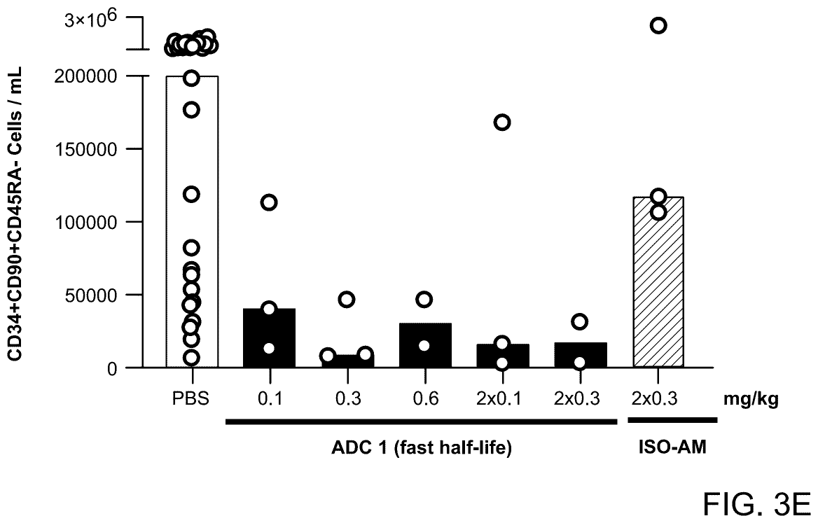

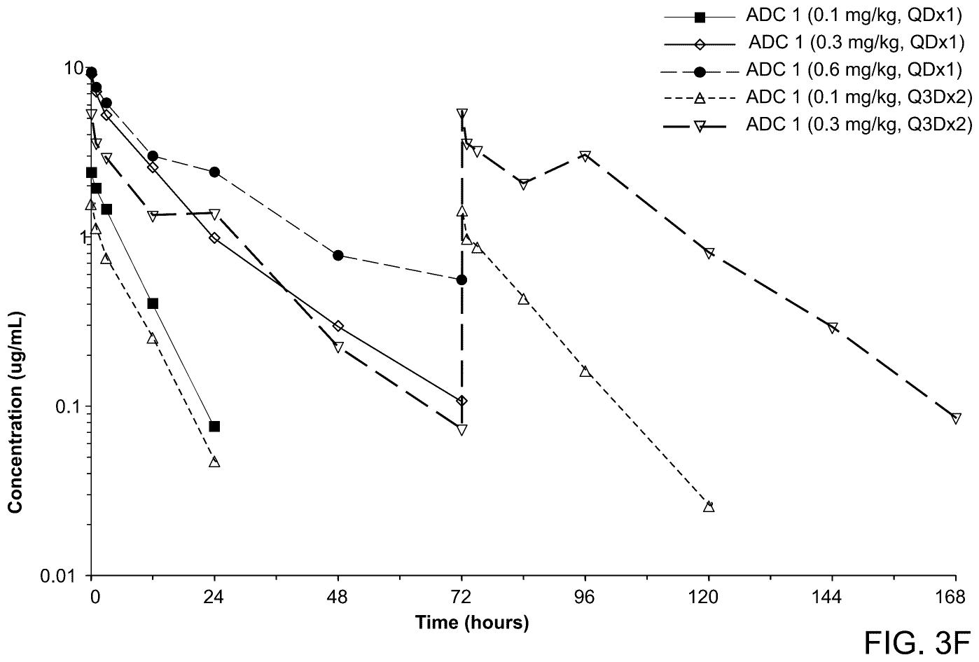

The antibody drug conjugate ADC 1 was used in this Example. Cohorts of cynomolgus monkeys (3 monkeys per cohort) were administered, via one hour infusion, varying doses of the ADC 1 (i.e., 0.1 mg/kg (QD×1); 0.3 mg/kg (QD×1); 0.6 mg/kg (QD×1); 0.1 mg/kg Q3D×2 (multi-dose); 0.3 mg/kg Q3D×2 (multi-dose); or a control (PBS(QD×1 (single dose)) or isotype ADC (0.3 mg/kg QD×1 (single dose))) on day 1. The multi-dose cohorts were administered either 0.1 mg/kg of the ADC or 0.3 mg/kg of the ADC on days 1 and 3. Plasma levels of ALT (alanine aminotransaminase), LDH (lactate dehydrogenase) and AST (aspartate aminotransaminase) were measured using a hematology analyzer and were graphically represented as a function of days post initial dose administration as shown in FIGS. 3A-3C. For the results described in FIGS. 3D and 3E, the bone aspirate was taken on day 15 for single doses and on day 18 for the multi-doses and hematopoietic stem cells and progenitor cells in the bone marrow were measured using a flow cytometric assay.

The results in FIGS. 3A, 3B and 3C indicate a significant transient increase in alanine aminotransaminase (ALT), lactate dehydrogenase (LDH), and aspartate aminotransaminase (AST) levels, respectively, upon administration of the ADC at a dose of 0.6 mg/kg dose as compared to the baseline ALT, LDH and AST counts (i.e., PBS) and the other dosing regimens tested. The results also indicate that no significant change in the levels of alanine aminotransaminase (ALT), lactate dehydrogenase (LDH) and aspartate aminotransaminase (AST) were observed upon administration of the ADC at a dose of 0.1 mg/kg dose compared to baseline (i.e., PBS). The results also indicate that the multidose regimens are well tolerated and result in significantly lower levels of LDH, AST, and ALT compared to the single dose of 0.6 mg/kg.

The results in FIG. 3D describe the hematopoietic stem and progenitor cell number in bone marrow comparing ADC 1 (fast half-life) with a wild-type (wt) half-life ADC 1 (lacking the D265C and H435A Fc mutations), a control IgG1 amanitin ADC (i.e., ISO-AM), and a control naked CD117 antibody (i.e., “CD117”). The results indicate efficient on-target depletion of hematopoietic stem cells and progenitor cells upon administration of the ADC 1 (fast half-life). The results also indicate that the administration of the multi-dose regimen of the fast half-life ADC 1 (i.e., 0.1 mg/kg Q3D×2) results in the depletion of hematopoietic stem cells and progenitor cells of roughly equivalent efficiency compared to that observed for a single 0.6 mg/kg dose of the fast half-life ADC 1. The results in FIG. 3e describe the hematopoietic stem and progenitor cell number in bone marrow comparing the fast half-life ADC 1 to a fast half-life isotype control (i.e., ISO-AM (D265C.H435A)). The results indicate that the administration of the multi-dose regimens of the fast half-life ADC 1 (i.e., 0.1 mg/kg Q3D×2 and 0.3 mg/kg Q3D×2) results in the depletion of hematopoietic stem cells and progenitor cells more efficiently than that observed for a single 0.6 mg/kg dose of the fast half-life ADC 1. In addition, the multi-dosing 0.3 mg/kg Q3D×2 was well tolerated.

Similar results were obtained in experiments performed using a fast half-life Ab85 antibody ADC (a heavy chain variable region as set forth in SEQ ID NO: 106, and a light chain variable region as set forth in SEQ ID NO: 107; data not shown) and a fast half-life Ab67 antibody ADC (a heavy chain variable region as set forth in SEQ ID NO: 150, and a light chain variable region as set forth in SEQ ID NO: 151; data not shown).

In addition, plasma concentrations of ADC 1 was measured using methods known to one of skill in the art and were used to calculate Cₘₐₓ and AUC using varying doses of ADC 1 (i.e., 0.1 mg/kg (QD×1); 0.3 mg/kg (QD×1); 0.6 mg/kg (QD×1); 0.1 mg/kg Q3D×2 (multi-dose); 0.3 mg/kg Q3D×2 (multi-dose)). The multi-dose cohorts were administered either 0.1 mg/kg of the ADC 1 or 0.3 mg/kg of the ADC 1 at t=0 and at t=72 hours, while the single dose cohorts were administered either 0.1 mg/kg of the ADC 1, 0.3 mg/kg of the ADC 1 or 0.6 mg/kg of the ADC 1 at t=0. The concentration of ADC 1 (μg/mL) as a function of time was measured and graphically depicted in FIG. 3F. The numerical results were provided in Table 4.

TABLE 4
Mean PK parameters for various dosing regimens of ADC 1

| | | | | | Half- |
|---|---|---|---|---|---|
| Dose | | AUC_{inf} | Cₘₐₓ | Tₘₐₓ | life |
| (mg/kg) | Regimen | (hour*mg/mL) | (mg/mL) | (hour) | (hour) |
| 0.1 | QD × 1 | 15.1 | 2.39 | 0.0833 | 4.31 |
| 0.1 | Q3D × 2 | 40.7 | 1.52 | 0.0833 | 8.12 |
| 0.3 | QD × 1 | 93.0 | 9.07 | 0.0833 | 12.4 |
| 0.3 | Q3D × 2 | 184 | 5.28 | 72.083 | 16.0 |
| 0.6 | QD × 1 | 161 | 9.57 | 0.389 | 20.7 |

The data indicate that the multi-dosing regimen of 0.3 mg/kg Q3D×2 resulted in a similar AUC, a lower Cₘₐₓ, and lower liver enzyme levels (e.g., lower ALT, LDH, AST), while resulting in a similar transient decrease in reticulocytes when compared to the 0.6 mg/kg QD×1 (single dose). These data indicated that the tolerability may be driven by the Cₘₐₓ while the efficacy may be driven by the AUC.

Similar trends in AUC and Cₘₐₓ were observed in experiments performed using a fast half-life Ab85 antibody ADC (a heavy chain variable region as set forth in SEQ ID NO: 106, and a light chain variable region as set forth in SEQ ID NO: 107; data not shown) and a fast half-life Ab67 antibody ADC (a heavy chain variable region as set forth in SEQ ID NO: 150, and a light chain variable region as set forth in SEQ ID NO: 151; data not shown).

As shown herein, increasing doses of ADCs resulted in increased exposure. When considered as total amount dosed, fractionated dosing of two doses resulted in similar AUC values as higher single doses, but similar Cₘₐₓ values as compared to a single dose of equivalent amounts. Fractionated dosing of these ADCs also resulted in lower liver enzyme levels and similar efficacy as compared to higher single doses, further indicating that, in certain embodiments, tolerability may be driven by Cₘₐₓ and that efficacy may be driven by AUC.

### Example 4. Analysis of Reticulocyte Cell Count and Plasma AST in an In Vivo Dose Escalation Study

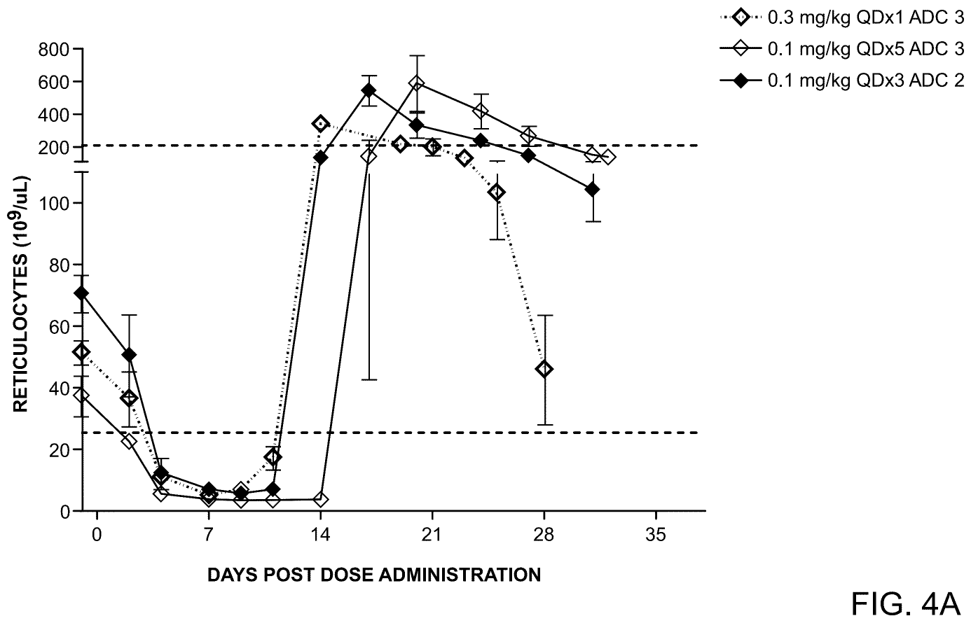

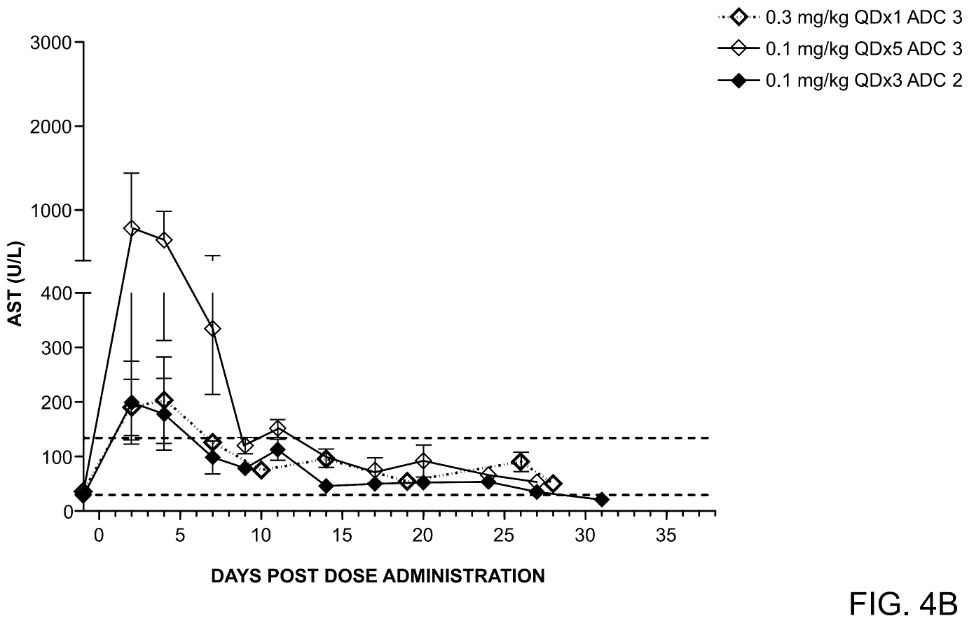

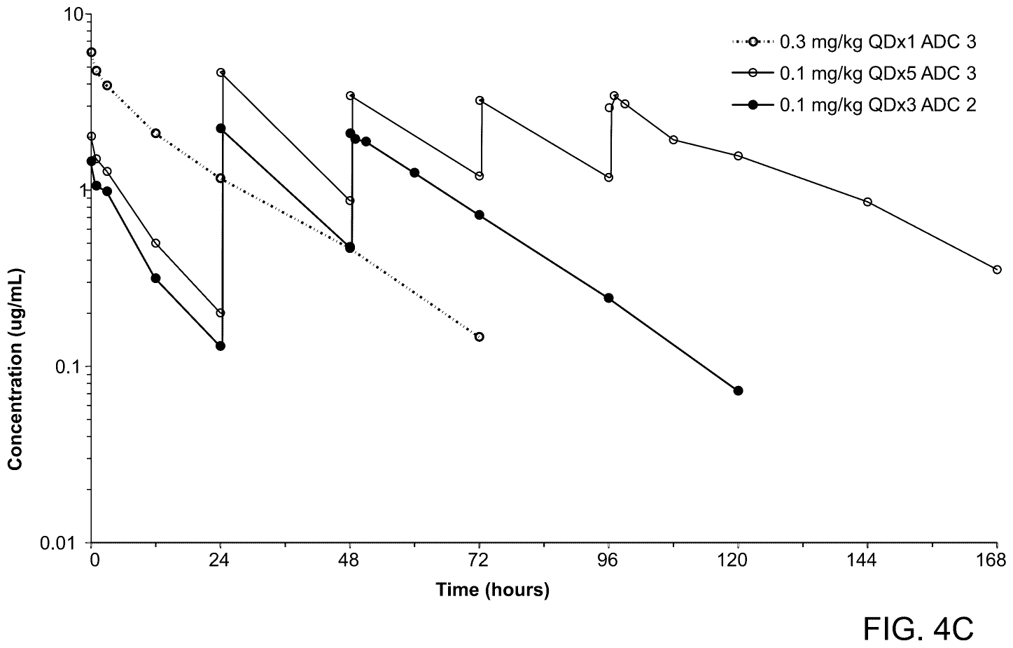

The antibody drug conjugates ADC 2 and ADC 3 were used in the following Example. Cohorts of cynomolgus monkeys (3 monkeys per cohort) were administered, via one hour infusion, a multi-dose of ADC 2 (i.e., 0.1 mg/kg QD×3) or varying doses of the ADC 3 (i.e., 0.3 mg/kg QD×1 (single dose) or 0.1 mg/kg QD×5 (multi-dose) on day 1. The ADC 2 multi-dose cohort was administered 0.1 mg/kg of the ADC on days 1 and 3. The ADC 3 multi-dose cohort was administered 0.1 mg/kg of the ADC on days 1, 2, 3, 4 and 5. Reticulocyte cell count (10⁹/μL) from whole blood was measured using a hematology analyzer and was graphically represented as a function of days post initial dose administration as shown in FIG. 4A. In addition, plasma levels of AST (aspartate aminotransaminase) were measured using a hematology analyzer and were graphically represented as a function of days post initial dose administration as shown in FIG. 4B.

The results in FIG. 4A indicate a transient decrease in reticulocyte count upon administration of the ADC 2 (i.e., 0.1 mg/kg QD×3 (multi-dose)) and the ADC 3 (i.e., 0.3 mg/kg QD×1 (single dose) and 0.1 mg/kg QD×5 (multi-dose)). A transient decrease in platelet count upon administration of the ADC 2 (i.e., 0.1 mg/kg QD×3 (multi-dose) and the ADC 3 (i.e., 0.3 mg/kg QD×1 (single dose) and 0.1 mg/kg QD×5 (multi-dose)) was also observed (data not shown). The results in FIG. 4B indicate that no significant change in the levels of aspartate aminotransaminase (AST) were observed upon administration the ADC 2 (i.e., 0.1 mg/kg QD×3 (multi-dose)) compared to a single dose of the ADC 3 (i.e., 0.3 mg/kg QD×1 (single dose)). In addition, a comparison of the results for ADC 3 (i.e., 0.3 mg/kg QD×1 (single dose) compared with 0.1 mg/kg QD×5 (multi-dose)) also suggest a threshold at which an increase in the elevation in plasma AST levels is observed. Similar results were obtained for plasma levels of alanine aminotransaminase (ALT) lactate dehydrogenase (LDH) in experiments performed using the ADC 2 (i.e., 0.1 mg/kg QD×3 (multi-dose)) and the ADC 3 (i.e., 0.3 mg/kg QD×1 (single dose) and 0.1 mg/kg QD×5 (multi-dose)) (data not shown).

In addition, plasma concentrations of ADC 2 and ADC3 were measured using methods known to one of skill in the art and were used to calculate Cₘₐₓ and AUC using the ADC 2 (i.e., 0.1 mg/kg QD×3 (multi-dose)) and varying doses of ADC 3 (i.e., 0.3 mg/kg QD×1 (single dose) and 0.1 mg/kg QD×5 (multi-dose)). The multi-dose cohorts was administered either 0.1 mg/kg of the ADC 2 at t=0, at t=24 hours and at t=48 hours or 0.1 mg/kg of the ADC 3 at t=0, at t=24 hours, at t=48 hours, at t=72 hours, and at t=96 hours, while the single dose cohort was administered 0.3 mg/kg of the ADC 3 at t=0. The concentration of ADC 2 (μg/mL) and ADC 3 (μg/mL) as a function of time was measured and graphically depicted in FIG. 4C. The numerical results were provided in Table 5 (ADC 2) and Table 6 (ADC 3).

TABLE 5
Mean PK parameters for various dosing regimens of ADC 2

| | | AUC_{inf} | | | Half- |
|---|---|---|---|---|---|
| Dose | | (hour*kg*m/ | Cₘₐₓ | Tₘₐₓ | life |
| (mg/kg) | Regimen | mL/mg) | (kg*mg/mL/mg) | (hour) | (hour) |
| 0.1 | QD × 3 | 85.8 | 2.24 | 24.08 | 12.8 |

TABLE 6
Mean PK parameters for various dosing regimens of ADC 3

| | | AUC_{inf} | | | Half- |
|---|---|---|---|---|---|
| Dose | | (hour*kg*m/ | Cₘₐₓ | Tₘₐₓ | life |
| (mg/kg) | Regimen | mL/mg) | (kg*mg/mL/mg) | (hour) | (hour) |
| 0.3 | QD × 1 | 87.9 | 6.07 | 0.08 | 15.8 |
| 0.1 | QD × 5 | 276 | 4.66 | 24.08 | 22.4 |

The data indicate that the multi-dosing regimen of 0.1 mg/kg QD×5 resulted in a higher AUC, a lower Cₘₐₓ, and higher liver enzyme levels (e.g., higher AST (FIG. 4B), higher ALT (data not shown), and higher LDH (data not shown)).

SEQUENCE TABLE

| Sequence | | |
|---|---|---|
| Identifier | Description | Sequence |
| SEQ ID NO: 1 | CK6 CDR-H1 | SYWIG |
| SEQ ID NO: 2 | CK6 CDR-H2 | IIYPGDSDTRYSPSFQG |
| SEQ ID NO: 3 | CK6 CDR-H3 | HGRGYNGYEGAFDI |
| SEQ ID NO: 4 | CK6 CDR-L1 | RASQGISSALA |
| SEQ ID NO: 5 | CK6 CDR-L2 | DASSLES |
| SEQ ID NO: 6 | CK6 CDR-L3 | CQQFNSYPLT |
| SEQ ID NO: 7 | Heavy chain variable | QVQLVQSGAAVKKPGESLKISCKG |
| | region amino acid | SGYRFTSYWIGWVRQMPGKGLE |
| | sequence of CK6 | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 8 | Light chain variable | AIQLTQSPSSLSASVGDRVTITCRA |
| | region amino acid | SQGISSALAWYQQKPGKAPKLLIY |
| | sequence of CK6 | DASSLESGVPSRFSGSGSGTDFT |
| | | LTISSLQPEDFATYYCQQFNSYPL |
| | | TFGGGTKVEIK |
| SEQ ID NO: 9 | Heavy chain variable | QVQLVQSGAAVKKPGESLKISCKG |
| | region of (anti- | SGYRFTTYWIGWVRQMPGKGLE |
| | CD117) HC-1 | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 10 | Light chain variable | AIQLTQSPSSLSASVGDRVTITCRA |
| | region of (anti- | SQGVSSALAWYQQKPGKAPKLLIY |
| | CD117) LC-1 | DASSLESGVPSRFSGSGSGTDFT |
| | | LTISSLQPEDFATYYCQQFNSYPL |
| | | TFGGGTKVEIK |
| SEQ ID NO: 9 | Heavy chain variable | QVQLVQSGAAVKKPGESLKISCKG |
| | region of (anti- | SGYRFTTYWIGWVRQMPGKGLE |
| | CD117) HC-2 | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 11 | Light chain variable | DIQLTQSPSSLSASVGDRVTITCRA |
| | region of (anti- | SQGIRTDLGWYQQKPGKAPKLLIY |
| | CD117) LC-2 | DASSLESGVPSRFSGSGSGTDFT |
| | | LTISSLQPEDFATYYCQQFNSYPL |
| | | TFGGGTKVEIK |
| SEQ ID NO: 9 | Heavy chain variable | QVQLVQSGAAVKKPGESLKISCKG |
| | region of (anti- | SGYRFTTYWIGWVRQMPGKGLE |
| | CD117) HC-3 | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 12 | Light chain variable | AIRMTQSPSSLSASVGDRVTITCR |
| | region of (anti- | ASQGIRNDLAWYQQKPGKTPKLLI |
| | CD117) LC-3 | YDASSLESGVPSRFSGSGSGTDF |
| | | TLTISSLQPEDFATYYCQQFNSYP |
| | | LTFGGGTKVEIK |
| SEQ ID NO: 9 | Heavy chain variable | QVQLVQSGAAVKKPGESLKISCKG |
| | region of (anti- | SGYRFTTYWIGWVRQMPGKGLE |
| | CD117) HC-4 | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 13 | Light chain variable | AIQMTQSPSSLSASVGDRVTITCR |
| | region of (anti- | ASQGIRNDLGWYQQKPGKAPKLLI |
| | CD117) LC-4 | YDASSLESGVPSRFSGSGSGTDF |
| | | TLTISSLQPEDFATYYCQQFNSYP |
| | | LTFGGGTKVDIK |
| SEQ ID NO: 9 | Heavy chain variable | QVQLVQSGAAVKKPGESLKISCKG |
| | region of (anti- | SGYRFTTYWIGWVRQMPGKGLE |
| | CD117) HC-5 | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 14 | Light chain variable | NIQMTQSPSSLSASVGDRVTITCR |
| | region of (anti- | ASQAISDYLAWFQQKPGKAPKLLI |
| | CD117) LC-5 | YDASNLETGVPSRFSGSGSGTDF |
| | | TLTISSLQPEDFATYYCQQLNSYPL |
| | | TFGGGTKVEIK |
| SEQ ID NO: 9 | Heavy chain variable | QVQLVQSGAAVKKPGESLKISCKG |
| | region of (anti- | SGYRFTTYWIGWVRQMPGKGLE |
| | CD117) HC-6 | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 15 | Light chain variable | AIRMTQSPSSLSASVGDRVIIACRA |
| | region of (anti- | SQGIGGALAWYQQKPGNAPKVLV |
| | CD117) LC-6 | YDASTLESGVPSRFSGGGSGTDF |
| | | TLTISSLQPEDFATYYCQQFNSYP |
| | | LTFGGGTKLEIK |
| SEQ ID NO: 9 | Heavy chain variable | QVQLVQSGAAVKKPGESLKISCKG |
| | region of (anti- | SGYRFTTYWIGWVRQMPGKGLE |
| | CD117) HC-7 | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 16 | Light chain variable | DIAMTQSPPSLSAFVGDRVTITCR |
| | region of (anti- | ASQGIISSLAWYQQKPGKAPKLLIY |
| | CD117) LC-7 | DASSLESGVPSRFSGSGSGTDFT |
| | | LTIRSLQPEDFATYYCQQFNSYPL |
| | | TFGGGTKLEIK |
| SEQ ID NO: 9 | Heavy chain variable | QVQLVQSGAAVKKPGESLKISCKG |
| | region of (anti- | SGYRFTTYWIGWVRQMPGKGLE |
| | CD117) HC-8 | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 17 | Light chain variable | DIQMTQSPSSLSASVGDRVTITCR |
| | region of (anti- | ASQGISSALAWYQQKAGKAPKVLI |
| | CD117) LC-8 | SDASSLESGVPSRFSGSGSGTDF |
| | | TLSISSLQPEDFATYYCQQFNGYP |
| | | LTFGGGTKVDIK |
| SEQ ID NO: 9 | Heavy chain variable | QVQLVQSGAAVKKPGESLKISCKG |
| | region of (anti- | SGYRFTTYWIGWVRQMPGKGLE |
| | CD117) HC-9 | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 18 | Light chain variable | AIRMTQSPSSLSASVGDRVTITCQ |
| | region of (anti- | ASQGIRNDLGWYQQKPGKAPKLLI |
| | CD117) LC-9 | YDASNLETGVPSRFSGSGSGTDF |
| | | TFTISSLQPEDIATYYCQQFNSYPL |
| | | TFGGGTKLEIK |
| SEQ ID NO: 9 | Heavy chain variable | QVQLVQSGAAVKKPGESLKISCKG |
| | region of (anti- | SGYRFTTYWIGWVRQMPGKGLE |
| | CD117) HC-10 | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 19 | Light chain variable | NIQMTQSPSSLSTSVGDRVTITCR |
| | region of (anti- | ASQGIGTSLAWYQQKPGKPPKLLI |
| | CD117) LC-10 | YDASSLESGVPSRLSGSGSGTDF |
| | | TLTISSLQPEDFATYYCQQSNSYPI |
| | | TFGQGTRLEIK |
| SEQ ID NO: 9 | Heavy chain variable | QVQLVQSGAAVKKPGESLKISCKG |
| | region of (anti- | SGYRFTTYWIGWVRQMPGKGLE |
| | CD117) HC-11 | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 20 | Light chain variable | AIQLTQSPSSLSASVGDRVTITCRA |
| | region of (anti- | SQSIGDYLTWYQQKPGKAPKVLIY |
| | CD117) LC-11 | GASSLQSGVPPRFSGSGSGTDFT |
| | | LTVSSLQPEDFATYYCQQLNSYPL |
| | | TFGGGTKLEIK |
| SEQ ID NO: 9 | Heavy chain variable | QVQLVQSGAAVKKPGESLKISCKG |
| | region of (anti- | SGYRFTTYWIGWVRQMPGKGLE |
| | CD117) HC-12 | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 21 | Light chain variable | DIQLTQSPSSLSASVGDRVTITCRA |
| | region of (anti- | SQGVRSTLAWYQQKPGKAPKLLIY |
| | CD117) LC-12 | DASILESGVPSRFSGSGSGTDFTL |
| | | TISSLQPEDFATYYCQQFNGYPLT |
| | | FGQGTRLEIK |
| SEQ ID NO: 9 | Heavy chain variable | QVQLVQSGAAVKKPGESLKISCKG |
| | region of (anti- | SGYRFTTYWIGWVRQMPGKGLE |
| | CD117) HC-13 | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 22 | Light chain variable | DIVMTQSPSSLSASVGDRVTITCR |
| | region of (anti- | ASQGIRNDLGWYQQKPGKAPKLLI |
| | CD117) LC-13 | YDASSLESGVPSRFSGSGSGTDF |
| | | TLTISSLQPEDFATYYCQQFNSYP |
| | | LTFGGGTKLEIK |
| SEQ ID NO: 9 | Heavy chain variable | QVQLVQSGAAVKKPGESLKISCKG |
| | region of (anti- | SGYRFTTYWIGWVRQMPGKGLE |
| | CD117) HC-14 | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 23 | Light chain variable | DIQLTQSPSSLSASVGDRVTITCRA |
| | region of (anti- | SQGISSFLAWYQQKPGKAPKLLIY |
| | CD117) LC-14 | DASTLQSGVPSRFSGSASGTDFTL |
| | | TISSLQPEDFATYYCQQLNGYPLT |
| | | FGGGTKVEIK |
| SEQ ID NO: 9 | Heavy chain variable | QVQLVQSGAAVKKPGESLKISCKG |
| | region of (anti- | SGYRFTTYWIGWVRQMPGKGLE |
| | CD117) HC-15 | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 24 | Light chain variable | AIQLTQSPSSLSASVGDRVTITCRA |
| | region of (anti- | SQGIGSALAWYQQKPGIGPKLLIY |
| | CD117) LC-15 | DASTLESGVPARFSGSGSRTDFTL |
| | | TITSLQPEDFATYYCQQFNGYPLT |
| | | FGGGTKLEIK |
| SEQ ID NO: 9 | Heavy chain variable | QVQLVQSGAAVKKPGESLKISCKG |
| | region of (anti- | SGYRFTTYWIGWVRQMPGKGLE |
| | CD117) HC-16 | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 25 | Light chain variable | AIQLTQSPSSLSASVGDRVTITCRA |
| | region of (anti- | SQGITSALAWYQEKPGKAPNLLIY |
| | CD117) LC-16 | DASSLESGVPSRFSGSGYGTDFT |
| | | LTISSLQPEDFATYYCQQLNSYPLT |
| | | FGGGTKVDIK |
| SEQ ID NO: 26 | Heavy chain variable | QIQLVQSGPELRKPGESVKISCKA |
| | region of (anti- | SGYTFTDYAMYWVKQAPGKGLK |
| | CD117) HC-17 | WMGWINTYTGKPTYADDFKGRFV |
| | | FSLEASANTANLQISNLKNEDTATY |
| | | FCARARGLVDDYVMDAWGQGTS |
| | | VTVSS |
| SEQ ID NO: 27 | Light chain variable | SYELIQPPSASVTLGNTVSLTCVG |
| | region of (anti- | DELSKRYAQWYQQKPDKTIVSVIY |
| | CD117) LC-17 | KDSERPSGISDRFSGSSSGTTATL |
| | | TIHGTLAEDEADYYCLSTYSDDNL |
| | | PVFGGGTKLTVL |
| SEQ ID NO: 28 | Heavy chain variable | EVQLQQYGAELGKPGTSVRLSCK |
| | region of (anti- | VSGYNIRNTYIHWVNQRPGEGLE |
| | CD117) HC-18 | WIGRIDPTNGNTISAEKFKTKATLT |
| | | ADTSSHTAYLQFSQLKSDDTAIYF |
| | | CALNYEGYADYWGQGVMVTGSS |
| SEQ ID NO: 29 | Light chain variable | DIQMTQSPSFLSASVGDRVTINCK |
| | region of (anti- | ASQNINKYLNWYQQKVGEAPKRLI |
| | CD117) LC-18 | FKTNSLQTGIPSRFSGSGSGTDYT |
| | | LTISSLQTEDVATYFCFQYNIGYTF |
| | | GAGTKVELK |
| SEQ ID NO: 30 | Heavy chain variable | EVQLQESGPGLVKPSQSLSLTCSV |
| | region of (anti- | TGYSISSNYRWNWIRKFPGNKVE |
| | CD117) HC-19 | WMGYINSAGSTNYNPSLKSRISMT |
| | | RDTSKNQFFLQVNSVTTEDTATYY |
| | | CARSLRGYITDYSGFFDYWGQGV |
| | | MVTVSS |
| SEQ ID NO: 31 | Light chain variable | DIRMTQSPASLSASLGETVNIECLA |
| | region of (anti- | SEDIFSDLAWYQQKPGKSPQLLIY |
| | CD117) LC-19 | NANSLQNGVPSRFSGSGSGTRYS |
| | | LKINSLQSEDVATYFCQQYKNYPL |
| | | TFGSGTKLEIK |
| SEQ ID NO: 32 | Heavy chain variable | EVQLQQYGAELGKPGTSVRLSCK |
| | region of (anti- | LSGYKIRNTYIHWVNQRPGKGLE |
| | CD117) HC-20 | WIGRIDPANGNTIYAEKFKSKVTLT |
| | | ADTSSNTAYMQLSQLKSDDTALYF |
| | | CAMNYEGYEDYWGQGVMVTVSS |
| SEQ ID NO: 33 | Light chain variable | DIQMTQSPSFLSASVGDSVTINCK |
| | region of (anti- | ASQNINKYLNWYQQKLGEAPKRLI |
| | CD117) LC-20 | HKTDSLQTGIPSRFSGSGSGTDYT |
| | | LTISSLQPEDVATYFCFQYKSGFM |
| | | FGAGTKLELK |
| SEQ ID NO: 34 | Heavy chain variable | QIQLVQSGPELKKPGESVKISCKA |
| | region of (anti- | SGYTFTDYAVYWVIQAPGKGLKW |
| | CD117) HC-21 | MGWINTYTGKPTYADDFKGRFVF |
| | | SLETSASTANLQISNLKNEDTATYF |
| | | CARGAGMTKDYVMDAWGRGVLV |
| | | TVS |
| SEQ ID NO: 35 | Light chain variable | SYELIQPPSASVTLGNTVSLTCVG |
| | region of (anti- | DELSKRYAQWYQQKPDKTIVSVIY |
| | CD117) LC-21 | KDSERPSDISDRFSGSSSGTTATL |
| | | TIHGTLAEDEADYYCLSTYSDDNL |
| | | PVFGGGTKLTVL |
| SEQ ID NO: 36 | Heavy chain variable | QVQLKESGPGLVQPSQTLSLTCTV |
| | region of (anti- | SGFSLTSYLVHWVRQPPGKTLEW |
| | CD117) HC-22 | VGLMWNDGDTSYNSALKSRLSIS |
| | | RDTSKSQVFLKMHSLQAEDTATY |
| | | YCARESNLGFTYWGHGTLVTVSS |
| SEQ ID NO: 37 | Light chain variable | DIQMTQSPASLSASLEEIVTITCKA |
| | region of (anti- | SQGIDDDLSWYQQKPGKSPQLLIY |
| | CD117) LC-22 | DVTRLADGVPSRFSGSRSGTQYS |
| | | LKISRPQVADSGIYYCLQSYSTPYT |
| | | FGAGTKLELK |
| SEQ ID NO: 38 | Heavy chain variable | EVQLQQYGAELGKPGTSVRLSCK |
| | region of (anti- | VSGYNIRNTYIHWVHQRPGEGLE |
| | CD117) HC-23 | WIGRIDPTNGNTISAEKFKSKATLT |
| | | ADTSSNTAYMQFSQLKSDDTAIYF |
| | | CAMNYEGYADYWGQGVMVTVSS |
| SEQ ID NO: 39 | Light chain variable | DIQMTQSPSFLSASVGDRLTINCK |
| | region of (anti- | ASQNINKYLNWYQQKLGEAPKRLI |
| | CD117) LC-23 | FKTNSLQTGIPSRFSGSGSGTDYT |
| | | LTISSLQPEDVATYFCFQYNIGFTF |
| | | GAGTKLELK |
| SEQ ID NO: 40 | Heavy chain variable | EVQLVESGGGLVQSGRSLKLSCA |
| | region of (anti- | ASGFTVSDYYMAWVRQAPTKGLE |
| | CD117) HC-24 | WVATINYDGSTTYHRDSVKGRFTI |
| | | SRDNAKSTLYLQMDSLRSEDTATY |
| | | YCARHGDYGYHYGAYYFDYWGQ |
| | | GVMVTVSS |
| SEQ ID NO: 41 | Light chain variable | DIVLTQSPALAVSLGQRATISCRAS |
| | region of (anti- | QTVSLSGYNLIHWYQQRTGQQPK |
| | CD117) LC-24 | LLIYRASNLAPGIPARFSGSGSGTD |
| | | FTLTISPVQSDDIATYYCQQSRES |
| | | WTFGGGTNLEMK |
| SEQ ID NO: 42 | Heavy chain variable | QIQLVQSGPELKKPGESVKISCKA |
| | region of (anti- | SGYTFTDYAIHWVKQAPGQGLRW |
| | CD117) HC-25 | MAWINTETGKPTYADDFKGRFVF |
| | | SLEASASTAHLQISNLKNEDTATFF |
| | | CAGGSHWFAYWGQGTLVTVSS |
| SEQ ID NO: 43 | Light chain variable | SYELIQPPSASVTLENTVSITCSGD |
| | region of (anti- | ELSNKYAHWYQQKPDKTILEVIYN |
| | CD117) LC-25 | DSERPSGISDRFSGSSSGTTAILTI |
| | | RDAQAEDEADYYCLSTFSDDDLPI |
| | | FGGGTKLTVL |
| SEQ ID NO: 34 | Heavy chain variable | QIQLVQSGPELKKPGESVKISCKA |
| | region of (anti- | SGYTFTDYAVYWVIQAPGKGLKW |
| | CD117) HC-26 | MGWINTYTGKPTYADDFKGRFVF |
| | | SLETSASTANLQISNLKNEDTATYF |
| | | CARGAGMTKDYVMDAWGRGVLV |
| | | TVS |
| SEQ ID NO: 44 | Light chain variable | SYELIQPPSTSVTLGNTVSLTCVG |
| | region of (anti- | NELPKRYAYWFQQKPDQSIVRLIY |
| | CD117) LC-26 | DDDRRPSGISDRFSGSSSGTTATL |
| | | TIRDAQAEDEAYYYCHSTYTDDKV |
| | | PIFGGGTKLTVL |
| SEQ ID NO: 45 | Heavy chain variable | EVQLVESGGGLVQPGRSMKLSCK |
| | region of (anti- | ASGFTFSNYDMAWVRQAPTRGLE |
| | CD117) HC-27 | WVASISYDGITAYYRDSVKGRFTIS |
| | | RENAKSTLYLQLVSLRSEDTATYY |
| | | CTTEGGYVYSGPHYFDYWGQGV |
| | | MVTVSS |
| SEQ ID NO: 46 | Light chain variable | DIQMTQSPSSMSVSLGDTVTITCR |
| | region of (anti- | ASQDVGIFVNWFQQKPGRSPRRM |
| | CD117) LC-27 | IYRATNLADGVPSRFSGSRSGSDY |
| | | SLTISSLESEDVADYHCLQYDEFP |
| | | RTFGGGTKLELK |
| SEQ ID NO: 47 | Heavy chain variable | EVQLQQYGAELGKPGTSVRLSCK |
| | region of (anti- | VSGYKIRNTYIHWVNQRPGKGLE |
| | CD117) HC-28 | WIGRIDPANGNTIYAEKFKSKVTLT |
| | | ADTSSNTAYMQLSQLKSDDTALYF |
| | | CAMNYEGYEDYWGQGVMVTVSS |
| SEQ ID NO: 48 | Light chain variable | DIQMTQSPSFLSASVGDSVTINCK |
| | region of (anti- | ASQNINKYLNWYQQKLGEAPKRLI |
| | CD117) LC-28 | HKTNSLQPGFPSRFSGSGSGTDY |
| | | TLTISSLQPEDVAAYFCFQYNSGF |
| | | TFGAGTKLELK |
| SEQ ID NO: 49 | Heavy chain variable | QVQLVQSGAEVKKPGASVKVSCK |
| | region of (anti- | ASGYTFTDYYIHWVRQAPGQGLE |
| | CD117) HC-29 | WMGWMNPHSGDTGYAQKFQGR |
| | | VTMTRDTSTSTVYMELSSLRSEDT |
| | | AVYYCARHGRGYNGYEGAFDIWG |
| | | QGTLVTVSSAS |
| SEQ ID NO: 50 | Light chain variable | DIQMTQSPSSLSASVGDRVTITCR |
| | region of (anti- | ASQGIGNELGWYQQKPGKAPKLLI |
| | CD117) LC-29 | YAASNLQSGVPSRFSGSGSGTDF |
| | | TLTISSLQPEDFATYYCQQYDNLP |
| | | LTFGQGTKVEIK |
| SEQ ID NO: 51 | Heavy chain variable | QVQLVQSGAEVKKPGASVKVSCK |
| | region of (anti- | ASGYTFTGYYLHWVRQAPGQGLE |
| | CD117) HC-30 | WMGWINPNSGDTNYAQNFQGRV |
| | | TMTRDTSTSTVYMELSSLRSEDTA |
| | | VYYCARHGRGYNGYEGAFDIWG |
| | | QGTLVTVSSAS |
| SEQ ID NO: 52 | Light chain variable | DIQMTQSPSSLSASVGDRVTITCR |
| | region of (anti- | ASQGIRNDLGWYQQKPGKAPKLLI |
| | CD117) LC-30 | YDASSLESGVPSRFSGSGSGTDF |
| | | TLTISSLQPEDFATYYCQQLNGYP |
| | | LTFGGGTKVEIK |
| SEQ ID NO: 53 | Heavy chain variable | QVQLVQSGAEVKKPGASVKVSCK |
| | region of (anti- | ASGYTFTGYYLHWVRQAPGQGLE |
| | CD117) HC-31 | WMGWINPNSGGTNYAQKFQGRV |
| | | TMTRDTSTSTVYMELSSLRSEDTA |
| | | VYYCARHGRGYEGYEGAFDIWGQ |
| | | GTLVTVSSAS |
| SEQ ID NO: 54 | Light chain variable | DIQMTQSPSSLSASVGDRVTITCR |
| | region of (anti- | ASQGIRNDLGWYQQKPGKAPKLLI |
| | CD117) LC-31 | YDASELETGVPSRFSGSGSGTDF |
| | | TLTISSLQPEDFATYYCQQLNGYPI |
| | | TFGQGTKVEIK |
| SEQ ID NO: 55 | Heavy chain variable | QVQLVQSGAEVKKPGASVKVSCK |
| | region of (anti- | ASGYTFTSYYIHWVRQAPGQGLE |
| | CD117) HC-32 | WMGWLNPSGGGTSYAQKFQGRV |
| | | TMTRDTSTSTVYMELSSLRSEDTA |
| | | VYYCARHGRGYDGYEGAFDIWG |
| | | QGTLVTVSSAS |
| SEQ ID NO: 56 | Light chain variable | DIQMTQSPSSLSASVGDRVTITCR |
| | region of (anti- | ASQGIRNDLGWYQQKPGKAPKLLI |
| | CD117) LC-32 | YDASNLETGVPSRFSGSGSGTDF |
| | | TLTISSLQPEDFATYYCQQLNGYP |
| | | LTFGGGTKVEIK |
| SEQ ID NO: 57 | Heavy chain variable | QVQLVQSGAEVKKPGASVKVSCK |
| | region of (anti- | ASGYTFSTYYMHWVRQAPGQGL |
| | CD117) HC-33 | EWMGIINPSGGSTSYAQKFQGRV |
| | | TMTRDTSTSTVYMKLSSLRSEDTA |
| | | VYYCARHGRGYEGYEGAFDIWGQ |
| | | GTLVTVSSAS |
| SEQ ID NO: 58 | Light chain variable | DIQMTQSPSSLSASVGDRVTITCR |
| | region of (anti- | ASQGIRDDLGWYQQKPGKAPKLLI |
| | CD117) LC-33 | YDASNLETGVPSRFSGSGSGTDF |
| | | TLTISSLQPEDFATYYCQQANGFP |
| | | LTFGGGTKVEIK |
| SEQ ID NO: 59 | Heavy chain variable | QVQLVQSGAEVKKPGASVKVSCK |
| | region of (anti- | ASGYTFTGYYIHWVRQAPGQGLE |
| | CD117) HC-34 | WMGIINPSGGNTNYAQNFQGRVT |
| | | MTRDTSTSTVYMELSSLRSEDTAV |
| | | YYCARHGRGYNAYEGAFDIWGQ |
| | | GTLVTVSSAS |
| SEQ ID NO: 60 | Light chain variable | DIQMTQSPSSLSASVGDRVTITCR |
| | region of (anti- | ASQGIRNDLGWYQQKPGKAPKLLI |
| | CD117) LC-34 | YDASNLETGVPSRFSGSGSGTDF |
| | | TLTISSLQPEDFATYYCQQVNGYP |
| | | LTFGGGTKVEIK |
| SEQ ID NO: 61 | Heavy chain variable | QVQLVQSGAEVKKPGASVKVSCK |
| | region of (anti- | ASGGTFSSYAISWVRQAPGQGLE |
| | CD117) HC-35 | WMGVINPTVGGANYAQKFQGRVT |
| | | MTRDTSTSTVYMELSSLRSEDTAV |
| | | YYCARHGRGYNEYEGAFDIWGQ |
| | | GTLVTVSSAS |
| SEQ ID NO: 62 | Light chain variable | DIQMTQSPSSLSASVGDRVTITCQ |
| | region of (anti- | ASQDISDYLNWYQQKPGKAPKLLI |
| | CD117) LC-35 | YDASNLETGVPSRFSGSGSGTDF |
| | | TLTISSLQPEDFATYYCQQGNSFP |
| | | LTFGGGTKLEIK |
| SEQ ID NO: 63 | Heavy chain variable | QVQLVQSGAEVKKLGASVKVSCK |
| | region of (anti- | ASGYTFSSYYMHWVRQAPGQGL |
| | CD117) HC-36 | EWMGVINPNGAGTNFAQKFQGRV |
| | | TMTRDTSTSTVYMELSSLRSEDTA |
| | | VYYCARHGRGYEGYEGAFDIWGQ |
| | | GTLVTVSSAS |
| SEQ ID NO: 52 | Light chain variable | DIQMTQSPSSLSASVGDRVTITCR |
| | region of (anti- | ASQGIRNDLGWYQQKPGKAPKLLI |
| | CD117) LC-36 | YDASSLESGVPSRFSGSGSGTDF |
| | | TLTISSLQPEDFATYYCQQLNGYP |
| | | LTFGGGTKVEIK |
| SEQ ID NO: 64 | Heavy chain variable | QVQLVQSGAEVKKPGASVKVSCK |
| | region of (anti- | ASGYTFTTYYMHWVRQAPGQGLE |
| | CD117) HC-37 | WMGWINPTGGGTNYAQNFQGRV |
| | | TMTRDTSTSTVYMELSSLRSEDTA |
| | | VYYCARHGRGYEGYEGAFDIWGQ |
| | | GTLVTVSSAS |
| SEQ ID NO: 65 | Light chain variable | DIQMTQSPSSLSASVGDRVTITCR |
| | region of (anti- | ASQGIRNDVSWYQQKPGKAPKLLI |
| | CD117) LC-37 | YDASNLETGVPSRFSGSGSGTDF |
| | | TLTISSLQPEDFATYYCQQLSGYPI |
| | | TFGQGTKLEIK |
| SEQ ID NO: 66 | Heavy chain variable | QVQLVQSGAEVKKPGASVKVSCK |
| | region of (anti- | ASGYTFTSYYIHWVRQAPGQGLE |
| | CD117) HC-38 | WMGMINPSGGSTNYAQKFQGRV |
| | | TMTRDTSTSTVYMELSSLRSEDTA |
| | | VYYCARHGRGYNDYEGAFDIWGQ |
| | | GTLVTVSSAS |
| SEQ ID NO: 67 | Light chain variable | DIQMTQSPSSLSASVGDRVTITCR |
| | region of (anti- | ASQSISDWLAWYQQKPGKAPKLLI |
| | CD117) LC-38 | YEASNLEGGVPSRFSGSGSGTDF |
| | | TLTISSLQPEDFATYYCQQANSFP |
| | | YTFGQGTKVEIK |
| SEQ ID NO: 68 | Heavy chain variable | QVQLVQSGAEVKKPGASVKVSCK |
| | region of (anti- | ASGYIFSAYYIHWVRQAPGQGLE |
| | CD117) HC-39 | WMGIINPSGGSTRYAQKFQGRVT |
| | | MTRDTSTSTVYMELSSLRSEDTAV |
| | | YYCARHGRGYGGYEGAFDIWDQ |
| | | GTLVTVSSAS |
| SEQ ID NO: 69 | Light chain variable | DIQMTQSPSSLSASVGDRVTITCR |
| | region of (anti- | ASQGIGDYVAWYQQKPGKAPKLLI |
| | CD117) LC-39 | YDASNLETGVPSRFSGSGSGTDF |
| | | TLTISSLQPEDFATYYCQQLNGYPI |
| | | TFGQGTRLEIK |
| SEQ ID NO: 70 | Heavy chain variable | EVQLVQSGAEVKKPGESLKISCKG |
| | region of (anti- | SGYRFTSYWIGWVRQMPGKGLE |
| | CD117) HC-40 | WMGIIYPDDSDTRYSPSFQGQVTI |
| | | SVDKSNSTAYLQWSSLKASDTAM |
| | | YYCARHGRGYNGYEGAFDIWGQ |
| | | GTLVTVSSAS |
| SEQ ID NO: 71 | Light chain variable | DIQMTQSPSSLSASVGDRVTITCR |
| | region of (anti- | ASQGISSYLAWYQQKPGKAPKLLI |
| | CD117) LC-40 | YDASNLETGVPSRFSGSGSGTYF |
| | | TLTISSLQPEDFATYYCQQGASFPI |
| | | TFGQGTKVEIK |
| SEQ ID NO: 72 | Heavy chain variable | EVQLVQSGAEVKKPGESLKISCKG |
| | region of (anti- | SGSSFPNSWIAWVRQMPGKGLE |
| | CD117) HC-41 | WMGIIYPSDSDTRYSPSFQGQVTI |
| | | SADKSISTAYLQWSSLEASDTAMY |
| | | YCARHGRGYNGYEGAFDIWGQG |
| | | TLVTVSSAS |
| SEQ ID NO: 73 | Light chain variable | DIQMTQSPSSLSASVGDRVTITCR |
| | region of (anti- | ASQGIRNYLAWYQQKPGKAPKLLI |
| | CD117) LC-41 | YDASSLQSGVPSRFSGSGSGTDF |
| | | TLTISSLQPEDFATYYCQQLNSYPL |
| | | TFGGGTKVEIK |
| SEQ ID NO: 74 | Heavy chain variable | EVQLVQSGAEVKKPGESLKISCKG |
| | region of (anti- | SGYSFDSYWIGWVRQMPGKGLE |
| | CD117) HC-42 | WMGIMYPGDSDTRYSPSFQGQVT |
| | | ISADKSISTAYLQWSSLKASDTAM |
| | | YYCARHGRGYNAYEGAFDIWGQ |
| | | GTLVTVSSAS |
| SEQ ID NO: 75 | Light chain variable | DIQMTQSPSSLSASVGDRVTITCR |
| | region of (anti- | ASQSINNWLAWYQQKPGKAPKLLI |
| | CD117) LC-42 | YDAFILQSGVPSRFSGSGSGTDFT |
| | | LTISSLQPEDFATYYCLQLNSYPLT |
| | | FGPGTKVDIK |
| SEQ ID NO: 76 | Heavy chain variable | EVQLVQSGAEVKKPGESLKISCKG |
| | region of (anti- | SGYSFTNWIAWVRQMPGKGLEW |
| | CD117) HC-43 | MGIIYPGDSETRYSPSFQGQVTIS |
| | | ADKSISTAYLQWSSLKASDTAMYY |
| | | CARHGRGYYGYEGAFDIWGQGTL |
| | | VTVSSAS |
| SEQ ID NO: 77 | Light chain variable | DIQMTQSPSSLSASVGDRVTITCR |
| | region of (anti- | ASQGISDNLNWYQQKPGKAPKLLI |
| | CD117) LC-43 | YDASNLETGVPSRFSGSGSGTDF |
| | | TLTISSLQPEDFATYYCQQAISFPL |
| | | TFGQGTKVEIK |
| SEQ ID NO: 78 | Heavy chain variable | EVQLVQSGAEVKKPGESLKISCKG |
| | region of (anti- | SGYNFTSYWIGWVRQMPGKGLE |
| | CD117) HC-44 | WMGVIYPDDSETRYSPSFQGQVTI |
| | | SADKSISTAYLQWSSLKASDTAMY |
| | | YCARHGRGYNGYEGAFDIWGQG |
| | | TLVTVSSAS |
| SEQ ID NO: 79 | Light chain variable | DIQMTQSPSSLSASVGDRVTITCR |
| | region of (anti- | ASRDIRDDLGWYQQKPGKAPKLLI |
| | CD117) LC-44 | YDASNLETGVPSRFSGSGSGTDF |
| | | TLTISSLQPEDFATYYCQQANSFP |
| | | LTFGGGTKVEIK |
| SEQ ID NO: 80 | Heavy chain variable | EVQLVQSGAEVKKPGESLKISCKG |
| | region of (anti- | SGYTFNTYIGWVRQMPGKGLEW |
| | CD117) HC-45 | MGIIYPGDSGTRYSPSFQGQVTIS |
| | | ADKAISTAYLQWSSLKASDTAMYY |
| | | CARHSRGYNGYEGAFDIWGQGTL |
| | | VTVSSAS |
| SEQ ID NO: 81 | Light chain variable | DIQMTQSPSSLSASVGDRVTITCR |
| | region of (anti- | ASQGISNYLAWYQQKPGKAPKLLI |
| | CD117) LC-45 | YDASNLETGVPSRFSGSGSGTDF |
| | | TLTISSLQPEDFATYYCQQANSFP |
| | | VTFGQGTKVEIK |
| SEQ ID NO: 82 | Heavy chain variable | EVQLVQSGAEVKKPGESLKISCKG |
| | region of (anti- | SGYNFTTYWIGWVRQMPGKGLE |
| | CD117) HC-46 | WMGIIHPADSDTRYNPSFQGQVTI |
| | | SADKSISTAYLQWSSLKASDTAMY |
| | | YCARHGRGYNGYEGAFDIWGQG |
| | | TLVTVSSAS |
| SEQ ID NO: 83 | Light chain variable | DIQMTQSPSSLSASVGDRVTITCR |
| | region of (anti- | VSQGISSYLAWYQQKPGKAPKLLI |
| | CD117) LC-46 | YDASNLETGVPSRFSGSGSGTDF |
| | | TLTISSLQPEDFATYYCQQANSFP |
| | | LTFGGGTKVEIK |
| SEQ ID NO: 84 | Heavy chain variable | EVQLVQSGAEVKKPGESLKISCKG |
| | region of (anti- | SGYRFSNYWIAWVRQMPGKGLE |
| | CD117) HC-47 | WMGIIYPDNSDTRYSPSFQGQVTI |
| | | SADKSISTAYLQWSSLKASDTAMY |
| | | YCARHGRGYDGYEGAFDIWGQG |
| | | TLVTVSSAS |
| SEQ ID NO: 85 | Light chain variable | DIQMTQSPSSLSASVGDRVTITCR |
| | region of (anti- | ASQGIRSDLAWYQQKPGKAPKLLI |
| | CD117) LC-47 | YGASSLQSGVPSRFSGSGSGTDF |
| | | TLTISSLQPEDFATYYCQQANSFP |
| | | LSFGQGTKVEIK |
| SEQ ID NO: 86 | Heavy chain variable | EVQLVQSGAEVKKPGESLKISCKG |
| | region of (anti- | SGYRFASYWIGWVRQMPGKGLE |
| | CD117) HC-48 | WMGITYPGDSETRYNPSQGQVTI |
| | | SADKSISTAYLQWSSLKASDTAMY |
| | | YCARHGRGYGGYEGAFDIWGQG |
| | | TLVTVSSAS |
| SEQ ID NO: 87 | Light chain variable | DIQMTQSPSSLSASVGDRVTITCR |
| | region of (anti- | ASQGIRNDLGWYQQKPGKAPKLLI |
| | CD117) LC-48 | YDASNLETGVPSRFSGSGSGTDF |
| | | TLTISSLQPEDFATYYCQQANSFP |
| | | LTFGGGTKVEIK |
| SEQ ID NO: 88 | Heavy chain variable | EVQLVQSGAEVKKPGESLKISCKG |
| | region of (anti- | SGYSFTSYWIGWVRQMPGKGLE |
| | CD117) HC-49 | WMGIIYPGDSDTRYSPSFQGQVTI |
| | | SADKSISTAYLQWSSLKASDTAMY |
| | | YCARHGRGYNGYEGAFDIWGQG |
| | | TLVTVSSAS |
| SEQ ID NO: 89 | Light chain variable | DIQMTQSPSSLSASVGDRVTITCR |
| | region of (anti- | ASQSISNWLAWYQQKPGKAPKLLI |
| | CD117) LC-49 | YDASNLETGVPSRFSGSGSGTDF |
| | | TLTISSLQPEDFATYYCQQTNSFPL |
| | | TFGQGTRLEIK |
| SEQ ID NO: 9 | Heavy chain variable | QVQLVQSGAAVKKPGESLKISCKG |
| | region of (anti- | SGYRFTTYWIGWVRQMPGKGLE |
| | CD117) HC-74 | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 90 | Light chain variable | DIQLTQSPSSLSASVGDRVTITCRA |
| | region of (anti- | SQGVISALAWYQQKPGKAPKLLIY |
| | CD117) LC-74 | DASSLESGVPSRFSGSGSGTDFT |
| | | LTISSLQPEDFATYYCQQFNSYPL |
| | | TFGGGTKVEIK |
| SEQ ID NO: 9 | Heavy chain variable | QVQLVQSGAAVKKPGESLKISCKG |
| | region of (anti- | SGYRFTTYWIGWVRQMPGKGLE |
| | CD117) HC-75 | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 91 | Light chain variable | DIQLTQSPSSLSASVGDRVTITCRA |
| | region of (anti- | SQGIRSALAWYQQKPGKAPKLLIY |
| | CD117) LC-75 | DASSLESGVPSRFSGSGSGTDFT |
| | | LTISSLQPEDFATYYCQQFNSYPL |
| | | TFGGGTKVEIK |
| SEQ ID NO: 9 | Heavy chain variable | QVQLVQSGAAVKKPGESLKISCKG |
| | region of (anti- | SGYRFTTYWIGWVRQMPGKGLE |
| | CD117) HC-76 | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 92 | Light chain variable | DIQLTQSPSSLSASVGDRVTITCRA |
| | region of (anti- | SQGVGSALAWYQQKPGKAPKLLI |
| | CD117) LC-76 | YDASSLESGVPSRFSGSGSGTDF |
| | | TLTISSLQPEDFATYYCQQFNSYP |
| | | LTFGGGTKVEIK |
| SEQ ID NO: 9 | Heavy chain variable | QVQLVQSGAAVKKPGESLKISCKG |
| | region of (anti- | SGYRFTTYWIGWVRQMPGKGLE |
| | CD117) HC-77 | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 93 | Light chain variable | DIQLTQSPSSLSASVGDRVTITCRA |
| | region of (anti- | SQGVISALAWYQQKPGKAPKLLIY |
| | CD117) LC-77 | DASILESGVPSRFSGSGSGTDFTL |
| | | TISSLQPEDFATYYCQQFNSYPLT |
| | | FGGGTKVEIK |
| SEQ ID NO: 94 | Ab77 (anti-CD117) | TYWIG |
| | CDR-H1 | |
| SEQ ID NO: 2 | Ab77 (anti-CD117) | IIYPGDSDTRYSPSFQG |
| | CDR-H2 | |
| SEQ ID NO: 3 | Ab77 (anti-CD117) | HGRGYNGYEGAFDI |
| | CDR-H3 | |
| SEQ ID NO: 95 | Ab77 (anti-CD117) | RASQGVISALA |
| | CDR-L1 | |
| SEQ ID NO: 96 | Ab77 (anti-CD117) | DASILES |
| | CDR-L2 | |
| SEQ ID NO: 97 | Ab77 (anti-CD117) | QQFNSYPLT |
| | CDR-L3 | |
| SEQ ID NO: 9 | Heavy chain variable | QVQLVQSGAAVKKPGESLKISCKG |
| | region of (anti- | SGYRFTTYWIGWVRQMPGKGLE |
| | CD117) HC-78 | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 98 | Light chain variable | DIQLTQSPSSLSASVGDRVTITCRA |
| | region of (anti- | SQGIRSALAWYQQKPGKAPKLLIY |
| | CD117) LC-78 | DASILESGVPSRFSGSGSGTDFTL |
| | | TISSLQPEDFATYYCQQFNSYPLT |
| | | FGGGTKVEIK |
| SEQ ID NO: 9 | Heavy chain variable | QVQLVQSGAAVKKPGESLKISCKG |
| | region of (anti- | SGYRFTTYWIGWVRQMPGKGLE |
| | CD117) HC-79 | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 99 | Light chain variable | DIQLTQSPSSLSASVGDRVTITCRA |
| | region of (anti- | SQGVGSALAWYQQKPGKAPKLLI |
| | CD117) LC-79 | YDASILESGVPSRFSGSGSGTDFT |
| | | LTISSLQPEDFATYYCQQFNSYPL |
| | | TFGGGTKVEIK |
| SEQ ID NO: 94 | Ab79 (anti-CD117) | TYWIG |
| | CDR-H1 | |
| SEQ ID NO: 2 | Ab79 (anti-CD117) | IIYPGDSDTRYSPSFQG |
| | CDR-H2 | |
| SEQ ID NO: 3 | Ab79 (anti-CD117) | HGRGYNGYEGAFDI |
| | CDR-H3 | |
| SEQ ID NO: | Ab79 (anti-CD117) | RASQGVGSALA |
| 100 | CDR-L1 | |
| SEQ ID NO: 96 | Ab79 (anti-CD117) | DASILES |
| | CDR-L2 | |
| SEQ ID NO: 97 | Ab79 (anti-CD117) | QQFNSYPLT |
| | CDR-L3 | |
| SEQ ID NO: 9 | Heavy chain variable | QVQLVQSGAAVKKPGESLKISCKG |
| | region of (anti- | SGYRFTTYWIGWVRQMPGKGLE |
| | CD117) HC-80 | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: | Light chain variable | DIQLTQSPSSLSASVGDRVTITCRA |
| 101 | region of (anti- | SQGISSALAWYQQKPGKAPKLLIY |
| | CD117) LC-80 | DASILESGVPSRFSGSGSGTDFTL |
| | | TISSLQPEDFATYYCQQFNSYPLT |
| | | FGGGTKVEIK |
| SEQ ID NO: 9 | Heavy chain variable | QVQLVQSGAAVKKPGESLKISCKG |
| | region of (anti- | SGYRFTTYWIGWVRQMPGKGLE |
| | CD117) HC-81 | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: | Light chain variable | DIQLTQSPSSLSASVGDRVTITCRA |
| 102 | region of (anti- | SQGVISALAWYQQKPGKAPKLLIY |
| | CD117) LC-81 | DASTLESGVPSRFSGSGSGTDFTL |
| | | TISSLQPEDFATYYCQQFNSYPLT |
| | | FGGGTKVEIK |
| SEQ ID NO: 94 | Ab81 (anti-CD117) | TYWIG |
| | CDR-H1 | |
| SEQ ID NO: 2 | Ab81 (anti-CD117) | IIYPGDSDTRYSPSFQG |
| | CDR-H2 | |
| SEQ ID NO: 3 | Ab81 (anti-CD117) | HGRGYNGYEGAFDI |
| | CDR-H3 | |
| SEQ ID NO: 95 | Ab81 (anti-CD117) | RASQGVISALA |
| | CDR-L1 | |
| SEQ ID NO: | Ab81 (anti-CD117) | DASTLES |
| 103 | CDR-L2 | |
| SEQ ID NO: 97 | Ab81 (anti-CD117) | QQFNSYPLT |
| | CDR-L3 | |
| SEQ ID NO: 9 | Heavy chain variable | QVQLVQSGAAVKKPGESLKISCKG |
| | region of (anti- | SGYRFTTYWIGWVRQMPGKGLE |
| | CD117) HC-82 | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: | Light chain variable | DIQLTQSPSSLSASVGDRVTITCRA |
| 104 | region of (anti- | SQGIRSALAWYQQKPGKAPKLLIY |
| | CD117) LC-82 | DASTLESGVPSRFSGSGSGTDFTL |
| | | TISSLQPEDFATYYCQQFNSYPLT |
| | | FGGGTKVEIK |
| SEQ ID NO: 9 | Heavy chain variable | QVQLVQSGAAVKKPGESLKISCKG |
| | region of (anti- | SGYRFTTYWIGWVRQMPGKGLE |
| | CD117) HC-83 | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: | Light chain variable | DIQLTQSPSSLSASVGDRVTITCRA |
| 105 | region of (anti- | SQGVGSALAWYQQKPGKAPKLLI |
| | CD117) LC-83 | YDASTLESGVPSRFSGSGSGTDF |
| | | TLTISSLQPEDFATYYCQQFNSYP |
| | | LTFGGGTKVEIK |
| SEQ ID NO: 9 | Heavy chain variable | QVQLVQSGAAVKKPGESLKISCKG |
| | region of (anti- | SGYRFTTYWIGWVRQMPGKGLE |
| | CD117) HC-84 | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: | Light chain variable | DIQLTQSPSSLSASVGDRVTITCRA |
| 105 | region of (anti- | SQGVGSALAWYQQKPGKAPKLLI |
| | CD117) LC-84 | YDASTLESGVPSRFSGSGSGTDF |
| | | TLTISSLQPEDFATYYCQQFNSYP |
| | | LTFGGGTKVEIK |
| SEQ ID NO: | Heavy chain variable | EVQLVQSGAEVKKPGESLKISCKG |
| 106 | region of (anti- | SGYSFTNYWIGWVRQMPGKGLE |
| | CD117) Ab 85 | WMAIINPRDSDTRYRPSFQGQVTI |
| | | SADKSISTAYLQWSSLKASDTAMY |
| | | YCARHGRGYEGYEGAFDIWGQG |
| | | TLVTVSS |
| SEQ ID NO: | Light chain variable | DIQMTQSPSSLSASVGDRVTITCR |
| 107 | region of (anti- | SSQGIRSDLGWYQQKPGKAPKLLI |
| | CD117) Ab 85 | YDASNLETGVPSRFSGSGSGTDF |
| | | TLTISSLQPEDFATYYCQQANGFP |
| | | LTFGGGTKVEIK |
| SEQ ID NO: | Ab85 (anti-CD117) | NYWIG |
| 108 | CDR-H1 | |
| SEQ ID NO: | Ab85 (anti-CD117) | IINPRDSDTRYRPSFQG |
| 109 | CDR-H2 | |
| SEQ ID NO: | Ab85 (anti-CD117) | HGRGYEGYEGAFDI |
| 110 | CDR-H3 | |
| SEQ ID NO: | Ab85 (anti-CD117) | RSSQGIRSDLG |
| 111 | CDR-L1 | |
| SEQ ID NO: | Ab85 (anti-CD117) | DASNLET |
| 112 | CDR-L2 | |
| SEQ ID NO: | Ab85 (anti-CD117) | QQANGFPLT |
| 113 | CDR-L3 | |
| SEQ ID NO: | Heavy chain variable | EVQLVQSGAEVKKPGESLKISCKG |
| 114 | region of (anti- | SGYSFTNYWIGWVRQMPGKGLE |
| | CD117) Ab 86 | WMGIIYPGDSDIRYSPSLQGQVTIS |
| | | VDTSTSTAYLQWNSLKPSDTAMY |
| | | YCARHGRGYNGYEGAFDIWGQG |
| | | TLVTVSS |
| SEQ ID NO: | Light chain variable | DIQMTQSPSSLSASVGDRVTITCR |
| 115 | region of (anti- | ASQGIGDSLAWYQQKPGKAPKLLI |
| | CD117) Ab 86 | YDASNLETGVPSRFSGSGSGTDF |
| | | TLTISSLQPEDFATYYCQQLNGYPI |
| | | TFGQGTKVEIK |
| SEQ ID NO: | Ab86 (anti-CD117) | NYWIG |
| 108 | CDR-H1 | |
| SEQ ID NO: | Ab86 (anti-CD117) | IIYPGDSDIRYSPSLQG |
| 116 | CDR-H2 | |
| SEQ ID NO: 3 | Ab86 (anti-CD117) | HGRGYNGYEGAFDI |
| | CDR-H3 | |
| SEQ ID NO: | Ab86 (anti-CD117) | RASQGIGDSLA |
| 117 | CDR-L1 | |
| SEQ ID NO: | Ab86 (anti-CD117) | DASNLET |
| 112 | CDR-L2 | |
| SEQ ID NO: | Ab86 (anti-CD117) | QQLNGYPIT |
| 118 | CDR-L3 | |
| SEQ ID NO: | Heavy chain variable | EVQLVQSGAEVKKPGESLKISCKG |
| 106 | region of (anti- | SGYSFTNYWIGWVRQMPGKGLE |
| | CD117) Ab 87 | WMAIINPRDSDTRYRPSFQGQVTI |
| | | SADKSISTAYLQWSSLKASDTAMY |
| | | YCARHGRGYEGYEGAFDIWGQG |
| | | TLVTVSS |
| SEQ ID NO: | Light chain variable | DIQMTQSPSSLSASVGDRVTITCR |
| 119 | region of (anti- | ASQGIRNDLGWYQQKPGKAPKLLI |
| | CD117) Ab 87 | YDASSLESGVPSRFSGSGSGTDF |
| | | TLTISSLQPEDFATYYCQQLNGYPI |
| | | TFGQGTKVEIK |
| SEQ ID NO: | Ab87 (anti-CD117) | NYWIG |
| 108 | CDR-H1 | |
| SEQ ID NO: | Ab87 (anti-CD117) | IINPRDSDTRYRPSFQG |
| 109 | CDR-H2 | |
| SEQ ID NO: | Ab87 (anti-CD117) | HGRGYEGYEGAFDI |
| 110 | CDR-H3 | |
| SEQ ID NO: | Ab87 (anti-CD117) | RASQGIRNDLG |
| 120 | CDR-L1 | |
| SEQ ID NO: 5 | Ab87 (anti-CD117) | DASSLES |
| | CDR-L2 | |
| SEQ ID NO: | Ab87 (anti-CD117) | QQLNGYPIT |
| 118 | CDR-L3 | |
| SEQ ID NO: | Heavy chain variable | EVQLVQSGAEVKKPGESLKISCKG |
| 121 | region of (anti- | SGYSFTNYWIGWVRQMPGKGLE |
| | CD117) Ab 88 | WMGIIYPGDSLTRYSPSFQGQVTI |
| | | SADKSISTAYLQWSSLKASDTAMY |
| | | YCARHGRGYNGYEGAFDIWGQG |
| | | TLVTVSS |
| SEQ ID NO: | Light chain variable | DIQMTQSPSSLSASVGDRVTITCR |
| 119 | region of (anti- | ASQGIRNDLGWYQQKPGKAPKLLI |
| | CD117) Ab 88 | YDASSLESGVPSRFSGSGSGTDF |
| | | TLTISSLQPEDFATYYCQQLNGYPI |
| | | TFGQGTKVEIK |
| SEQ ID NO: | Ab88 (anti-CD117) | NYWIG |
| 108 | CDR-H1 | |
| SEQ ID NO: | Ab88 (anti-CD117) | IIYPGDSLTRYSPSFQG |
| 122 | CDR-H2 | |
| SEQ ID NO: 3 | Ab88 (anti-CD117) | HGRGYNGYEGAFDI |
| | CDR-H3 | |
| SEQ ID NO: | Ab88 (anti-CD117) | RASQGIRNDLG |
| 120 | CDR-L1 | |
| SEQ ID NO: 5 | Ab88 (anti-CD117) | DASSLES |
| | CDR-L2 | |
| SEQ ID NO: | Ab88 (anti-CD117) | QQLNGYPIT |
| 118 | CDR-L3 | |
| SEQ ID NO: | Heavy chain variable | EVQLVQSGAEVKKPGESLKISCKG |
| 123 | region of (anti- | SGYSFTNYWIGWVRQMPGKGLE |
| | CD117) Ab89 | WMGIIYPGDSDTRYSPSFQGQVTI |
| | | SADKSISTAYLQWSSLKASDTAMY |
| | | YCARHGRGYNGYEGAFDIWGQG |
| | | TLVTVSS |
| SEQ ID NO: | Light chain variable | DIQMTQSPSSLSASVGDRVTITCR |
| 115 | region of (anti- | ASQGIGDSLAWYQQKPGKAPKLLI |
| | CD117) Ab89 | YDASNLETGVPSRFSGSGSGTDF |
| | | TLTISSLQPEDFATYYCQQLNGYPI |
| | | TFGQGTKVEIK |
| SEQ ID NO: | Ab89 (anti-CD117) | NYWIG |
| 108 | CDR-H1 | |
| SEQ ID NO: 2 | Ab89 (anti-CD117) | IIYPGDSDTRYSPSFQG |
| | CDR-H2 | |
| SEQ ID NO: 3 | Ab89 (anti-CD117) | HGRGYNGYEGAFDI |
| | CDR-H3 | |
| SEQ ID NO: | Ab89 (anti-CD117) | RASQGIGDSLA |
| 117 | CDR-L1 | |
| SEQ ID NO: | Ab89 (anti-CD117) | DASNLET |
| 112 | CDR-L2 | |
| SEQ ID NO: | Ab89 (anti-CD117) | QQLNGYPIT |
| 118 | CDR-L3 | |
| SEQ ID NO: | Heavy chain variable | EVQLVQSGAEVKKPGESLKISCKG |
| 124 | region of (anti- | SGYRFTTSWIGWVRQMPGKGLE |
| | CD117) HC-245 | WMGIIYPGDSDTRYSPSFQGQVTI |
| | | SADKSISTAYLQWSSLKASDTAMY |
| | | YCARHGLGYNGYEGAFDIWGQGT |
| | | LVTVSS |
| SEQ ID NO: | Light chain variable | DIQMTQSPSSLSASVGDRVTITCR |
| 125 | region of (anti- | ASQGIGSALAWYQQKPGKAPKLLI |
| | CD117) LC-245 | YDASTLESGVPSRFSGSGSGTDF |
| | | TLTISSLQPEDFATYYCQQFNGYP |
| | | LTFGQGTRLEIK |
| SEQ ID NO: 9 | Heavy chain variable | QVQLVQSGAAVKKPGESLKISCKG |
| | region of (anti- | SGYRFTTYWIGWVRQMPGKGLE |
| | CD117) HC-246 | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: | Light chain variable | DIQMTQSPSSLSASVGDRVTITCR |
| 125 | region of (anti- | ASQGIGSALAWYQQKPGKAPKLLI |
| | CD117) LC-246 | YDASTLESGVPSRFSGSGSGTDF |
| | | TLTISSLQPEDFATYYCQQFNGYP |
| | | LTFGQGTRLEIK |
| SEQ ID NO: 9 | Heavy chain variable | QVQLVQSGAAVKKPGESLKISCKG |
| | region of (anti- | SGYRFTTYWIGWVRQMPGKGLE |
| | CD117) HC-247 | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: | Light chain variable | DIQMTQSPSSLSASVGDRVTITCR |
| 126 | region of (anti- | ASRGISDYLAWYQQKPGKAPKLLI |
| | CD117) LC-247 | YDASNLETGVPSRFSGSGSGTDF |
| | | TLTISSLQPEDFATYYCQQANSFPI |
| | | TFGQGTRLEIK |
| SEQ ID NO: | Heavy chain variable | EVQLVQSGAEVKKPGESLKISCKG |
| 124 | region of (anti- | SGYRFTTSWIGWVRQMPGKGLE |
| | CD117) HC-248 | WMGIIYPGDSDTRYSPSFQGQVTI |
| | | SADKSISTAYLQWSSLKASDTAMY |
| | | YCARHGLGYNGYEGAFDIWGQGT |
| | | LVTVSS |
| SEQ ID NO: | Light chain variable | DIQMTQSPSSLSASVGDRVTITCR |
| 127 | region of (anti- | ASQGIGSALAWYQQKPGKAPKLLI |
| | CD117) LC-248 | YDASTLESGVPSRFSGSGSGTDF |
| | | TLTISSLQPEDFATYYCQQLNGYP |
| | | LTFGQGTRLEIK |
| SEQ ID NO: | Heavy chain variable | EVQLVQSGAEVKKPGESLKISCKG |
| 124 | region of (anti- | SGYRFTTSWIGWVRQMPGKGLE |
| | CD117) HC-249 | WMGIIYPGDSDTRYSPSFQGQVTI |
| | | SADKSISTAYLQWSSLKASDTAMY |
| | | YCARHGLGYNGYEGAFDIWGQGT |
| | | LVTVSS |
| SEQ ID NO: | Light chain variable | DIQMTQSPSSLSASVGDRVTITCR |
| 128 | region of (anti- | ASQGIGSALAWYQQKPGKAPKLLI |
| | CD117) LC-249 | YDASNLETGVPSRFSGSGSGTDF |
| | | TLTISSLQPEDFATYYCQQLNGYP |
| | | LTFGQGTRLEIK |
| SEQ ID NO: | Ab249 (anti-CD117) | TSWIG |
| 129 | HC-CDR1 | |
| SEQ ID NO: 2 | Ab249 (anti-CD117) | IIYPGDSDTRYSPSFQG |
| | HC-CDR2 | |
| SEQ ID NO: | Ab249 (anti-CD117) | HGLGYNGYEGAFDI |
| 130 | HC-CDR3 | |
| SEQ ID NO: | Ab249 (anti-CD117) | RASQGIGSALA |
| 131 | LC-CDR1 | |
| SEQ ID NO: | Ab249 (anti-CD117) | DASNLET |
| 112 | LC-CDR2 | |
| SEQ ID NO: | Ab249 (anti-CD117) | CQQLNGYPLT |
| 132 | LC-CDR3 | |
| SEQ ID NO: | Ab85 (anti-CD117) | EVQLVQSGAEVKKPGESLKISCKG |
| 133 | full length heavy | SGYSFTNYWIGWVRQMPGKGLE |
| | chain sequence; | WMAIINPRDSDTRYRPSFQGQVTI |
| | constant region | SADKSISTAYLQWSSLKASDTAMY |
| | underlined | YCARHGRGYEGYEGAFDIWGQG |
| | | TLVTVSSASTKGPSVFPLAPSSKS |
| | | TSGGTAALGCLVKDYFPEPVTVS |
| | | WNSGALTSGVHTFPAVLQSSGLY |
| | | SLSSVVTVPSSSLGTQTYICNVNH |
| | | KPSNTKVDKKVEPKSCDKTHTCP |
| | | PCPAPELLGGPSVFLFPPKPKDTL |
| | | MISRTPEVTCVVVDVSHEDPEVKF |
| | | NWYVDGVEVHNAKTKPREEQYNS |
| | | TYRVVSVLTVLHQDWLNGKEYKC |
| | | KVSNKALPAPIEKTISKAKGQPREP |
| | | QVYTLPPSRDELTKNQVSLTCLVK |
| | | GFYPSDIAVEWESNGQPENNYKT |
| | | TPPVLDSDGSFFLYSKLTVDKSRW |
| | | QQGNVFSCSVMHEALHNHYTQKS |
| | | LSLSPGK |
| SEQ ID NO: | Ab85 (anti-CD117) | EVQLVQSGAEVKKPGESLKISCKG |
| 134 | full length heavy | SGYSFTNYWIGWVRQMPGKGLE |
| | chain sequence; | WMAIINPRDSDTRYRPSFQGQVTI |
| | constant region | SADKSISTAYLQWSSLKASDTAMY |
| | underlined; modified | YCARHGRGYEGYEGAFDIWGQG |
| | Fc region with | TLVTVSSASTKGPSVFPLAPSSKS |
| | L234A, L235A | TSGGTAALGCLVKDYFPEPVTVS |
| | mutations (mutations | WNSGALTSGVHTFPAVLQSSGLY |
| | in bold)* | SLSSVVTVPSSSLGTQTYICNVNH |
| | | KPSNTKVDKKVEPKSCDKTHTCP |
| | | PCPAPEAAGGPSVFLFPPKPKDTL |
| | | MISRTPEVTCVVVDVSHEDPEVKF |
| | | NWYVDGVEVHNAKTKPREEQYNS |
| | | TYRVVSVLTVLHQDWLNGKEYKC |
| | | KVSNKALPAPIEKTISKAKGQPREP |
| | | QVYTLPPSRDELTKNQVSLTCLVK |
| | | GFYPSDIAVEWESNGQPENNYKT |
| | | TPPVLDSDGSFFLYSKLTVDKSRW |
| | | QQGNVFSCSVMHEALHNHYTQKS |
| | | LSLSPGK |
| SEQ ID NO: | Ab85 (anti-CD117) | EVQLVQSGAEVKKPGESLKISCKG |
| 135 | full length heavy | SGYSFTNYWIGWVRQMPGKGLE |
| | chain sequence: | WMAIINPRDSDTRYRPSFQGQVTI |
| | constant region | SADKSISTAYLQWSSLKASDTAMY |
| | underlined; modified | YCARHGRGYEGYEGAFDIWGQG |
| | Fc region with | TLVTVSSASTKGPSVFPLAPSSKS |
| | L234A, L235A, | TSGGTAALGCLVKDYFPEPVTVS |
| | D265C mutations | WNSGALTSGVHTFPAVLQSSGLY |
| | (mutations in bold)* | SLSSVVTVPSSSLGTQTYICNVNH |
| | | KPSNTKVDKKVEPKSCDKTHTCP |
| | | PCPAPEAAGGPSVFLFPPKPKDTL |
| | | MISRTPEVTCVVVCVSHEDPEVKF |
| | | NWYVDGVEVHNAKTKPREEQYNS |
| | | TYRVVSVLTVLHQDWLNGKEYKC |
| | | KVSNKALPAPIEKTISKAKGQPREP |
| | | QVYTLPPSRDELTKNQVSLTCLVK |
| | | GFYPSDIAVEWESNGQPENNYKT |
| | | TPPVLDSDGSFFLYSKLTVDKSRW |
| | | QQGNVFSCSVMHEALHNHYTQKS |
| | | LSLSPGK |
| SEQ ID NO: | Ab85 (anti-CD117) | EVQLVQSGAEVKKPGESLKISCKG |
| 136 | full length heavy | SGYSFTNYWIGWVRQMPGKGLE |
| | chain sequence | WMAIINPRDSDTRYRPSFQGQVTI |
| | (LALA - D265C - | SADKSISTAYLQWSSLKASDTAMY |
| | H435A mutant); | YCARHGRGYEGYEGAFDIWGQG |
| | constant region | TLVTVSSASTKGPSVFPLAPSSKS |
| | underlined | TSGGTAALGCLVKDYFPEPVTVS |
| | | WNSGALTSGVHTFPAVLQSSGLY |
| | | SLSSVVTVPSSSLGTQTYICNVNH |
| | | KPSNTKVDKKVEPKSCDKTHTCP |
| | | PCPAPEAAGGPSVFLFPPKPKDTL |
| | | MISRTPEVTCVVVCVSHEDPEVKF |
| | | NWYVDGVEVHNAKTKPREEQYNS |
| | | TYRVVSVLTVLHQDWLNGKEYKC |
| | | KVSNKALPAPIEKTISKAKGQPREP |
| | | QVYTLPPSRDELTKNQVSLTCLVK |
| | | GFYPSDIAVEWESNGQPENNYKT |
| | | TPPVLDSDGSFFLYSKLTVDKSRW |
| | | QQGNVFSCSVMHEALHNAYTQKS |
| | | LSLSPGK |
| SEQ ID NO: | Ab249 (anti-CD117) | EVQLVQSGAEVKKPGESLKISCKG |
| 137 | full length heavy | SGYRFTTSWIGWVRQMPGKGLE |
| | chain sequence; | WMGIIYPGDSDTRYSPSFQGQVTI |
| | constant region | SADKSISTAYLQWSSLKASDTAMY |
| | underlined | YCARHGLGYNGYEGAFDIWGQGT |
| | | LVTVSSASTKGPSVFPLAPSSKST |
| | | SGGTAALGCLVKDYFPEPVTVSW |
| | | NSGALTSGVHTFPAVLQSSGLYSL |
| | | SSVVTVPSSSLGTQTYICNVNHKP |
| | | SNTKVDKKVEPKSCDKTHTCPPC |
| | | PAPELLGGPSVFLFPPKPKDTLMIS |
| | | RTPEVTCVVVDVSHEDPEVKFNW |
| | | YVDGVEVHNAKTKPREEQYNSTY |
| | | RVVSVLTVLHQDWLNGKEYKCKV |
| | | SNKALPAPIEKTISKAKGQPREPQ |
| | | VYTLPPSRDELTKNQVSLTCLVKG |
| | | FYPSDIAVEWESNGQPENNYKTT |
| | | PPVLDSDGSFFLYSKLTVDKSRW |
| | | QQGNVFSCSVMHEALHNHYTQKS |
| | | LSLSPGK |
| SEQ ID NO: | Ab249 (anti-CD117) | EVQLVQSGAEVKKPGESLKISCKG |
| 138 | full length heavy | SGYRFTTSWIGWVRQMPGKGLE |
| | chain sequence; | WMGIIYPGDSDTRYSPSFQGQVTI |
| | constant region | SADKSISTAYLQWSSLKASDTAMY |
| | underlined (LALA | YCARHGLGYNGYEGAFDIWGQGT |
| | mutations)* | LVTVSSASTKGPSVFPLAPSSKST |
| | | SGGTAALGCLVKDYFPEPVTVSW |
| | | NSGALTSGVHTFPAVLQSSGLYSL |
| | | SSVVTVPSSSLGTQTYICNVNHKP |
| | | SNTKVDKKVEPKSCDKTHTCPPC |
| | | PAPEAAGGPSVFLFPPKPKDTLMI |
| | | SRTPEVTCVVVDVSHEDPEVKFN |
| | | WYVDGVEVHNAKTKPREEQYNST |
| | | YRVVSVLTVLHQDWLNGKEYKCK |
| | | VSNKALPAPIEKTISKAKGQPREP |
| | | QVYTLPPSRDELTKNQVSLTCLVK |
| | | GFYPSDIAVEWESNGQPENNYKT |
| | | TPPVLDSDGSFFLYSKLTVDKSRW |
| | | QQGNVFSCSVMHEALHNHYTQKS |
| | | LSLSPGK |
| SEQ ID NO: | Ab249 (anti-CD117) | EVQLVQSGAEVKKPGESLKISCKG |
| 139 | full length heavy | SGYRFTTSWIGWVRQMPGKGLE |
| | chain sequence; | WMGIIYPGDSDTRYSPSFQGQVTI |
| | constant region | SADKSISTAYLQWSSLKASDTAMY |
| | underlined (LALA - | YCARHGLGYNGYEGAFDIWGQGT |
| | D265C mutations)* | LVTVSSASTKGPSVFPLAPSSKST |
| | | SGGTAALGCLVKDYFPEPVTVSW |
| | | NSGALTSGVHTFPAVLQSSGLYSL |
| | | SSVVTVPSSSLGTQTYICNVNHKP |
| | | SNTKVDKKVEPKSCDKTHTCPPC |
| | | PAPEAAGGPSVFLFPPKPKDTLMI |
| | | SRTPEVTCVVVCVSHEDPEVKFN |
| | | WYVDGVEVHNAKTKPREEQYNST |
| | | YRVVSVLTVLHQDWLNGKEYKCK |
| | | VSNKALPAPIEKTISKAKGQPREP |
| | | QVYTLPPSRDELTKNQVSLTCLVK |
| | | GFYPSDIAVEWESNGQPENNYKT |
| | | TPPVLDSDGSFFLYSKLTVDKSRW |
| | | QQGNVFSCSVMHEALHNHYTQKS |
| | | LSLSPGK |
| SEQ ID NO: | Ab249 (anti-CD117) | EVQLVQSGAEVKKPGESLKISCKG |
| 140 | full length heavy | SGYRFTTSWIGWVRQMPGKGLE |
| | chain sequence; | WMGIIYPGDSDTRYSPSFQGQVTI |
| | constant region | SADKSISTAYLQWSSLKASDTAMY |
| | underlined; (LALA - | YCARHGLGYNGYEGAFDIWGQGT |
| | D265C - H435A | LVTVSSASTKGPSVFPLAPSSKST |
| | mutations)* | SGGTAALGCLVKDYFPEPVTVSW |
| | | NSGALTSGVHTFPAVLQSSGLYSL |
| | | SSVVTVPSSSLGTQTYICNVNHKP |
| | | SNTKVDKKVEPKSCDKTHTCPPC |
| | | PAPEAAGGPSVFLFPPKPKDTLMI |
| | | SRTPEVTCVVVCVSHEDPEVKFN |
| | | WYVDGVEVHNAKTKPREEQYNST |
| | | YRVVSVLTVLHQDWLNGKEYKCK |
| | | VSNKALPAPIEKTISKAKGQPREP |
| | | QVYTLPPSRDELTKNQVSLTCLVK |
| | | GFYPSDIAVEWESNGQPENNYKT |
| | | TPPVLDSDGSFFLYSKLTVDKSRW |
| | | QQGNVFSCSVMHEALHNAYTQKS |
| | | LSLSPGK |
| SEQ ID NO: | Ab85 (anti-CD117) | DIQMTQSPSSLSASVGDRVTITCR |
| 141 | full length light | SSQGIRSDLGWYQQKPGKAPKLLI |
| | chain; constant | YDASNLETGVPSRFSGSGSGTDF |
| | region underlined | TLTISSLQPEDFATYYCQQANGFP |
| | | LTFGGGTKVEIKRTVAAPSVFIFPP |
| | | SDEQLKSGTASVVCLLNNFYPREA |
| | | KVQWKVDNALQSGNSQESVTEQ |
| | | DSKDSTYSLSSTLTLSKADYEKHK |
| | | VYACEVTHQGLSSPVTKSFNRGE |
| | | C |
| SEQ ID NO: | Ab249 (anti-CD117) | DIQMTQSPSSLSASVGDRVTITCR |
| 142 | light chain; constant | ASQGIGSALAWYQQKPGKAPKLLI |
| | region underlined | YDASNLETGVPSRFSGSGSGTDF |
| | | TLTISSLQPEDFATYYCQQLNGYP |
| | | LTFGQGTRLEIKRTVAAPSVFIFPP |
| | | SDEQLKSGTASVVCLLNNFYPREA |
| | | KVQWKVDNALQSGNSQESVTEQ |
| | | DSKDSTYSLSSTLTLSKADYEKHK |
| | | VYACEVTHQGLSSPVTKSFNRGE |
| | | C |
| SEQ ID NO: | Heavy chain | ASTKGPSVFPLAPSSKSTSGGTAA |
| 143 | constant region | LGCLVKDYFPEPVTVSWNSGALT |
| | (Wild type (WT)) | SGVHTFPAVLQSSGLYSLSSVVTV |
| | | PSSSLGTQTYICNVNHKPSNTKVD |
| | | KKVEPKSCDKTHTCPPCPAPELL |
| | | GGPSVFLFPPKPKDTLMISRTPEV |
| | | TCVVVDVSHEDPEVKFNWYVDGV |
| | | EVHNAKTKPREEQYNSTYRVVSV |
| | | LTVLHQDWLNGKEYKCKVSNKAL |
| | | PAPIEKTISKAKGQPREPQVYTLPP |
| | | SRDELTKNQVSLTCLVKGFYPSDI |
| | | AVEWESNGQPENNYKTTPPVLDS |
| | | DGSFFLYSKLTVDKSRWQQGNVF |
| | | SCSVMHEALHNHYTQKSLSLSPG |
| | | K |
| SEQ ID NO: | Heavy chain | ASTKGPSVFPLAPSSKSTSGGTAA |
| 144 | constant region with | LGCLVKDYFPEPVTVSWNSGALT |
| | L234A, L235A | SGVHTFPAVLQSSGLYSLSSVVTV |
| | (LALA) mutations | PSSSLGTQTYICNVNHKPSNTKVD |
| | (mutations in bold)* | KKVEPKSCDKTHTCPPCPAPEAA |
| | | GGPSVFLFPPKPKDTLMISRTPEV |
| | | TCVVVDVSHEDPEVKFNWYVDGV |
| | | EVHNAKTKPREEQYNSTYRVVSV |
| | | LTVLHQDWLNGKEYKCKVSNKAL |
| | | PAPIEKTISKAKGQPREPQVYTLPP |
| | | SRDELTKNQVSLTCLVKGFYPSDI |
| | | AVEWESNGQPENNYKTTPPVLDS |
| | | DGSFFLYSKLTVDKSRWQQGNVF |
| | | SCSVMHEALHNHYTQKSLSLSPG |
| | | K |
| SEQ ID NO: | Heavy chain constant | ASTKGPSVFPLAPSSKSTSGGTAA |
| 145 | region with D265C | LGCLVKDYFPEPVTVSWNSGALT |
| | mutation | SGVHTFPAVLQSSGLYSLSSVVTV |
| | (mutation in bold)* | PSSSLGTQTYICNVNHKPSNTKVD |
| | | KKVEPKSCDKTHTCPPCPAPELLG |
| | | GPSVFLFPPKPKDTLMISRTPEVT |
| | | CVVVCVSHEDPEVKFNWYVDGVE |
| | | VHNAKTKPREEQYNSTYRVVSVLT |
| | | VLHQDWLNGKEYKCKVSNKALPA |
| | | PIEKTISKAKGQPREPQVYTLPPSR |
| | | DELTKNQVSLTCLVKGFYPSDIAV |
| | | EWESNGQPENNYKTTPPVLDSDG |
| | | SFFLYSKLTVDKSRWQQGNVFSC |
| | | SVMHEALHNHYTQKSLSLSPGK |
| SEQ ID NO: | Heavy chain constant | ASTKGPSVFPLAPSSKSTSGGTAA |
| 146 | region with H435A | LGCLVKDYFPEPVTVSWNSGALT |
| | mutation | SGVHTFPAVLQSSGLYSLSSVVTV |
| | (mutation in bold)* | PSSSLGTQTYICNVNHKPSNTKVD |
| | | KKVEPKSCDKTHTCPPCPAPELLG |
| | | GPSVFLFPPKPKDTLMISRTPEVT |
| | | CVVVDVSHEDPEVKFNWYVDGVE |
| | | VHNAKTKPREEQYNSTYRVVSVLT |
| | | VLHQDWLNGKEYKCKVSNKALPA |
| | | PIEKTISKAKGQPREPQVYTLPPSR |
| | | DELTKNQVSLTCLVKGFYPSDIAV |
| | | EWESNGQPENNYKTTPPVLDSDG |
| | | SFFLYSKLTVDKSRWQQGNVFSC |
| | | SVMHEALHNAYTQKSLSLSPGK |
| SEQ ID NO: | Heavy chain | ASTKGPSVFPLAPSSKSTSGGTAA |
| 147 | constant region: | LGCLVKDYFPEPVTVSWNSGALT |
| | modified Fc region | SGVHTFPAVLQSSGLYSLSSVVTV |
| | with L234A, L235A, | PSSSLGTQTYICNVNHKPSNTKVD |
| | D265C mutations | KKVEPKSCDKTHTCPPCPAPEAA |
| | (mutations in bold)* | GGPSVFLFPPKPKDTLMISRTPEV |
| | | TCVVVCVSHEDPEVKFNWYVDGV |
| | | EVHNAKTKPREEQYNSTYRVVSV |
| | | LTVLHQDWLNGKEYKCKVSNKAL |
| | | PAPIEKTISKAKGQPREPQVYTLPP |
| | | SRDELTKNQVSLTCLVKGFYPSDI |
| | | AVEWESNGQPENNYKTTPPVLDS |
| | | DGSFFLYSKLTVDKSRWQQGNVF |
| | | SCSVMHEALHNHYTQKSLSLSPG |
| | | K |
| SEQ ID NO: | Heavy chain | ASTKGPSVFPLAPSSKSTSGGTAA |
| 148 | constant region: | LGCLVKDYFPEPVTVSWNSGALT |
| | modified Fc region | SGVHTFPAVLQSSGLYSLSSVVTV |
| | with L234A, L235A, | PSSSLGTQTYICNVNHKPSNTKVD |
| | D265C, H435A | KKVEPKSCDKTHTCPPCPAPEAA |
| | mutations (mutations | GGPSVFLFPPKPKDTLMISRTPEV |
| | in bold)* | TCVVVCVSHEDPEVKFNWYVDGV |
| | | EVHNAKTKPREEQYNSTYRVVSV |
| | | LTVLHQDWLNGKEYKCKVSNKAL |
| | | PAPIEKTISKAKGQPREPQVYTLPP |
| | | SRDELTKNQVSLTCLVKGFYPSDI |
| | | AVEWESNGQPENNYKTTPPVLDS |
| | | DGSFFLYSKLTVDKSRWQQGNVF |
| | | SCSVMHEALHNAYTQKSLSLSPG |
| | | K |
| SEQ ID NO: | Light chain constant | RTVAAPSVFIFPPSDEQLKSGTAS |
| 149 | region | VVCLLNNFYPREAKVQWKVDNAL |
| | | QSGNSQESVTEQDSKDSTYSLSS |
| | | TLTLSKADYEKHKVYACEVTHQGL |
| | | SSPVTKSFNRGEC |
| SEQ ID NO: | Ab67 (anti-CD117) | EVQLVESGGGLVQPGGSLRLSCA |
| 150 | Heavy chain variable | ASGFTFSDADMDWVRQAPGKGL |
| | region (e.g., as | EWVGRTRNKAGSYTTEYAASVK |
| | found in HC-67) | GRFTISRDDSKNSLYLQMNSLKTE |
| | (CDRs in bold) | DTAVYYCAREPKYWIDFDLWGRG |
| | | TLVTVSS |
| SEQ ID NO: | Ab67 (anti-CD117) | DIQMTQSPSSLSASVGDRVTITCR |
| 151 | Light chain variable | ASQSISSYLNWYQQKPGKAPKLLI |
| | region (e.g., as | YAASSLQSGVPSRFSGSGSGTDF |
| | found in LC-67) | TLTISSLQPEDFATYYCQQSYIAPY |
| | (CDRs in bold) | TFGGGTKVEIK |
| SEQ ID NO: | Ab67 (anti-CD117) | FTFSDADMD |
| 152 | CDR-H1 | |
| SEQ ID NO: | Ab67 (anti-CD117) | RTRNKAGSYTTEYAASVKG |
| 153 | CDR-H2 | |
| SEQ ID NO: | Ab67 (anti-CD117) | AREPKYWIDFDL |
| 154 | CDR-H3 | |
| SEQ ID NO: | Ab67 (anti-CD117) | RASQSISSYLN |
| 155 | CDR-L1 | |
| SEQ ID NO: | Ab67 (anti-CD117) | AASSLQS |
| 156 | CDR-L2 | |
| SEQ ID NO: | Ab67 (anti-CD117) | QQSYIAPYT |
| 157 | CDR-L3 | |
| SEQ ID NO: | Ab55 (anti-CD117) | QVQLVQSGAEVKKPGSSVKVSCK |
| 158 | Heavy chain variable | ASGGTFRIYAISWVRQAPGQGLE |
| | region (e.g., as | WMGGIIPDFGVANYAQKFQGRVTI |
| | found in HC-55) | TADESTSTAYMELSSLRSEDTAVY |
| | (CDRs in bold) | YCARGGLDTDEFDLWGRGTLVTV |
| | | SS |
| SEQ ID NO: | Ab55 (anti-CD117) | DIQMTQSPSSLSASVGDRVTITCR |
| 159 | Light chain variable | ASQSINSYLNWYQQKPGKAPKLLI |
| | region (e.g., as | YAASSLQSGVPSRFSGSGSGTDF |
| | found in LC-55) | TLTISSLQPEDFATYYCQQGVSDIT |
| | (CDRs in bold) | FGGGTKVEIK |
| SEQ ID NO: | Ab55 (anti-CD117) | GTFRIYAIS |
| 160 | CDR-H1 | |
| SEQ ID NO: | Ab55 (anti-CD117) | GIIPDFGVANYAQKFQG |
| 161 | CDR-H2 | |
| SEQ ID NO: | Ab55 (anti-CD117) | ARGGLDTDEFDL |
| 162 | CDR-H3 | |
| SEQ ID NO: | Ab55 (anti-CD117) | RASQSINSYLN |
| 163 | CDR-L1 | |
| SEQ ID NO: | Ab55 (anti-CD117) | AASSLQS |
| 156 | CDR-L2 | |
| SEQ ID NO: | Ab55 (anti-CD117) | QQGVSDIT |
| 164 | CDR-L3 | |
| SEQ ID NO: | Ab54 (anti-CD117) | QVQLVQSGAEVKKPGSSVKVSCK |
| 165 | Heavy chain variable | ASGGTFSSYAISWVRQAPGQGLE |
| | region (e.g., as | WMGGIIPIFGTANYAQKFQGRVTI |
| | found in HC-54) | TADESTSTAYMELSSLRSEDTAVY |
| | hlgG1 backbone | YCARGGLDTDEFDLWGRGTLVTV |
| | (CDRs in bold) | SS |
| SEQ ID NO: | Ab54 (anti-CD117) | DIQMTQSPSSLSASVGDRVTITCR |
| 159 | Light chain variable | ASQSINSYLNWYQQKPGKAPKLLI |
| | region (e.g., as | YAASSLQSGVPSRFSGSGSGTDF |
| | found in LC-54) | TLTISSLQPEDFATYYCQQGVSDIT |
| | (CDRs in bold) | FGGGTKVEIK |
| SEQ ID NO: | Ab54 (anti-CD117) | GTFSSYAIS |
| 166 | CDR-H1 | |
| SEQ ID NO: | Ab54 (anti-CD117) | GIIPIFGTANYAQKFQG |
| 167 | CDR-H2 | |
| SEQ ID NO: | Ab54 (anti-CD117) | ARGGLDTDEFDL |
| 162 | CDR-H3 | |
| SEQ ID NO: | Ab54 (anti-CD117) | RASQSINSYLN |
| 163 | CDR-L1 | |
| SEQ ID NO: | Ab54 (anti-CD117) | AASSLQS |
| 156 | CDR-L2 | |
| SEQ ID NO: | Ab54 (anti-CD117) | QQGVSDIT |
| 164 | CDR-L3 | |
| SEQ ID NO: | Ab56 (anti-CD117) | QVQLVQSGAEVKKPGSSVKVSCK |
| 168 | Heavy chain variable | ASGGTFSLYAISWVRQAPGQGLE |
| | region | WMGGIIPAFGTANYAQKFQGRVTI |
| | (e.g., as found in | TADESTSTAYMELSSLRSEDTAVY |
| | HC-56) | YCARGGLDTDEFDLWGRGTLVTV |
| | (CDRs in bold) | SS |
| SEQ ID NO: | Ab56 (anti-CD117) | DIQMTQSPSSLSASVGDRVTITCR |
| 159 | Light chain variable | ASQSINSYLNWYQQKPGKAPKLLI |
| | region (e.g., as | YAASSLQSGVPSRFSGSGSGTDF |
| | found in LC-56) | TLTISSLQPEDFATYYCQQGVSDIT |
| | (CDRs in bold) | FGGGTKVEIK |
| SEQ ID NO: | Ab56 (anti-CD117) | GTFSLYAIS |
| 169 | CDR-H1 | |
| SEQ ID NO: | Ab56 (anti-CD117) | GIIPAFGTANYAQKFQG |
| 170 | CDR-H2 | |
| SEQ ID NO: | Ab56 (anti-CD117) | ARGGLDTDEFDL |
| 162 | CDR-H3 | |
| SEQ ID NO: | Ab56 (anti-CD117) | RASQSINSYLN |
| 163 | CDR-L1 | |
| SEQ ID NO: | Ab56 (anti-CD117) | AASSLQS |
| 156 | CDR-L2 | |
| SEQ ID NO: | Ab56 (anti-CD117) | QQGVSDIT |
| 164 | CDR-L3 | |
| SEQ ID NO: | Ab57 (anti-CD117) | QVQLVQSGAEVKKPGSSVKVSCK |
| 171 | Heavy chain variable | ASGGTFSLYAISWVRQAPGQGLE |
| | region (e.g., as | WMGGIIPHFGLANYAQKFQGRVTI |
| | found in HC-57) | TADESTSTAYMELSSLRSEDTAVY |
| | (CDRs in bold) | YCARGGLDTDEFDLWGRGTLVTV |
| | | SS |
| SEQ ID NO: | Ab57 (anti-CD117) | DIQMTQSPSSLSASVGDRVTITCR |
| 159 | Light chain variable | ASQSINSYLNWYQQKPGKAPKLLI |
| | region (e.g., as | YAASSLQSGVPSRFSGSGSGTDF |
| | found in LC-57) | TLTISSLQPEDFATYYCQQGVSDIT |
| | (CDRs in bold) | FGGGTKVEIK |
| SEQ ID NO: | Ab57 (anti-CD117) | GTFSLYAIS |
| 169 | CDR-H1 | |
| SEQ ID NO: | Ab57 (anti-CD117) | GIIPHFGLANYAQKFQG |
| 172 | CDR-H2 | |
| SEQ ID NO: | Ab57 (anti-CD117) | ARGGLDTDEFDL |
| 162 | CDR-H3 | |
| SEQ ID NO: | Ab57 (anti-CD117) | RASQSINSYLN |
| 163 | CDR-L1 | |
| SEQ ID NO: | Ab57 (anti-CD117) | AASSLQS |
| 156 | CDR-L2 | |
| SEQ ID NO: | Ab57 (anti-CD117) | QQGVSDIT |
| 164 | CDR-L3 | |
| SEQ ID NO: | Ab58 (anti-CD117) | EVQLLESGGGLVQPGGSLRLSCA |
| 173 | Heavy chain variable | ASGFTFSNYAMSWVRQAPGKGLE |
| | region (e.g., as | WVSAISGSGGSTYYADSVKGRFTI |
| | found in HC-58) | SRDNSKNTLYLQMNSLRAEDTAV |
| | (CDRs in bold) | YYCAKGPPTYHTNYYYMDVWGK |
| | | GTTVTVSS |
| SEQ ID NO: | Ab58 (anti-CD117) | DIQMTQSPSSVSASVGDRVTITCR |
| 174 | Light chain variable | ASQGISSWLAWYQQKPGKAPKLL |
| | region (e.g., as | IYAASSLQSGVPSRFSGSGSGTD |
| | found in LC-58) | FTLTISSLQPEDFATYYCQQTNSF |
| | (CDRs in bold) | PYTFGGGTKVEIK |
| SEQ ID NO: | Ab58 (anti-CD117) | FTFSNYAMS |
| 175 | CDR-H1 | |
| SEQ ID NO: | Ab58 (anti-CD117) | AISGSGGSTYYADSVKG |
| 176 | CDR-H2 | |
| SEQ ID NO: | Ab58 (anti-CD117) | AKGPPTYHTNYYYMDV |
| 177 | CDR-H3 | |
| SEQ ID NO: | Ab58 (anti-CD117) | RASQGISSWLA |
| 178 | CDR-L1 | |
| SEQ ID NO: | Ab58 (anti-CD117) | AASSLQS |
| 156 | CDR-L2 | |
| SEQ ID NO: | Ab58 (anti-CD117) | QQTNSFPYT |
| 179 | CDR-L3 | |
| SEQ ID NO: | Ab61 (anti-CD117) | EVQLLESGGGLVQPGGSLRLSCA |
| 180 | Heavy chain variable | ASGFTFSSYVMIWVRQAPGKGLE |
| | region (e.g., as | WVSSISGDSVTTYYADSVKGRFTI |
| | found in HC-61) | SRDNSKNTLYLQMNSLRAEDTAV |
| | (CDRs in bold) | YYCAKGPPTYHTNYYYMDVWGK |
| | | GTTVTVSS |
| SEQ ID NO: | Ab61 (anti-CD117) | DIQMTQSPSSVSASVGDRVTITCR |
| 174 | Light chain variable | ASQGISSWLAWYQQKPGKAPKLL |
| | region (e.g., as | IYAASSLQSGVPSRFSGSGSGTD |
| | found in LC-61) | FTLTISSLQPEDFATYYCQQTNSF |
| | (CDRs in bold) | PYTFGGGTKVEIK |
| SEQ ID NO: | Ab61 (anti-CD117) | FTFSSYVMI |
| 181 | CDR-H1 | |
| SEQ ID NO: | Ab61 (anti-CD117) | SISGDSVTTYYADSVKG |
| 182 | CDR-H2 | |
| SEQ ID NO: | Ab61 (anti-CD117) | AKGPPTYHTNYYYMDV |
| 177 | CDR-H3 | |
| SEQ ID NO: | Ab61 (anti-CD117) | RASQGISSWLA |
| 178 | CDR-L1 | |
| SEQ ID NO: | Ab61 (anti-CD117) | AASSLQS |
| 156 | CDR-L2 | |
| SEQ ID NO: | Ab61 (anti-CD117) | QQTNSFPYT |
| 179 | CDR-L3 | |
| SEQ ID NO: | Ab66 (anti-CD117) | EVQLVESGGGLVQPGGSLRLSCA |
| 183 | Heavy chain variable | ASGFTFSDHYMDWVRQAPGKGL |
| | region (e.g., as | EWVGRTRNKASSYTTEYAASVKG |
| | found in HC-66) | RFTISRDDSKNSLYLQMNSLKTED |
| | (CDRs in bold) | TAVYYCAREPKYWIDFDLWGRGT |
| | | LVTVSS |
| SEQ ID NO: | Ab66 Light chain | DIQMTQSPSSLSASVGDRVTITCR |
| 151 | variable region (e.g., | ASQSISSYLNWYQQKPGKAPKLLI |
| | as found in LC-66) | YAASSLQSGVPSRFSGSGSGTDF |
| | (CDRs in bold) | TLTISSLQPEDFATYYCQQSYIAPY |
| | | TFGGGTKVEIK |
| SEQ ID NO: | Ab66 (anti-CD117) | FTFSDHYMD |
| 184 | CDR-H1 | |
| SEQ ID NO: | Ab66 (anti-CD117) | RTRNKASSYTTEYAASVKG |
| 185 | CDR-H2 | |
| SEQ ID NO: | Ab66 (anti-CD117) | AREPKYWIDFDL |
| 154 | CDR-H3 | |
| SEQ ID NO: | Ab66 (anti-CD117) | RASQSISSYLN |
| 155 | CDR-L1 | |
| SEQ ID NO: | Ab66 (anti-CD117) | AASSLQS |
| 156 | CDR-L2 | |
| SEQ ID NO: | Ab66 (anti-CD117) | QQSYIAPYT |
| 157 | CDR-L3 | |
| SEQ ID NO: | Ab68 (anti-CD117) | EVQLVESGGGLVQPGRSLRLSCT |
| 186 | Heavy chain variable | ASGFTFSDHDMNWVRQAPGKGL |
| | region (e.g., as | EWVGRTRNAAGSYTTEYAASVK |
| | found in HC-68) | GRFTISRDDSKNSLYLQMNSLKTE |
| | (CDRs in bold) | DTAVYYCAREPKYWIDFDLWGRG |
| | | TLVTVSS |
| SEQ ID NO: | Ab68 (anti-CD117) | DIQMTQSPSSLSASVGDRVTITCR |
| 151 | Light chain variable | ASQSISSYLNWYQQKPGKAPKLLI |
| | region (e.g., as | YAASSLQSGVPSRFSGSGSGTDF |
| | found in LC-68) | TLTISSLQPEDFATYYCQQSYIAPY |
| | (CDRs in bold) | TFGGGTKVEIK |
| SEQ ID NO: | Ab68 (anti-CD117) | FTFSDHDMN |
| 187 | CDR-H1 | |
| SEQ ID NO: | Ab68 (anti-CD117) | RTRNAAGSYTTEYAASVKG |
| 188 | CDR-H2 | |
| SEQ ID NO: | Ab68 (anti-CD117) | AREPKYWIDFDL |
| 154 | CDR-H3 | |
| SEQ ID NO: | Ab68 (anti-CD117) | RASQSISSYLN |
| 155 | CDR-L1 | |
| SEQ ID NO: | Ab68 (anti-CD117) | AASSLQS |
| 156 | CDR-L2 | |
| SEQ ID NO: | Ab68 (anti-CD117) | QQSYIAPYT |
| 157 | CDR-L3 | |
| SEQ ID NO: | Ab69 (anti-CD117) | EVQLVESGGGLVQPGGSLRLSCA |
| 189 | Heavy chain variable | ASGFTFVDHDMDWVRQAPGKGL |
| | region (e.g., as | EWVGRTRNKLGSYTTEYAASVKG |
| | found in HC-69) | RFTISRDDSKNSLYLQMNSLKTED |
| | (CDRs in bold) | TAVYYCAREPKYWIDFDLWGRGT |
| | | LVTVSS |
| SEQ ID NO: | Ab69 (anti-CD117) | DIQMTQSPSSLSASVGDRVTITCR |
| 151 | Light chain variable | ASQSISSYLNWYQQKPGKAPKLLI |
| | region (e.g., as | YAASSLQSGVPSRFSGSGSGTDF |
| | found in LC-69) | TLTISSLQPEDFATYYCQQSYIAPY |
| | (CDRs in bold) | TFGGGTKVEIK |
| SEQ ID NO: | Ab69 (anti-CD117) | FTFVDHDMD |
| 190 | CDR-H1 | |
| SEQ ID NO: | Ab69 (anti-CD117) | RTRNKLGSYTTEYAASVKG |
| 191 | CDR-H2 | |
| SEQ ID NO: | Ab69 (anti-CD117) | AREPKYWIDFDL |
| 154 | CDR-H3 | |
| SEQ ID NO: | Ab69 (anti-CD117) | RASQSISSYLN |
| 155 | CDR-L1 | |
| SEQ ID NO: | Ab69 (anti-CD117) | AASSLQS |
| 156 | CDR-L2 | |
| SEQ ID NO: | Ab69 (anti-CD117) | QQSYIAPYT |
| 157 | CDR-L3 | |
| SEQ ID NO: | Consensus human | EVQLVESGGGLVQPGGSLRLSCA |
| 192 | Ab | ASGFTFSDYAMSWVRQAPGKGLE |
| | Heavy chain variable | WVAVISENGSDTYYADSVKGRFTI |
| | domain | SRDDSKNTLYLQMNSLRAEDTAV |
| | | YYCARDRGGAVSYFDVWGQGTL |
| | | VTVSS |
| SEQ ID NO: | Consensus human | DIQMTQSPSSLSASVGDRVTITCR |
| 193 | Ab | ASQDVSSYLAWYQQKPGKAPKLL |
| | Light chain variable | IYAASSLESGVPSRFSGSGSGTDF |
| | domain | TLTISSLQPEDFATYYCQQYNSLP |
| | | YTFGQGTKVEIKRT |
| SEQ ID NO: | Ab67 (anti-CD117) | DIQMTQSPSSLSASVGDRVTIT |
| 194 | Light chain | CRASQSISSYLNWYQQKPGKA |
| | LC constant region | PKLLIYAASSLQSGVPSRFSGS |
| | underlined | GSGTDFTLTISSLQPEDFATYY |
| | | CQQSYIAPYTFGGGTKVEIKRT |
| | | VAAPSVFIFPPSDEQLKSGTAS |
| | | VVCLLNNFYPREAKVQWKVDN |
| | | ALQSGNSQESVTEQDSKDSTY |
| | | SLSSTLTLSKADYEKHKVYACE |
| | | VTHQGLSSPVTKSFNRGEC |
| SEQ ID NO: | Ab67 (anti-CD117) | EVQLVESGGGLVQPGGSLRLS |
| 195 | Heavy chain | CAASGFTFSDADMDWVRQAP |
| | HC constant region | GKGLEWVGRTRNKAGSYTTEY |
| | underlined | AASVKGRFTISRDDSKNSLYLQ |
| | | MNSLKTEDTAVYYCAREPKYW |
| | | IDFDLWGRGTLVTVSSASTKGP |
| | | SVFPLAPSSKSTSGGTAALGCL |
| | | VKDYFPEPVTVSWNSGALTSG |
| | | VHTFPAVLQSSGLYSLSSVVTV |
| | | PSSSLGTQTYICNVNHKPSNTK |
| | | VDKKVEPKSCDKTHTCPPCPA |
| | | PELLGGPSVFLFPPKPKDTLMI |
| | | SRTPEVTCVVVDVSHEDPEVK |
| | | FNWYVDGVEVHNAKTKPREEQ |
| | | YNSTYRVVSVLTVLHQDWLNG |
| | | KEYKCKVSNKALPAPIEKTISKA |
| | | KGQPREPQVYTLPPSRDELTK |
| | | NQVSLTCLVKGFYPSDIAVEWE |
| | | SNGQPENNYKTTPPVLDSDGS |
| | | FFLYSKLTVDKSRWQQGNVFS |
| | | CSVMHEALHNHYTQKSLSLSP |
| | | GK |
| SEQ ID NO: | Ab67 (anti-CD117) | EVQLVESGGGLVQPGGSLRLS |
| 196 | Heavy chain | CAASGFTFSDADMDWVRQAP |
| | (D265C)* | GKGLEWVGRTRNKAGSYTTEY |
| | HC constant region | AASVKGRFTISRDDSKNSLYLQ |
| | underlined | MNSLKTEDTAVYYCAREPKYW |
| | | IDFDLWGRGTLVTVSSASTKGP |
| | | SVFPLAPSSKSTSGGTAALGCL |
| | | VKDYFPEPVTVSWNSGALTSG |
| | | VHTFPAVLQSSGLYSLSSVVTV |
| | | PSSSLGTQTYICNVNHKPSNTK |
| | | VDKKVEPKSCDKTHTCPPCPA |
| | | PELLGGPSVFLFPPKPKDTLMI |
| | | SRTPEVTCVVVCVSHEDPEVK |
| | | FNWYVDGVEVHNAKTKPREEQ |
| | | YNSTYRVVSVLTVLHQDWLNG |
| | | KEYKCKVSNKALPAPIEKTISKA |
| | | KGQPREPQVYTLPPSRDELTK |
| | | NQVSLTCLVKGFYPSDIAVEWE |
| | | SNGQPENNYKTTPPVLDSDGS |
| | | FFLYSKLTVDKSRWQQGNVFS |
| | | CSVMHEALHNHYTQKSLSLSP |
| | | GK |
| SEQ ID NO: | Ab67 (anti-CD117) | EVQLVESGGGLVQPGGSLRLS |
| 197 | Heavy chain (L234A / | CAASGFTFSDADMDWVRQAP |
| | L235A / D265C)* | GKGLEWVGRTRNKAGSYTTEY |
| | HC constant region | AASVKGRFTISRDDSKNSLYLQ |
| | underlined | MNSLKTEDTAVYYCAREPKYW |
| | | IDFDLWGRGTLVTVSSASTKGP |
| | | SVFPLAPSSKSTSGGTAALGCL |
| | | VKDYFPEPVTVSWNSGALTSG |
| | | VHTFPAVLQSSGLYSLSSVVTV |
| | | PSSSLGTQTYICNVNHKPSNTK |
| | | VDKKVEPKSCDKTHTCPPCPA |
| | | PEAAGGPSVFLFPPKPKDTLMI |
| | | SRTPEVTCVVVCVSHEDPEVK |
| | | FNWYVDGVEVHNAKTKPREEQ |
| | | YNSTYRVVSVLTVLHQDWLNG |
| | | KEYKCKVSNKALPAPIEKTISKA |
| | | KGQPREPQVYTLPPSRDELTK |
| | | NQVSLTCLVKGFYPSDIAVEWE |
| | | SNGQPENNYKTTPPVLDSDGS |
| | | FFLYSKLTVDKSRWQQGNVFS |
| | | CSVMHEALHNHYTQKSLSLSP |
| | | GK |
| SEQ ID NO: | Ab67 (anti-CD117) | EVQLVESGGGLVQPGGSLRLS |
| 198 | Heavy chain (D265C / | CAASGFTFSDADMDWVRQAP |
| | H435A)* | GKGLEWVGRTRNKAGSYTTEY |
| | HC constant region | AASVKGRFTISRDDSKNSLYLQ |
| | underlined | MNSLKTEDTAVYYCAREPKYW |
| | | IDFDLWGRGTLVTVSSASTKGP |
| | | SVFPLAPSSKSTSGGTAALGCL |
| | | VKDYFPEPVTVSWNSGALTSG |
| | | VHTFPAVLQSSGLYSLSSVVTV |
| | | PSSSLGTQTYICNVNHKPSNTK |
| | | VDKKVEPKSCDKTHTCPPCPA |
| | | PELLGGPSVFLFPPKPKDTLMI |
| | | SRTPEVTCVVVCVSHEDPEVK |
| | | FNWYVDGVEVHNAKTKPREEQ |
| | | YNSTYRVVSVLTVLHQDWLNG |
| | | KEYKCKVSNKALPAPIEKTISKA |
| | | KGQPREPQVYTLPPSRDELTK |
| | | NQVSLTCLVKGFYPSDIAVEWE |
| | | SNGQPENNYKTTPPVLDSDGS |
| | | FFLYSKLTVDKSRWQQGNVFS |
| | | CSVMHEALHNAYTQKSLSLSP |
| | | GK |
| SEQ ID NO: | Ab67 (anti-CD117) | EVQLVESGGGLVQPGGSLRLS |
| 199 | Heavy chain (L234A / | CAASGFTFSDADMDWVRQAP |
| | L235A / D265C / | GKGLEWVGRTRNKAGSYTTEY |
| | H435A)* | AASVKGRFTISRDDSKNSLYLQ |
| | HC constant region | MNSLKTEDTAVYYCAREPKYW |
| | underlined | IDFDLWGRGTLVTVSSASTKGP |
| | | SVFPLAPSSKSTSGGTAALGCL |
| | | VKDYFPEPVTVSWNSGALTSG |
| | | VHTFPAVLQSSGLYSLSSVVTV |
| | | PSSSLGTQTYICNVNHKPSNTK |
| | | VDKKVEPKSCDKTHTCPPCPA |
| | | PEAAGGPSVFLFPPKPKDTLMI |
| | | SRTPEVTCVVVCVSHEDPEVK |
| | | FNWYVDGVEVHNAKTKPREEQ |
| | | YNSTYRVVSVLTVLHQDWLNG |
| | | KEYKCKVSNKALPAPIEKTISKA |
| | | KGQPREPQVYTLPPSRDELTK |
| | | NQVSLTCLVKGFYPSDIAVEWE |
| | | SNGQPENNYKTTPPVLDSDGS |
| | | FFLYSKLTVDKSRWQQGNVFS |
| | | CSVMHEALHNAYTQKSLSLSP |
| | | GK |
| SEQ ID NO: | Ab55 (anti-CD117) | DIQMTQSPSSLSASVGDRVTITCR |
| 200 | Light chain | ASQSINSYLNWYQQKPGKAPKLLI |
| | LC constant region | YAASSLQSGVPSRFSGSGSGTDF |
| | underlined | TLTISSLQPEDFATYYCQQGVSDIT |
| | | FGGGTKVEIKRTVAAPSVFIFPP |
| | | SDEQLKSGTASVVCLLNNFYP |
| | | REAKVQWKVDNALQSGNSQE |
| | | SVTEQDSKDSTYSLSSTLTLSK |
| | | ADYEKHKVYACEVTHQGLSSP |
| | | VTKSFNRGEC |
| SEQ ID NO: | Ab55 (anti-CD117) | QVQLVQSGAEVKKPGSSVKVSCK |
| 201 | Heavy chain | ASGGTFRIYAISWVRQAPGQGLE |
| | HC constant region | WMGGIIPDFGVANYAQKFQGRVTI |
| | underlined | TADESTSTAYMELSSLRSEDTAVY |
| | | YCARGGLDTDEFDLWGRGTLVTV |
| | | SSASTKGPSVFPLAPSSKSTSG |
| | | GTAALGCLVKDYFPEPVTVSW |
| | | NSGALTSGVHTFPAVLQSSGL |
| | | YSLSSVVTVPSSSLGTQTYICN |
| | | VNHKPSNTKVDKKVEPKSCDK |
| | | THTCPPCPAPELLGGPSVFLFP |
| | | PKPKDTLMISRTPEVTCVVVDV |
| | | SHEDPEVKFNWYVDGVEVHNA |
| | | KTKPREEQYNSTYRVVSVLTVL |
| | | HQDWLNGKEYKCKVSNKALPA |
| | | PIEKTISKAKGQPREPQVYTLP |
| | | PSRDELTKNQVSLTCLVKGFYP |
| | | SDIAVEWESNGQPENNYKTTP |
| | | PVLDSDGSFFLYSKLTVDKSR |
| | | WQQGNVFSCSVMHEALHNHY |
| | | TQKSLSLSPGK |
| SEQ ID NO: | Ab55 (anti-CD117) | QVQLVQSGAEVKKPGSSVKVSCK |
| 202 | Heavy chain | ASGGTFRIYAISWVRQAPGQGLE |
| | (D265C)* | WMGGIIPDFGVANYAQKFQGRVTI |
| | HC constant region | TADESTSTAYMELSSLRSEDTAVY |
| | underlined | YCARGGLDTDEFDLWGRGTLVTV |
| | | SSASTKGPSVFPLAPSSKSTSG |
| | | GTAALGCLVKDYFPEPVTVSW |
| | | NSGALTSGVHTFPAVLQSSGL |
| | | YSLSSVVTVPSSSLGTQTYICN |
| | | VNHKPSNTKVDKKVEPKSCDK |
| | | THTCPPCPAPELLGGPSVFLFP |
| | | PKPKDTLMISRTPEVTCVVVCV |
| | | SHEDPEVKFNWYVDGVEVHNA |
| | | KTKPREEQYNSTYRVVSVLTVL |
| | | HQDWLNGKEYKCKVSNKALPA |
| | | PIEKTISKAKGQPREPQVYTLP |
| | | PSRDELTKNQVSLTCLVKGFYP |
| | | SDIAVEWESNGQPENNYKTTP |
| | | PVLDSDGSFFLYSKLTVDKSR |
| | | WQQGNVFSCSVMHEALHNHY |
| | | TQKSLSLSPGK |
| SEQ ID NO: | Ab55 (anti-CD117) | QVQLVQSGAEVKKPGSSVKVSCK |
| 203 | Heavy chain (L234A / | ASGGTFRIYAISWVRQAPGQGLE |
| | L235A / D265C)* | WMGGIIPDFGVANYAQKFQGRVTI |
| | HC constant region | TADESTSTAYMELSSLRSEDTAVY |
| | underlined | YCARGGLDTDEFDLWGRGTLVTV |
| | | SSASTKGPSVFPLAPSSKSTSG |
| | | GTAALGCLVKDYFPEPVTVSW |
| | | NSGALTSGVHTFPAVLQSSGL |
| | | YSLSSVVTVPSSSLGTQTYICN |
| | | VNHKPSNTKVDKKVEPKSCDK |
| | | THTCPPCPAPEAAGGPSVFLF |
| | | PPKPKDTLMISRTPEVTCVVVC |
| | | VSHEDPEVKFNWYVDGVEVHN |
| | | AKTKPREEQYNSTYRVVSVLT |
| | | VLHQDWLNGKEYKCKVSNKAL |
| | | PAPIEKTISKAKGQPREPQVYT |
| | | LPPSRDELTKNQVSLTCLVKGF |
| | | YPSDIAVEWESNGQPENNYKT |
| | | TPPVLDSDGSFFLYSKLTVDKS |
| | | RWQQGNVFSCSVMHEALHNH |
| | | YTQKSLSLSPGK |
| SEQ ID NO: | Ab55 (anti-CD117) | QVQLVQSGAEVKKPGSSVKVSCK |
| 204 | Heavy chain (D265C / | ASGGTFRIYAISWVRQAPGQGLE |
| | H435A)* | WMGGIIPDFGVANYAQKFQGRVTI |
| | HC constant region | TADESTSTAYMELSSLRSEDTAVY |
| | underlined | YCARGGLDTDEFDLWGRGTLVTV |
| | | SSASTKGPSVFPLAPSSKSTSG |
| | | GTAALGCLVKDYFPEPVTVSW |
| | | NSGALTSGVHTFPAVLQSSGL |
| | | YSLSSVVTVPSSSLGTQTYICN |
| | | VNHKPSNTKVDKKVEPKSCDK |
| | | THTCPPCPAPELLGGPSVFLFP |
| | | PKPKDTLMISRTPEVTCVVVCV |
| | | SHEDPEVKFNWYVDGVEVHNA |
| | | KTKPREEQYNSTYRVVSVLTVL |
| | | HQDWLNGKEYKCKVSNKALPA |
| | | PIEKTISKAKGQPREPQVYTLP |
| | | PSRDELTKNQVSLTCLVKGFYP |
| | | SDIAVEWESNGQPENNYKTTP |
| | | PVLDSDGSFFLYSKLTVDKSR |
| | | WQQGNVFSCSVMHEALHNAY |
| | | TQKSLSLSPGK |
| SEQ ID NO: | Ab55 (anti-CD117) | QVQLVQSGAEVKKPGSSVKVSCK |
| 205 | Heavy chain (L234A / | ASGGTFRIYAISWVRQAPGQGLE |
| | L235A / D265C / | WMGGIIPDFGVANYAQKFQGRVTI |
| | H435A)* | TADESTSTAYMELSSLRSEDTAVY |
| | HC constant region | YCARGGLDTDEFDLWGRGTLVTV |
| | underlined | SSASTKGPSVFPLAPSSKSTSG |
| | | GTAALGCLVKDYFPEPVTVSW |
| | | NSGALTSGVHTFPAVLQSSGL |
| | | YSLSSVVTVPSSSLGTQTYICN |
| | | VNHKPSNTKVDKKVEPKSCDK |
| | | THTCPPCPAPEAAGGPSVFLF |
| | | PPKPKDTLMISRTPEVTCVVVC |
| | | VSHEDPEVKFNWYVDGVEVHN |
| | | AKTKPREEQYNSTYRVVSVLT |
| | | VLHQDWLNGKEYKCKVSNKAL |
| | | PAPIEKTISKAKGQPREPQVYT |
| | | LPPSRDELTKNQVSLTCLVKGF |
| | | YPSDIAVEWESNGQPENNYKT |
| | | TPPVLDSDGSFFLYSKLTVDKS |
| | | RWQQGNVFSCSVMHEALHNA |
| | | YTQKSLSLSPGK |
| SEQ ID NO: | Light chain constant | RTVAAPSVFIFPPSDEQLKSGT |
| 149 | region of LC-54, | ASVVCLLNNFYPREAKVQWKV |
| | LC-55, LC-56, LC- | DNALQSGNSQESVTEQDSKDS |
| | 57, LC-58, LC-61, | TYSLSSTLTLSKADYEKHKVYA |
| | LC-66, LC-67, LC- | CEVTHQGLSSPVTKSFNRGEC |
| | 68, LC-69 | |
| SEQ ID NO: | Consensus | GTF(S/R)(S/I/L)YAIS |
| 206 | sequence of variable | |
| | heavy chain CDR1 | |
| | (Abs 54-57) | |
| SEQ ID NO: | Consensus | GIIP(I/D/A/H)FG(T/V/L)ANYAQKFQ |
| 207 | sequence of variable | G |
| | heavy chain CDR2 | |
| | (Abs 54-57) | |
| SEQ ID NO: | Variable heavy chain | ARGGLDTDEFDL |
| 162 | CDR3 (Abs 54-57) | |
| SEQ ID NO: | Variable light chain | RASQSINSYLN |
| 163 | CDR1 (Abs 54-57) | |
| SEQ ID NO: | Variable light chain | AASSLQS |
| 156 | CDR2 (Abs 54-57) | |
| SEQ ID NO: | Variable light chain | QQGVSDIT |
| 164 | CDR3 (Abs 54-57) | |
| SEQ ID NO: | Consensus | FTFS(N/S)Y(A/V)M(S/I) |
| 208 | sequence of variable | |
| | heavy chain CDR1 | |
| | (Abs 58, 61) | |
| SEQ ID NO: | Consensus | (A/S)ISG(S/D)(G/S)(G/V)(S/T)TYYA |
| 209 | sequence of variable | DSVKG |
| | heavy chain CDR2 | |
| | (Abs 58, 61) | |
| SEQ ID NO: | Variable heavy chain | AKGPPTYHTNYYYMDV |
| 177 | CDR3 (Abs 58, 61) | |
| SEQ ID NO: | Variable light chain | RASQGISSWLA |
| 178 | CDR1 (Abs 58, 61) | |
| SEQ ID NO: | Variable light chain | AASSLQS |
| 156 | CDR2 (Abs 58, 61) | |
| SEQ ID NO: | Variable light chain | QQTNSFPYT |
| 179 | CDR3 (Abs 58, 61) | |
| SEQ ID NO: | Consensus | FTF(S/V)D(H/A)(Y/D)M(D/N) |
| 210 | sequence of variable | |
| | heavy chain CDR1 | |
| | (Abs 66-69) | |
| SEQ ID NO: | Consensus | RTRN(K/A)(A/L)(S/G)SYTTEYAAS |
| 211 | sequence of variable | VKG |
| | heavy chain CDR2 | |
| | (Abs 66-69) | |
| SEQ ID NO: | Variable heavy chain | AREPKYWIDFDL |
| 154 | CDR3 (Abs 66-69) | |
| SEQ ID NO: | Variable light chain | RASQSISSYLN |
| 155 | CDR1 (Abs 66-69) | |
| SEQ ID NO: | Variable light chain | AASSLQS |
| 156 | CDR2 (Abs 66-69) | |
| SEQ ID NO: | Variable light chain | QQSYIAPYT |
| 157 | CDR3 (Abs 66-69) | |
| SEQ ID NO: | Human CD117 | MRGARGAWDFLCVLLLLLRVQ |
| 212 | (mast/stem cell | TGSSQPSVSPGEPSPPSIHPG |
| | growth factor | KSDLIVRVGDEIRLLCTDPGFV |
| | receptor Kit isoform | KWTFEILDETNENKQNEWITEK |
| | 1 precursor) | AEATNTGKYTCTNKHGLSNSIY |
| | Protein NCBI | VFVRDPAKLFLVDRSLYGKED |
| | Reference | NDTLVRCPLTDPEVTNYSLKG |
| | Sequence: | CQGKPLPKDLRFIPDPKAGIMI |
| | NP_000213.1 | KSVKRAYHRLCLHCSVDQEGK |
| | | SVLSEKFILKVRPAFKAVPVVS |
| | | VSKASYLLREGEEFTVTCTIKD |
| | | VSSSVYSTWKRENSQTKLQEK |
| | | YNSWHHGDFNYERQATLTISS |
| | | ARVNDSGVFMCYANNTFGSAN |
| | | VTTTLEVVDKGFINIFPMINTTV |
| | | FVNDGENVDLIVEYEAFPKPEH |
| | | QQWIYMNRTFTDKWEDYPKSE |
| | | NESNIRYVSELHLTRLKGTEGG |
| | | TYTFLVSNSDVNAAIAFNVYVN |
| | | TKPEILTYDRLVNGMLQCVAAG |
| | | FPEPTIDWYFCPGTEQRCSAS |
| | | VLPVDVQTLNSSGPPFGKLVV |
| | | QSSIDSSAFKHNGTVECKAYN |
| | | DVGKTSAYFNFAFKGNNKEQI |
| | | HPHTLFTPLLIGFVIVAGMMCII |
| | | VMILTYKYLQKPMYEVQWKVV |
| | | EEINGNNYVYIDPTQLPYDHKW |
| | | EFPRNRLSFGKTLGAGAFGKV |
| | | VEATAYGLIKSDAAMTVAVKML |
| | | KPSAHLTEREALMSELKVLSYL |
| | | GNHMNIVNLLGACTIGGPTLVI |
| | | TEYCCYGDLLNFLRRKRDSFIC |
| | | SKQEDHAEAALYKNLLHSKES |
| | | SCSDSTNEYMDMKPGVSYVVP |
| | | TKADKRRSVRIGSYIERDVTPAI |
| | | MEDDELALDLEDLLSFSYQVAK |
| | | GMAFLASKNCIHRDLAARNILL |
| | | THGRITKICDFGLARDIKNDSN |
| | | YVVKGNARLPVKWMAPESIFN |
| | | CVYTFESDVWSYGIFLWELFSL |
| | | GSSPYPGMPVDSKFYKMIKEG |
| | | FRMLSPEHAPAEMYDIMKTCW |
| | | DADPLKRPTFKQIVQLIEKQISE |
| | | STNHIYSNLANCSPNRQKPVV |
| | | DHSVRINSVGSTASSSQPLLVH |
| | | DDV |
| SEQ ID NO: | Human CD117 | MRGARGAWDFLCVLLLLLRVQ |
| 213 | (mast/stem cell | TGSSQPSVSPGEPSPPSIHPG |
| | growth factor | KSDLIVRVGDEIRLLCTDPGFV |
| | receptor Kit isoform | KWTFEILDETNENKQNEWITEK |
| | 2 precursor) | AEATNTGKYTCTNKHGLSNSIY |
| | Protein NCBI | VFVRDPAKLFLVDRSLYGKED |
| | Reference | NDTLVRCPLTDPEVTNYSLKG |
| | Sequence: | CQGKPLPKDLRFIPDPKAGIMI |
| | NP_001087241.1 | KSVKRAYHRLCLHCSVDQEGK |
| | | SVLSEKFILKVRPAFKAVPVVS |
| | | VSKASYLLREGEEFTVTCTIKD |
| | | VSSSVYSTWKRENSQTKLQEK |
| | | YNSWHHGDFNYERQATLTISS |
| | | ARVNDSGVFMCYANNTFGSAN |
| | | VTTTLEVVDKGFINIFPMINTTV |
| | | FVNDGENVDLIVEYEAFPKPEH |
| | | QQWIYMNRTFTDKWEDYPKSE |
| | | NESNIRYVSELHLTRLKGTEGG |
| | | TYTFLVSNSDVNAAIAFNVYVN |
| | | TKPEILTYDRLVNGMLQCVAAG |
| | | FPEPTIDWYFCPGTEQRCSAS |
| | | VLPVDVQTLNSSGPPFGKLVV |
| | | QSSIDSSAFKHNGTVECKAYN |
| | | DVGKTSAYFNFAFKEQIHPHTL |
| | | FTPLLIGFVIVAGMMCIIVMILTY |
| | | KYLQKPMYEVQWKVVEEINGN |
| | | NYVYIDPTQLPYDHKWEFPRN |
| | | RLSFGKTLGAGAFGKVVEATA |
| | | YGLIKSDAAMTVAVKMLKPSAH |
| | | LTEREALMSELKVLSYLGNHM |
| | | NIVNLLGACTIGGPTLVITEYCC |
| | | YGDLLNFLRRKRDSFICSKQED |
| | | HAEAALYKNLLHSKESSCSDST |
| | | NEYMDMKPGVSYVVPTKADKR |
| | | RSVRIGSYIERDVTPAIMEDDE |
| | | LALDLEDLLSFSYQVAKGMAFL |
| | | ASKNCIHRDLAARNILLTHGRIT |
| | | KICDFGLARDIKNDSNYVVKGN |
| | | ARLPVKWMAPESIFNCVYTFES |
| | | DVWSYGIFLWELFSLGSSPYP |
| | | GMPVDSKFYKMIKEGFRMLSP |
| | | EHAPAEMYDIMKTCWDADPLK |
| | | RPTFKQIVQLIEKQISESTNHIY |
| | | SNLANCSPNRQKPVVDHSVRI |
| | | NSVGSTASSSQPLLVHDDV |
| SEQ ID NO: | ch-BBK2 heavy | QVQLQQPGAELVRPGASVKLS |
| 214 | chain (anti-CD137) | CKASGYTFTSYWINWVKQRPG |
| | | QGLEWIGNIYPSDSYTNYNQKF |
| | | KDKATLTVDKSSNTVYMQLNS |
| | | PTSEDSAVYYCTRNGVEGYPH |
| | | YYAMEYWGQGTSVTVSSASTK |
| | | GPSVFPLAPSSKSTSGGTAAL |
| | | GCLVKDYFPEPVTVSWNSGAL |
| | | TSGVHTFPAVLQSSGLYSLSSV |
| | | VTVPSSSLGTQTYICNVNHKPS |
| | | NTKVDKKVEPKSCDKTHTCPP |
| | | CPAPELLGGPSVFLFPPKPKDT |
| | | LMISRTPEVTCVVVDVSHEDPE |
| | | VKFNWYVDGVEVHNAKTKPRE |
| | | EQYNSTYRVVSVLTVLHQDWL |
| | | NGKEYKCKVSNKALPAPIEKTI |
| | | SKAKGQPREPQVYTLPPSRDE |
| | | LTKNQVSLTCLVKGFYPSDIAV |
| | | EWESNGQPENNYKTTPPVLDS |
| | | DGSFFLYSKLTVDKSRWQQGN |
| | | VFSCSVMHEALHNHYTQKSLS |
| | | LSPGK |
| SEQ ID NO: | ch-BBK2 light chain | DIQMTQTTSALSASLGDRVTIG |
| 215 | (anti-CD137) | CRASQDLSNHLYWYQQKPDG |
| | | TVKLLIYYTSRLHSGVPSRFSG |
| | | SGSGTDYSLTIRNLEQEDVATY |
| | | FCQQGYTLPYTFGGGTKLEIKR |
| | | TVAAPSVFIFPPSDEQLKSGTA |
| | | SVVCLLNNFYPREAKVQWKVD |
| | | NALQSGNSQESVTEQDSKDST |
| | | YSLSSTLTLSKADYEKHKVYAC |
| | | EVTHQGLSSPVTKSFNRGEC |
| SEQ ID NO: | BBK2 light chain | QVQLQQPGAELVRPGASVKLS |
| 216 | variable region (anti- | CKASGYTFTSYWINWVKQRPG |
| | CD137) | QGLEWIGNIYPSDSYTNYNQKF |
| | | KDKATLTVDKSSNTVYMQLNS |
| | | PTSEDSAVYYCTRNGVEGYPH |
| | | YYAMEYWGQGTSVTVSS |
| SEQ ID NO: | BBK2 light chain | DIQMTQTTSALSASLGDRVTIG |
| 217 | variable region (anti- | CRASQDLSNHLYWYQQKPDG |
| | CD137) | TVKLLIYYTSRLHSGVPSRFSG |
| | | SGSGTDYSLTIRNLEQEDVATY |
| | | FCQQGYTLPYTFGGGTKLEIK |
| SEQ ID NO: | BBK2 (anti-CD137) | SYWIN |
| 218 | CDR-H1 | |
| SEQ ID NO: | BBK2 (anti-CD137) | NIYPSDSYTNYNQKFKD |
| 219 | CDR-H2 | |
| SEQ ID NO: | BBK2 (anti-CD137) | NGVEGYPHYYAMEY |
| 220 | CDR-H3 | |
| SEQ ID NO: | BBK2 (anti-CD137) | RASQDLSNHLY |
| 221 | CDR-L1 | |
| SEQ ID NO: | BBK2 (anti-CD137) | YTSRLHS |
| 222 | CDR-L2 | |
| SEQ ID NO: | BBK2 (anti-CD137) | QQGYTLPYT |
| 223 | CDR-L3 | |
| SEQ ID NO: | 5D7v (anti-CD5) | QVTLKESGPVLVKPTETLTLTC |
| 224 | heavy chain variable | TFSGFSLSTSGMGVGWIRQAP |
| | region | GKGLEWVAHIWWDDDVYYNP |
| | | SLKSRLTITKDASKDQVSLKLS |
| | | SVTAADTAVYYCVRRRATGTG |
| | | FDYWGQGTLVTVSS |
| SEQ ID NO: | 5D7v (anti-CD5) | NIVMTQSPSSLSASVGDRVTIT |
| 225 | light chain variable | CQASQDVGTAVAWYQQKPDQ |
| | region | SPKLLIYWTSTRHTGVPDRFTG |
| | | SGSGTDFTLTISSLQPEDIATYF |
| | | CHQYNSYNTFGSGTKLEIK |
| SEQ ID NO: | 5D7v (anti-CD5) | FSLSTSGMG |
| 226 | CDR-H1 | |
| SEQ ID NO: | 5D7v (anti-CD5) | WWDDD |
| 227 | CDR-H2 | |
| SEQ ID NO: | 5D7v (anti-CD5) | RRATGTGFDY |
| 228 | CDR-H3 | |
| SEQ ID NO: | 5D7v (anti-CD5) | QDVGTA |
| 229 | CDR-L1 | |
| SEQ ID NO: | 5D7v (anti-CD5) | WTSTRHT |
| 230 | CDR-L2 | |
| SEQ ID NO: | 5D7v (anti-CD5) | YNSYNT |
| 231 | CDR-L3 | |
| SEQ ID NO: | Human CD117 | MRGARGAWDFLCVLLLLLRVQTG |
| 232 | amino acid | SSQPSVSPGEPSPPSIHPGKSDLI |
| | sequence | VRVGDEIRLLCTDPGFVKWTFEIL |
| | | DETNENKQNEWITEKAEATNTGK |
| | | YTCTNKHGLSNSIYVFVRDPAKLF |
| | | LVDRSLYGKEDNDTLVRCPLTDPE |
| | | VTNYSLKGCQGKPLPKDLRFIPDP |
| | | KAGIMIKSVKRAYHRLCLHCSVDQ |
| | | EGKSVLSEKFILKVRPAFKAVPVVS |
| | | VSKASYLLREGEEFTVTCTIKDVSS |
| | | SVYSTWKRENSQTKLQEKYNSWH |
| | | HGDFNYERQATLTISSARVNDSGV |
| | | FMCYANNTFGSANVTTTLEVVDK |
| | | GFINIFPMINTTVFVNDGENVDLIVE |
| | | YEAFPKPEHQQWIYMNRTFTDKW |
| | | EDYPKSENESNIRYVSELHLTRLK |
| | | GTEGGTYTFLVSNSDVNAAIAFNV |
| | | YVNTKPEILTYDRLVNGMLQCVAA |
| | | GFPEPTIDWYFCPGTEQRCSASVL |
| | | PVDVQTLNSSGPPFGKLVVQSSID |
| | | SSAFKHNGTVECKAYNDVGKTSA |
| | | YFNFAFKGNNKEQIHPHTLFTPLLI |
| | | GFVIVAGMMCIIVMILTYKYLQKPM |
| | | YEVQWKVVEEINGNNYVYIDPTQL |
| | | PYDHKWEFPRNRLSFGKTLGAGA |
| | | FGKVVEATAYGLIKSDAAMTVAVK |
| | | MLKPSAHLTEREALMSELKVLSYL |
| | | GNHMNIVNLLGACTIGGPTLVITEY |
| | | CCYGDLLNFLRRKRDSFICSKQED |
| | | HAEAALYKNLLHSKESSCSDSTNE |
| | | YMDMKPGVSYVVPTKADKRRSVR |
| | | IGSYIERDVTPAIMEDDELALDLED |
| | | LLSFSYQVAKGMAFLASKNCIHRD |
| | | LAARNILLTHGRITKICDFGLARDIK |
| | | NDSNYVVKGNARLPVKWMAPESI |
| | | FNCVYTFESDVWSYGIFLWELFSL |
| | | GSSPYPGMPVDSKFYKMIKEGFR |
| | | MLSPEHAPAEMYDIMKTCWDADP |
| | | LKRPTFKQIVQLIEKQISESTNHIYS |
| | | NLANCSPNRQKPVVDHSVRINSV |
| | | GSTASSSQPL LVHDDV |
| SEQ ID NO: | Extracellular domain | MGNSCYNIVATLLLVLNFERTRSL |
| 233 | of Human CD137 | QDPCSNCPAGTFCDNNRNQICSP |
| | amino acid | CPPNSFSSAGGQRTCDICRQCKG |
| | sequence | VFRTRKECSSTSNAECDCTPGFH |
| | | CLGAGCSMCEQDCKQGQELTKK |
| | | GCKDCCFGTFNDQKRGICRPWTN |
| | | CSLDGKSVLVNGTKERDVVCGPS |
| | | PADLSPGASSVTPPAPAREPGHS |
| | | PQIISFFLALTSTALLFLLFFLTLRFS |
| | | VVKRGRKKLLYIFKQPFMRPVQTT |
| | | QEEDGCSCRFPEEEEGGCEL |
| SEQ ID NO: | Human CD5 amino | MVCSQSWGRSSKQWEDPSQASK |
| 234 | acid sequence | VCQRLNCGVPLSLGPFLVTYTPQS |
| | | SIICYGQLGSFSNCSHSRNDMCHS |
| | | LGLTCLEPQKTTPPTTRPPPTTTP |
| | | EPTAPPRLQLVAQSGGQHCAGVV |
| | | EFYSGSLGGTISYEAQDKTQDLEN |
| | | FLCNNLQCGSFLKHLPETEAGRA |
| | | QDPGEPREHQPLPIQWKIQNSSC |
| | | TSLEHCFRKIKPQKSGRVLALLCS |
| | | GFQPKVQSRLVGGSSICEGTVEV |
| | | RQGAQWAALCDSSSARSSLRWE |
| | | EVCREQQCGSVNSYRVLDAGDPT |
| | | SRGLFCPHQKLSQCHELWERNSY |
| | | CKKVFVTCQDPNPAGLAAGTVASI |
| | | ILALVLLVVLLVVCGPLAYKKLVKK |
| | | FRQKKQRQWIGPTGMNQNMSFH |
| | | RNHTATVRSHAENPTASHVDNEY |
| | | SQPPRNSHLSAYPALEGALHRSS |
| | | MQPDNSSDSDYDLHGAQRL |
| SEQ ID NO: | Human CD2 amino | MSFPCKFVASFLLIFNVSSKGAVS |
| 235 | acid sequence | KEITNALETWGALGQDINLDIPSFQ |
| | | MSDDIDDIKWEKTSDKKKIAQ |
| | | FRKEKETFKE |
| | | KDTYKLFKNGTLKIKHLKTDDQDIY |
| | | KVSIYDTKGKNVLEKIFDLKIQERV |
| | | SKPKISWTCINTTLTCEVMNGTDP |
| | | ELNLYQDGKHLKLSQRVITHKWTT |
| | | SLSAKFKCTAGNKVSKESSVEPVS |
| | | CPEKGLDIYLIIGICGGGSLLMVFV |
| | | ALLVFYITKRKKQRSRRNDEELET |
| | | RAHRVATEERGRKPHQIPASTPQ |
| | | NPATSQHPPPPPGHRSQAPSHRP |
| | | PPPGHRVQHQPQKRPPAPSGTQ |
| | | VHQQKGPPLPRPRVQPKPPHGAA |
| | | ENSLSPSS N |
| SEQ ID NO: | CD45RO (Human | MTMYLWLKLLAFGFAFLDTEVFVT |
| 236 | CD45 Isoform) | GQSPTPSPTDAYLNASETTTLSPS |
| | | GSAVISTTTIATTPSKPTCDEKYANI |
| | | TVDYLYNKETKLFTAKLNVNENVE |
| | | CGNNTCTNNEVHNLTECKNASVSI |
| | | SHNSCTAPDKTLILDVPPGVEKFQ |
| | | LHDCTQVEKADTTICLKWKNIETFT |
| | | CDTQNITYRFQCGNMIFDNKEIKLE |
| | | NLEPEHEYKCDSEILYNNHKFTNA |
| | | SKIIKTDFGSPGEPQIIFCRSEAAH |
| | | QGVITWNPPQRSFHNFTLCYIKET |
| | | EKDCLNLDKNLIKYDLQNLKPYTK |
| | | YVLSLHAYIIAKVQRNGSAAMCHF |
| | | TTKSAPPSQVWNMTVSMTSDNS |
| | | MHVKCRPPRDRNGPHERYHLEVE |
| | | AGNTLVRNESHKNCDFRVKDLQY |
| | | STDYTFKAYFHNGDYPGEPFILHH |
| | | STSYNSKALIAFLAFLIIVTSIALLVV |
| | | LYKIYDLHKKRSCNLDEQQELVER |
| | | DDEKQLMNVEPIHADILLETYKRKI |
| | | ADEGRLFLAEFQSIPRVFSKFPIKE |
| | | ARKPFNQNKNRYVDILPYDYNRVE |
| | | LSEINGDAGSNYINASYIDGFKEPR |
| | | KYIAAQGPRDETVDDFWRMIWEQ |
| | | KATVIVMVTRCEEGNRNKCAEYW |
| | | PSMEEGTRAFGDVVVKINQHKRC |
| | | PDYIIQKLNIVNKKEKATGREVTHI |
| | | QFTSWPDHGVPEDPHLLLKLRRR |
| | | VNAFSNFFSGPIVVHCSAGVGRTG |
| | | TYIGIDAMLEGLEAENKVDVYGYV |
| | | VKLRRQRCLMVQVEAQYILIHQAL |
| | | VEYNQFGETEVNLSELHPYLHNM |
| | | KKRDPPSEPSPLEAEFQRLPSYRS |
| | | WRTQHIGNQEENKSKNRNSNVIP |
| | | YDYNRVPLKHELEMSKESEHDSD |
| | | ESSDDDSDSEEPSKYINASFIMSY |
| | | WKPEVMIAAQGPLKETIGDFWQMI |
| | | FQRKVKVIVMLTELKHGDQEICAQ |
| | | YWGEGKQTYGDIEVDLKDTDKSS |
| | | TYTLRVFELRHSKRKDSRTVYQYQ |
| | | YTNWSVEQLPAEPKELISMIQVVK |
| | | QKLPQKNSSEGNKHHKSTPLLIHC |
| | | RDGSQQTGIFCALLNLLESAETEE |
| | | VVDIFQVVKALRKARPGMVSTFEQ |
| | | YQFLYDVIASTYPAQNGQVKKNNH |
| | | QEDKIEFDNEVDKVKQDANCVNPL |
| | | GAPEKLPEAKEQAEGSEPTSGTE |
| | | GPEHSVNGPASPALNQGS |
| SEQ ID NO: | CD45RA (Human | MTMYLWLKLLAFGFAFLDTEVFVT |
| 237 | CD45 Isoform) | GQSPTPSPTGLTTAKMPSVPLSSD |
| | | PLPTHTTAFSPASTFERENDFSET |
| | | TTSLSPDNTSTQVSPDSLDNASAF |
| | | NTTDAYLNASETTTLSPSGSAVIST |
| | | TTIATTPSKPTCDEKYANITVDYLY |
| | | NKETKLFTAKLNVNENVECGNNTC |
| | | TNNEVHNLTECKNASVSISHNSCT |
| | | APDKTLILDVPPGVEKFQLHDCTQ |
| | | VEKADTTICLKWKNIETFTCDTQNI |
| | | TYRFQCGNMIFDNKEIKLENLEPE |
| | | HEYKCDSEILYNNHKFTNASKIIKT |
| | | DFGSPGEPQIIFCRSEAAHQGVIT |
| | | WNPPQRSFHNFTLCYIKETEKDCL |
| | | NLDKNLIKYDLQNLKPYTKYVLSLH |
| | | AYIIAKVQRNGSAAMCHFTTKSAP |
| | | PSQVWNMTVSMTSDNSMHVKCR |
| | | PPRDRNGPHERYHLEVEAGNTLV |
| | | RNESHKNCDFRVKDLQYSTDYTF |
| | | KAYFHNGDYPGEPFILHHSTSYNS |
| | | KALIAFLAFLIIVTSIALLVVLYKIYDL |
| | | HKKRSCNLDEQQELVERDDEKQL |
| | | MNVEPIHADILLETYKRKIADEGRL |
| | | FLAEFQSIPRVFSKFPIKEARKPFN |
| | | QNKNRYVDILPYDYNRVELSEING |
| | | DAGSNYINASYIDGFKEPRKYIAAQ |
| | | GPRDETVDDFWRMIWEQKATVIV |
| | | MVTRCEEGNRNKCAEYWPSMEE |
| | | GTRAFGDVVVKINQHKRCPDYIIQ |
| | | KLNIVNKKEKATGREVTHIQFTSW |
| | | PDHGVPEDPHLLLKLRRRVNAFSN |
| | | FFSGPIVVHCSAGVGRTGTYIGIDA |
| | | MLEGLEAENKVDVYGYVVKLRRQ |
| | | RCLMVQVEAQYILIHQALVEYNQF |
| | | GETEVNLSELHPYLHNMKKRDPP |
| | | SEPSPLEAEFQRLPSYRSWRTQHI |
| | | GNQEENKSKNRNSNVIPYDYNRV |
| | | PLKHELEMSKESEHDSDESSDDD |
| | | SDSEEPSKYINASFIMSYWKPEVM |
| | | IAAQGPLKETIGDFWQMIFQRKVK |
| | | VIVMLTELKHGDQEICAQYWGEGK |
| | | QTYGDIEVDLKDTDKSSTYTLRVF |
| | | ELRHSKRKDSRTVYQYQYTNWSV |
| | | EQLPAEPKELISMIQVVKQKLPQK |
| | | NSSEGNKHHKSTPLLIHCRDGSQ |
| | | QTGIFCALLNLLESAETEEVVDIFQ |
| | | VVKALRKARPGMVSTFEQYQFLY |
| | | DVIASTYPAQNGQVKKNNHQEDKI |
| | | EFDNEVDKVKQDANCVNPLGAPE |
| | | KLPEAKEQAEGSEPTSGTEGPEH |
| | | SVNGPASPALNQGS |
| SEQ ID NO: | CD45RB (Human | MTMYLWLKLLAFGFAFLDTEVFVT |
| 238 | CD45 Isoform) | GQSPTPSPTGVSSVQTPHLPTHA |
| | | DSQTPSAGTDTQTFSGSAANAKL |
| | | NPTPGSNAISDAYLNASETTTLSP |
| | | SGSAVISTTTIATTPSKPTCDEKYA |
| | | NITVDYLYNKETKLFTAKLNVNENV |
| | | ECGNNTCTNNEVHNLTECKNASV |
| | | SISHNSCTAPDKTLILDVPPGVEKF |
| | | QLHDCTQVEKADTTICLKWKNIET |
| | | FTCDTQNITYRFQCGNMIFDNKEIK |
| | | LENLEPEHEYKCDSEILYNNHKFT |
| | | NASKIIKTDFGSPGEPQIIFCRSEA |
| | | AHQGVITWNPPQRSFHNFTLCYIK |
| | | ETEKDCLNLDKNLIKYDLQNLKPYT |
| | | KYVLSLHAYIIAKVQRNGSAAMCH |
| | | FTTKSAPPSQVWNMTVSMTSDNS |
| | | MHVKCRPPRDRNGPHERYHLEVE |
| | | AGNTLVRNESHKNCDFRVKDLQY |
| | | STDYTFKAYFHNGDYPGEPFILHH |
| | | STSYNSKALIAFLAFLIIVTSIALLVV |
| | | LYKIYDLHKKRSCNLDEQQELVER |
| | | DDEKQLMNVEPIHADILLETYKRKI |
| | | ADEGRLFLAEFQSIPRVFSKFPIKE |
| | | ARKPFNQNKNRYVDILPYDYNRVE |
| | | LSEINGDAGSNYINASYIDGFKEPR |
| | | KYIAAQGPRDETVDDFWRMIWEQ |
| | | KATVIVMVTRCEEGNRNKCAEYW |
| | | PSMEEGTRAFGDVVVKINQHKRC |
| | | PDYIIQKLNIVNKKEKATGREVTHI |
| | | QFTSWPDHGVPEDPHLLLKLRRR |
| | | VNAFSNFFSGPIVVHCSAGVGRTG |
| | | TYIGIDAMLEGLEAENKVDVYGYV |
| | | VKLRRQRCLMVQVEAQYILIHQAL |
| | | VEYNQFGETEVNLSELHPYLHNM |
| | | KKRDPPSEPSPLEAEFQRLPSYRS |
| | | WRTQHIGNQEENKSKNRNSNVIP |
| | | YDYNRVPLKHELEMSKESEHDSD |
| | | ESSDDDSDSEEPSKYINASFIMSY |
| | | WKPEVMIAAQGPLKETIGDFWQMI |
| | | FQRKVKVIVMLTELKHGDQEICAQ |
| | | YWGEGKQTYGDIEVDLKDTDKSS |
| | | TYTLRVFELRHSKRKDSRTVYQYQ |
| | | YTNWSVEQLPAEPKELISMIQVVK |
| | | QKLPQKNSSEGNKHHKSTPLLIHC |
| | | RDGSQQTGIFCALLNLLESAETEE |
| | | VVDIFQVVKALRKARPGMVSTFEQ |
| | | YQFLYDVIASTYPAQNGQVKKNNH |
| | | QEDKIEFDNEVDKVKQDANCVNPL |
| | | GAPEKLPEAKEQAEGSEPTSGTE |
| | | GPEHSVNGPASPALNQGS |
| SEQ ID NO: | CD45RC (Human | MTMYLWLKLLAFGFAFLDTEVFVT |
| 239 | CD45 Isoform) | GQSPTPSPTDVPGERSTASTFPT |
| | | DPVSPLTTTLSLAHHSSAALPART |
| | | SNTTITANTSDAYLNASETTTLSPS |
| | | GSAVISTTTIATTPSKPTCDEKYANI |
| | | TVDYLYNKETKLFTAKLNVNENVE |
| | | CGNNTCTNNEVHNLTECKNASVSI |
| | | SHNSCTAPDKTLILDVPPGVEKFQ |
| | | LHDCTQVEKADTTICLKWKNIETFT |
| | | CDTQNITYRFQCGNMIFDNKEIKLE |
| | | NLEPEHEYKCDSEILYNNHKFTNA |
| | | SKIIKTDFGSPGEPQIIFCRSEAAH |
| | | QGVITWNPPQRSFHNFTLCYIKET |
| | | EKDCLNLDKNLIKYDLQNLKPYTK |
| | | YVLSLHAYIIAKVQRNGSAAMCHF |
| | | TTKSAPPSQVWNMTVSMTSDNS |
| | | MHVKCRPPRDRNGPHERYHLEVE |
| | | AGNTLVRNESHKNCDFRVKDLQY |
| | | STDYTFKAYFHNGDYPGEPFILHH |
| | | STSYNSKALIAFLAFLIIVTSIALLVV |
| | | LYKIYDLHKKRSCNLDEQQELVER |
| | | DDEKQLMNVEPIHADILLETYKRKI |
| | | ADEGRLFLAEFQSIPRVFSKFPIKE |
| | | ARKPFNQNKNRYVDILPYDYNRVE |
| | | LSEINGDAGSNYINASYIDGFKEPR |
| | | KYIAAQGPRDETVDDFWRMIWEQ |
| | | KATVIVMVTRCEEGNRNKCAEYW |
| | | PSMEEGTRAFGDVVVKINQHKRC |
| | | PDYIIQKLNIVNKKEKATGREVTHI |
| | | QFTSWPDHGVPEDPHLLLKLRRR |
| | | VNAFSNFFSGPIVVHCSAGVGRTG |
| | | TYIGIDAMLEGLEAENKVDVYGYV |
| | | VKLRRQRCLMVQVEAQYILIHQAL |
| | | VEYNQFGETEVNLSELHPYLHNM |
| | | KKRDPPSEPSPLEAEFQRLPSYRS |
| | | WRTQHIGNQEENKSKNRNSNVIP |
| | | YDYNRVPLKHELEMSKESEHDSD |
| | | ESSDDDSDSEEPSKYINASFIMSY |
| | | WKPEVMIAAQGPLKETIGDFWQMI |
| | | FQRKVKVIVMLTELKHGDQEICAQ |
| | | YWGEGKQTYGDIEVDLKDTDKSS |
| | | TYTLRVFELRHSKRKDSRTVYQYQ |
| | | YTNWSVEQLPAEPKELISMIQVVK |
| | | QKLPQKNSSEGNKHHKSTPLLIHC |
| | | RDGSQQTGIFCALLNLLESAETEE |
| | | VVDIFQVVKALRKARPGMVSTFEQ |
| | | YQFLYDVIASTYPAQNGQVKKNNH |
| | | QEDKIEFDNEVDKVKQDANCVNPL |
| | | GAPEKLPEAKEQAEGSEPTSGTE |
| | | GPEHSVNGPASPALNQGS |
| SEQ ID NO: | Heavy chain variable | QVQLVQSGAEVKKPGASVKVSCK |
| 240 | region of anti-CD2 | ASGYTFTEYYMYWVRQAPGQGLE |
| | AbA | LMGRIDPEDGSIDYVEKFKKKVTLT |
| | | ADTSSSTAYMELSSLTSDDTAVYY |
| | | CARGKFNYRFAYWGQGTLVTVSS |
| SEQ ID NO: | Light chain variable | DVVMTQSPPSLLVTLGQPASISCR |
| 241 | region of anti-CD2 | SSQSLLHSSGNTYLNWLLQRPGQ |
| | AbA | SPQPLIYLVSKLESGVPDRFSGSG |
| | | SGTDFTLKISGVEAEDVGVYYCMQ |
| | | FTHYPYTFGQGTKLE IK |
| SEQ ID NO: | Heavy chain variable | QVQLVQSGAEVQRPGASVKVSCK |
| 242 | region of anti-CD2 | ASGYIFTEYYMYWVRQAPGQGLE |
| | AbB | LVGRIDPEDGSIDYVEKFKKKVTLT |
| | | ADTSSSTAYMELSSLTSDDTAVYY |
| | | CARGKFNYRFAYWGQGTLVTVSS |
| SEQ ID NO: | Light chain variable | DVVMTQSPPSLLVTLGQPASISCR |
| 243 | region of anti-CD2 | SSQSLLHSSGNTYLNWLLQRPGQ |
| | AbB | SPQPLIYLVSKLESGVPDRFSGSG |
| | | SGTDFTLKISGVEAEDVGVYYCMQ |
| | | FTHYPYTFGQGTKLEIK |
| SEQ ID NO: | Heavy chain variable | EVKLVESGGGLVKPGGSLKLSCA |
| 244 | region of anti-CD2 | ASGFTFSSYDMSWVRQTPEKRLE |
| | AbC | WVASISGGGFLYYLDSVKGRFTIS |
| | | RDNARNILYLHMTSLRSEDTAMYY |
| | | CARSSYGEIMDYWGQGTSVTVSS |
| SEQ ID NO: | Light chain variable | DILLTQSPAILSVSPGERVSFSCRA |
| 245 | region of anti-CD2 | SQRIGTSIHWYQQRTTGSPRLLIK |
| | AbC | YASESISGIPSRFSGSGSGTDFTLS |
| | | INSVESEDVADYYCQQSHGWPFT |
| | | FGGGTKLEIE |
| SEQ ID NO: | Heavy chain variable | EVKLVESGGGLVKPGGSLKLSCA |
| 246 | region of anti-CD2 | ASGFTFSSYDMSWVRQTPEKRLE |
| | AbD | WVASISGGGFLYYLDSVKGRFTIS |
| | | RDNARNILYLHMTSLRSEDTAMYY |
| | | CARSSYGELMDYWGQGTSVTVS |
| | | S |
| SEQ ID NO: | Light chain variable | DILLTQSPAILSVSPGERVSFSCRA |
| 247 | region of anti-CD2 | SQRIGTSIHWYQQRTTGSPRLLIK |
| | AbD | YASESISGIPSRFSGSGSGTDFTLS |
| | | INSVESEDVADYYCQQSHGWPFT |
| | | FGGGTKLEIE |
| SEQ ID NO: | Apamistamab Heavy | EVKLLESGGGLVQPGGSLKLSCA |
| 248 | Chain | ASGFDFSRYWMSWVRQAPGKGL |
| | | EWIGEINPTSSTINFTPSLKDKVFIS |
| | | RDNAKNTLYLQMSKVRSEDTALY |
| | | YCARGNYYRYGDAMDYWGQGTS |
| | | VTVSSAKTTPPSVYPLAPGSAAQT |
| | | NSMVTLGCLVKGYFPEPVTVTWN |
| | | SGSLSSGVHTFPAVLQSDLYTLSS |
| | | SVTVPSSTWPSETVTCNVAHPAS |
| | | STKVDKKIVPRDCGCKPCICTVPE |
| | | VSSVFIFPPKPKDVLTITLTPKVTCV |
| | | VVDISKDDPEVQFSWFVDDVEVH |
| | | TAQTQPREEQFNSTERSVSELPIM |
| | | HQDWLNGKEFKCRVNSAAFPAPI |
| | | EKTISKTKGRPKAPQVYTIPPPKEQ |
| | | MAKDKVSLTCMITDFFPEDITVEW |
| | | QWNGQPAENYKNTQPIMDTDGSY |
| | | FVYSKLNVQKSNWEAGNTFTCSV |
| | | LHEGLHNHHTEKSLSHSPGK |
| SEQ ID NO: | Apamistamab Light | DIALTQSPASLAVSLGQRATISCRA |
| 249 | Chain | SKSVSTSGYSYLHWYQQKPGQPP |
| | | KLLIYLASNLESGVPARFSGSGSG |
| | | TDFTLNIHPVEEEDAATYYCQHSR |
| | | ELPFTFGSGTKLEIKRADAAPTVSI |
| | | FPPSSEQLTSGGASVVCFLNNEYP |
| | | KDINVKWKIDGSERQNGVLNSWT |
| | | DQDSKDSTYSMSSTLTLTKDEYER |
| | | HNSYTCEATHKTSTSPIVKSFNRN |
| | | EC |
| SEQ ID NO: | Apamistamab Heavy | EVKLLESGGGLVQPGGSLKLSCA |
| 250 | Chain Variable | ASGFDFSRYWMSWVRQAPGKGL |
| | Region | EWIGEINPTSSTINFTPSLKDKVFIS |
| | | RDNAKNTLYLQMSKVRSEDTALY |
| | | YCARGNYYRYGDAMDYWGQGTS |
| | | VTVSSA |
| SEQ ID NO: | Apamistamab Light | DIALTQSPASLAVSLGQRATISCRA |
| 251 | Chain Variable | SKSVSTSGYSYLHWYQQKPGQPP |
| | Region | KLLIYLASNLESGVPARFSGSGSG |
| | | TDFTLNIHPVEEEDAATYYCQHSR |
| | | ELPFTFGSGTKLEIKR |
| SEQ ID NO: | ch-BBK2 VH CDR1 | SGYTFTSYW |
| 252 | (as in Lee et al.) | |
| SEQ ID NO: | ch-BBK2 VH CDR2 | NIYPSDSYT |
| 253 | (as in Lee et al.) | |
| SEQ ID NO: | ch-BBK2 VH CDR3 | TRNGVEGYPHYYAME |
| 254 | (as in Lee et al.) | |
| SEQ ID NO: | ch-BBK2 VL CDR1 | SQDLSNH |
| 255 | (as in Lee et al.) | |
| SEQ ID NO: | ch-BBK2 VL CDR2 | YYTS |
| 256 | (as in Lee et al.) | |
| SEQ ID NO: | ch-BBK2 VL CDR3 | CQQGYTLPY |
| 257 | (as in Lee) | |
| SEQ ID NO: | Heavy chain | ASTKGPSVFPLAPSSKSTSGG |
| 258 | constant region: | TAALGCLVKDYFPEPVTVSWN |
| | modified Fc region | SGALTSGVHTFPAVLQSSGLY |
| | with H435A / D265C | SLSSVVTVPSSSLGTQTYICNV |
| | mutations (mutations | NHKPSNTKVDKKVEPKSCDKT |
| | in bold) | HTCPPCPAPELLGGPSVFLFPP |
| | | KPKDTLMISRTPEVTCVVVCVS |
| | | HEDPEVKFNWYVDGVEVHNAK |
| | | TKPREEQYNSTYRVVSVLTVLH |
| | | QDWLNGKEYKCKVSNKALPAP |
| | | IEKTISKAKGQPREPQVYTLPP |
| | | SRDELTKNQVSLTCLVKGFYPS |
| | | DIAVEWESNGQPENNYKTTPP |
| | | VLDSDGSFFLYSKLTVDKSRW |
| | | QQGNVFSCSVMHEALHNAYT |
| | | QKSLSLSPGK |

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A method of depleting a population of target cells in a human patient, wherein the method comprises a dosing regimen comprising the steps of:
(a) administering to the patient a first dose of an antibody drug conjugate (ADC) on day 1; and
(b) administering to the patient a second dose of the ADC after the first dose;
wherein the ADC comprises an antibody, or antigen-binding fragment thereof, conjugated to an amatoxin via a linker,
wherein the ADC is specific for a cell surface marker, and
wherein the dosing regimen induces a decrease in the population of the target cells in a human patient.

2. The method of claim 1, wherein the first dose is selected from the group consisting of about 2-12 mg of the ADC, about 2-11 mg of the ADC, about 2-10 mg of the ADC, about 2-9 mg of the ADC, about 2-8 mg of the ADC, about 2-7 mg of the ADC, about 2-6 mg of the ADC, about 2-5 mg of the ADC, about 2-4 mg of the ADC, about 1.9-11.5 mg of the ADC, and about 2-3 mg of the ADC.

3. (canceled)

4. The method of claim 1, wherein the second dose is within 10% by weight of the first dose or wherein the second dose is the same as the first dose and/or is administered between 1-12 days after the first dose is administered or between 1-24 hours after the first dose is administered.

5.-6. (canceled)

7. The method of claim 1, wherein the target cell is selected from the group consisting of a stem cell, an immune cell, or a disease causing cell.

8.-9. (canceled)

10. The method of claim 1, wherein the cell surface marker is selected from the group consisting of CD117, CD45, CD2, CD5, CD252, and CD137.

11. The method of claim 1, wherein the antibody, or antigen-binding fragment thereof, comprises a heavy chain variable region having an amino acid sequence as set forth in SEQ ID NO: 106 and a light chain variable region having an amino acid sequence as set forth in SEQ ID NO: 107; or
wherein the antibody, or antigen-binding fragment thereof, comprises a heavy chain variable region having an amino acid sequence as set forth in SEQ ID NO: 150 and a light chain variable region having an amino acid sequence as set forth in SEQ ID NO: 151.

12. (canceled)

13. The method of claim 1,
wherein the ADC comprises an antibody, or antigen-binding fragment thereof, conjugated to an amatoxin via a linker.

14.-24. (canceled)

25. A method of depleting a population of target cells in a human patient for transplant conditioning, wherein the method comprises a dosing regimen comprising the steps of:
(a) administering to the patient a first dose of an antibody drug conjugate (ADC) on day 1; and
(b) administering to the patient a second dose of the ADC after the first dose;
wherein the ADC comprises an antibody, or antigen-binding fragment thereof, conjugated to a cytotoxin via a linker,
wherein the ADC is specific for a cell surface marker selected from CD117, CD45, CD137, CD2, CD252, or CD5, and
wherein the dosing regimen induces a decrease in the population of the target cells in a human patient for transplant conditioning.

26. The method of claim 25, wherein the cytotoxin is an antimitotic agent or an RNA polymerase inhibitor.

27. The method of claim 26, wherein the RNA polymerase inhibitor is an amatoxin.

28.-29. (canceled)

30. The method of claim 1, wherein the amatoxin is represented by formula (I) 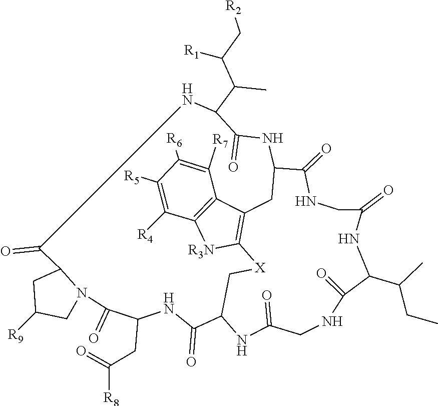
wherein R₁ is H, OH, OR_{A}, or OR_{C};
R₂ is H, OH, OR_{B}, or OR_{C};
R_{A} and R_{B}, together with the oxygen atoms to which they are bound, combine to form an optionally substituted 5-membered heterocyclolalkyl group;
R₃ is H, R_{C}, or R_{D};
R₄, R₅, R₆, and R₇ are each independently H, OH, OR_{C}, OR_{D}, R_{C}, or R_{D};
R₈ is OH, NH₂, OR_{C}, OR_{D}, NHR_{C}, or NR_{C}R_{D};
R₉ is H, OH, OR_{C}, or OR_{D};
X is —S—, —S(O)—, or —SO₂—;
R_{C} is -L-Z;
R_{D} is substituted C₁-C₆ alkyl, substituted C₁-C₆ heteroalkyl, substituted C₂-C₆ alkenyl, substituted C₂-C₆ heteroalkenyl, substituted C₂-C₆ alkynyl, substituted C₂-C₆ heteroalkynyl, substituted cycloalkyl, substituted heterocycloalkyl, substituted aryl, or substituted heteroaryl;
L is a peptide containing linker; and
Z is a chemical moiety formed from a coupling reaction between a reactive substituent present on L and a reactive substituent present within the antibody or antigen-binding fragment thereof.

31. The method of claim 25, wherein the cytotoxin is a PBD, a maytansine or an auristatin.

32.-33. (canceled)

34. The method of claim 25, wherein the antibody binds CD117 and
a) comprises a heavy chain variable region having an amino acid sequence as set forth in SEQ ID NO: 106 and a light chain variable region having an amino acid sequence as set forth in SEQ ID NO: 107; or
b) comprises a heavy chain variable region having an amino acid sequence as set forth in SEQ ID NO: 150 and a light chain variable region having an amino acid sequence as set forth in SEQ ID NO: 151.

35.-37. (canceled)

38. The method claim 25, wherein the second dose is administered about 1 hour, about 2 hours, about 3 hours, about 4 hours, about 5 hours, about 6 hours, about 7 hours, about 8 hours, about 1 day, about 2 days, or about 3 days, after the first dose is administered.

39.-48. (canceled)

49. The method of claim 25, wherein the patient further receives a stem cell transplant when the ADC is substantially cleared from the blood of the patient.

50. (canceled)

51. The method of claim 25, further comprising administering a cell transplantation to the human patient.

52. The method of claim 51, wherein the cell transplant is a hematopoietic stem cell (HSC) transplantation.

53. The method of claim 25, wherein the method is non-myeloablative.

54. The method of claim 25, wherein a liver marker determined from the patient does not reach a toxic level.

55. (canceled)

56. The method of claim 25, wherein
a liver marker determined from the patient is elevated for no more than 5 to 7 days following administration of the first dose, or
a liver marker determined from the patient is elevated for no more than 6 days following administration of the first dose.

57.-59. (canceled)

60. A method of depleting a population of target cells in a human patient, wherein the method comprises a dosing regimen comprising:
administering to the patient a second dose of an antibody drug conjugate (ADC) within 1 to 14 days after the patient was administered a first dose of the ADC;
wherein the ADC comprises an antibody, or antigen-binding fragment thereof, conjugated to cytotoxin via a linker;
wherein the ADC is specific for a cell surface marker;
wherein the dosing regimen induces a decrease in the population of the target cells in a human patient; and
wherein at least one of the patient's blood AST, ALT, or LDH levels does not reach a toxic level between administration of the first dose and 14 days after administration of the first dose to the patient or wherein at least one of the patient's blood AST, ALT, or LDH levels does not rise more than 3-fold above normal levels between administration of the first dose and 14 days after administration of the first dose to the patient.

61. (canceled)

62. The method of claim 60, wherein the cytotoxin is an antimitotic agent or an RNA polymerase inhibitor.

63. The method of claim 62, wherein the RNA polymerase inhibitor is an amatoxin.

64. (canceled)

65. The method of claim 60, wherein the cytotoxin is a PBD, a maytansine or an auristatin.

66. (canceled)

67. The method of claim 60, wherein the cell surface marker is CD117, CD45, CD137, CD2, CD252, or CD5.

68. The method of claim 67, wherein the antibody binds CD117 and
a) comprises a heavy chain variable region having an amino acid sequence as set forth in SEQ ID NO: 106 and a light chain variable region having an amino acid sequence as set forth in SEQ ID NO: 107; or
b) comprises a heavy chain variable region having an amino acid sequence as set forth in SEQ ID NO: 150 and a light chain variable region having an amino acid sequence as set forth in SEQ ID NO: 151.

69.-72. (canceled)

73. The method of claim 60, wherein the patient further receives a stem cell transplant when the ADC is substantially cleared from the blood of the patient.

74. The method of claim 73, wherein the cell transplant is a hematopoietic stem cell (HSC) transplantation.


SEQUENCE LISTING (258 sequences)

SEQ ID NO: 1 - protein, 5 aa, synthetic construct
  REGION 1..5: note = Description of Artificial Sequence: Synthetic peptide
SYWIG

SEQ ID NO: 2 - protein, 17 aa, synthetic construct
  REGION 1..17: note = Description of Artificial Sequence: Synthetic peptide
IIYPGDSDTRYSPSFQG

SEQ ID NO: 3 - protein, 14 aa, synthetic construct
  REGION 1..14: note = Description of Artificial Sequence: Synthetic peptide
HGRGYNGYEGAFDI

SEQ ID NO: 4 - protein, 11 aa, synthetic construct
  REGION 1..11: note = Description of Artificial Sequence: Synthetic peptide
RASQGISSALA

SEQ ID NO: 5 - protein, 7 aa, synthetic construct
  REGION 1..7: note = Description of Artificial Sequence: Synthetic peptide
DASSLES

SEQ ID NO: 6 - protein, 10 aa, synthetic construct
  REGION 1..10: note = Description of Artificial Sequence: Synthetic peptide
CQQFNSYPLT

SEQ ID NO: 7 - protein, 123 aa, synthetic construct
  REGION 1..123: note = Description of Artificial Sequence: Synthetic polypeptide
QVQLVQSGAAVKKPGESLKISCKGSGYRFTSYWIGWVRQMPGKGLEWMGIIYPGDSDTRYSPSFQGQVTISAGKSISTAYLQWSSLKASDTAMYYCARHGRGYNGYEGAFDIWGQGTMVTVSS

SEQ ID NO: 8 - protein, 107 aa, synthetic construct [sequence omitted]

SEQ ID NO: 9 - protein, 123 aa, synthetic construct
  REGION 1..123: note = Description of Artificial Sequence: Synthetic polypeptide
QVQLVQSGAAVKKPGESLKISCKGSGYRFTTYWIGWVRQMPGKGLEWMGIIYPGDSDTRYSPSFQGQVTISAGKSISTAYLQWSSLKASDTAMYYCARHGRGYNGYEGAFDIWGQGTMVTVSS

SEQ ID NO: 10 - protein, 107 aa, synthetic construct
  REGION 1..107: note = Description of Artificial Sequence: Synthetic polypeptide
AIQLTQSPSSLSASVGDRVTITCRASQGVSSALAWYQQKPGKAPKLLIYDASSLESGVPSRFSGSGSGTDFTLTISSLQPEDFATYYCQQFNSYPLTFGGGTKVEIK

SEQ ID NO: 11 - protein, 107 aa, synthetic construct
  REGION 1..107: note = Description of Artificial Sequence: Synthetic polypeptide
DIQLTQSPSSLSASVGDRVTITCRASQGIRTDLGWYQQKPGKAPKLLIYDASSLESGVPSRFSGSGSGTDFTLTISSLQPEDFATYYCQQFNSYPLTFGGGTKVEIK

SEQ ID NO: 12 - protein, 107 aa, synthetic construct
  REGION 1..107: note = Description of Artificial Sequence: Synthetic polypeptide
AIRMTQSPSSLSASVGDRVTITCRASQGIRNDLAWYQQKPGKTPKLLIYDASSLESGVPSRFSGSGSGTDFTLTISSLQPEDFATYYCQQFNSYPLTFGGGTKVEIK

SEQ ID NO: 13 - protein, 107 aa, synthetic construct
  REGION 1..107: note = Description of Artificial Sequence: Synthetic polypeptide
AIQMTQSPSSLSASVGDRVTITCRASQGIRNDLGWYQQKPGKAPKLLIYDASSLESGVPSRFSGSGSGTDFTLTISSLQPEDFATYYCQQFNSYPLTFGGGTKVDIK

SEQ ID NO: 14 - protein, 107 aa, synthetic construct
  REGION 1..107: note = Description of Artificial Sequence: Synthetic polypeptide
NIQMTQSPSSLSASVGDRVTITCRASQAISDYLAWFQQKPGKAPKLLIYDASNLETGVPSRFSGSGSGTDFTLTISSLQPEDFATYYCQQLNSYPLTFGGGTKVEIK

SEQ ID NO: 15 - protein, 107 aa, synthetic construct
  REGION 1..107: note = Description of Artificial Sequence: Synthetic polypeptide
AIRMTQSPSSLSASVGDRVIIACRASQGIGGALAWYQQKPGNAPKVLVYDASTLESGVPSRFSGGGSGTDFTLTISSLQPEDFATYYCQQFNSYPLTFGGGTKLEIK

SEQ ID NO: 16 - protein, 107 aa, synthetic construct
  REGION 1..107: note = Description of Artificial Sequence: Synthetic polypeptide
DIAMTQSPPSLSAFVGDRVTITCRASQGIISSLAWYQQKPGKAPKLLIYDASSLESGVPSRFSGSGSGTDFTLTIRSLQPEDFATYYCQQFNSYPLTFGGGTKLEIK

SEQ ID NO: 17 - protein, 107 aa, synthetic construct
  REGION 1..107: note = Description of Artificial Sequence: Synthetic polypeptide
DIQMTQSPSSLSASVGDRVTITCRASQGISSALAWYQQKAGKAPKVLISDASSLESGVPSRFSGSGSGTDFTLSISSLQPEDFATYYCQQFNGYPLTFGGGTKVDIK

SEQ ID NO: 18 - protein, 107 aa, synthetic construct
  REGION 1..107: note = Description of Artificial Sequence: Synthetic polypeptide
AIRMTQSPSSLSASVGDRVTITCQASQGIRNDLGWYQQKPGKAPKLLIYDASNLETGVPSRFSGSGSGTDFTFTISSLQPEDIATYYCQQFNSYPLTFGGGTKLEIK

SEQ ID NO: 19 - protein, 107 aa, synthetic construct
  REGION 1..107: note = Description of Artificial Sequence: Synthetic polypeptide
NIQMTQSPSSLSTSVGDRVTITCRASQGIGTSLAWYQQKPGKPPKLLIYDASSLESGVPSRLSGSGSGTDFTLTISSLQPEDFATYYCQQSNSYPITFGQGTRLEIK

SEQ ID NO: 20 - protein, 107 aa, synthetic construct
  REGION 1..107: note = Description of Artificial Sequence: Synthetic polypeptide
AIQLTQSPSSLSASVGDRVTITCRASQSIGDYLTWYQQKPGKAPKVLIYGASSLQSGVPPRFSGSGSGTDFTLTVSSLQPEDFATYYCQQLNSYPLTFGGGTKLEIK

SEQ ID NO: 21 - protein, 107 aa, synthetic construct
  REGION 1..107: note = Description of Artificial Sequence: Synthetic polypeptide
DIQLTQSPSSLSASVGDRVTITCRASQGVRSTLAWYQQKPGKAPKLLIYDASILESGVPSRFSGSGSGTDFTLTISSLQPEDFATYYCQQFNGYPLTFGQGTRLEIK

SEQ ID NO: 22 - protein, 107 aa, synthetic construct
  REGION 1..107: note = Description of Artificial Sequence: Synthetic polypeptide
DIVMTQSPSSLSASVGDRVTITCRASQGIRNDLGWYQQKPGKAPKLLIYDASSLESGVPSRFSGSGSGTDFTLTISSLQPEDFATYYCQQFNSYPLTFGGGTKLEIK

SEQ ID NO: 23 - protein, 107 aa, synthetic construct
  REGION 1..107: note = Description of Artificial Sequence: Synthetic polypeptide
DIQLTQSPSSLSASVGDRVTITCRASQGISSFLAWYQQKPGKAPKLLIYDASTLQSGVPSRFSGSASGTDFTLTISSLQPEDFATYYCQQLNGYPLTFGGGTKVEIK

SEQ ID NO: 24 - protein, 107 aa, synthetic construct
  REGION 1..107: note = Description of Artificial Sequence: Synthetic polypeptide
AIQLTQSPSSLSASVGDRVTITCRASQGIGSALAWYQQKPGIGPKLLIYDASTLESGVPARFSGSGSRTDFTLTITSLQPEDFATYYCQQFNGYPLTFGGGTKLEIK

SEQ ID NO: 25 - protein, 107 aa, synthetic construct
  REGION 1..107: note = Description of Artificial Sequence: Synthetic polypeptide
AIQLTQSPSSLSASVGDRVTITCRASQGITSALAWYQEKPGKAPNLLIYDASSLESGVPSRFSGSGYGTDFTLTISSLQPEDFATYYCQQLNSYPLTFGGGTKVDIK

SEQ ID NO: 26 - protein, 121 aa, synthetic construct
  REGION 1..121: note = Description of Artificial Sequence: Synthetic polypeptide
QIQLVQSGPELRKPGESVKISCKASGYTFTDYAMYWVKQAPGKGLKWMGWINTYTGKPTYADDFKGRFVFSLEASANTANLQISNLKNEDTATYFCARARGLVDDYVMDAWGQGTSVTVSS

SEQ ID NO: 27 - protein, 108 aa, synthetic construct
  REGION 1..108: note = Description of Artificial Sequence: Synthetic polypeptide
SYELIQPPSASVTLGNTVSLTCVGDELSKRYAQWYQQKPDKTIVSVIYKDSERPSGISDRFSGSSSGTTATLTIHGTLAEDEADYYCLSTYSDDNLPVFGGGTKLTVL

SEQ ID NO: 28 - protein, 117 aa, synthetic construct
  REGION 1..117: note = Description of Artificial Sequence: Synthetic polypeptide
EVQLQQYGAELGKPGTSVRLSCKVSGYNIRNTYIHWVNQRPGEGLEWIGRIDPTNGNTISAEKFKTKATLTADTSSHTAYLQFSQLKSDDTAIYFCALNYEGYADYWGQGVMVTGSS

SEQ ID NO: 29 - protein, 106 aa, synthetic construct
  REGION 1..106: note = Description of Artificial Sequence: Synthetic polypeptide
DIQMTQSPSFLSASVGDRVTINCKASQNINKYLNWYQQKVGEAPKRLIFKTNSLQTGIPSRFSGSGSGTDYTLTISSLQTEDVATYFCFQYNIGYTFGAGTKVELK

SEQ ID NO: 30 - protein, 124 aa, synthetic construct
  REGION 1..124: note = Description of Artificial Sequence: Synthetic polypeptide
EVQLQESGPGLVKPSQSLSLTCSVTGYSISSNYRWNWIRKFPGNKVEWMGYINSAGSTNYNPSLKSRISMTRDTSKNQFFLQVNSVTTEDTATYYCARSLRGYITDYSGFFDYWGQGVMVTVSS

SEQ ID NO: 31 - protein, 107 aa, synthetic construct
  REGION 1..107: note = Description of Artificial Sequence: Synthetic polypeptide
DIRMTQSPASLSASLGETVNIECLASEDIFSDLAWYQQKPGKSPQLLIYNANSLQNGVPSRFSGSGSGTRYSLKINSLQSEDVATYFCQQYKNYPLTFGSGTKLEIK

SEQ ID NO: 32 - protein, 117 aa, synthetic construct
  REGION 1..117: note = Description of Artificial Sequence: Synthetic polypeptide
EVQLQQYGAELGKPGTSVRLSCKLSGYKIRNTYIHWVNQRPGKGLEWIGRIDPANGNTIYAEKFKSKVTLTADTSSNTAYMQLSQLKSDDTALYFCAMNYEGYEDYWGQGVMVTVSS

SEQ ID NO: 33 - protein, 106 aa, synthetic construct
  REGION 1..106: note = Description of Artificial Sequence: Synthetic polypeptide
DIQMTQSPSFLSASVGDSVTINCKASQNINKYLNWYQQKLGEAPKRLIHKTDSLQTGIPSRFSGSGSGTDYTLTISSLQPEDVATYFCFQYKSGFMFGAGTKLELK

SEQ ID NO: 34 - protein, 120 aa, synthetic construct
  REGION 1..120: note = Description of Artificial Sequence: Synthetic polypeptide
QIQLVQSGPELKKPGESVKISCKASGYTFTDYAVYWVIQAPGKGLKWMGWINTYTGKPTYADDFKGRFVFSLETSASTANLQISNLKNEDTATYFCARGAGMTKDYVMDAWGRGVLVTVS

SEQ ID NO: 35 - protein, 108 aa, synthetic construct
  REGION 1..108: note = Description of Artificial Sequence: Synthetic polypeptide
SYELIQPPSASVTLGNTVSLTCVGDELSKRYAQWYQQKPDKTIVSVIYKDSERPSDISDRFSGSSSGTTATLTIHGTLAEDEADYYCLSTYSDDNLPVFGGGTKLTVL

SEQ ID NO: 36 - protein, 116 aa, synthetic construct
  REGION 1..116: note = Description of Artificial Sequence: Synthetic polypeptide
QVQLKESGPGLVQPSQTLSLTCTVSGFSLTSYLVHWVRQPPGKTLEWVGLMWNDGDTSYNSALKSRLSISRDTSKSQVFLKMHSLQAEDTATYYCARESNLGFTYWGHGTLVTVSS

SEQ ID NO: 37 - protein, 107 aa, synthetic construct
  REGION 1..107: note = Description of Artificial Sequence: Synthetic polypeptide
DIQMTQSPASLSASLEEIVTITCKASQGIDDDLSWYQQKPGKSPQLLIYDVTRLADGVPSRFSGSRSGTQYSLKISRPQVADSGIYYCLQSYSTPYTFGAGTKLELK

SEQ ID NO: 38 - protein, 117 aa, synthetic construct
  REGION 1..117: note = Description of Artificial Sequence: Synthetic polypeptide
EVQLQQYGAELGKPGTSVRLSCKVSGYNIRNTYIHWVHQRPGEGLEWIGRIDPTNGNTISAEKFKSKATLTADTSSNTAYMQFSQLKSDDTAIYFCAMNYEGYADYWGQGVMVTVSS

SEQ ID NO: 39 - protein, 106 aa, synthetic construct
  REGION 1..106: note = Description of Artificial Sequence: Synthetic polypeptide
DIQMTQSPSFLSASVGDRLTINCKASQNINKYLNWYQQKLGEAPKRLIFKTNSLQTGIPSRFSGSGSGTDYTLTISSLQPEDVATYFCFQYNIGFTFGAGTKLELK

SEQ ID NO: 40 - protein, 124 aa, synthetic construct
  REGION 1..124: note = Description of Artificial Sequence: Synthetic polypeptide
EVQLVESGGGLVQSGRSLKLSCAASGFTVSDYYMAWVRQAPTKGLEWVATINYDGSTTYHRDSVKGRFTISRDNAKSTLYLQMDSLRSEDTATYYCARHGDYGYHYGAYYFDYWGQGVMVTVSS

SEQ ID NO: 41 - protein, 109 aa, synthetic construct
  REGION 1..109: note = Description of Artificial Sequence: Synthetic polypeptide
DIVLTQSPALAVSLGQRATISCRASQTVSLSGYNLIHWYQQRTGQQPKLLIYRASNLAPGIPARFSGSGSGTDFTLTISPVQSDDIATYYCQQSRESWTFGGGTNLEMK

SEQ ID NO: 42 - protein, 116 aa, synthetic construct
  REGION 1..116: note = Description of Artificial Sequence: Synthetic polypeptide
QIQLVQSGPELKKPGESVKISCKASGYTFTDYAIHWVKQAPGQGLRWMAWINTETGKPTYADDFKGRFVFSLEASASTAHLQISNLKNEDTATFFCAGGSHWFAYWGQGTLVTVSS

SEQ ID NO: 43 - protein, 108 aa, synthetic construct
  REGION 1..108: note = Description of Artificial Sequence: Synthetic polypeptide
SYELIQPPSASVTLENTVSITCSGDELSNKYAHWYQQKPDKTILEVIYNDSERPSGISDRFSGSSSGTTAILTIRDAQAEDEADYYCLSTFSDDDLPIFGGGTKLTVL

SEQ ID NO: 44 - protein, 108 aa, synthetic construct
  REGION 1..108: note = Description of Artificial Sequence: Synthetic polypeptide
SYELIQPPSTSVTLGNTVSLTCVGNELPKRYAYWFQQKPDQSIVRLIYDDDRRPSGISDRFSGSSSGTTATLTIRDAQAEDEAYYYCHSTYTDDKVPIFGGGTKLTVL

SEQ ID NO: 45 - protein, 123 aa, synthetic construct
  REGION 1..123: note = Description of Artificial Sequence: Synthetic polypeptide
EVQLVESGGGLVQPGRSMKLSCKASGFTFSNYDMAWVRQAPTRGLEWVASISYDGITAYYRDSVKGRFTISRENAKSTLYLQLVSLRSEDTATYYCTTEGGYVYSGPHYFDYWGQGVMVTVSS

SEQ ID NO: 46 - protein, 107 aa, synthetic construct
  REGION 1..107: note = Description of Artificial Sequence: Synthetic polypeptide
DIQMTQSPSSMSVSLGDTVTITCRASQDVGIFVNWFQQKPGRSPRRMIYRATNLADGVPSRFSGSRSGSDYSLTISSLESEDVADYHCLQYDEFPRTFGGGTKLELK

SEQ ID NO: 47 - protein, 117 aa, synthetic construct
  REGION 1..117: note = Description of Artificial Sequence: Synthetic polypeptide
EVQLQQYGAELGKPGTSVRLSCKVSGYKIRNTYIHWVNQRPGKGLEWIGRIDPANGNTIYAEKFKSKVTLTADTSSNTAYMQLSQLKSDDTALYFCAMNYEGYEDYWGQGVMVTVSS

SEQ ID NO: 48 - protein, 106 aa, synthetic construct
  REGION 1..106: note = Description of Artificial Sequence: Synthetic polypeptide
DIQMTQSPSFLSASVGDSVTINCKASQNINKYLNWYQQKLGEAPKRLIHKTNSLQPGFPSRFSGSGSGTDYTLTISSLQPEDVAAYFCFQYNSGFTFGAGTKLELK

SEQ ID NO: 49 - protein, 125 aa, synthetic construct
  REGION 1..125: note = Description of Artificial Sequence: Synthetic polypeptide
QVQLVQSGAEVKKPGASVKVSCKASGYTFTDYYIHWVRQAPGQGLEWMGWMNPHSGDTGYAQKFQGRVTMTRDTSTSTVYMELSSLRSEDTAVYYCARHGRGYNGYEGAFDIWGQGTLVTVSSAS

SEQ ID NO: 50 - protein, 107 aa, synthetic construct
  REGION 1..107: note = Description of Artificial Sequence: Synthetic polypeptide
DIQMTQSPSSLSASVGDRVTITCRASQGIGNELGWYQQKPGKAPKLLIYAASNLQSGVPSRFSGSGSGTDFTLTISSLQPEDFATYYCQQYDNLPLTFGQGTKVEIK

SEQ ID NO: 51 - protein, 125 aa, synthetic construct
  REGION 1..125: note = Description of Artificial Sequence: Synthetic polypeptide
QVQLVQSGAEVKKPGASVKVSCKASGYTFTGYYLHWVRQAPGQGLEWMGWINPNSGDTNYAQNFQGRVTMTRDTSTSTVYMELSSLRSEDTAVYYCARHGRGYNGYEGAFDIWGQGTLVTVSSAS

SEQ ID NO: 52 - protein, 107 aa, synthetic construct
  REGION 1..107: note = Description of Artificial Sequence: Synthetic polypeptide
DIQMTQSPSSLSASVGDRVTITCRASQGIRNDLGWYQQKPGKAPKLLIYDASSLESGVPSRFSGSGSGTDFTLTISSLQPEDFATYYCQQLNGYPLTFGGGTKVEIK

SEQ ID NO: 53 - protein, 125 aa, synthetic construct
  REGION 1..125: note = Description of Artificial Sequence: Synthetic polypeptide
QVQLVQSGAEVKKPGASVKVSCKASGYTFTGYYLHWVRQAPGQGLEWMGWINPNSGGTNYAQKFQGRVTMTRDTSTSTVYMELSSLRSEDTAVYYCARHGRGYEGYEGAFDIWGQGTLVTVSSAS

SEQ ID NO: 54 - protein, 107 aa, synthetic construct
  REGION 1..107: note = Description of Artificial Sequence: Synthetic polypeptide
DIQMTQSPSSLSASVGDRVTITCRASQGIRNDLGWYQQKPGKAPKLLIYDASELETGVPSRFSGSGSGTDFTLTISSLQPEDFATYYCQQLNGYPITFGQGTKVEIK

SEQ ID NO: 55 - protein, 125 aa, synthetic construct
  REGION 1..125: note = Description of Artificial Sequence: Synthetic polypeptide
QVQLVQSGAEVKKPGASVKVSCKASGYTFTSYYIHWVRQAPGQGLEWMGWLNPSGGGTSYAQKFQGRVTMTRDTSTSTVYMELSSLRSEDTAVYYCARHGRGYDGYEGAFDIWGQGTLVTVSSAS

SEQ ID NO: 56 - protein, 107 aa, synthetic construct
  REGION 1..107: note = Description of Artificial Sequence: Synthetic polypeptide
DIQMTQSPSSLSASVGDRVTITCRASQGIRNDLGWYQQKPGKAPKLLIYDASNLETGVPSRFSGSGSGTDFTLTISSLQPEDFATYYCQQLNGYPLTFGGGTKVEIK

SEQ ID NO: 57 - protein, 125 aa, synthetic construct
  REGION 1..125: note = Description of Artificial Sequence: Synthetic polypeptide
QVQLVQSGAEVKKPGASVKVSCKASGYTFSTYYMHWVRQAPGQGLEWMGIINPSGGSTSYAQKFQGRVTMTRDTSTSTVYMKLSSLRSEDTAVYYCARHGRGYEGYEGAFDIWGQGTLVTVSSAS

SEQ ID NO: 58 - protein, 107 aa, synthetic construct
  REGION 1..107: note = Description of Artificial Sequence: Synthetic polypeptide
DIQMTQSPSSLSASVGDRVTITCRASQGIRDDLGWYQQKPGKAPKLLIYDASNLETGVPSRFSGSGSGTDFTLTISSLQPEDFATYYCQQANGFPLTFGGGTKVEIK

SEQ ID NO: 59 - protein, 125 aa, synthetic construct
  REGION 1..125: note = Description of Artificial Sequence: Synthetic polypeptide
QVQLVQSGAEVKKPGASVKVSCKASGYTFTGYYIHWVRQAPGQGLEWMGIINPSGGNTNYAQNFQGRVTMTRDTSTSTVYMELSSLRSEDTAVYYCARHGRGYNAYEGAFDIWGQGTLVTVSSAS

SEQ ID NO: 60 - protein, 107 aa, synthetic construct
  REGION 1..107: note = Description of Artificial Sequence: Synthetic polypeptide
DIQMTQSPSSLSASVGDRVTITCRASQGIRNDLGWYQQKPGKAPKLLIYDASNLETGVPSRFSGSGSGTDFTLTISSLQPEDFATYYCQQVNGYPLTFGGGTKVEIK

SEQ ID NO: 61 - protein, 125 aa, synthetic construct
  REGION 1..125: note = Description of Artificial Sequence: Synthetic polypeptide
QVQLVQSGAEVKKPGASVKVSCKASGGTFSSYAISWVRQAPGQGLEWMGVINPTVGGANYAQKFQGRVTMTRDTSTSTVYMELSSLRSEDTAVYYCARHGRGYNEYEGAFDIWGQGTLVTVSSAS

SEQ ID NO: 62 - protein, 107 aa, synthetic construct
  REGION 1..107: note = Description of Artificial Sequence: Synthetic polypeptide
DIQMTQSPSSLSASVGDRVTITCQASQDISDYLNWYQQKPGKAPKLLIYDASNLETGVPSRFSGSGSGTDFTLTISSLQPEDFATYYCQQGNSFPLTFGGGTKLEIK

SEQ ID NO: 63 - protein, 125 aa, synthetic construct
  REGION 1..125: note = Description of Artificial Sequence: Synthetic polypeptide
QVQLVQSGAEVKKLGASVKVSCKASGYTFSSYYMHWVRQAPGQGLEWMGVINPNGAGTNFAQKFQGRVTMTRDTSTSTVYMELSSLRSEDTAVYYCARHGRGYEGYEGAFDIWGQGTLVTVSSAS

SEQ ID NO: 64 - protein, 125 aa, synthetic construct
  REGION 1..125: note = Description of Artificial Sequence: Synthetic polypeptide
QVQLVQSGAEVKKPGASVKVSCKASGYTFTTYYMHWVRQAPGQGLEWMGWINPTGGGTNYAQNFQGRVTMTRDTSTSTVYMELSSLRSEDTAVYYCARHGRGYEGYEGAFDIWGQGTLVTVSSAS

SEQ ID NO: 65 - protein, 107 aa, synthetic construct
  REGION 1..107: note = Description of Artificial Sequence: Synthetic polypeptide
DIQMTQSPSSLSASVGDRVTITCRASQGIRNDVSWYQQKPGKAPKLLIYDASNLETGVPSRFSGSGSGTDFTLTISSLQPEDFATYYCQQLSGYPITFGQGTKLEIK

SEQ ID NO: 66 - protein, 125 aa, synthetic construct
  REGION 1..125: note = Description of Artificial Sequence: Synthetic polypeptide
QVQLVQSGAEVKKPGASVKVSCKASGYTFTSYYIHWVRQAPGQGLEWMGMINPSGGSTNYAQKFQGRVTMTRDTSTSTVYMELSSLRSEDTAVYYCARHGRGYNDYEGAFDIWGQGTLVTVSSAS

SEQ ID NO: 67 - protein, 107 aa, synthetic construct
  REGION 1..107: note = Description of Artificial Sequence: Synthetic polypeptide
DIQMTQSPSSLSASVGDRVTITCRASQSISDWLAWYQQKPGKAPKLLIYEASNLEGGVPSRFSGSGSGTDFTLTISSLQPEDFATYYCQQANSFPYTFGQGTKVEIK

SEQ ID NO: 68 - protein, 125 aa, synthetic construct
  REGION 1..125: note = Description of Artificial Sequence: Synthetic polypeptide
QVQLVQSGAEVKKPGASVKVSCKASGYIFSAYYIHWVRQAPGQGLEWMGIINPSGGSTRYAQKFQGRVTMTRDTSTSTVYMELSSLRSEDTAVYYCARHGRGYGGYEGAFDIWDQGTLVTVSSAS

SEQ ID NO: 69 - protein, 107 aa, synthetic construct
  REGION 1..107: note = Description of Artificial Sequence: Synthetic polypeptide
DIQMTQSPSSLSASVGDRVTITCRASQGIGDYVAWYQQKPGKAPKLLIYDASNLETGVPSRFSGSGSGTDFTLTISSLQPEDFATYYCQQLNGYPITFGQGTRLEIK

SEQ ID NO: 70 - protein, 125 aa, synthetic construct
  REGION 1..125: note = Description of Artificial Sequence: Synthetic polypeptide
EVQLVQSGAEVKKPGESLKISCKGSGYRFTSYWIGWVRQMPGKGLEWMGIIYPDDSDTRYSPSFQGQVTISVDKSNSTAYLQWSSLKASDTAMYYCARHGRGYNGYEGAFDIWGQGTLVTVSSAS

SEQ ID NO: 71 - protein, 107 aa, synthetic construct
  REGION 1..107: note = Description of Artificial Sequence: Synthetic polypeptide
DIQMTQSPSSLSASVGDRVTITCRASQGISSYLAWYQQKPGKAPKLLIYDASNLETGVPSRFSGSGSGTYFTLTISSLQPEDFATYYCQQGASFPITFGQGTKVEIK

SEQ ID NO: 72 - protein, 125 aa, synthetic construct
  REGION 1..125: note = Description of Artificial Sequence: Synthetic polypeptide
EVQLVQSGAEVKKPGESLKISCKGSGSSFPNSWIAWVRQMPGKGLEWMGIIYPSDSDTRYSPSFQGQVTISADKSISTAYLQWSSLEASDTAMYYCARHGRGYNGYEGAFDIWGQGTLVTVSSAS

SEQ ID NO: 73 - protein, 107 aa, synthetic construct
  REGION 1..107: note = Description of Artificial Sequence: Synthetic polypeptide
DIQMTQSPSSLSASVGDRVTITCRASQGIRNYLAWYQQKPGKAPKLLIYDASSLQSGVPSRFSGSGSGTDFTLTISSLQPEDFATYYCQQLNSYPLTFGGGTKVEIK

SEQ ID NO: 74 - protein, 125 aa, synthetic construct
  REGION 1..125: note = Description of Artificial Sequence: Synthetic polypeptide
EVQLVQSGAEVKKPGESLKISCKGSGYSFDSYWIGWVRQMPGKGLEWMGIMYPGDSDTRYSPSFQGQVTISADKSISTAYLQWSSLKASDTAMYYCARHGRGYNAYEGAFDIWGQGTLVTVSSAS

SEQ ID NO: 75 - protein, 107 aa, synthetic construct
  REGION 1..107: note = Description of Artificial Sequence: Synthetic polypeptide
DIQMTQSPSSLSASVGDRVTITCRASQSINNWLAWYQQKPGKAPKLLIYDAFILQSGVPSRFSGSGSGTDFTLTISSLQPEDFATYYCLQLNSYPLTFGPGTKVDIK

SEQ ID NO: 76 - protein, 124 aa, synthetic construct
  REGION 1..124: note = Description of Artificial Sequence: Synthetic polypeptide
EVQLVQSGAEVKKPGESLKISCKGSGYSFTNWIAWVRQMPGKGLEWMGIIYPGDSETRYSPSFQGQVTISADKSISTAYLQWSSLKASDTAMYYCARHGRGYYGYEGAFDIWGQGTLVTVSSAS

SEQ ID NO: 77 - protein, 107 aa, synthetic construct
  REGION 1..107: note = Description of Artificial Sequence: Synthetic polypeptide
DIQMTQSPSSLSASVGDRVTITCRASQGISDNLNWYQQKPGKAPKLLIYDASNLETGVPSRFSGSGSGTDFTLTISSLQPEDFATYYCQQAISFPLTFGQGTKVEIK

SEQ ID NO: 78 - protein, 125 aa, synthetic construct
  REGION 1..125: note = Description of Artificial Sequence: Synthetic polypeptide
EVQLVQSGAEVKKPGESLKISCKGSGYNFTSYWIGWVRQMPGKGLEWMGVIYPDDSETRYSPSFQGQVTISADKSISTAYLQWSSLKASDTAMYYCARHGRGYNGYEGAFDIWGQGTLVTVSSAS

SEQ ID NO: 79 - protein, 107 aa, synthetic construct
  REGION 1..107: note = Description of Artificial Sequence: Synthetic polypeptide
DIQMTQSPSSLSASVGDRVTITCRASRDIRDDLGWYQQKPGKAPKLLIYDASNLETGVPSRFSGSGSGTDFTLTISSLQPEDFATYYCQQANSFPLTFGGGTKVEIK

SEQ ID NO: 80 - protein, 124 aa, synthetic construct
  REGION 1..124: note = Description of Artificial Sequence: Synthetic polypeptide
EVQLVQSGAEVKKPGESLKISCKGSGYTFNTYIGWVRQMPGKGLEWMGIIYPGDSGTRYSPSFQGQVTISADKAISTAYLQWSSLKASDTAMYYCARHSRGYNGYEGAFDIWGQGTLVTVSSAS

SEQ ID NO: 81 - protein, 107 aa, synthetic construct
  REGION 1..107: note = Description of Artificial Sequence: Synthetic polypeptide
DIQMTQSPSSLSASVGDRVTITCRASQGISNYLAWYQQKPGKAPKLLIYDASNLETGVPSRFSGSGSGTDFTLTISSLQPEDFATYYCQQANSFPVTFGQGTKVEIK

SEQ ID NO: 82 - protein, 125 aa, synthetic construct
  REGION 1..125: note = Description of Artificial Sequence: Synthetic polypeptide
EVQLVQSGAEVKKPGESLKISCKGSGYNFTTYWIGWVRQMPGKGLEWMGIIHPADSDTRYNPSFQGQVTISADKSISTAYLQWSSLKASDTAMYYCARHGRGYNGYEGAFDIWGQGTLVTVSSAS

SEQ ID NO: 83 - protein, 107 aa, synthetic construct
  REGION 1..107: note = Description of Artificial Sequence: Synthetic polypeptide
DIQMTQSPSSLSASVGDRVTITCRVSQGISSYLAWYQQKPGKAPKLLIYDASNLETGVPSRFSGSGSGTDFTLTISSLQPEDFATYYCQQANSFPLTFGGGTKVEIK

SEQ ID NO: 84 - protein, 125 aa, synthetic construct
  REGION 1..125: note = Description of Artificial Sequence: Synthetic polypeptide
EVQLVQSGAEVKKPGESLKISCKGSGYRFSNYWIAWVRQMPGKGLEWMGIIYPDNSDTRYSPSFQGQVTISADKSISTAYLQWSSLKASDTAMYYCARHGRGYDGYEGAFDIWGQGTLVTVSSAS

SEQ ID NO: 85 - protein, 107 aa, synthetic construct
  REGION 1..107: note = Description of Artificial Sequence: Synthetic polypeptide
DIQMTQSPSSLSASVGDRVTITCRASQGIRSDLAWYQQKPGKAPKLLIYGASSLQSGVPSRFSGSGSGTDFTLTISSLQPEDFATYYCQQANSFPLSFGQGTKVEIK

SEQ ID NO: 86 - protein, 124 aa, synthetic construct
  REGION 1..124: note = Description of Artificial Sequence: Synthetic polypeptide
EVQLVQSGAEVKKPGESLKISCKGSGYRFASYWIGWVRQMPGKGLEWMGITYPGDSETRYNPSQGQVTISADKSISTAYLQWSSLKASDTAMYYCARHGRGYGGYEGAFDIWGQGTLVTVSSAS

SEQ ID NO: 87 - protein, 107 aa, synthetic construct
  REGION 1..107: note = Description of Artificial Sequence: Synthetic polypeptide
DIQMTQSPSSLSASVGDRVTITCRASQGIRNDLGWYQQKPGKAPKLLIYDASNLETGVPSRFSGSGSGTDFTLTISSLQPEDFATYYCQQANSFPLTFGGGTKVEIK

SEQ ID NO: 88 - protein, 125 aa, synthetic construct
  REGION 1..125: note = Description of Artificial Sequence: Synthetic polypeptide
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARHGRGYNGYEGAFDIWGQGTLVTVSSAS

SEQ ID NO: 89 - protein, 107 aa, synthetic construct
  REGION 1..107: note = Description of Artificial Sequence: Synthetic polypeptide
DIQMTQSPSSLSASVGDRVTITCRASQSISNWLAWYQQKPGKAPKLLIYDASNLETGVPSRFSGSGSGTDFTLTISSLQPEDFATYYCQQTNSFPLTFGQGTRLEIK

SEQ ID NO: 90 - protein, 107 aa, synthetic construct
  REGION 1..107: note = Description of Artificial Sequence: Synthetic polypeptide
DIQLTQSPSSLSASVGDRVTITCRASQGVISALAWYQQKPGKAPKLLIYDASSLESGVPSRFSGSGSGTDFTLTISSLQPEDFATYYCQQFNSYPLTFGGGTKVEIK

SEQ ID NO: 91 - protein, 107 aa, synthetic construct
  REGION 1..107: note = Description of Artificial Sequence: Synthetic polypeptide
DIQLTQSPSSLSASVGDRVTITCRASQGIRSALAWYQQKPGKAPKLLIYDASSLESGVPSRFSGSGSGTDFTLTISSLQPEDFATYYCQQFNSYPLTFGGGTKVEIK

SEQ ID NO: 92 - protein, 107 aa, synthetic construct
  REGION 1..107: note = Description of Artificial Sequence: Synthetic polypeptide
DIQLTQSPSSLSASVGDRVTITCRASQGVGSALAWYQQKPGKAPKLLIYDASSLESGVPSRFSGSGSGTDFTLTISSLQPEDFATYYCQQFNSYPLTFGGGTKVEIK

SEQ ID NO: 93 - protein, 107 aa, synthetic construct
  REGION 1..107: note = Description of Artificial Sequence: Synthetic polypeptide
DIQLTQSPSSLSASVGDRVTITCRASQGVISALAWYQQKPGKAPKLLIYDASILESGVPSRFSGSGSGTDFTLTISSLQPEDFATYYCQQFNSYPLTFGGGTKVEIK

SEQ ID NO: 94 - protein, 5 aa, synthetic construct
  REGION 1..5: note = Description of Artificial Sequence: Synthetic peptide
TYWIG

SEQ ID NO: 95 - protein, 11 aa, synthetic construct
  REGION 1..11: note = Description of Artificial Sequence: Synthetic peptide
RASQGVISALA

SEQ ID NO: 96 - protein, 7 aa, synthetic construct
  REGION 1..7: note = Description of Artificial Sequence: Synthetic peptide
DASILES

SEQ ID NO: 97 - protein, 9 aa, synthetic construct
  REGION 1..9: note = Description of Artificial Sequence: Synthetic peptide
QQFNSYPLT

SEQ ID NO: 98 - protein, 107 aa, synthetic construct
  REGION 1..107: note = Description of Artificial Sequence: Synthetic polypeptide
DIQLTQSPSSLSASVGDRVTITCRASQGIRSALAWYQQKPGKAPKLLIYDASILESGVPSRFSGSGSGTDFTLTISSLQPEDFATYYCQQFNSYPLTFGGGTKVEIK

SEQ ID NO: 99 - protein, 107 aa, synthetic construct
  REGION 1..107: note = Description of Artificial Sequence: Synthetic polypeptide
DIQLTQSPSSLSASVGDRVTITCRASQGVGSALAWYQQKPGKAPKLLIYDASILESGVPSRFSGSGSGTDFTLTISSLQPEDFATYYCQQFNSYPLTFGGGTKVEIK

SEQ ID NO: 100 - protein, 11 aa, synthetic construct
  REGION 1..11: note = Description of Artificial Sequence: Synthetic peptide
RASQGVGSALA

SEQ ID NO: 101 - protein, 107 aa, synthetic construct
  REGION 1..107: note = Description of Artificial Sequence: Synthetic polypeptide
DIQLTQSPSSLSASVGDRVTITCRASQGISSALAWYQQKPGKAPKLLIYDASILESGVPSRFSGSGSGTDFTLTISSLQPEDFATYYCQQFNSYPLTFGGGTKVEIK

SEQ ID NO: 102 - protein, 107 aa, synthetic construct
  REGION 1..107: note = Description of Artificial Sequence: Synthetic polypeptide
DIQLTQSPSSLSASVGDRVTITCRASQGVISALAWYQQKPGKAPKLLIYDASTLESGVPSRFSGSGSGTDFTLTISSLQPEDFATYYCQQFNSYPLTFGGGTKVEIK

SEQ ID NO: 103 - protein, 7 aa, synthetic construct
  REGION 1..7: note = Description of Artificial Sequence: Synthetic peptide
DASTLES

SEQ ID NO: 104 - protein, 107 aa, synthetic construct
  REGION 1..107: note = Description of Artificial Sequence: Synthetic polypeptide
DIQLTQSPSSLSASVGDRVTITCRASQGIRSALAWYQQKPGKAPKLLIYDASTLESGVPSRFSGSGSGTDFTLTISSLQPEDFATYYCQQFNSYPLTFGGGTKVEIK

SEQ ID NO: 105 - protein, 107 aa, synthetic construct
  REGION 1..107: note = Description of Artificial Sequence: Synthetic polypeptide
DIQLTQSPSSLSASVGDRVTITCRASQGVGSALAWYQQKPGKAPKLLIYDASTLESGVPSRFSGSGSGTDFTLTISSLQPEDFATYYCQQFNSYPLTFGGGTKVEIK

SEQ ID NO: 106 - protein, 123 aa, synthetic construct
  REGION 1..123: note = Description of Artificial Sequence: Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSS

SEQ ID NO: 107 - protein, 107 aa, synthetic construct
  REGION 1..107: note = Description of Artificial Sequence: Synthetic polypeptide
DIQMTQSPSSLSASVGDRVTITCRSSQGIRSDLGWYQQKPGKAPKLLIYDASNLETGVPSRFSGSGSGTDFTLTISSLQPEDFATYYCQQANGFPLTFGGGTKVEIK

SEQ ID NO: 108 - protein, 5 aa, synthetic construct
  REGION 1..5: note = Description of Artificial Sequence: Synthetic peptide
NYWIG

SEQ ID NO: 109 - protein, 17 aa, synthetic construct
  REGION 1..17: note = Description of Artificial Sequence: Synthetic peptide
IINPRDSDTRYRPSFQG

SEQ ID NO: 110 - protein, 14 aa, synthetic construct
  REGION 1..14: note = Description of Artificial Sequence: Synthetic peptide
HGRGYEGYEGAFDI

SEQ ID NO: 111 - protein, 11 aa, synthetic construct
  REGION 1..11: note = Description of Artificial Sequence: Synthetic peptide
RSSQGIRSDLG

SEQ ID NO: 112 - protein, 7 aa, synthetic construct
  REGION 1..7: note = Description of Artificial Sequence: Synthetic peptide
DASNLET

SEQ ID NO: 113 - protein, 9 aa, synthetic construct
  REGION 1..9: note = Description of Artificial Sequence: Synthetic peptide
QQANGFPLT

SEQ ID NO: 114 - protein, 123 aa, synthetic construct
  REGION 1..123: note = Description of Artificial Sequence: Synthetic polypeptide
EVQLVQSGAEVKKPGESLKISCKGSGYSFTNYWIGWVRQMPGKGLEWMGIIYPGDSDIRYSPSLQGQVTISVDTSTSTAYLQWNSLKPSDTAMYYCARHGRGYNGYEGAFDIWGQGTLVTVSS

SEQ ID NO: 115 - protein, 107 aa, synthetic construct
  REGION 1..107: note = Description of Artificial Sequence: Synthetic polypeptide
DIQMTQSPSSLSASVGDRVTITCRASQGIGDSLAWYQQKPGKAPKLLIYDASNLETGVPSRFSGSGSGTDFTLTISSLQPEDFATYYCQQLNGYPITFGQGTKVEIK

SEQ ID NO: 116 - protein, 17 aa, synthetic construct
  REGION 1..17: note = Description of Artificial Sequence: Synthetic peptide
IIYPGDSDIRYSPSLQG

SEQ ID NO: 117 - protein, 11 aa, synthetic construct
  REGION 1..11: note = Description of Artificial Sequence: Synthetic peptide
RASQGIGDSLA

SEQ ID NO: 118 - protein, 9 aa, synthetic construct
  REGION 1..9: note = Description of Artificial Sequence: Synthetic peptide
QQLNGYPIT

SEQ ID NO: 119 - protein, 107 aa, synthetic construct
  REGION 1..107: note = Description of Artificial Sequence: Synthetic polypeptide
DIQMTQSPSSLSASVGDRVTITCRASQGIRNDLGWYQQKPGKAPKLLIYDASSLESGVPSRFSGSGSGTDFTLTISSLQPEDFATYYCQQLNGYPITFGQGTKVEIK

SEQ ID NO: 120 - protein, 11 aa, synthetic construct
  REGION 1..11: note = Description of Artificial Sequence: Synthetic peptide
RASQGIRNDLG

SEQ ID NO: 121 - protein, 123 aa, synthetic construct
  REGION 1..123: note = Description of Artificial Sequence: Synthetic polypeptide
EVQLVQSGAEVKKPGESLKISCKGSGYSFTNYWIGWVRQMPGKGLEWMGIIYPGDSLTRYSPSFQGQVTISADKSISTAYLQWSSLKASDTAMYYCARHGRGYNGYEGAFDIWGQGTLVTVSS

SEQ ID NO: 122 - protein, 17 aa, synthetic construct
  REGION 1..17: note = Description of Artificial Sequence: Synthetic peptide
IIYPGDSLTRYSPSFQG

SEQ ID NO: 123 - protein, 123 aa, synthetic construct
  REGION 1..123: note = Description of Artificial Sequence: Synthetic polypeptide
EVQLVQSGAEVKKPGESLKISCKGSGYSFTNYWIGWVRQMPGKGLEWMGIIYPGDSDTRYSPSFQGQVTISADKSISTAYLQWSSLKASDTAMYYCARHGRGYNGYEGAFDIWGQGTLVTVSS

SEQ ID NO: 124 - protein, 123 aa, synthetic construct
  REGION 1..123: note = Description of Artificial Sequence: Synthetic polypeptide
EVQLVQSGAEVKKPGESLKISCKGSGYRFTTSWIGWVRQMPGKGLEWMGIIYPGDSDTRYSPSFQGQVTISADKSISTAYLQWSSLKASDTAMYYCARHGLGYNGYEGAFDIWGQGTLVTVSS

SEQ ID NO: 125 - protein, 107 aa, synthetic construct
  REGION 1..107: note = Description of Artificial Sequence: Synthetic polypeptide
DIQMTQSPSSLSASVGDRVTITCRASQGIGSALAWYQQKPGKAPKLLIYDASTLESGVPSRFSGSGSGTDFTLTISSLQPEDFATYYCQQFNGYPLTFGQGTRLEIK

SEQ ID NO: 126 - protein, 107 aa, synthetic construct
  REGION 1..107: note = Description of Artificial Sequence: Synthetic polypeptide
DIQMTQSPSSLSASVGDRVTITCRASRGISDYLAWYQQKPGKAPKLLIYDASNLETGVPSRFSGSGSGTDFTLTISSLQPEDFATYYCQQANSFPITFGQGTRLEIK

SEQ ID NO: 127 - protein, 107 aa, synthetic construct
  REGION 1..107: note = Description of Artificial Sequence: Synthetic polypeptide
DIQMTQSPSSLSASVGDRVTITCRASQGIGSALAWYQQKPGKAPKLLIYDASTLESGVPSRFSGSGSGTDFTLTISSLQPEDFATYYCQQLNGYPLTFGQGTRLEIK

SEQ ID NO: 128 - protein, 107 aa, synthetic construct
  REGION 1..107: note = Description of Artificial Sequence: Synthetic polypeptide
DIQMTQSPSSLSASVGDRVTITCRASQGIGSALAWYQQKPGKAPKLLIYDASNLETGVPSRFSGSGSGTDFTLTISSLQPEDFATYYCQQLNGYPLTFGQGTRLEIK

SEQ ID NO: 129 - protein, 5 aa, synthetic construct
  REGION 1..5: note = Description of Artificial Sequence: Synthetic peptide
TSWIG

SEQ ID NO: 130 - protein, 14 aa, synthetic construct
  REGION 1..14: note = Description of Artificial Sequence: Synthetic peptide
HGLGYNGYEGAFDI

SEQ ID NO: 131 - protein, 11 aa, synthetic construct
  REGION 1..11: note = Description of Artificial Sequence: Synthetic peptide
RASQGIGSALA

SEQ ID NO: 132 - protein, 10 aa, synthetic construct
  REGION 1..10: note = Description of Artificial Sequence: Synthetic peptide
CQQLNGYPLT

SEQ ID NO: 133 - protein, 453 aa, synthetic construct
  REGION 1..453: note = Description of Artificial Sequence: Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 453 aa, synthetic construct
  REGION 1..453: note = Description of Artificial Sequence: Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 453 aa, synthetic construct
  REGION 1..453: note = Description of Artificial Sequence: Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 453 aa, synthetic construct
  REGION 1..453: note = Description of Artificial Sequence: Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 137 - protein, 453 aa, synthetic construct
  REGION 1..453: note = Description of Artificial Sequence: Synthetic polypeptide
EVQLVQSGAEVKKPGESLKISCKGSGYRFTTSWIGWVRQMPGKGLEWMGIIYPGDSDTRYSPSFQGQVTISADKSISTAYLQWSSLKASDTAMYYCARHGLGYNGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 453 aa, synthetic construct
  REGION 1..453: note = Description of Artificial Sequence: Synthetic polypeptide
EVQLVQSGAEVKKPGESLKISCKGSGYRFTTSWIGWVRQMPGKGLEWMGIIYPGDSDTRYSPSFQGQVTISADKSISTAYLQWSSLKASDTAMYYCARHGLGYNGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 453 aa, synthetic construct
  REGION 1..453: note = Description of Artificial Sequence: Synthetic polypeptide
EVQLVQSGAEVKKPGESLKISCKGSGYRFTTSWIGWVRQMPGKGLEWMGIIYPGDSDTRYSPSFQGQVTISADKSISTAYLQWSSLKASDTAMYYCARHGLGYNGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 453 aa, synthetic construct
  REGION 1..453: note = Description of Artificial Sequence: Synthetic polypeptide
EVQLVQSGAEVKKPGESLKISCKGSGYRFTTSWIGWVRQMPGKGLEWMGIIYPGDSDTRYSPSFQGQVTISADKSISTAYLQWSSLKASDTAMYYCARHGLGYNGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 141 - protein, 214 aa, synthetic construct
  REGION 1..214: note = Description of Artificial Sequence: Synthetic polypeptide
DIQMTQSPSSLSASVGDRVTITCRSSQGIRSDLGWYQQKPGKAPKLLIYDASNLETGVPSRFSGSGSGTDFTLTISSLQPEDFATYYCQQANGFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 142 - protein, 214 aa, synthetic construct
  REGION 1..214: note = Description of Artificial Sequence: Synthetic polypeptide
DIQMTQSPSSLSASVGDRVTITCRASQGIGSALAWYQQKPGKAPKLLIYDASNLETGVPSRFSGSGSGTDFTLTISSLQPEDFATYYCQQLNGYPLTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 143 - protein, 330 aa, unidentified [sequence omitted]

SEQ ID NO: 144 - protein, 330 aa, synthetic construct
  REGION 1..330: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 330 aa, synthetic construct
  REGION 1..330: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 330 aa, synthetic construct
  REGION 1..330: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 147 - protein, 330 aa, synthetic construct
  REGION 1..330: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 330 aa, synthetic construct
  REGION 1..330: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 149 - protein, 107 aa, synthetic construct [sequence omitted]

SEQ ID NO: 150 - protein, 121 aa, synthetic construct
  REGION 1..121: note = Description of Artificial Sequence: Synthetic polypeptide
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSS

SEQ ID NO: 151 - protein, 107 aa, synthetic construct
  REGION 1..107: note = Description of Artificial Sequence: Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYIAPYTFGGGTKVEIK

SEQ ID NO: 152 - protein, 9 aa, synthetic construct
  REGION 1..9: note = Description of Artificial Sequence: Synthetic peptide
FTFSDADMD

SEQ ID NO: 153 - protein, 19 aa, synthetic construct
  REGION 1..19: note = Description of Artificial Sequence: Synthetic peptide
RTRNKAGSYTTEYAASVKG

SEQ ID NO: 154 - protein, 12 aa, synthetic construct
  REGION 1..12: note = Description of Artificial Sequence: Synthetic peptide
AREPKYWIDFDL

SEQ ID NO: 155 - protein, 11 aa, synthetic construct
  REGION 1..11: note = Description of Artificial Sequence: Synthetic peptide
RASQSISSYLN

SEQ ID NO: 156 - protein, 7 aa, synthetic construct
  REGION 1..7: note = Description of Artificial Sequence: Synthetic peptide
AASSLQS

SEQ ID NO: 157 - protein, 9 aa, synthetic construct
  REGION 1..9: note = Description of Artificial Sequence: Synthetic peptide
QQSYIAPYT

SEQ ID NO: 158 - protein, 119 aa, synthetic construct
  REGION 1..119: note = Description of Artificial Sequence: Synthetic polypeptide
QVQLVQSGAEVKKPGSSVKVSCKASGGTFRIYAISWVRQAPGQGLEWMGGIIPDFGVANYAQKFQGRVTITADESTSTAYMELSSLRSEDTAVYYCARGGLDTDEFDLWGRGTLVTVSS

SEQ ID NO: 159 - protein, 106 aa, synthetic construct
  REGION 1..106: note = Description of Artificial Sequence: Synthetic polypeptide
DIQMTQSPSSLSASVGDRVTITCRASQSINSYLNWYQQKPGKAPKLLIYAASSLQSGVPSRFSGSGSGTDFTLTISSLQPEDFATYYCQQGVSDITFGGGTKVEIK

SEQ ID NO: 160 - protein, 9 aa, synthetic construct
  REGION 1..9: note = Description of Artificial Sequence: Synthetic peptide
GTFRIYAIS

SEQ ID NO: 161 - protein, 17 aa, synthetic construct
  REGION 1..17: note = Description of Artificial Sequence: Synthetic peptide
GIIPDFGVANYAQKFQG

SEQ ID NO: 162 - protein, 12 aa, synthetic construct
  REGION 1..12: note = Description of Artificial Sequence: Synthetic peptide
ARGGLDTDEFDL

SEQ ID NO: 163 - protein, 11 aa, synthetic construct
  REGION 1..11: note = Description of Artificial Sequence: Synthetic peptide
RASQSINSYLN

SEQ ID NO: 164 - protein, 8 aa, synthetic construct
  REGION 1..8: note = Description of Artificial Sequence: Synthetic peptide
QQGVSDIT

SEQ ID NO: 165 - protein, 119 aa, synthetic construct
  REGION 1..119: note = Description of Artificial Sequence: Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGGLDTDEFDLWGRGTLVTVSS

SEQ ID NO: 166 - protein, 9 aa, synthetic construct
  REGION 1..9: note = Description of Artificial Sequence: Synthetic peptide
GTFSSYAIS

SEQ ID NO: 167 - protein, 17 aa, synthetic construct
  REGION 1..17: note = Description of Artificial Sequence: Synthetic peptide
GIIPIFGTANYAQKFQG

SEQ ID NO: 168 - protein, 119 aa, synthetic construct
  REGION 1..119: note = Description of Artificial Sequence: Synthetic polypeptide
QVQLVQSGAEVKKPGSSVKVSCKASGGTFSLYAISWVRQAPGQGLEWMGGIIPAFGTANYAQKFQGRVTITADESTSTAYMELSSLRSEDTAVYYCARGGLDTDEFDLWGRGTLVTVSS

SEQ ID NO: 169 - protein, 9 aa, synthetic construct
  REGION 1..9: note = Description of Artificial Sequence: Synthetic peptide
GTFSLYAIS

SEQ ID NO: 170 - protein, 17 aa, synthetic construct
  REGION 1..17: note = Description of Artificial Sequence: Synthetic peptide
GIIPAFGTANYAQKFQG

SEQ ID NO: 171 - protein, 119 aa, synthetic construct
  REGION 1..119: note = Description of Artificial Sequence: Synthetic polypeptide
QVQLVQSGAEVKKPGSSVKVSCKASGGTFSLYAISWVRQAPGQGLEWMGGIIPHFGLANYAQKFQGRVTITADESTSTAYMELSSLRSEDTAVYYCARGGLDTDEFDLWGRGTLVTVSS

SEQ ID NO: 172 - protein, 17 aa, synthetic construct
  REGION 1..17: note = Description of Artificial Sequence: Synthetic peptide
GIIPHFGLANYAQKFQG

SEQ ID NO: 173 - protein, 123 aa, synthetic construct
  REGION 1..123: note = Description of Artificial Sequence: Synthetic polypeptide
EVQLLESGGGLVQPGGSLRLSCAASGFTFSNYAMSWVRQAPGKGLEWVSAISGSGGSTYYADSVKGRFTISRDNSKNTLYLQMNSLRAEDTAVYYCAKGPPTYHTNYYYMDVWGKGTTVTVSS

SEQ ID NO: 174 - protein, 107 aa, synthetic construct
  REGION 1..107: note = Description of Artificial Sequence: Synthetic polypeptide
DIQMTQSPSSVSASVGDRVTITCRASQGISSWLAWYQQKPGKAPKLLIYAASSLQSGVPSRFSGSGSGTDFTLTISSLQPEDFATYYCQQTNSFPYTFGGGTKVEIK

SEQ ID NO: 175 - protein, 9 aa, synthetic construct
  REGION 1..9: note = Description of Artificial Sequence: Synthetic peptide
FTFSNYAMS

SEQ ID NO: 176 - protein, 17 aa, synthetic construct
  REGION 1..17: note = Description of Artificial Sequence: Synthetic peptide
AISGSGGSTYYADSVKG

SEQ ID NO: 177 - protein, 16 aa, synthetic construct
  REGION 1..16: note = Description of Artificial Sequence: Synthetic peptide
AKGPPTYHTNYYYMDV

SEQ ID NO: 178 - protein, 11 aa, synthetic construct
  REGION 1..11: note = Description of Artificial Sequence: Synthetic peptide
RASQGISSWLA

SEQ ID NO: 179 - protein, 9 aa, synthetic construct
  REGION 1..9: note = Description of Artificial Sequence: Synthetic peptide
QQTNSFPYT

SEQ ID NO: 180 - protein, 123 aa, synthetic construct
  REGION 1..123: note = Description of Artificial Sequence: Synthetic polypeptide
EVQLLESGGGLVQPGGSLRLSCAASGFTFSSYVMIWVRQAPGKGLEWVSSISGDSVTTYYADSVKGRFTISRDNSKNTLYLQMNSLRAEDTAVYYCAKGPPTYHTNYYYMDVWGKGTTVTVSS

SEQ ID NO: 181 - protein, 9 aa, synthetic construct
  REGION 1..9: note = Description of Artificial Sequence: Synthetic peptide
FTFSSYVMI

SEQ ID NO: 182 - protein, 17 aa, synthetic construct
  REGION 1..17: note = Description of Artificial Sequence: Synthetic peptide
SISGDSVTTYYADSVKG

SEQ ID NO: 183 - protein, 121 aa, synthetic construct
  REGION 1..121: note = Description of Artificial Sequence: Synthetic polypeptide
EVQLVESGGGLVQPGGSLRLSCAASGFTFSDHYMDWVRQAPGKGLEWVGRTRNKASSYTTEYAASVKGRFTISRDDSKNSLYLQMNSLKTEDTAVYYCAREPKYWIDFDLWGRGTLVTVSS

SEQ ID NO: 184 - protein, 9 aa, synthetic construct
  REGION 1..9: note = Description of Artificial Sequence: Synthetic peptide
FTFSDHYMD

SEQ ID NO: 185 - protein, 19 aa, synthetic construct
  REGION 1..19: note = Description of Artificial Sequence: Synthetic peptide
RTRNKASSYTTEYAASVKG

SEQ ID NO: 186 - protein, 121 aa, synthetic construct
  REGION 1..121: note = Description of Artificial Sequence: Synthetic polypeptide
EVQLVESGGGLVQPGRSLRLSCTASGFTFSDHDMNWVRQAPGKGLEWVGRTRNAAGSYTTEYAASVKGRFTISRDDSKNSLYLQMNSLKTEDTAVYYCAREPKYWIDFDLWGRGTLVTVSS

SEQ ID NO: 187 - protein, 9 aa, synthetic construct
  REGION 1..9: note = Description of Artificial Sequence: Synthetic peptide
FTFSDHDMN

SEQ ID NO: 188 - protein, 19 aa, synthetic construct
  REGION 1..19: note = Description of Artificial Sequence: Synthetic peptide
RTRNAAGSYTTEYAASVKG

SEQ ID NO: 189 - protein, 121 aa, synthetic construct
  REGION 1..121: note = Description of Artificial Sequence: Synthetic polypeptide
EVQLVESGGGLVQPGGSLRLSCAASGFTFVDHDMDWVRQAPGKGLEWVGRTRNKLGSYTTEYAASVKGRFTISRDDSKNSLYLQMNSLKTEDTAVYYCAREPKYWIDFDLWGRGTLVTVSS

SEQ ID NO: 190 - protein, 9 aa, synthetic construct
  REGION 1..9: note = Description of Artificial Sequence: Synthetic peptide
FTFVDHDMD

SEQ ID NO: 191 - protein, 19 aa, synthetic construct
  REGION 1..19: note = Description of Artificial Sequence: Synthetic peptide
RTRNKLGSYTTEYAASVKG

SEQ ID NO: 192 - protein, 120 aa, synthetic construct
  REGION 1..120: note = Description of Artificial Sequence: Synthetic polypeptide
EVQLVESGGGLVQPGGSLRLSCAASGFTFSDYAMSWVRQAPGKGLEWVAVISENGSDTYYADSVKGRFTISRDDSKNTLYLQMNSLRAEDTAVYYCARDRGGAVSYFDVWGQGTLVTVSS

SEQ ID NO: 193 - protein, 109 aa, synthetic construct
  REGION 1..109: note = Description of Artificial Sequence: Synthetic polypeptide
DIQMTQSPSSLSASVGDRVTITCRASQDVSSYLAWYQQKPGKAPKLLIYAASSLESGVPSRFSGSGSGTDFTLTISSLQPEDFATYYCQQYNSLPYTFGQGTKVEIKRT

SEQ ID NO: 194 - protein, 214 aa, synthetic construct
  REGION 1..214: note = Description of Artificial Sequence: Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYIAPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 195 - protein, 451 aa, synthetic construct
  REGION 1..451: note = Description of Artificial Sequence: Synthetic polypeptide
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 196 - protein, 451 aa, synthetic construct
  REGION 1..451: note = Description of Artificial Sequence: Synthetic polypeptide
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 197 - protein, 451 aa, synthetic construct
  REGION 1..451: note = Description of Artificial Sequence: Synthetic polypeptide
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 198 - protein, 451 aa, synthetic construct
  REGION 1..451: note = Description of Artificial Sequence: Synthetic polypeptide
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 199 - protein, 451 aa, synthetic construct
  REGION 1..451: note = Description of Artificial Sequence: Synthetic polypeptide
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 200 - protein, 213 aa, synthetic construct
  REGION 1..213: note = Description of Artificial Sequence: Synthetic polypeptide
DIQMTQSPSSLSASVGDRVTITCRASQSINSYLNWYQQKPGKAPKLLIYAASSLQSGVPSRFSGSGSGTDFTLTISSLQPEDFATYYCQQGVSDITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 201 - protein, 449 aa, synthetic construct
  REGION 1..449: note = Description of Artificial Sequence: Synthetic polypeptide
QVQLVQSGAEVKKPGSSVKVSCKASGGTFRIYAISWVRQAPGQGLEWMGGIIPDFGVANYAQKFQGRVTITADESTSTAYMELSSLRSEDTAVYYCARGGLDTDEFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 202 - protein, 449 aa, synthetic construct
  REGION 1..449: note = Description of Artificial Sequence: Synthetic polypeptide
QVQLVQSGAEVKKPGSSVKVSCKASGGTFRIYAISWVRQAPGQGLEWMGGIIPDFGVANYAQKFQGRVTITADESTSTAYMELSSLRSEDTAVYYCARGGLDTDEFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 203 - protein, 449 aa, synthetic construct
  REGION 1..449: note = Description of Artificial Sequence: Synthetic polypeptide
QVQLVQSGAEVKKPGSSVKVSCKASGGTFRIYAISWVRQAPGQGLEWMGGIIPDFGVANYAQKFQGRVTITADESTSTAYMELSSLRSEDTAVYYCARGGLDTDEFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 204 - protein, 449 aa, synthetic construct
  REGION 1..449: note = Description of Artificial Sequence: Synthetic polypeptide
QVQLVQSGAEVKKPGSSVKVSCKASGGTFRIYAISWVRQAPGQGLEWMGGIIPDFGVANYAQKFQGRVTITADESTSTAYMELSSLRSEDTAVYYCARGGLDTDEFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 205 - protein, 449 aa, synthetic construct
  REGION 1..449: note = Description of Artificial Sequence: Synthetic polypeptide
QVQLVQSGAEVKKPGSSVKVSCKASGGTFRIYAISWVRQAPGQGLEWMGGIIPDFGVANYAQKFQGRVTITADESTSTAYMELSSLRSEDTAVYYCARGGLDTDEFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 206 - protein, 9 aa, synthetic construct
  REGION 1..9: note = Description of Artificial Sequence: Synthetic peptide
  VARIANT 4: note = Ser or Arg
  VARIANT 5: note = Ser, Ile or Leu
GTFXXYAIS

SEQ ID NO: 207 - protein, 17 aa, synthetic construct
  REGION 1..17: note = Description of Artificial Sequence: Synthetic peptide
  VARIANT 5: note = Ile, Asp, Ala or His
  VARIANT 8: note = Thr, Val or Leu
GIIPXFGXANYAQKFQG

SEQ ID NO: 208 - protein, 9 aa, synthetic construct
  REGION 1..9: note = Description of Artificial Sequence: Synthetic peptide
  VARIANT 5: note = Asn or Ser
  VARIANT 7: note = Ala or Val
  VARIANT 9: note = Ser or Ile
FTFSXYXMX

SEQ ID NO: 209 - protein, 17 aa, synthetic construct
  REGION 1..17: note = Description of Artificial Sequence: Synthetic peptide
  VARIANT 1: note = Ala or Ser
  VARIANT 5: note = Ser or Asp
  VARIANT 6: note = Gly or Ser
  VARIANT 7: note = Gly or Val
  VARIANT 8: note = Ser or Thr
XISGXXXXTYYADSVKG

SEQ ID NO: 210 - protein, 9 aa, synthetic construct
  REGION 1..9: note = Description of Artificial Sequence: Synthetic peptide
  VARIANT 4: note = Ser or Val
  VARIANT 6: note = His or Ala
  VARIANT 7: note = Tyr or Asp
  VARIANT 9: note = Asp or Asn
FTFXDXXMX

SEQ ID NO: 211 - protein, 19 aa, synthetic construct
  REGION 1..19: note = Description of Artificial Sequence: Synthetic peptide
  VARIANT 5: note = Lys or Ala
  VARIANT 6: note = Ala or Leu
  VARIANT 7: note = Ser or Gly
RTRNXXXSYTTEYAASVKG

SEQ ID NO: 212 - protein, 976 aa, Homo sapiens [sequence omitted]

SEQ ID NO: 213 - protein, 972 aa, Homo sapiens
MRGARGAWDFLCVLLLLLRVQTGSSQPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKEQIHPHTLFTPLLIGFVIVAGMMCIIVMILTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 214 - protein, 453 aa, synthetic construct
  REGION 1..453: note = Description of Artificial Sequence: Synthetic polypeptide
QVQLQQPGAELVRPGASVKLSCKASGYTFTSYWINWVKQRPGQGLEWIGNIYPSDSYTNYNQKFKDKATLTVDKSSNTVYMQLNSPTSEDSAVYYCTRNGVEGYPHYYAMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 215 - protein, 214 aa, synthetic construct
  REGION 1..214: note = Description of Artificial Sequence: Synthetic polypeptide
DIQMTQTTSALSASLGDRVTIGCRASQDLSNHLYWYQQKPDGTVKLLIYYTSRLHSGVPSRFSGSGSGTDYSLTIRNLEQEDVATYFCQQGYTLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 216 - protein, 123 aa, synthetic construct
  REGION 1..123: note = Description of Artificial Sequence: Synthetic polypeptide
QVQLQQPGAELVRPGASVKLSCKASGYTFTSYWINWVKQRPGQGLEWIGNIYPSDSYTNYNQKFKDKATLTVDKSSNTVYMQLNSPTSEDSAVYYCTRNGVEGYPHYYAMEYWGQGTSVTVSS

SEQ ID NO: 217 - protein, 107 aa, synthetic construct
  REGION 1..107: note = Description of Artificial Sequence: Synthetic polypeptide
DIQMTQTTSALSASLGDRVTIGCRASQDLSNHLYWYQQKPDGTVKLLIYYTSRLHSGVPSRFSGSGSGTDYSLTIRNLEQEDVATYFCQQGYTLPYTFGGGTKLEIK

SEQ ID NO: 218 - protein, 5 aa, synthetic construct
  REGION 1..5: note = Description of Artificial Sequence: Synthetic peptide
SYWIN

SEQ ID NO: 219 - protein, 17 aa, synthetic construct
  REGION 1..17: note = Description of Artificial Sequence: Synthetic peptide
NIYPSDSYTNYNQKFKD

SEQ ID NO: 220 - protein, 14 aa, synthetic construct
  REGION 1..14: note = Description of Artificial Sequence: Synthetic peptide
NGVEGYPHYYAMEY

SEQ ID NO: 221 - protein, 11 aa, synthetic construct
  REGION 1..11: note = Description of Artificial Sequence: Synthetic peptide
RASQDLSNHLY

SEQ ID NO: 222 - protein, 7 aa, synthetic construct
  REGION 1..7: note = Description of Artificial Sequence: Synthetic peptide
YTSRLHS

SEQ ID NO: 223 - protein, 9 aa, synthetic construct
  REGION 1..9: note = Description of Artificial Sequence: Synthetic peptide
QQGYTLPYT

SEQ ID NO: 224 - protein, 120 aa, synthetic construct
  REGION 1..120: note = Description of Artificial Sequence: Synthetic polypeptide
QVTLKESGPVLVKPTETLTLTCTFSGFSLSTSGMGVGWIRQAPGKGLEWVAHIWWDDDVYYNPSLKSRLTITKDASKDQVSLKLSSVTAADTAVYYCVRRRATGTGFDYWGQGTLVTVSS

SEQ ID NO: 225 - protein, 106 aa, synthetic construct
  REGION 1..106: note = Description of Artificial Sequence: Synthetic polypeptide
NIVMTQSPSSLSASVGDRVTITCQASQDVGTAVAWYQQKPDQSPKLLIYWTSTRHTGVPDRFTGSGSGTDFTLTISSLQPEDIATYFCHQYNSYNTFGSGTKLEIK

SEQ ID NO: 226 - protein, 9 aa, synthetic construct
  REGION 1..9: note = Description of Artificial Sequence: Synthetic peptide
FSLSTSGMG

SEQ ID NO: 227 - protein, 5 aa, synthetic construct
  REGION 1..5: note = Description of Artificial Sequence: Synthetic peptide
WWDDD

SEQ ID NO: 228 - protein, 10 aa, synthetic construct
  REGION 1..10: note = Description of Artificial Sequence: Synthetic peptide
RRATGTGFDY

SEQ ID NO: 229 - protein, 6 aa, synthetic construct
  REGION 1..6: note = Description of Artificial Sequence: Synthetic peptide
QDVGTA

SEQ ID NO: 230 - protein, 7 aa, synthetic construct
  REGION 1..7: note = Description of Artificial Sequence: Synthetic peptide
WTSTRHT

SEQ ID NO: 231 - protein, 6 aa, synthetic construct
  REGION 1..6: note = Description of Artificial Sequence: Synthetic peptide
YNSYNT

SEQ ID NO: 232 - protein, 976 aa, Homo sapiens [sequence omitted]

SEQ ID NO: 233 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 234 - protein, 438 aa, Homo sapiens
MVCSQSWGRSSKQWEDPSQASKVCQRLNCGVPLSLGPFLVTYTPQSSIICYGQLGSFSNCSHSRNDMCHSLGLTCLEPQKTTPPTTRPPPTTTPEPTAPPRLQLVAQSGGQHCAGVVEFYSGSLGGTISYEAQDKTQDLENFLCNNLQCGSFLKHLPETEAGRAQDPGEPREHQPLPIQWKIQNSSCTSLEHCFRKIKPQKSGRVLALLCSGFQPKVQSRLVGGSSICEGTVEVRQGAQWAALCDSSSARSSLRWEEVCREQQCGSVNSYRVLDAGDPTSRGLFCPHQKLSQCHELWERNSYCKKVFVTCQDPNPAGLAAGTVASIILALVLLVVLLVVCGPLAYKKLVKKFRQKKQRQWIGPTGMNQNMSFHRNHTATVRSHAENPTASHVDNEYSQPPRNSHLSAYPALEGALHRSSMQPDNSSDSDYDLHGAQRL

SEQ ID NO: 235 - protein, 351 aa, Homo sapiens
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 236 - protein, 1145 aa, Homo sapiens
MTMYLWLKLLAFGFAFLDTEVFVTGQSPTPSPTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 237 - protein, 1211 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 238 - protein, 1192 aa, Homo sapiens
MTMYLWLKLLAFGFAFLDTEVFVTGQSPTPSPTGVSSVQTPHLPTHADSQTPSAGTDTQTFSGSAANAKLNPTPGSNAISDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 239 - protein, 1193 aa, Homo sapiens
MTMYLWLKLLAFGFAFLDTEVFVTGQSPTPSPTDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 240 - protein, 118 aa, synthetic construct
  REGION 1..118: note = Description of Artificial Sequence: Synthetic polypeptide
QVQLVQSGAEVKKPGASVKVSCKASGYTFTEYYMYWVRQAPGQGLELMGRIDPEDGSIDYVEKFKKKVTLTADTSSSTAYMELSSLTSDDTAVYYCARGKFNYRFAYWGQGTLVTVSS

SEQ ID NO: 241 - protein, 112 aa, synthetic construct
  REGION 1..112: note = Description of Artificial Sequence: Synthetic polypeptide
DVVMTQSPPSLLVTLGQPASISCRSSQSLLHSSGNTYLNWLLQRPGQSPQPLIYLVSKLESGVPDRFSGSGSGTDFTLKISGVEAEDVGVYYCMQFTHYPYTFGQGTKLEIK

SEQ ID NO: 242 - protein, 118 aa, synthetic construct
  REGION 1..118: note = Description of Artificial Sequence: Synthetic polypeptide
QVQLVQSGAEVQRPGASVKVSCKASGYIFTEYYMYWVRQAPGQGLELVGRIDPEDGSIDYVEKFKKKVTLTADTSSSTAYMELSSLTSDDTAVYYCARGKFNYRFAYWGQGTLVTVSS

SEQ ID NO: 243 - protein, 112 aa, synthetic construct; identical to SEQ ID NO: 241
  REGION 1..112: note = Description of Artificial Sequence: Synthetic polypeptide

SEQ ID NO: 244 - protein, 117 aa, synthetic construct
  REGION 1..117: note = Description of Artificial Sequence: Synthetic polypeptide
EVKLVESGGGLVKPGGSLKLSCAASGFTFSSYDMSWVRQTPEKRLEWVASISGGGFLYYLDSVKGRFTISRDNARNILYLHMTSLRSEDTAMYYCARSSYGEIMDYWGQGTSVTVSS

SEQ ID NO: 245 - protein, 107 aa, synthetic construct
  REGION 1..107: note = Description of Artificial Sequence: Synthetic polypeptide
DILLTQSPAILSVSPGERVSFSCRASQRIGTSIHWYQQRTTGSPRLLIKYASESISGIPSRFSGSGSGTDFTLSINSVESEDVADYYCQQSHGWPFTFGGGTKLEIE

SEQ ID NO: 246 - protein, 117 aa, synthetic construct
  REGION 1..117: note = Description of Artificial Sequence: Synthetic polypeptide
EVKLVESGGGLVKPGGSLKLSCAASGFTFSSYDMSWVRQTPEKRLEWVASISGGGFLYYLDSVKGRFTISRDNARNILYLHMTSLRSEDTAMYYCARSSYGELMDYWGQGTSVTVSS

SEQ ID NO: 247 - protein, 107 aa, synthetic construct; identical to SEQ ID NO: 245
  REGION 1..107: note = Description of Artificial Sequence: Synthetic polypeptide

SEQ ID NO: 248 - protein, 445 aa, synthetic construct
  REGION 1..445: note = Description of Artificial Sequence: Synthetic polypeptide
EVKLLESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTERSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 249 - protein, 218 aa, synthetic construct
  REGION 1..218: note = Description of Artificial Sequence: Synthetic polypeptide
DIALTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKRADAAPTVSIFPPSSEQLTSGGASVVCFLNNEYPKDINVKWKIDGSERQNGVLNSWTDQDSKDSTYSMSSTLTLTKDEYERHNSYTCEATHKTSTSPIVKSFNRNEC

SEQ ID NO: 250 - protein, 122 aa, synthetic construct
  REGION 1..122: note = Description of Artificial Sequence: Synthetic polypeptide
EVKLLESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSA

SEQ ID NO: 251 - protein, 112 aa, synthetic construct
  REGION 1..112: note = Description of Artificial Sequence: Synthetic polypeptide
DIALTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKR

SEQ ID NO: 252 - protein, 9 aa, synthetic construct
  REGION 1..9: note = Description of Artificial Sequence: Synthetic peptide
SGYTFTSYW

SEQ ID NO: 253 - protein, 9 aa, synthetic construct
  REGION 1..9: note = Description of Artificial Sequence: Synthetic peptide
NIYPSDSYT

SEQ ID NO: 254 - protein, 15 aa, synthetic construct
  REGION 1..15: note = Description of Artificial Sequence: Synthetic peptide
TRNGVEGYPHYYAME

SEQ ID NO: 255 - protein, 7 aa, synthetic construct
  REGION 1..7: note = Description of Artificial Sequence: Synthetic peptide
SQDLSNH

SEQ ID NO: 256 - protein, 4 aa, synthetic construct
  REGION 1..4: note = Description of Artificial Sequence: Synthetic peptide
YYTS

SEQ ID NO: 257 - protein, 9 aa, synthetic construct
  REGION 1..9: note = Description of Artificial Sequence: Synthetic peptide
CQQGYTLPY

SEQ ID NO: 258 - protein, 330 aa, synthetic construct
  REGION 1..330: note = Description of Artificial Sequence: Synthetic peptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK